SEQUENCE LISTING (301 sequences)

SEQ ID NO: 1 - protein, 199 aa, Homo Sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 2 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 3 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 4 - protein, 10 aa, Artificial Sequence
  ICOS (009) CDR-H1
GFTFSDYWMN

SEQ ID NO: 5 - protein, 19 aa, Artificial Sequence
  ICOS (009) CDR-H2
QIRNKPYNYETYYSDSVKG

SEQ ID NO: 6 - protein, 12 aa, Artificial Sequence
  ICOS (009) CDR-H3
PRLRSSDWHFDV

SEQ ID NO: 7 - protein, 11 aa, Artificial Sequence
  ICOS (009) CDR-L1
KASQDINKNIA

SEQ ID NO: 8 - protein, 7 aa, Artificial Sequence
  ICOS (009) CDR-L2
YTSTLQT

SEQ ID NO: 9 - protein, 8 aa, Artificial Sequence
  ICOS (009) CDR-L3
LQFDNLYT

SEQ ID NO: 10 - protein, 123 aa, Artificial Sequence
  ICOS (009) VH
EVRLDETGGGVVQPGRPMELSCVASGFTFSDYWMNWVRQSPEKGLEWVAQIRNKPYNYETYYSDSVKGRFTISRDDSKSRVYLQMNNLRAEDMGIYYCTWPRLRSSDWHFDVWGAGTTVTVSS

SEQ ID NO: 11 - protein, 106 aa, Artificial Sequence
  ICOS (009) VL
AIQMTQSPSSLSASLGGEVTITCKASQDINKNIAWYQHKPGRGPRLLIWYTSTLQTGIPSRFSGSGSGRDYSFTISNLEPEDFATYYCLQFDNLYTFGSGTKLEIR

SEQ ID NO: 12 - protein, 10 aa, Artificial Sequence
  ICOS (1167) CDR-H1
GFTFNTYAVH

SEQ ID NO: 13 - protein, 17 aa, Artificial Sequence
  ICOS (1167) CDR-H2
GIGGSGVRTYYADSVKG

SEQ ID NO: 14 - protein, 14 aa, Artificial Sequence
  ICOS (1167) CDR-H3
DIYVADFTGYAFDI

SEQ ID NO: 15 - protein, 11 aa, Artificial Sequence
  ICOS (1167) CDR-L1
RASQGINNFLA

SEQ ID NO: 16 - protein, 7 aa, Artificial Sequence
  ICOS (1167) CDR-L2
DASSLQS

SEQ ID NO: 17 - protein, 9 aa, Artificial Sequence
  ICOS (1167) CDR-L3
QQYNFYPLT

SEQ ID NO: 18 - protein, 123 aa, Artificial Sequence
  ICOS (1167) VH
EVRLLESGGGLVQPGGSLRLSCAASGFTFNTYAVHWVRQAPGKGLEWVSGIGGSGVRTYYADSVKGRLTISRDNSKNTLYLQMNSLRAEDTAIYFCAKDIYVADFTGYAFDIWGQGTMVTVSS

SEQ ID NO: 19 - protein, 107 aa, Artificial Sequence
  ICOS (1167) VL
DIQMTQSPSSVSASVGDRVTITCRASQGINNFLAWYQQKPGKAPKLLIYDASSLQSGVPSRFAGSGSGTDFTLTISSLQPEDFATYYCQQYNFYPLTFGGGTMVEIK

SEQ ID NO: 20 - protein, 11 aa, Artificial Sequence
  ICOS (1143) CDR-H1
GFDFSSAYDMC

SEQ ID NO: 21 - protein, 17 aa, Artificial Sequence
  ICOS (1143) CDR-H2
CVYYGDGITYYATWAKG

SEQ ID NO: 22 - protein, 12 aa, Artificial Sequence
  ICOS (1143) CDR-H3
GAFLGSSYYLSL

SEQ ID NO: 23 - protein, 11 aa, Artificial Sequence
  ICOS (1143) CDR-L1
QASENIYNWLA

SEQ ID NO: 24 - protein, 7 aa, Artificial Sequence
  ICOS (1143) CDR-L2
DASKLAS

SEQ ID NO: 25 - protein, 12 aa, Artificial Sequence
  ICOS (1143) CDR-L3
QQAYTYGNIDNA

SEQ ID NO: 26 - protein, 120 aa, Artificial Sequence
  ICOS (1143) VH
QSLEESGGDLVKPGASLTLTCKASGFDFSSAYDMCWVRQAPGKGLEWIGCVYYGDGITYYATWAKGRFTISKTSSTTVPLQMTSLTAADTATYFCARGAFLGSSYYLSLWGQGTLVTVSS

SEQ ID NO: 27 - protein, 110 aa, Artificial Sequence
  ICOS (1143) VL
AIDMTQTPASVEAAVGGTVTINCQASENIYNWLAWYQQKPGQPPKLLIYDASKLASGVPSRFSASGSGTQFTLTISAVECADAATYYCQQAYTYGNIDNAFGGGTEVVVS

SEQ ID NO: 28 - protein, 11 aa, Artificial Sequence
  ICOS (1138) CDR-H1
GFDLSSYYYMC

SEQ ID NO: 29 - protein, 19 aa, Artificial Sequence
  ICOS (1138) CDR-H2
CIYADIYGGTTHYASWAKG

SEQ ID NO: 30 - protein, 14 aa, Artificial Sequence
  ICOS (1138) CDR-H3
EDGSRYGGSGYYNL

SEQ ID NO: 31 - protein, 11 aa, Artificial Sequence
  ICOS (1138) CDR-L1
QASQNIYSNLA

SEQ ID NO: 32 - protein, 7 aa, Artificial Sequence
  ICOS (1138) CDR-L2
AASYLTS

SEQ ID NO: 33 - protein, 12 aa, Artificial Sequence
  ICOS (1138) CDR-L3
QQGHTTDNIDNA

SEQ ID NO: 34 - protein, 124 aa, Artificial Sequence
  ICOS (1138) VH
QSLEESGGDLVKPGASLTLTCTASGFDLSSYYYMCWVRQAPGKGLEWIACIYADIYGGTTHYASWAKGRFTISKTSSTTVTLQMTSLTAADTATYFCAREDGSRYGGSGYYNLWGPGTLVTVSS

SEQ ID NO: 35 - protein, 110 aa, Artificial Sequence
  ICOS (1138) VL
ALVMTQTPSSVSAAVGGTVTINCQASQNIYSNLAWYQQKPGQPPKLLIYAASYLTSGVSSRFKGSGAGTQFTLTISGVECADAATYYCQQGHTTDNIDNAFGGGTEVVVK

SEQ ID NO: 36 - protein, 5 aa, Artificial Sequence
  FAP(4B9)  CDR-H1
SYAMS

SEQ ID NO: 37 - protein, 17 aa, Artificial Sequence
  FAP(4B9)  CDR-H2
AIIGSGASTYYADSVKG

SEQ ID NO: 38 - protein, 8 aa, Artificial Sequence
  FAP(4B9)  CDR-H3
GWFGGFNY

SEQ ID NO: 39 - protein, 12 aa, Artificial Sequence
  FAP(4B9)  CDR-L1
RASQSVTSSYLA

SEQ ID NO: 40 - protein, 7 aa, Artificial Sequence
  FAP(4B9)  CDR-L2
VGSRRAT

SEQ ID NO: 41 - protein, 9 aa, Artificial Sequence
  FAP(4B9)  CDR-L3
QQGIMLPPT

SEQ ID NO: 42 - protein, 117 aa, Artificial Sequence
  FAP(4B9)  VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 43 - protein, 108 aa, Artificial Sequence
  FAP(4B9)  VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 44 - protein, 5 aa, Artificial Sequence
  FAP (28H1) CDR-H1
SHAMS

SEQ ID NO: 45 - protein, 16 aa, Artificial Sequence
  FAP (28H1) CDR-H2
AIWASGEQYYADSVKG

SEQ ID NO: 46 - protein, 8 aa, Artificial Sequence
  FAP (28H1) CDR-H3
GWLGNFDY

SEQ ID NO: 47 - protein, 12 aa, Artificial Sequence
  FAP (28H1) CDR-L1
RASQSVSRSYLA

SEQ ID NO: 48 - protein, 7 aa, Artificial Sequence
  FAP (28H1) CDR-L2
GASTRAT

SEQ ID NO: 49 - protein, 9 aa, Artificial Sequence
  FAP (28H1) CDR-L3
QQGQVIPPT

SEQ ID NO: 50 - protein, 116 aa, Artificial Sequence
  FAP (28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 51 - protein, 108 aa, Artificial Sequence
  FAP (28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 52 - protein, 5 aa, Artificial Sequence
  CEA (MEDI-565)- CDR-H1
SYWMH

SEQ ID NO: 53 - protein, 16 aa, Artificial Sequence
  CEA (MEDI-565)- CDR-H2
FIRNKANGGTTEYAAS

SEQ ID NO: 54 - protein, 10 aa, Artificial Sequence
  CEA (MEDI-565)- CDR-H3
DRGLRFYFDY

SEQ ID NO: 55 - protein, 14 aa, Artificial Sequence
  CEA (MEDI-565)- CDR-L1
TLRRGINVGAYSIY

SEQ ID NO: 56 - protein, 11 aa, Artificial Sequence
  CEA (MEDI-565)- CDR-L2
YKSDSDKQQGS

SEQ ID NO: 57 - protein, 10 aa, Artificial Sequence
  CEA (MEDI-565)- CDR-L3
MIWHSGASAV

SEQ ID NO: 58 - protein, 121 aa, Artificial Sequence
  CEA (MEDI-565)- VH
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 59 - protein, 116 aa, Artificial Sequence
  CEA (MEDI-565)- VL
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 60 - protein, 5 aa, Artificial Sequence
  CEA (A5H1EL1D)- CDR-H1
DYYMN

SEQ ID NO: 61 - protein, 19 aa, Artificial Sequence
  CEA (A5H1EL1D)- CDR-H2
FIGNKANAYTTEYSASVKG

SEQ ID NO: 62 - protein, 10 aa, Artificial Sequence
  CEA (A5H1EL1D)- CDR-H3
DRGLRFYFDY

SEQ ID NO: 63 - protein, 10 aa, Artificial Sequence
  CEA (A5H1EL1D)- CDR-L1
RASSSVTYIH

SEQ ID NO: 64 - protein, 7 aa, Artificial Sequence
  CEA (A5H1EL1D)- CDR-L2
ATSNLAS

SEQ ID NO: 65 - protein, 9 aa, Artificial Sequence
  CEA (A5H1EL1D)- CDR-L3
QHWSSKPPT

SEQ ID NO: 66 - protein, 121 aa, Artificial Sequence
  CEA (A5B7) VH
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 67 - protein, 106 aa, Artificial Sequence
  CEA (A5B7) VL
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIK

SEQ ID NO: 68 - protein, 121 aa, Artificial Sequence
  CEA (A5H1EL1D) VH (3-23A5-1E)
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSS

SEQ ID NO: 69 - protein, 106 aa, Artificial Sequence
  CEA (A5H1EL1D) VL (A5-L1D)
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIK

SEQ ID NO: 70 - protein, 352 aa, Artificial Sequence
  human ICOS antigen Fc hole chain (dimeric)
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKSADVDDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 71 - protein, 385 aa, Artificial Sequence
  human ICOS antigen Fc knob chain (dimeric)
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKSADVDASGGSPTPPTPGGGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 72 - protein, 227 aa, Artificial Sequence
  human ICOS antigen Fc hole chain (monomeric)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 73 - protein, 352 aa, Artificial Sequence
  cynomolgus ICOS antigen Fc hole chain
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKSADVDDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 74 - protein, 385 aa, Artificial Sequence
  cynomolgus ICOS antigen Fc knob chain
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKSADVDASGGSPTPPTPGGGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 75 - protein, 356 aa, Artificial Sequence
  murine ICOS antigen Fc hole chain
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLSADVDDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 76 - protein, 389 aa, Artificial Sequence
  murine ICOS antigen Fc knob chain
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLSADVDASGGSPTPPTPGGGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 77 - DNA, 37 bp, Artificial Sequence
  rbHC.up
aagcttgccaccatggagactgggctgcgctggcttc

SEQ ID NO: 78 - DNA, 21 bp, Artificial Sequence
  rbHCf.do
ccattggtgagggtgcccgag

SEQ ID NO: 79 - DNA, 34 bp, Artificial Sequence
  rbLC.up
aagcttgccaccatggacaygagggcccccactc

SEQ ID NO: 80 - DNA, 26 bp, Artificial Sequence
  rbLC.do
cagagtrctgctgaggttgtaggtac

SEQ ID NO: 81 - DNA, 20 bp, Artificial Sequence
  BcPCR_FHLC_leader.fw
atggacatgagggtccccgc

SEQ ID NO: 82 - DNA, 24 bp, Artificial Sequence
  BcPCR_huCkappa.rev
gatttcaactgctcatcagatggc

SEQ ID NO: 83 - protein, 214 aa, Artificial Sequence
  1167 light chain (rabbit IgG)
DIQMTQSPSSVSASVGDRVTITCRASQGINNFLAWYQQKPGKAPKLLIYDASSLQSGVPSRFAGSGSGTDFTLTISSLQPEDFATYYCQQYNFYPLTFGGGTMVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 446 aa, Artificial Sequence
  1167 heavy chain (rabbit IgG)
EVRLLESGGGLVQPGGSLRLSCAASGFTFNTYAVHWVRQAPGKGLEWVSGIGGSGVRTYYADSVKGRLTISRDNSKNTLYLQMNSLRAEDTAIYFCAKDIYVADFTGYAFDIWGQGTMVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 85 - protein, 214 aa, Artificial Sequence
  1143 light chain (rabbit IgG)
AIDMTQTPASVEAAVGGTVTINCQASENIYNWLAWYQQKPGQPPKLLIYDASKLASGVPSRFSASGSGTQFTLTISAVECADAATYYCQQAYTYGNIDNAFGGGTEVVVSGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 86 - protein, 443 aa, Artificial Sequence
  1143 heavy chain (rabbit IgG)
QSLEESGGDLVKPGASLTLTCKASGFDFSSAYDMCWVRQAPGKGLEWIGCVYYGDGITYYATWAKGRFTISKTSSTTVPLQMTSLTAADTATYFCARGAFLGSSYYLSLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 87 - protein, 214 aa, Artificial Sequence
  1138 light chain (rabbit IgG)
ALVMTQTPSSVSAAVGGTVTINCQASQNIYSNLAWYQQKPGQPPKLLIYAASYLTSGVSSRFKGSGAGTQFTLTISGVECADAATYYCQQGHTTDNIDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 88 - protein, 447 aa, Artificial Sequence
  1138 heavy chain (rabbit IgG)
QSLEESGGDLVKPGASLTLTCTASGFDLSSYYYMCWVRQAPGKGLEWIACIYADIYGGTTHYASWAKGRFTISKTSSTTVTLQMTSLTAADTATYFCAREDGSRYGGSGYYNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 89 - protein, 385 aa, Artificial Sequence; identical to SEQ ID NO: 71
  human ICOS Fc knob Avi-tag

SEQ ID NO: 90 - protein, 227 aa, Artificial Sequence; identical to SEQ ID NO: 72
  human ICOS Fc hole

SEQ ID NO: 91 - protein, 438 aa, Artificial Sequence
  (FAP 4B9) VLCH1-Fc hole
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 92 - protein, 224 aa, Artificial Sequence
  (FAP 4B9) VHCL-Light chain 1
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 451 aa, Artificial Sequence
  (1167) VHCH1-Fc knob
EVRLLESGGGLVQPGGSLRLSCAASGFTFNTYAVHWVRQAPGKGLEWVSGIGGSGVRTYYADSVKGRLTISRDNSKNTLYLQMNSLRAEDTAIYFCAKDIYVADFTGYAFDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 94 - protein, 214 aa, Artificial Sequence
  (1167) VLCL-Light chain 2
DIQMTQSPSSVSASVGDRVTITCRASQGINNFLAWYQQKPGKAPKLLIYDASSLQSGVPSRFAGSGSGTDFTLTISSLQPEDFATYYCQQYNFYPLTFGGGTMVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 354 aa, Artificial Sequence
  Fc hole VH (FAP 4B9)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 96 - protein, 589 aa, Artificial Sequence
  (1167)VHCH1 Fc knob VL (4B9)
EVRLLESGGGLVQPGGSLRLSCAASGFTFNTYAVHWVRQAPGKGLEWVSGIGGSGVRTYYADSVKGRLTISRDNSKNTLYLQMNSLRAEDTAIYFCAKDIYVADFTGYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 97 - protein, 580 aa, Artificial Sequence
  (ICOS 1167) VHCH1 Fc hole VH (FAP 4B9)
EVRLLESGGGLVQPGGSLRLSCAASGFTFNTYAVHWVRQAPGKGLEWVSGIGGSGVRTYYADSVKGRLTISRDNSKNTLYLQMNSLRAEDTAIYFCAKDIYVADFTGYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 98 - protein, 580 aa, Artificial Sequence
  (ICOS 009) VHCH1 Fc hole VH (FAP 4B9)
EVRLDETGGGVVQPGRPMELSCVASGFTFSDYWMNWVRQSPEKGLEWVAQIRNKPYNYETYYSDSVKGRFTISRDDSKSRVYLQMNNLRAEDMGIYYCTWPRLRSSDWHFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 99 - protein, 589 aa, Artificial Sequence
  (ICOS 009) VHCH1 Fc knob VL (FAP 4B9)
EVRLDETGGGVVQPGRPMELSCVASGFTFSDYWMNWVRQSPEKGLEWVAQIRNKPYNYETYYSDSVKGRFTISRDDSKSRVYLQMNNLRAEDMGIYYCTWPRLRSSDWHFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 100 - protein, 213 aa, Artificial Sequence
  (ICOS 009) VLCL-light chain
AIQMTQSPSSLSASLGGEVTITCKASQDINKNIAWYQHKPGRGPRLLIWYTSTLQTGIPSRFSGSGSGRDYSFTISNLEPEDFATYYCLQFDNLYTFGSGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 588 aa, Artificial Sequence
  (ICOS 009v1)VHCH1 Fc knob VL (FAP 4B9)
EVRLDETGGGVVQPGRPMELSCVASGFTFSDYWMNWVRQSPKGLEWVAQIRNKPYNYETYYSDSVKGRFTISRDDSKSRVYLQMNNLRAEDMGIYYCTWPRLRSSDWHFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 102 - protein, 581 aa, Artificial Sequence
  (ICOS 1138) VHCH1 Fc hole VH (FAP 4B9)
QSLEESGGDLVKPGASLTLTCTASGFDLSSYYYMSWVRQAPGKGLEWIASIYADIYGGTTHYASWAKGRFTISKTSSTTVTLQMTSLTAADTATYFCAREDGSRYGGSGYYNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 103 - protein, 590 aa, Artificial Sequence
  (ICOS 1138)VHCH1 Fc knob VL (FAP 4B9)
QSLEESGGDLVKPGASLTLTCTASGFDLSSYYYMSWVRQAPGKGLEWIASIYADIYGGTTHYASWAKGRFTISKTSSTTVTLQMTSLTAADTATYFCAREDGSRYGGSGYYNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 104 - protein, 217 aa, Artificial Sequence
  (ICOS 1138) VLCL-light chain
ALVMTQTPSSVSAAVGGTVTINCQASQNIYSNLAWYQQKPGQPPKLLIYAASYLTSGVSSRFKGSGAGTQFTLTISGVESADAATYYCQQGHTTDNIDNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 577 aa, Artificial Sequence
  (ICOS 1143) VHCH1 Fc hole VH (FAP 4B9)
QSLEESGGDLVKPGASLTLTCKASGFDFSSAYDMSWVRQAPGKGLEWIGSVYYGDGITYYATWAKGRFTISKTSSTTVPLQMTSLTAADTATYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 106 - protein, 586 aa, Artificial Sequence
  (ICOS 1143)VHCH1 Fc knob VL (4B9)
QSLEESGGDLVKPGASLTLTCKASGFDFSSAYDMSWVRQAPGKGLEWIGSVYYGDGITYYATWAKGRFTISKTSSTTVPLQMTSLTAADTATYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 107 - protein, 217 aa, Artificial Sequence
  (ICOS 1143) VLCL-light chain
AIDMTQTPASVEAAVGGTVTINCQASENIYNWLAWYQQKPGQPPKLLIYDASKLASGVPSRFSASGSGTQFTLTISAVESADAATYYCQQAYTYGNIDNAFGGGTEVVVSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 577 aa, Artificial Sequence
  (ICOS 1143v1) VHCH1 Fc hole VH (FAP 4B9)
QSLEESGGDLVKPGASLTLTCKASGFDFSSAYDMSWVRQAPGKGLEWIGVVYYGDGITYYATWAKGRFTISKTSSTTVPLQMTSLTAADTATYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 109 - protein, 586 aa, Artificial Sequence
  (ICOS 1143v1) VHCH1 Fc knob VL (4B9)
QSLEESGGDLVKPGASLTLTCKASGFDFSSAYDMSWVRQAPGKGLEWIGVVYYGDGITYYATWAKGRFTISKTSSTTVPLQMTSLTAADTATYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 110 - protein, 577 aa, Artificial Sequence
  (ICOS 1143v2) VHCH1 Fc hole VH (FAP 4B9)
QSLEESGGDLVKPGASLTLTCKASGFDFSSAYDMSWVRQAPGKGLEWIGVIYYGDGITYYATSVKGRFTISKTSSTTVPLQMTSLTAADTATYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 111 - protein, 586 aa, Artificial Sequence
  (ICOS 1143v2) VHCH1 Fc knob VL (FAP 4B9)
QSLEESGGDLVKPGASLTLTCKASGFDFSSAYDMSWVRQAPGKGLEWIGVIYYGDGITYYATSVKGRFTISKTSSTTVPLQMTSLTAADTATYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 112 - protein, 451 aa, Artificial Sequence
  (ICOS 1167) VHCH1 Fc hole
EVRLLESGGGLVQPGGSLRLSCAASGFTFNTYAVHWVRQAPGKGLEWVSGIGGSGVRTYYADSVKGRLTISRDNSKNTLYLQMNSLRAEDTAIYFCAKDIYVADFTGYAFDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 113 - protein, 214 aa, Artificial Sequence; identical to SEQ ID NO: 94
  (ICOS 1167) VLCL-light chain 1

SEQ ID NO: 114 - protein, 675 aa, Artificial Sequence
  (FAP 4B9) VLCH1 - (ICOS 1167) VHCH1 Fc knob
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVRLLESGGGLVQPGGSLRLSCAASGFTFNTYAVHWVRQAPGKGLEWVSGIGGSGVRTYYADSVKGRLTISRDNSKNTLYLQMNSLRAEDTAIYFCAKDIYVADFTGYAFDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 115 - protein, 224 aa, Artificial Sequence; identical to SEQ ID NO: 92
  (FAP 4B9) VHCL-light chain 2

SEQ ID NO: 116 - protein, 451 aa, Artificial Sequence; identical to SEQ ID NO: 112
  (ICOS 1167) VHCH1 Fc hole

SEQ ID NO: 117 - protein, 214 aa, Artificial Sequence; identical to SEQ ID NO: 94
  (ICOS 1167) VLCL-light chain 1

SEQ ID NO: 118 - protein, 675 aa, Artificial Sequence
  (ICOS 1167) VHCH1 - (FAP 4B9) VLCH1 Fc knob
EVRLLESGGGLVQPGGSLRLSCAASGFTFNTYAVHWVRQAPGKGLEWVSGIGGSGVRTYYADSVKGRLTISRDNSKNTLYLQMNSLRAEDTAIYFCAKDIYVADFTGYAFDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 119 - protein, 224 aa, Artificial Sequence; identical to SEQ ID NO: 92
  (FAP 4B9) VHCL-light chain 2

SEQ ID NO: 120 - protein, 20 aa, Artificial Sequence
  Post-CDR3 from IGHJ6*01/02
YYYYYGMDVWGQGTTVTVSS

SEQ ID NO: 121 - protein, 12 aa, Artificial Sequence
  Post-CDR3 from IGKJ2*01
YTFGQGTKLEIK

SEQ ID NO: 122 - protein, 17 aa, Artificial Sequence
  Post-CDR3 from IGHJ1*01
AEYFQHWGQGTLVTVSS

SEQ ID NO: 123 - protein, 12 aa, Artificial Sequence
  Post-CDR3 from IGKJ4*01/02 1
LTFGGGTKVEIK

SEQ ID NO: 124 - protein, 123 aa, Artificial Sequence
  ICOS (009)- VHG1a
EVQLVESGGGLVQPGRSLRLSCTASGFTFSDYWMNWVRQAPGKGLEWVAQIRNKPYNYETYYSDSVKGRFTISRDDSKSIAYLQMNSLKTEDTAVYYCTWPRLRSSDWHFDVWGKGTTVTVSS

SEQ ID NO: 125 - protein, 123 aa, Artificial Sequence
  ICOS (009)- VHG1b
EVQLVESGGGLVQPGRSLRLSCTASGFTFSDYWMNWVRQAPGKGLEWVAQIRNKPYNYETYYSDSVKGRFTISRDDSKSIAYLQMNSLKTEDTAVYYCTWPRLRSSDWHFDVWGAGTTVTVSS

SEQ ID NO: 126 - protein, 123 aa, Artificial Sequence
  ICOS (009)- VHG1c
EVQLVESGGGLVQPGRSLRLSCTASGFTFSDYWMNWVRQSPEKGLEWVAQIRNKPYNYETYYSDSVKGRFTISRDDSKSIAYLQMNSLKTEDTAVYYCTWPRLRSSDWHFDVWGAGTTVTVSS

SEQ ID NO: 127 - protein, 123 aa, Artificial Sequence
  ICOS (009)- VHG1d
EVQLVESGGGLVQPGRSLRLSCTASGFTFSDYWMNWVRQAPGKGLEWVAQIRNKPYNYETYYAASVKGRFTISRDDSKSIAYLQMNSLKTEDTAVYYCTWPRLRSSDWHFDVWGKGTTVTVSS

SEQ ID NO: 128 - protein, 123 aa, Artificial Sequence
  ICOS (009)- VHG2a
QVQLVESGGGVVQPGRSLRLSCAASGFTFSDYWMNWVRQAPGKGLEWVAQIRNKPYNYETYYSDSVKGRFTISRDNSKNRLYLQMNSLRAEDTAVYYCTWPRLRSSDWHFDVWGKGTTVTVSS

SEQ ID NO: 129 - protein, 123 aa, Artificial Sequence
  ICOS (009)- VHG2b
QVQLVESGGGVVQPGRSLRLSCAASGFTFSDYWMNWVRQAPGKGLEWVAQIRNKPYNYETYYSDSVKGRFTISRDNSKNRLYLQMNSLRAEDTAVYYCTWPRLRSSDWHFDVWGAGTTVTVSS

SEQ ID NO: 130 - protein, 123 aa, Artificial Sequence
  ICOS (009)- VHG2c
QVQLVESGGGVVQPGRSLRLSCAASGFTFSDYWMNWVRQSPEKGLEWVAQIRNKPYNYETYYSDSVKGRFTISRDNSKNRLYLQMNSLRAEDTAVYYCTWPRLRSSDWHFDVWGAGTTVTVSS

SEQ ID NO: 131 - protein, 123 aa, Artificial Sequence
  ICOS (009)- VHG2d
QVQLVESGGGVVQPGRSLRLSCAASGFTFSDYWMNWVRQAPGKGLEWVAQIRNKPYNYETYYADSVKGRFTISRDNSKNRLYLQMNSLRAEDTAVYYCTWPRLRSSDWHFDVWGKGTTVTVSS

SEQ ID NO: 132 - protein, 106 aa, Artificial Sequence
  ICOS (009)- VLG1a
DIQMTQSPSSLSASVGDRVTITCQASQDINKNIAWYQQKPGKAPKLLIWYTSTLQTGVPSRFSGSGSGTDFTFTISSLQPEDIATYYCLQFDNLYTFGQGTKLEIK

SEQ ID NO: 133 - protein, 106 aa, Artificial Sequence
  ICOS (009)- VLG1b
DIQMTQSPSSLSASVGDRVTITCQASQDINKNIAWYQHKPGKGPKLLIWYTSTLQTGVPSRFSGSGSGTDFTFTISSLQPEDIATYYCLQFDNLYTFGSGTKLEIK

SEQ ID NO: 134 - protein, 106 aa, Artificial Sequence
  ICOS (009)- VLG2a
DIQMTQSPSSLSASVGDRVTITCRASQDINKNIAWYQQKPGKAPKLLIWYTSTLQTGVPSRFSGSGSGTDFTLTISSLQPEDFATYYCLQFDNLYTFGQGTKLEIK

SEQ ID NO: 135 - protein, 106 aa, Artificial Sequence
  ICOS (009)- VLG2b
DIQMTQSPSSLSASVGDRVTITCRASQDINKNIAWYQHKPGKGPKLLIWYTSTLQTGVPSRFSGSGSGTDFTLTISSLQPEDFATYYCLQFDNLYTFGSGTKLEIK

SEQ ID NO: 136 - protein, 126 aa, Artificial Sequence
  ICOS (1138)- VHG1a
EVQLLESGGGLVQPGGSLRLSCAASGFDLSSYYYMSWVRQAPGKGLEWVSSIYADIYGGTTHYASWAKGRFTISRDNSKNTLYLQMNSLRAEDTAVYFCAREDGSRYGGSGYYNLWGQGTLVTVSS

SEQ ID NO: 137 - protein, 126 aa, Artificial Sequence
  ICOS (1138)- VHG1b
EVQLLESGGGLVQPGGSLRLSCAASGFDLSSYYYMSWVRQAPGKGLEWVSSIYADIYGGTTHYADSVKGRFTISRDNSKNTLYLQMNSLRAEDTAVYFCAREDGSRYGGSGYYNLWGQGTLVTVSS

SEQ ID NO: 138 - protein, 126 aa, Artificial Sequence
  ICOS (1138)- VHG1c
EVQLLESGGGLVQPGGSLRLSCAASGFDLSSYYYMSWVRQAPGKGLEWVSSIYADIYGGTTHYASWAKGRFTISKTSSKTTLYLQMNSLRAEDTAVYFCAREDGSRYGGSGYYNLWGQGTLVTVSS

SEQ ID NO: 139 - protein, 125 aa, Artificial Sequence
  ICOS (1138)- VHG1d
QQLLESGGGLVQPGGSLRLSCAASGFDLSSYYYMSWVRQAPGKGLEWVSSIYADIYGGTTHYASWAKGRFTISRDNSKNTLYLQMNSLRAEDTAVYFCAREDGSRYGGSGYYNLWGQGTLVTVSS

SEQ ID NO: 140 - protein, 125 aa, Artificial Sequence
  ICOS (1138)- VHG1e
EVQLLESGGGLVQPGGSLRLSCAASGFDLSSYYYMSWVRQAPGKGLEWVSSIYADIYGGTTHYASWAKGRFTISKTSSTTLYLQMNSLRAEDTAVYFCAREDGSRYGGSGYYNLWGQGTLVTVSS

SEQ ID NO: 141 - protein, 110 aa, Artificial Sequence
  ICOS (1138)- VLG1a
DIQMTQSPSSLSASVGDRVTITCRASQNIYSNLAWYQQKPGKAPKLLIYAASYLTSGVPSRFSGSGSGTDFTLTISSLQPEDFATYYCQQGHTTDNIDNAFGGGTKVEIK

SEQ ID NO: 142 - protein, 110 aa, Artificial Sequence
  ICOS (1138)- VLG1b
DIQMTQSPSSLSASVGDRVTITCRASQNIYSNLAWYQQKPGQPPKLLIYAASYLTSGVPSRFSGSGSGTDFTLTISSLQPEDFATYYCQQGHTTDNIDNAFGGGTKVEIK

SEQ ID NO: 143 - protein, 110 aa, Artificial Sequence
  ICOS (1138)- VLG1c
AIQMTQSPSSLSASVGDRVTITCRASQNIYSNLAWYQQKPGKPPKLLIYAASYLTSGVSSRFSGSGSGTDFTLTISSLQPEDFATYYCQQGHTTDNIDNAFGGGTKVEIK

SEQ ID NO: 144 - protein, 122 aa, Artificial Sequence
  ICOS (1143)- VHG1a
EVQLLESGGGLVQPGGSLRLSCAASGFDFSSAYDMSWVRQAPGKGLEWVSVIYYGDGITYYADSVKGRFTISRDNSKNTLYLQMNSLRAEDTAVYFCARGAFLGSSYYLSLWGQGTLVTVSS

SEQ ID NO: 145 - protein, 122 aa, Artificial Sequence
  ICOS (1143)- VHG1b
EVQLLESGGGLVQPGGSLRLSCAASGFDFSSAYDMSWVRQAPGKGLEWIGVIYYGDGITYYADSVKGRFTISRDNSKNTLYLQMNSLRAEDTAVYFCARGAFLGSSYYLSLWGQGTLVTVSS

SEQ ID NO: 146 - protein, 122 aa, Artificial Sequence
  ICOS (1143)- VHG1c
EVQLLESGGGLVQPGGSLRLSCAASGFDFSSAYDMSWVRQAPGKGLEWVSVIYYGDGITYYADSVKGRFTISKTSSKTTLYLQMNSLRAEDTAVYFCARGAFLGSSYYLSLWGQGTLVTVSS

SEQ ID NO: 147 - protein, 121 aa, Artificial Sequence
  ICOS (1143)- VHG1d
QQLLESGGGLVQPGGSLRLSCAASGFDFSSAYDMSWVRQAPGKGLEWVSVIYYGDGITYYADSVKGRFTISRDNSKNTLYLQMNSLRAEDTAVYFCARGAFLGSSYYLSLWGQGTLVTVSS

SEQ ID NO: 148 - protein, 121 aa, Artificial Sequence
  ICOS (1143)- VHG1e
EVQLLESGGGLVQPGGSLRLSCAASGFDFSSAYDMSWVRQAPGKGLEWVSVIYYGDGITYYADSVKGRFTISRDNSNTLYLQMNSLRAEDTAVYFCARGAFLGSSYYLSLWGQGTLVTVSS

SEQ ID NO: 149 - protein, 122 aa, Artificial Sequence
  ICOS (1143)- VHG1f
EVQLLESGGGLVQPGGSLRLSCAASGFDFSSAYDMSWVRQAPGKGLEWIGVIYYGDGITYYADSVKGRFTISKTSSKTTLYLQMNSLRAEDTAVYFCARGAFLGSSYYLSLWGQGTLVTVSS

SEQ ID NO: 150 - protein, 121 aa, Artificial Sequence
  ICOS (1143)- VHG1g
QQLLESGGGLVQPGGSLRLSCAASGFDFSSAYDMSWVRQAPGKGLEWIGVIYYGDGITYYADSVKGRFTISRDNSKNTLYLQMNSLRAEDTAVYFCARGAFLGSSYYLSLWGQGTLVTVSS

SEQ ID NO: 151 - protein, 121 aa, Artificial Sequence
  ICOS (1143)- VHG1h
EVQLLESGGGLVQPGGSLRLSCAASGFDFSSAYDMSWVRQAPGKGLEWIGVIYYGDGITYYADSVKGRFTISRDNSNTLYLQMNSLRAEDTAVYFCARGAFLGSSYYLSLWGQGTLVTVSS

SEQ ID NO: 152 - protein, 110 aa, Artificial Sequence
  ICOS (1143)- VLG1a
DIQMTQSPSSLSASVGDRVTITCRASENIYNWLAWYQQKPGKAPKLLIYDASKLASGVPSRFSGSGSGTDFTLTISSLQPEDFATYYCQQAYTYGNIDNAFGGGTKVEIK

SEQ ID NO: 153 - protein, 110 aa, Artificial Sequence
  ICOS (1143)- VLG1b
DIQMTQSPSSLSASVGDRVTITCRASENIYNWLAWYQQKPGQPPKLLIYDASKLASGVPSRFSGSGSGTDFTLTISSLQPEDFATYYCQQAYTYGNIDNAFGGGTKVEIK

SEQ ID NO: 154 - protein, 451 aa, Artificial Sequence
  Molecule 25 (ICOS H009v1_1) VH
EVQLVESGGGLVQPGRSLRLSCTASGFTFSDYWMNWVRQAPGKGLEWVAQIRNKPYNYETYYSDSVKGRFTISRDDSKSIAYLQMNSLKTEDTAVYYCTWPRLRSSDWHFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 155 - protein, 213 aa, Artificial Sequence
  Molecule 25 (ICOS H009v1_1) VL
DIQMTQSPSSLSASVGDRVTITCQASQDINKNIAWYQQKPGKAPKLLIWYTSTLQTGVPSRFSGSGSGTDFTFTISSLQPEDIATYYCLQFDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 451 aa, Artificial Sequence
  Molecule 26 (ICOS H009v1_2) VH
EVQLVESGGGLVQPGRSLRLSCTASGFTFSDYWMNWVRQAPGKGLEWVAQIRNKPYNYETYYSDSVKGRFTISRDDSKSIAYLQMNSLKTEDTAVYYCTWPRLRSSDWHFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 157 - protein, 213 aa, Artificial Sequence; identical to SEQ ID NO: 155
  Molecule 26 (ICOS H009v1_2) VL

SEQ ID NO: 158 - protein, 451 aa, Artificial Sequence
  Molecule 27 (ICOS H009v1_3) VH
EVQLVESGGGLVQPGRSLRLSCTASGFTFSDYWMNWVRQAPGKGLEWVAQIRNKPYNYETYYAASVKGRFTISRDDSKSIAYLQMNSLKTEDTAVYYCTWPRLRSSDWHFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 159 - protein, 213 aa, Artificial Sequence; identical to SEQ ID NO: 155
  Molecule 27  (ICOS H009v1_3) VL

SEQ ID NO: 160 - protein, 452 aa, Artificial Sequence
  Molecule 32 (ICOS 1138) VH
QSLEESGGDLVKPGASLTLTCTASGFDLSSYYYMSWVRQAPGKGLEWIASIYADIYGGTTHYASWAKGRFTISKTSSTTVTLQMTSLTAADTATYFCAREDGSRYGGSGYYNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 161 - protein, 217 aa, Artificial Sequence; identical to SEQ ID NO: 104
  Molecule 32 (ICOS 1138) VL

SEQ ID NO: 162 - protein, 454 aa, Artificial Sequence
  Molecule 33 (ICOS 1138_1) VH
EVQLLESGGGLVQPGGSLRLSCAASGFDLSSYYYMSWVRQAPGKGLEWVSSIYADIYGGTTHYASWAKGRFTISKTSSKTTLYLQMNSLRAEDTAVYFCAREDGSRYGGSGYYNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 163 - protein, 217 aa, Artificial Sequence
  Molecule 33 (ICOS 1138_1) VL
DIQMTQSPSSLSASVGDRVTITCRASQNIYSNLAWYQQKPGQPPKLLIYAASYLTSGVPSRFSGSGSGTDFTLTISSLQPEDFATYYCQQGHTTDNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 454 aa, Artificial Sequence
  Molecule 34 (ICOS 1138_2) VH
EVQLLESGGGLVQPGGSLRLSCAASGFDLSSYYYMSWVRQAPGKGLEWVSSIYADIYGGTTHYASWAKGRFTISRDNSKNTLYLQMNSLRAEDTAVYFCAREDGSRYGGSGYYNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 165 - protein, 217 aa, Artificial Sequence
  Molecule 34 (ICOS 1138_2) VL
AIQMTQSPSSLSASVGDRVTITCRASQNIYSNLAWYQQKPGKPPKLLIYAASYLTSGVSSRFSGSGSGTDFTLTISSLQPEDFATYYCQQGHTTDNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 453 aa, Artificial Sequence
  Molecule 35 (ICOS 1138_3) VH
EVQLLESGGGLVQPGGSLRLSCAASGFDLSSYYYMSWVRQAPGKGLEWVSSIYADIYGGTTHYASWAKGRFTISKTSSTTLYLQMNSLRAEDTAVYFCAREDGSRYGGSGYYNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 167 - protein, 217 aa, Artificial Sequence; identical to SEQ ID NO: 165
  Molecule 35 (ICOS 1138_3) VL

SEQ ID NO: 168 - protein, 448 aa, Artificial Sequence
  Molecule 28 (ICOS 1143v2) VH
QSLEESGGDLVKPGASLTLTCKASGFDFSSAYDMSWVRQAPGKGLEWIGVIYYGDGITYYATSVKGRFTISKTSSTTVPLQMTSLTAADTATYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 169 - protein, 217 aa, Artificial Sequence; identical to SEQ ID NO: 107
  Molecule 28 (ICOS 1143v2) VL

SEQ ID NO: 170 - protein, 450 aa, Artificial Sequence
  Molecule 29 (ICOS 1143v2_1) VH
EVQLLESGGGLVQPGGSLRLSCAASGFDFSSAYDMSWVRQAPGKGLEWVSVIYYGDGITYYADSVKGRFTISRDNSKNTLYLQMNSLRAEDTAVYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 171 - protein, 217 aa, Artificial Sequence
  Molecule 29 (ICOS 1143v2_1) VL
DIQMTQSPSSLSASVGDRVTITCRASENIYNWLAWYQQKPGKAPKLLIYDASKLASGVPSRFSGSGSGTDFTLTISSLQPEDFATYYCQQAYTYGNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 450 aa, Artificial Sequence
  Molecule 30 (ICOS 1143v2_2) VH
EVQLLESGGGLVQPGGSLRLSCAASGFDFSSAYDMSWVRQAPGKGLEWVSVIYYGDGITYYADSVKGRFTISKTSSKTTLYLQMNSLRAEDTAVYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 173 - protein, 217 aa, Artificial Sequence; identical to SEQ ID NO: 171
  Molecule 30 (ICOS 1143v2_2) VL

SEQ ID NO: 174 - protein, 449 aa, Artificial Sequence
  Molecule 31 (ICOS 1143v2_3) VH
EVQLLESGGGLVQPGGSLRLSCAASGFDFSSAYDMSWVRQAPGKGLEWIGVIYYGDGITYYADSVKGRFTISRDNSNTLYLQMNSLRAEDTAVYFCARGAFLGSSYYLSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 175 - protein, 217 aa, Artificial Sequence; identical to SEQ ID NO: 171
  Molecule 31 (ICOS 1143v2_3) VL

SEQ ID NO: 176 - protein, 121 aa, Artificial Sequence; identical to SEQ ID NO: 66
  Murine A5B7 VH

SEQ ID NO: 177 - protein, 98 aa, Artificial Sequence
  IGHV3-23-02
EVQLLESGGGLVQPGGSLRLSCAASGFTFSSYAMSWVRQAPGKGLEWVSAISGSGGSTYYGDSVKGRFTISRDNSKNTLYLQMNSLRAEDTAVYYCAK

SEQ ID NO: 178 - protein, 100 aa, Artificial Sequence
  IGHV3-15*01
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 179 - protein, 121 aa, Artificial Sequence
  3-23A5-1
EVQLLESGGGLVQPGGSLRLSCAASGFTFTDYYMNWVRQAPGKGLEWVGFIGNKANGYTTEYSASVKGRFTISRDNSKNTLYLQMNSLRAEDTAVYYCARDRGLRFYFDYWGQGTTVTVSS

SEQ ID NO: 180 - protein, 121 aa, Artificial Sequence
  3-23A5-2
EVQLLESGGGLVQPGGSLRLSCAASGFTFTDYYMNWVRQAPGKGLEWVGFIGNKANGYTTYYGDSVKGRFTISRDNSKNTLYLQMNSLRAEDTAVYYCARDRGLRFYFDYWGQGTTVTVSS

SEQ ID NO: 181 - protein, 119 aa, Artificial Sequence
  3-23A5-3
EVQLLESGGGLVQPGGSLRLSCAASGFTFTDYYMNWVRQAPGKGLEWVGFIGNKGYTTEYSASVKGRFTISRDNSKNTLYLQMNSLRAEDTAVYYCARDRGLRFYFDYWGQGTTVTVSS

SEQ ID NO: 182 - protein, 121 aa, Artificial Sequence
  3-23A5-4
EVQLLESGGGLVQPGGSLRLSCAASGFTFTDYYMSWVRQAPGKGLEWVGFIGNKANGYTTEYSASVKGRFTISRDNSKNTLYLQMNSLRAEDTAVYYCARDRGLRFYFDYWGQGTTVTVSS

SEQ ID NO: 183 - protein, 121 aa, Artificial Sequence
  3-23A5-1A (all_backmutations)
EVQLLESGGGLVQPGGSLRLSCAASGFTFTDYYMNWVRQAPGKGLEWLGFIGNKANGYTTEYSASVKGRFTISRDKSKNTLYLQMNSLRAEDTATYYCTRDRGLRFYFDYWGQGTTVTVSS

SEQ ID NO: 184 - protein, 121 aa, Artificial Sequence
  3-23A5-1C (A93T)
EVQLLESGGGLVQPGGSLRLSCAASGFTFTDYYMNWVRQAPGKGLEWVGFIGNKANGYTTEYSASVKGRFTISRDNSKNTLYLQMNSLRAEDTAVYYCTRDRGLRFYFDYWGQGTTVTVSS

SEQ ID NO: 185 - protein, 121 aa, Artificial Sequence
  3-23A5-1D (K73)
EVQLLESGGGLVQPGGSLRLSCAASGFTFTDYYMNWVRQAPGKGLEWVGFIGNKANGYTTEYSASVKGRFTISRDKSKNTLYLQMNSLRAEDTAVYYCARDRGLRFYFDYWGQGTTVTVSS

SEQ ID NO: 186 - protein, 121 aa, Artificial Sequence
  3-15A5-1
EVQLVESGGGLVKPGGSLRLSCAASGFTFTDYYMNWVRQAPGKGLEWVGFIGNKANGYTTEYSASVKGRFTISRDDSKNTLYLQMNSLKTEDTAVYYCTRDRGLRFYFDYWGQGTTVTVSS

SEQ ID NO: 187 - protein, 121 aa, Artificial Sequence
  3-15A5-2
EVQLVESGGGLVKPGGSLRLSCAASGFTFTDYYMNWVRQAPGKGLEWVGFIGNKANGYTTEYAAPVKGRFTISRDDSKNTLYLQMNSLKTEDTAVYYCTRDRGLRFYFDYWGQGTTVTVSS

SEQ ID NO: 188 - protein, 121 aa, Artificial Sequence
  3-15A5-3
EVQLVESGGGLVKPGGSLRLSCAASGFTFTDYYMNWVRQAPGKGLEWVGFIGNKANGGTTDYAAPVKGRFTISRDDSKNTLYLQMNSLKTEDTAVYYCTRDRGLRFYFDYWGQGTTVTVSS

SEQ ID NO: 189 - protein, 106 aa, Artificial Sequence; identical to SEQ ID NO: 67
  Murine A5B7 VL

SEQ ID NO: 190 - protein, 95 aa, Artificial Sequence
  IGKV3-11
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 191 - protein, 106 aa, Artificial Sequence
  A5-L1
EIVLTQSPATLSLSPGERATLSCRASSSVTYIHWYQQKPGQAPRLLIYATSNLASGIPARFSGSGSGTDFTLTISSLEPEDFAVYYCQHWSSKPPTFGQGTKLEIK

SEQ ID NO: 192 - protein, 107 aa, Artificial Sequence
  A5-L2
EIVLTQSPATLSLSPGERATLSCRASQSVSSYIHWYQQKPGQAPRLLIYATSNLASGIPARFSGSGSGTDFTLTISSLEPEDFAVYYCQHWSSKPPTFGQGTKLEIK

SEQ ID NO: 193 - protein, 106 aa, Artificial Sequence
  A5-L3
EIVLTQSPATLSLSPGERATLSCRASSSVTYIHWYQQKPGQAPRLLIYDASNRATGIPARFSGSGSGTDFTLTISSLEPEDFAVYYCQHWSSKPPTFGQGTKLEIK

SEQ ID NO: 194 - protein, 106 aa, Artificial Sequence
  A5-L4
EIVLTQSPATLSLSPGERATLSCRASSSVTYIHWYQQKPGQAPRLLIYATSNLASGIPARFSGSGSGTDFTLTISSLEPEDFAVYYCQQWSSKPPTFGQGTKLEIK

SEQ ID NO: 195 - protein, 106 aa, Artificial Sequence
  A5-L1A (all_backmutations)
QTVLTQSPATLSLSPGERATLSCRASSSVTYIHWYQQKPGSSPKSWIYATSNLASGIPARFSGSGSGTDYTLTISSLEPEDFAVYYCQHWSSKPPTFGQGTKLEIK

SEQ ID NO: 196 - protein, 106 aa, Artificial Sequence
  A5-L1B (Q1T2)
QTVLTQSPATLSLSPGERATLSCRASSSVTYIHWYQQKPGQAPRLLIYATSNLASGIPARFSGSGSGTDFTLTISSLEPEDFAVYYCQHWSSKPPTFGQGTKLEIK

SEQ ID NO: 197 - protein, 106 aa, Artificial Sequence
  A5-L1C (FR2)
EIVLTQSPATLSLSPGERATLSCRASSSVTYIHWYQQKPGSSPKSWIYATSNLASGIPARFSGSGSGTDFTLTISSLEPEDFAVYYCQHWSSKPPTFGQGTKLEIK

SEQ ID NO: 198 - protein, 453 aa, Artificial Sequence
  ICOS (JMAb136) VHCH1-Fc hole
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 199 - protein, 214 aa, Artificial Sequence
  ICOS (JMAb136) VLCL-Light chain 1
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 446 aa, Artificial Sequence
  CEA (MEDI-565) VLCH1-Fc knob
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 201 - protein, 228 aa, Artificial Sequence
  CEA (MEDI-565) VHCL-Light chain 2
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 436 aa, Artificial Sequence
  CEA (A5H1EL1D) VLCH1-Fc hole
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 203 - protein, 228 aa, Artificial Sequence
  CEA (A5H1EL1D) VHCL-Light chain 1
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 451 aa, Artificial Sequence; identical to SEQ ID NO: 93
  ICOS (1167) VHCH1-Fc knob

SEQ ID NO: 205 - protein, 214 aa, Artificial Sequence; identical to SEQ ID NO: 94
  ICOS (1167) VLCL-Light chain 2

SEQ ID NO: 206 - protein, 451 aa, Artificial Sequence
  CEA (A5H1EL1D) VHCH1-Fc knob
EVQLVESGGGLVQPGRSLRLSCTASGFTFSDYWMNWVRQAPGKGLEWVAQIRNKPYNYETYYSDSVKGRFTISRDDSKSIAYLQMNSLKTEDTAVYYCTWPRLRSSDWHFDVWGAGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 207 - protein, 213 aa, Artificial Sequence
  CEA (A5H1EL1D) VLCL-Light chain 1
DIQMTQSPSSLSASVGDRVTITCQASQDINKNIAWYQQKPGKAPKLLIWYTSTLQTGVPSRFSGSGSGTDFTFTISSLQPEDIATYYCLQFDNLYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - protein, 436 aa, Artificial Sequence; identical to SEQ ID NO: 202
  ICOS (H009v1_2) VLCH1-Fc hole

SEQ ID NO: 209 - protein, 228 aa, Artificial Sequence; identical to SEQ ID NO: 203
  ICOS (H009v1_2) VHCL-Light chain 2

SEQ ID NO: 210 - protein, 436 aa, Artificial Sequence; identical to SEQ ID NO: 202
  ICOS (H1143v2_1) VLCH1-Fc hole

SEQ ID NO: 211 - protein, 228 aa, Artificial Sequence; identical to SEQ ID NO: 203
  ICOS (H1143v2_1) VHCL-Light chain 2

SEQ ID NO: 212 - protein, 585 aa, Artificial Sequence
  ICOS (JMAb136) VHCH1 Fc hole VH CEA (MEDI-565)
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 213 - protein, 590 aa, Artificial Sequence
  ICOS (JMAb136) VHCH1 Fc knob VL CEA (MEDI-565)
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 214 - protein, 214 aa, Artificial Sequence
  ICOS (JMAb136) VLCL-light chain
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 302 aa, Homo Sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 216 - protein, 125 aa, Artificial Sequence
  ICOS (JMab 136) VH
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSS

SEQ ID NO: 217 - protein, 107 aa, Artificial Sequence
  ICOS (JMab 136) VL
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 218 - protein, 5 aa, Artificial Sequence
  CD3 CDR-H1
TYAMN

SEQ ID NO: 219 - protein, 19 aa, Artificial Sequence
  CD3 CDR-H2
RIRSKYNNYATYYADSVKG

SEQ ID NO: 220 - protein, 14 aa, Artificial Sequence
  CD3 CDR-H3
HGNFGNSYVSWFAY

SEQ ID NO: 221 - protein, 14 aa, Artificial Sequence
  CD3 CDR-L1
GSSTGAVTTSNYAN

SEQ ID NO: 222 - protein, 7 aa, Artificial Sequence
  CD3 CDR-L2
GTNKRAP

SEQ ID NO: 223 - protein, 9 aa, Artificial Sequence
  CD3 CDR-L3
ALWYSNLWV

SEQ ID NO: 224 - protein, 125 aa, Artificial Sequence
  CD3 VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 225 - protein, 109 aa, Artificial Sequence
  CD3 VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 226 - protein, 5 aa, Artificial Sequence
  CEA CDR-H1
EFGMN

SEQ ID NO: 227 - protein, 17 aa, Artificial Sequence
  CEA CDR-H2
WINTKTGEATYVEEFKG

SEQ ID NO: 228 - protein, 12 aa, Artificial Sequence
  CEA CDR-H3
WDFAYYVEAMDY

SEQ ID NO: 229 - protein, 11 aa, Artificial Sequence
  CEA CDR-L1
KASAAVGTYVA

SEQ ID NO: 230 - protein, 7 aa, Artificial Sequence
  CEA CDR-L2
SASYRKR

SEQ ID NO: 231 - protein, 10 aa, Artificial Sequence
  CEA CDR-L3
HQYYTYPLFT

SEQ ID NO: 232 - protein, 121 aa, Artificial Sequence
  CEA VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 233 - protein, 108 aa, Artificial Sequence
  CEA VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 234 - protein, 5 aa, Artificial Sequence
  CEA CDR-H1 (CEACAM5)
DTYMH

SEQ ID NO: 235 - protein, 17 aa, Artificial Sequence
  CEA CDR-H2 (CEACAM5)
RIDPANGNSKYVPKFQG

SEQ ID NO: 236 - protein, 12 aa, Artificial Sequence
  CEA CDR-H3 (CEACAM5)
FGYYVSDYAMAY

SEQ ID NO: 237 - protein, 15 aa, Artificial Sequence
  CEA CDR-L1 (CEACAM5)
RAGESVDIFGVGFLH

SEQ ID NO: 238 - protein, 7 aa, Artificial Sequence
  CEA CDR-L2 (CEACAM5)
RASNRAT

SEQ ID NO: 239 - protein, 9 aa, Artificial Sequence
  CEA CDR-L3 (CEACAM5)
QQTNEDPYT

SEQ ID NO: 240 - protein, 121 aa, Artificial Sequence
  CEA VH (CEACAM5)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 241 - protein, 111 aa, Artificial Sequence
  CEA VL (CEACAM5)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 242 - protein, 215 aa, Artificial Sequence
  Light chain “CEA 2F1” (CEA TCB)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 214 aa, Artificial Sequence
  Light Chain humanized CD3 CH2527 (Crossfab, VL-CH1) (CEA TCB)
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 244 - protein, 694 aa, Artificial Sequence
  CEA CH1A1A 98/99 - humanized CD3 CH2527 (Crossfab VH-Ck)-Fc(knob) P329GLALA (CEA TCB)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 451 aa, Artificial Sequence
  CEA CH1A1A 98/99 (VH-CH1)-Fc(hole) P329GLALA (CEA TCB)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 246 - protein, 232 aa, Artificial Sequence
  CD3 VH-CL (CEACAM5 TCB)
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 449 aa, Artificial Sequence
  humanized CEA VH-CH1(EE)-Fc (hole, P329G LALA) (CEACAM5 TCB)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 248 - protein, 674 aa, Artificial Sequence
  humanized CEA VH-CH1(EE)-CD3 VL-CH1-Fc (knob, P329G LALA) (CEACAM5 TCB)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 249 - protein, 218 aa, Artificial Sequence
  humanized CEA VL-CL(RK) (CEACAM5 TCB)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 443 aa, Artificial Sequence
  VHCH1(CH1A1A 98/99 2F1)- Fc(KK) DAPG chain
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSP

SEQ ID NO: 251 - protein, 215 aa, Artificial Sequence
  VLCL (CH1A1A 98/99 2F1) Light chain
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 252 - protein, 676 aa, Artificial Sequence
  VHCL VHCH1 (2C11- CH1A1A 98/99 2F1)- Fc(DD) DAPG chain
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSQVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSP

SEQ ID NO: 253 - protein, 211 aa, Artificial Sequence
  VLCH1 (2C11) Light chain
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 254 - protein, 760 aa, Homo Sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 255 - protein, 748 aa, Artificial Sequence
  His-tagged human FAP ECD
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 256 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 257 - protein, 749 aa, Artificial Sequence
  His-tagged mouse FAP ECD
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 258 - protein, 748 aa, Artificial Sequence
  His-tagged cynomolgus FAP ECD
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 259 - protein, 702 aa, Homo Sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 260 - protein, 257 aa, Homo Sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 261 - protein, 255 aa, Mus musculus
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 262 - protein, 257 aa, Macaca fascicularis
MAQRMTTQLLLLLVWVAVVGEAQTRTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGAGPWAAWPLLLSLALTLLWLLS

SEQ ID NO: 263 - protein, 2322 aa, Homo Sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 264 - protein, 1210 aa, Homo Sapiens [sequence omitted]

SEQ ID NO: 265 - protein, 1255 aa, Homo Sapiens [sequence omitted]

SEQ ID NO: 266 - protein, 645 aa, Homo Sapiens
MPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 267 - protein, 98 aa, Artificial Sequence
  CH1 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 268 - protein, 5 aa, Artificial Sequence
  CH1 to hinge
EPKSC

SEQ ID NO: 269 - protein, 107 aa, Artificial Sequence
  CH2 domain
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 270 - protein, 106 aa, Artificial Sequence [sequence omitted]

SEQ ID NO: 271 - protein, 5 aa, Artificial Sequence
  Peptide linker (G4S)
GGGGS

SEQ ID NO: 272 - protein, 10 aa, Artificial Sequence
  Peptide linker (G4S)2
GGGGSGGGGS

SEQ ID NO: 273 - protein, 10 aa, Artificial Sequence
  Peptide linker (SG4)2
SGGGGSGGGG

SEQ ID NO: 274 - protein, 14 aa, Artificial Sequence
  Peptide linker G4(SG4)2
GGGGSGGGGSGGGG

SEQ ID NO: 275 - protein, 10 aa, Artificial Sequence
  Peptide linker
GSPGSSSSGS

SEQ ID NO: 276 - protein, 15 aa, Artificial Sequence
  (G4S)3 peptide linker
GGGGSGGGGSGGGGS

SEQ ID NO: 277 - protein, 20 aa, Artificial Sequence
  (G4S)4 peptide linker
GGGGSGGGGSGGGGSGGGGS

SEQ ID NO: 278 - protein, 8 aa, Artificial Sequence
  peptide linker
GSGSGSGS

SEQ ID NO: 279 - protein, 8 aa, Artificial Sequence
  peptide linker
GSGSGNGS

SEQ ID NO: 280 - protein, 8 aa, Artificial Sequence
  peptide linker
GGSGSGSG

SEQ ID NO: 281 - protein, 6 aa, Artificial Sequence
  peptide linker
GGSGSG

SEQ ID NO: 282 - protein, 4 aa, Artificial Sequence
  peptide linker
GGSG

SEQ ID NO: 283 - protein, 8 aa, Artificial Sequence
  peptide linker
GGSGNGSG

SEQ ID NO: 284 - protein, 8 aa, Artificial Sequence
  peptide linker
GGNGSGSG

SEQ ID NO: 285 - protein, 6 aa, Artificial Sequence
  peptide linker
GGNGSG

SEQ ID NO: 286 - protein, 290 aa, Homo Sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 287 - protein, 288 aa, Homo Sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 288 - protein, 118 aa, Artificial Sequence [sequence omitted]

SEQ ID NO: 289 - protein, 107 aa, Artificial Sequence [sequence omitted]

SEQ ID NO: 290 - protein, 121 aa, Artificial Sequence
  VH (PD-L1)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 291 - protein, 108 aa, Artificial Sequence
  VL (PD-L1)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 292 - protein, 120 aa, Artificial Sequence [sequence omitted]

SEQ ID NO: 293 - protein, 111 aa, Artificial Sequence [sequence omitted]

SEQ ID NO: 294 - protein, 113 aa, Artificial Sequence [sequence omitted]

SEQ ID NO: 295 - protein, 107 aa, Artificial Sequence [sequence omitted]

SEQ ID NO: 296 - protein, 122 aa, Artificial Sequence
  ICOS (009v1) VH
EVRLDETGGGVVQPGRPMELSCVASGFTFSDYWMNWVRQSPKGLEWVAQIRNKPYNYETYYSDSVKGRFTISRDDSKSRVYLQMNNLRAEDMGIYYCTWPRLRSSDWHFDVWGAGTTVTVSS

SEQ ID NO: 297 - protein, 106 aa, Artificial Sequence; identical to SEQ ID NO: 11
  ICOS (009v1) VL

SEQ ID NO: 298 - protein, 120 aa, Artificial Sequence
  ICOS (1143v1) VH
QSLEESGGDLVKPGASLTLTCKASGFDFSSAYDMSWVRQAPGKGLEWIGVVYYGDGITYYATWAKGRFTISKTSSTTVPLQMTSLTAADTATYFCARGAFLGSSYYLSLWGQGTLVTVSS

SEQ ID NO: 299 - protein, 110 aa, Artificial Sequence; identical to SEQ ID NO: 27
  ICOS (1143v1) VL

SEQ ID NO: 300 - protein, 120 aa, Artificial Sequence
  ICOS (1143v2) VH
QSLEESGGDLVKPGASLTLTCKASGFDFSSAYDMSWVRQAPGKGLEWIGVIYYGDGITYYATSVKGRFTISKTSSTTVPLQMTSLTAADTATYFCARGAFLGSSYYLSLWGQGTLVTVSS

SEQ ID NO: 301 - protein, 110 aa, Artificial Sequence; identical to SEQ ID NO: 27
  ICOS (1143v2) VL

(19) United States
(12) Patent Application Publication
(10) Pub. No.: US 2022/0098305 A1
(43) Pub. Date: Mar. 31, 2022

(54) NOVEL ICOS ANTIBODIES AND TUMOR-TARGETED ANTIGEN BINDING MOLECULES COMPRISING THEM

(71) Applicant: Hoffmann-La Roche Inc., Little Falls, NJ (US)

(72) Inventors: Stefan DENGL, Geretsried (DE); Tanja FAUTI, Zuerich (CH); Jens FISCHER, Weilheim (DE); Lucas HABEGGER, Bülach (CH); Christian KLEIN, Bonstetten (CH); Esther KOENIGSBERGER, Kochel a. See (DE); Jens NIEWOEHNER, Muenchen (DE); Johannes SAM, Baden (DE); Pablo UMAÑA, Wollerau (CH); Joerg ZIELONKA, Bülach (CH)

(73) Assignee: Hoffmann-La Roche Inc., Little Falls, NJ (US)

(21) Appl. No.: 17/644,526

(22) Filed: Dec. 15, 2021

Related U.S. Application Data
(63) Continuation of application No. PCT/EP2020/067572, filed on Jun. 24, 2020.

(30) Foreign Application Priority Data
Jun. 27, 2019 (EP) 19182810.2

(51) Int. Cl. C07K 16/28; C07K 16/30; C07K 16/40; A61P 35/00

(52) U.S. Cl. CPC C07K 16/2818; C07K 16/3007; C07K 16/40; C07K 16/2809; C07K 16/2827; A61K 39/00; C07K 2317/75; C07K 2317/565; C07K 2317/52; C07K 2317/31; C07K 2317/56; A61P 35/00

(57) ABSTRACT

The present invention relates to novel ICOS antibodies and tumor-targeted agonistic ICOS antigen binding molecules comprising them, pharmaceutical compositions comprising these molecules, and methods of using the same.

### CROSS REFERENCE TO RELATED APPLICATIONS

This application is a continuation of International Patent Application No. PCT/US2020/067572, filed Jun. 24, 2020, which claims priority to European Patent Application number 19182810.0 filed Jun. 27, 2019, which are incorporated herein by reference in its entirety.

### SEQUENCE LISTING

This application contains a Sequence Listing which has been submitted electronically in ASCII format and is hereby incorporated by reference in its entirety. Said ASCII copy, created on Dec. 15, 2021, is named P35614-US_ST25.txt and is 548,961 bytes in size.

### FIELD OF THE INVENTION

The present invention relates to novel ICOS antibodies and tumor-targeted agonistic ICOS antigen binding molecules comprising them as well as to their use as immunomodulators in the treatment of cancer.

### BACKGROUND

Modulating immune inhibitory pathways has been a major recent breakthrough in cancer treatment. Checkpoint blockade antibodies targeting cytotoxic T-lymphocyte antigen 4 (CTLA-4, YERVOY/ipilimumab) and programmed cell-death protein 1 (PD-1, OPDIVO/nivolumab or KEYTRUDA/pembrolizumab), respective PD-L1 (atezolizumab) have demonstrated acceptable toxicity, promising clinical responses, durable disease control, and improved survival in patients of various tumor indications. However, only a minority of patients experience durable responses to immune checkpoint blockade (ICB) therapy, the remainder of patients show primary or secondary resistance, demonstrating a clear need for regulating additional pathways to provide survival benefit for greater numbers of patients. Thus, combination strategies are needed to improve therapeutic benefit.

ICOS (CD278) is an inducible T-cell co-stimulator and belongs to the B7/CD28/CTLA-4 immunoglobulin superfamily (Hutloff, et al., Nature 1999, 397). Its expression seems to be restricted mainly to T cells with only weak expression on NK cells (Ogasawara et al., J Immunol. 2002, 169 and unpublished own data using human NK cells). Unlike CD28, which is constitutively expressed on T cells, ICOS is hardly expressed on naïve T_{H}1 and T_{H}2 effector T cell populations (Paulos C M et al., Sci Transl Med 2010, 2), but on resting T_{H}17, T follicular helper (T_{FH}) and regulatory T (Treg) cells. However, ICOS is strongly induced on all T cell subsets upon previous antigen priming, respective TCR/CD3-engagement (Wakamatsu et al., Proc Natal Acad Sci USA, 2013, 110).

Signaling through the ICOS pathway occurs upon binding of its ligand, the so-called ICOS-L (B7h, B7RP-1, CD275), which is expressed on B cells, macrophages, dendritic cells, and on non-immune cells treated with TNF-α (Simpson et al., Current Opinion in Immunology 2010, 22). Neither B7-1 nor B7-2, the ligands for CD28 and CTLA4, are able to bind or activate ICOS. Nonetheless, ICOS-L has been shown to bind weakly to both CD28 and CTLA-4 (Yao et al., Immunity 2011, 34). Upon activation, ICOS, a disulfide-linked homodimer, induces a signal through the PI3K and AKT pathways. In contrast to CD28, ICOS has a unique YMFM SH2 binding motif, which recruits a PI3K variant with elevated lipid kinase activity compared to the isoform recruited by CD28. As a consequence, greater production of Phosphatidylinositol (3, 4, 5)-triphosphate and concomitant increase in AKT signaling can be observed, suggesting an important role of ICOS in T cell survival (Simpson et al., Current Opinion in Immunology 2010, 22).

As reviewed by Sharpe (Immunol Rev., 2009, 229), the ICOS/ICOS ligand pathway has critical roles in stimulating effector T-cell responses, T-dependent B-cell responses, and regulating T-cell tolerance by controlling IL-10 producing Tregs. Moreover, ICOS is important for generation of chemokine (C-X-C motif) receptor 5 (CXCR5)⁺ follicular helper T cells (T_{FH}), a unique T-cell subset that regulates germinal center reactions and humoral immunity. Recent studies in ICOS-deficient mice indicate that ICOS can regulate interleukin-21 (IL-21) production, which in turn regulates the expansion of T helper (Th) type 17 (T_{H}17) cells and T_{FH}. In this context, ICOS is described to bipolarize CD4 T cells towards a T_{H}1-like T_{H}17 phenotype, which has been shown to correlate with improved survival of patients in several cancer indications, including melanoma, early stage ovarian cancer and more (Rita Young, J Clin Cell Immunol. 2016, 7).

ICOS-deficient mice show impaired germinal center formation and have decreased production of IL-10 and IL-17, which become manifest in an impaired development of autoimmunity phenotypes in various disease models, such as diabetes (T_{H}1), airway inflammation (T_{H}2) and EAE neuro-inflammatory models (T_{H}17) (Warnatz et al, Blood 2006). In line with this, human common variable immunodeficiency patients with mutated ICOS show profound hypogammaglobulinemia and a disturbed B-cell homeostatsis (Sharpe, Immunol Rev., 2009, 229). Important to note, that efficient co-stimulatory signaling via ICOS receptor only occurs in T cells receiving a concurrent TCR activation signal (Wakamatsu et al., Proc Natal Acad Sci USA, 2013, 110).

T-cell bispecific (TCB) molecules are appealing immune cell engagers, since they bypass the need for recognition of MHCI-peptide by corresponding T-cell receptors, but enable a polyclonal T-cell response to cell-surface tumor-associated antigens (Yuraszeck et al., Clinical Pharmacology & Therapeutics 2017, 101). CEA CD3 TCB, an anti-CEA/anti-CD3 bispecific antibody, is an investigational, immunoglobulin G1 (IgG1) T-cell bispecific antibody to engage the immune system against cancer. It is designed to redirect T cells to tumor cells by simultaneous binding to human CD3ε on T cells and carcinoembryonic antigen (CEA), expressed by various cancer cells, including CRC (colorectal cancer), GC (gastric cancer), NSCLC (non-small-cell lung cancer) and BC (breast cancer). The cross-linking of T- and tumor cells, leads to CD3/TCR downstream signaling and to the formation of immunologic synapses, T-cell activation, secretion of cytotoxic granules and other cytokines and ultimately to a dose- and time-dependent lysis of tumor cells. Furthermore, CEA CD3 TCB is proposed to increase T-cell infiltration and generate a highly inflamed tumor microenvironment, making it an ideal combination partner for immune checkpoint blockade therapy (ICB), especially for tumors showing primary resistance to ICB because of the lack of sufficient endogenous adaptive and functional immune infiltrate. However, turning-off the brakes by blocking single or multiple inhibitory pathways on T cells might not be sufficient, given the paradoxical expression of several co-stimulatory receptors, such as 4-1BB (CD137), ICOS and OX40 on dysfunctional T cells in the tumor microenvironment (TME). It has been found that a better anti-tumor effect is achieved when an anti-CEA/anti-CD3 bispecific antibody, i.e. a CEA TCB, is combined with a tumor-targeted agonistic ICOS antigen binding molecule. The T-cell bispecific antibody provides the initial TCR activating signalling to T cells, and then the combination with the tumor-targeted agonistic ICOS antigen binding molecule leads to a further boost of anti-tumor T cell immunity.

For ICOS, a growing body of literature actually supports the idea that engaging CD278 on CD4⁺ and CD8⁺ effector T cells has anti-tumor potential. Activating the ICOS-ICOS-L signaling has induced effective anti-tumor responses in several syngeneic mouse models both as monotherapy, as well in the context of anti-CTLA4 treatment, where activation of ICOS downstream signaling increased the efficacy of anti-CTLA4 therapy significantly (Fu T et al., Cancer Res, 2011, 71 and Allison et al., WO2011/041613 A2, 2009). Emerging data from patients treated with anti-CTLA4 antibodies also point to a correlation of sustained elevated levels of ICOS expression on CD4 and CD8 T cells and improved overall survival of tumor patients, e.g. with metastatic melanoma, urothelial, breast or prostate cancer (Giacomo et al., Cancer Immunol Immunother. 2013, 62; Carthon et al., Clin Cancer Res. 2010, 16; Vonderheide et al., Clin Cancer Res. 2010, 16; Liakou et al, Proc Natl Acad Sci USA 2008, 105 and Vonderheide et al., Clin Cancer Res. 2010, 16). Therefore, ICOS positive T effector cells are seen as a positive predictive biomarker of ipilimumab response. A humanized anti-ICOS IgG1 antibody JTX 2011 (vopratelimab) is currently tested in patients with advanced non-small cell lung cancer or urothelial cancer. Its mechanism of action is dependent on Fcγ cross-linking. Recently, a clinical trial of KY1044, a fully human anti-ICOS IgG4 antibody, in combination with atezolizumab has been started (NCT03829501). However, there is an ongoing need for agonistic ICOS antigen binding molecules, that are particularly suitable for combination treatments with other therapeutic agents for the treatment of diseases, in particular cancer.

### SUMMARY OF THE INVENTION

The present invention relates to novel ICOS antibodies and agonistic ICOS antigen binding molecules comprising at least one antigen binding domain capable of specific binding to a tumor-associated antigen and at least one antigen binding domain capable of specific binding to ICOS comprising said novel ICOS antibodies. The invention also relates to these new agonistic ICOS antigen binding molecules and their use in combination with other therapeutic agents, in particular T-cell activating anti-CD3 bispecific antibodies, in particular for the use in treating or delaying progression of cancer. It has been found that the combination therapy described herein is more effective in inducing early T-cell activation, T-cell proliferation, induction of T memory cell and ultimatively inhibiting tumor growth and eliminating tumor cells than treatment with the anti-CD3 bispecific antibodies alone.

In one aspect, the invention provides an agonistic ICOS antigen binding molecule comprising at least one antigen binding domain capable of specific binding to a tumor-associated antigen and at least one antigen binding domain capable of specific binding to ICOS comprising
(a) a heavy chain variable region (V_{H}ICOS) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}ICOS) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}ICOS) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}ICOS) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or
(c) a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25, or
(d) a heavy chain variable region (V_{H}ICOS) comprising (i) CDR-H1 comprising the amino acid sequence of SEQ ID NO:28, (ii) CDR-H2 comprising the amino acid sequence of SEQ ID NO:29, and (iii) CDR-H3 comprising the amino acid sequence of SEQ ID NO:30, and a light chain variable region (V_{L}ICOS) comprising (iv) CDR-L1 comprising the amino acid sequence of SEQ ID NO:31, (v) CDR-L2 comprising the amino acid sequence of SEQ ID NO:32, and (vi) CDR-L3 comprising the amino acid sequence of SEQ ID NO:33.

In a further aspect, provided is an agonistic ICOS antigen binding molecule as defined above, further comprising a Fc domain composed of a first and a second subunit capable of stable association which comprises one or more amino acid substitution that reduces the binding affinity of the antigen binding molecule to an Fc receptor and/or effector function. In particular, the agonistic ICOS antigen binding molecule comprises a Fc domain of human IgG1 subclass which comprises the amino acid mutations L234A, L235A and P329G (numbering according to Kabat EU index).

In another aspect, the invention provides an agonistic ICOS antigen binding molecule comprising at least one antigen binding domain capable of specific binding to a tumor-associated antigen as defined herein before, wherein the tumor-associated antigen is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epidermal growth factor receptor 2 (HER2) and p95HER2.

In one aspect, there is provided an agonistic ICOS-binding molecule comprising at least one antigen binding domain capable of specific binding to a tumor-associated antigen as defined above, wherein the antigen binding domain capable of specific binding to a tumor-associated antigen is an antigen binding domain capable of specific binding to Carcinoembryonic Antigen (CEA). In one aspect, the antigen binding domain capable of specific binding to CEA comprises
(a) a heavy chain variable region (V_{H}CEA) comprising (i) CDR-H1 comprising the amino acid sequence of SEQ ID NO:52, (ii) CDR-H2 comprising the amino acid sequence of SEQ ID NO:53, and (iii) CDR-H3 comprising the amino acid sequence of SEQ ID NO:54, and a light chain variable region (V_{L}CEA) comprising (iv) CDR-L1 comprising the amino acid sequence of SEQ ID NO:55, (v) CDR-L2 comprising the amino acid sequence of SEQ ID NO:56, and (vi) CDR-L3 comprising the amino acid sequence of SEQ ID NO:57, or (b) a heavy chain variable region (V_{H}CEA) comprising (i) CDR-H1 comprising the amino acid sequence of SEQ ID NO:60, (ii) CDR-H2 comprising the amino acid sequence of SEQ ID NO:61, and (iii) CDR-H3 comprising the amino acid sequence of SEQ ID NO:62, and a light chain variable region (V_{L}CEA) comprising (iv) CDR-L1 comprising the amino acid sequence of SEQ ID NO:63, (v) CDR-L2 comprising the amino acid sequence of SEQ ID NO:64, and (vi) CDR-L3 comprising the amino acid sequence of SEQ ID NO:65. In one particular aspect, the antigen binding domain capable of specific binding to CEA comprises a heavy chain variable region (V_{H}CEA) comprising (i) CDR-H1 comprising the amino acid sequence of SEQ ID NO:60, (ii) CDR-H2 comprising the amino acid sequence of SEQ ID NO:61, and (iii) CDR-H3 comprising the amino acid sequence of SEQ ID NO:62, and a light chain variable region (V_{L}CEA) comprising (iv) CDR-L1 comprising the amino acid sequence of SEQ ID NO:63, (v) CDR-L2 comprising the amino acid sequence of SEQ ID NO:64, and (vi) CDR-L3 comprising the amino acid sequence of SEQ ID NO:65.

In another aspect, provided is an agonistic ICOS antigen binding molecule as defined above, wherein the antigen binding domain capable of specific binding to CEA comprises a heavy chain variable region (V_{H}CEA) comprising an amino acid sequence that is at least about 95%, 96%, 97%, 98%, 99% or 100% identical to the amino acid sequence of SEQ ID NO:58, and a light chain variable region (V_{L}CEA) comprising an amino acid sequence that is at least about 95%, 96%, 97%, 98%, 99% or 100% identical to the amino acid sequence of SEQ ID NO:59, or a heavy chain variable region (V_{H}CEA) comprising an amino acid sequence that is at least about 95%, 96%, 97%, 98%, 99% or 100% identical to the amino acid sequence of SEQ ID NO:68, and a light chain variable region (V_{L}CEA) comprising an amino acid sequence that is at least about 95%, 96%, 97%, 98%, 99% or 100% identical to the amino acid sequence of SEQ ID NO:69. In one aspect, the antigen binding domain capable of specific binding to CEA comprises a heavy chain variable region (V_{H}CEA) comprising the amino acid sequence of SEQ ID NO:58, and a light chain variable region (V_{L}CEA) comprising the amino acid sequence of SEQ ID NO:59. In particular, the antigen binding domain capable of specific binding to CEA comprises a heavy chain variable region (V_{H}CEA) comprising the amino acid sequence of SEQ ID NO:68, and a light chain variable region (V_{L}CEA) comprising the amino acid sequence of SEQ ID NO:69.

In a further aspect, there is provided agonistic ICOS antigen binding molecule of any one of claims 1 to 3, wherein the antigen binding domain capable of specific binding to a tumor-associated antigen is an antigen binding domain capable of specific binding to Fibroblast Activation Protein (FAP). In one aspect, the antigen binding domain capable of specific binding to FAP comprises (a) a heavy chain variable region (V_{H}FAP) comprising (i) CDR-H1 comprising the amino acid sequence of SEQ ID NO:36, (ii) CDR-H2 comprising the amino acid sequence of SEQ ID NO:37, and (iii) CDR-H3 comprising the amino acid sequence of SEQ ID NO:38, and a light chain variable region (V_{L}FAP) comprising (iv) CDR-L1 comprising the amino acid sequence of SEQ ID NO:39, (v) CDR-L2 comprising the amino acid sequence of SEQ ID NO:40, and (vi) CDR-L3 comprising the amino acid sequence of SEQ ID NO:41, or
(b) a heavy chain variable region (V_{H}FAP) comprising (i) CDR-H1 comprising the amino acid sequence of SEQ ID NO:44, (ii) CDR-H2 comprising the amino acid sequence of SEQ ID NO:45, and (iii) CDR-H3 comprising the amino acid sequence of SEQ ID NO:46, and a light chain variable region (V_{L}FAP) comprising (iv) CDR-L1 comprising the amino acid sequence of SEQ ID NO:47, (v) CDR-L2 comprising the amino acid sequence of SEQ ID NO:48, and (vi) CDR-L3 comprising the amino acid sequence of SEQ ID NO:49. In one particular aspect, the antigen binding domain capable of specific binding to FAP comprises (a) a heavy chain variable region (V_{H}FAP) comprising (i) CDR-H1 comprising the amino acid sequence of SEQ ID NO:36, (ii) CDR-H2 comprising the amino acid sequence of SEQ ID NO:37, and (iii) CDR-H3 comprising the amino acid sequence of SEQ ID NO:38, and a light chain variable region (V_{L}FAP) comprising (iv) CDR-L1 comprising the amino acid sequence of SEQ ID NO:39, (v) CDR-L2 comprising the amino acid sequence of SEQ ID NO:40, and (vi) CDR-L3 comprising the amino acid sequence of SEQ ID NO:41.

In another aspect, provided is an agonistic ICOS antigen binding molecule comprising at least one antigen binding domain capable of specific binding to FAP, wherein the antigen binding domain capable of specific binding to FAP comprises (a) a heavy chain variable region (V_{H}FAP) comprising an amino acid sequence that is at least about 95%, 96%, 97%, 98%, 99% or 100% identical to the amino acid sequence of SEQ ID NO:42, and a light chain variable region (V_{L}FAP) comprising an amino acid sequence that is at least about 95%, 96%, 97%, 98%, 99% or 100% identical to the amino acid sequence of SEQ ID NO:43, or (b) a heavy chain variable region (V_{H}FAP) comprising an amino acid sequence that is at least about 95%, 96%, 97%, 98%, 99% or 100% identical to the amino acid sequence of SEQ ID NO:50, and a light chain variable region (V_{L}FAP) comprising an amino acid sequence that is at least about 95%, 96%, 97%, 98%, 99% or 100% identical to the amino acid sequence of SEQ ID NO:51. In a particular aspect, the antigen binding domain capable of specific binding to FAP comprises a heavy chain variable region (V_{H}FAP) comprising the amino acid sequence of SEQ ID NO:42, and a light chain variable region (V_{L}FAP) comprising the amino acid sequence of SEQ ID NO:43. In a further aspect, the antigen binding domain capable of specific binding to FAP comprises a heavy chain variable region (V_{H}FAP) comprising the amino acid sequence of SEQ ID NO:50, and a light chain variable region (V_{L}FAP) comprising the amino acid sequence of SEQ ID NO:51.

Furthermore, there is provided an agonistic ICOS-binding molecule comprising at least one antigen binding domain capable of specific binding to a tumor-associated antigen, wherein the antigen binding domain capable of specific binding to ICOS comprises
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

Thus, in one aspect, provided is an agonistic ICOS-binding molecule comprising at least one antigen binding domain capable of specific binding to a tumor-associated antigen and at least one antigen binding domain capable of specific binding to ICOS that originates from mouse immunization, comprising a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11. In particular, an agonistic ICOS-binding molecule comprising at least one antigen binding domain capable of specific binding to a tumor-associated antigen and at least one antigen binding domain capable of specific binding to ICOS that originates from mouse immunization is provided which comprises a heavy chain variable region (V_{H}ICOS) comprising the amino acid sequence of SEQ ID NO:10, and a light chain variable region (V_{L}ICOS) comprising the amino acid sequence of SEQ ID NO:11.

In another aspect, the invention provides an agonistic ICOS-binding molecule comprising at least one antigen binding domain capable of specific binding to a tumor-associated antigen and at least one antigen binding domain capable of specific binding to ICOS that originates from rabbit immunization, comprising a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35. In one aspect, the antigen binding domain capable of specific binding to ICOS that originates from rabbit immunization comprises a heavy chain variable region (V_{H}ICOS) comprising the amino acid sequence of SEQ ID NO:18, and a light chain variable region (V_{L}ICOS) comprising the amino acid sequence of SEQ ID NO:19. In a another aspect, the antigen binding domain capable of specific binding to ICOS that originates from rabbit immunization comprises a heavy chain variable region (V_{H}ICOS) comprising the amino acid sequence of SEQ ID NO:26, and a light chain variable region (V_{L}ICOS) comprising the amino acid sequence of SEQ ID NO:27. In yet another aspect, the antigen binding domain capable of specific binding to ICOS that originates from rabbit immunization comprises a heavy chain variable region (V_{H}ICOS) comprising the amino acid sequence of SEQ ID NO:34, and a light chain variable region (V_{L}ICOS) comprising the amino acid sequence of SEQ ID NO:35.

In one aspect, the invention provides an agonistic ICOS antigen binding molecule as defined herein before, comprising
(a) one antigen binding domain capable of specific binding to a tumor-associated antigen,
(b) one Fab fragment capable of specific binding to ICOS, and
(c) a Fc domain composed of a first and a second subunit capable of stable association comprising one or more amino acid substitution that reduces the binding affinity of the antigen binding molecule to an Fc receptor and/or effector function. In particular, the agonistic ICOS antigen binding molecule comprises a Fc domain of human IgG1 subclass which comprises the amino acid mutations L234A, L235A and P329G (numbering according to Kabat EU index).

In another aspect, the invention provides an agonistic ICOS antigen binding molecule as defined herein before, comprising
(a) one antigen binding domain capable of specific binding to a tumor-associated antigen,
(b) two Fab fragments capable of specific binding to ICOS, and
(c) a Fc domain composed of a first and a second subunit capable of stable association comprising one or more amino acid substitution that reduces the binding affinity of the antigen binding molecule to an Fc receptor and/or effector function. In particular, the Fc domain of human IgG1 subclass comprises the amino acid mutations L234A, L235A and P329G (numbering according to Kabat EU index).

In particular aspects, the antigen binding domain capable of specific binding to a tumor-associated antigen is a crossFab fragment.

In a further aspect, provided is agonistic ICOS antigen binding molecule, in particular an antibody, comprising (a) a heavy chain variable region (V_{H}ICOS) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}ICOS) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}ICOS) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}ICOS) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or
(c) a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25, or
(d) a heavy chain variable region (V_{H}ICOS) comprising (i) CDR-H1 comprising the amino acid sequence of SEQ ID NO:28, (ii) CDR-H2 comprising the amino acid sequence of SEQ ID NO:29, and (iii) CDR-H3 comprising the amino acid sequence of SEQ ID NO:30, and a light chain variable region (V_{L}ICOS) comprising (iv) CDR-L1 comprising the amino acid sequence of SEQ ID NO:31, (v) CDR-L2 comprising the amino acid sequence of SEQ ID NO:32, and (vi) CDR-L3 comprising the amino acid sequence of SEQ ID NO:33.

In one aspect, the agonistic ICOS antigen binding molecule, in particular an antibody, is derived from mouse immunization and comprises a heavy chain variable region (V_{H}ICOS) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}ICOS) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9. In another aspect, the agonistic ICOS antigen binding molecule, in particular an antibody, is derived from rabbit immunization and comprises a heavy chain variable region (V_{H}ICOS) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}ICOS) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25, or a heavy chain variable region (V_{H}ICOS) comprising (i) CDR-H1 comprising the amino acid sequence of SEQ ID NO:28, (ii) CDR-H2 comprising the amino acid sequence of SEQ ID NO:29, and (iii) CDR-H3 comprising the amino acid sequence of SEQ ID NO:30, and a light chain variable region (V_{L}ICOS) comprising (iv) CDR-L1 comprising the amino acid sequence of SEQ ID NO:31, (v) CDR-L2 comprising the amino acid sequence of SEQ ID NO:32, and (vi) CDR-L3 comprising the amino acid sequence of SEQ ID NO:33.

In one aspect, provided is agonistic ICOS antigen binding molecule, in particular an antibody, which comprises
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

In one aspect, the agonistic ICOS antigen binding molecule is full-length antibody. In another aspect, the agonistic ICOS antigen binding molecule is a Fab or crossFab fragment. In a particular aspect, the agonistic ICOS antigen binding molecule is a humanized antibody.

According to another aspect of the invention, there is provided an isolated nucleic acid encoding an agonistic ICOS antigen binding molecule comprising at least one antigen binding domain capable of specific binding to a tumor-associated antigen or an ICOS antibody as described herein before. The invention further provides a vector, particularly an expression vector, comprising the isolated nucleic acid of the invention and a host cell comprising the isolated nucleic acid or the vector of the invention. In some aspects the host cell is a eukaryotic cell, particularly a mammalian cell.

In another aspect, provided is a method for producing an agonistic ICOS antigen binding molecule comprising at least one antigen binding domain capable of specific binding to a tumor-associated antigen as described herein before, comprising culturing the host cell of the invention under conditions suitable for expression of the agonistic ICOS antigen binding molecule, and recovering the antigen binding molecule from the host cell. The invention also encompasses the agonistic ICOS antigen binding molecule comprising at least one antigen binding domain capable of specific binding to a tumor-associated antigen or the ICOS antibody as described herein produced by the method of the invention.

The invention further provides a pharmaceutical composition comprising an agonistic ICOS antigen binding molecule comprising at least one antigen binding domain capable of specific binding to a tumor-associated antigen as described herein before and at least one pharmaceutically acceptable excipient. In particular, the pharmaceutical composition is for use in the treatment of cancer.

Also encompassed by the invention is the agonistic ICOS antigen binding molecule comprising at least one antigen binding domain capable of specific binding to a tumor-associated antigen as described herein before, or the pharmaceutical composition comprising the agonistic ICOS-binding molecule comprising at least one antigen binding domain capable of specific binding to a tumor-associated antigen, for use as a medicament.

In one aspect, provided is the agonistic ICOS antigen binding molecule comprising at least one antigen binding domain capable of specific binding to a tumor-associated antigen as described herein before or the pharmaceutical composition comprising the agonistic ICOS-binding molecule comprising at least one antigen binding domain capable of specific binding to a tumor-associated antigen, for use
(i) in stimulating T cell response,
(ii) in supporting survival of activated T cells,
(iii) in the treatment of infections,
(iv) in the treatment of cancer,
(v) in delaying progression of cancer, or
(vi) in prolonging the survival of a patient suffering from cancer.

In a specific aspect, there is provided the agonistic ICOS antigen binding molecule comprising at least one antigen binding domain that binds to a tumor-associated antigen as described herein before, or the pharmaceutical composition comprising the agonistic ICOS-binding molecule comprising at least one antigen binding domain capable of specific binding to a tumor-associated antigen as described herein before, for use in the treatment of cancer.

In another specific aspect, the invention provides the agonistic ICOS-binding molecule comprising at least one antigen binding domain capable of specific binding to a tumor-associated antigen as described herein before as described herein for use in the treatment of cancer, wherein the agonistic ICOS-binding molecule comprising at least one antigen binding domain capable of specific binding to a tumor-associated antigen is for administration in combination with a chemotherapeutic agent, radiation therapy and/or other agents for use in cancer immunotherapy.

In one aspect, provided is the agonistic ICOS antigen binding molecule comprising at least one antigen binding domain capable of specific binding to a tumor-associated antigen as described herein before for use in the treatment of cancer, wherein the agonistic ICOS antigen binding molecule comprising at least one antigen binding domain capable of specific binding to a tumor-associated antigen is for administration in combination with a T-cell activating anti-CD3 bispecific antibody. In particular, the T-cell activating anti-CD3 bispecific antibody is an anti-CEA/anti-CD3 bispecific antibody.

In a further aspect, provided is the agonistic ICOS antigen binding molecule comprising at least one antigen binding domain capable of specific binding to a tumor-associated antigen as described herein before for use in the treatment of cancer, wherein the agonistic ICOS antigen binding molecule comprising at least one antigen binding domain capable of specific binding to a tumor-associated antigen is for administration in combination with an agent blocking PD-L1/PD-1 interaction. In one aspect, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab.

In a further aspect, the invention provides a method of inhibiting the growth of tumor cells in an individual comprising administering to the individual an effective amount of the agonistic ICOS antigen binding molecule comprising at least one antigen binding domain that binds to a tumor-associated antigen as described herein before, or the pharmaceutical composition comprising the agonistic ICOS antigen binding molecule comprising at least one antigen binding domain that binds to a tumor-associated antigen as described herein before, to inhibit the growth of the tumor cells. In another aspect, the invention provides a method of treating cancer in an individual comprising administering to the individual an effective amount of the agonistic ICOS antigen binding molecule comprising at least one antigen binding domain that binds to a tumor-associated antigen as described herein before.

Also provided is the use of the agonistic ICOS antigen binding molecule comprising at least one antigen binding domain capable of specific binding to a tumor-associated antigen as described herein before for the manufacture of a medicament for the treatment of a disease in an individual in need thereof, in particular for the manufacture of a medicament for the treatment of cancer. In any of the above aspects the individual is a mammal, particularly a human.

### BRIEF DESCRIPTION OF THE DRAWINGS

FIGS. 1A-H: Schematic Figures of the bispecific agonistic ICOS antigen binding molecules. In FIG. 1A and FIG. 1B different types of FAP-ICOS bispecific antibodies in 1+1 format are shown (1+1 means monovalent binding to ICOS as well as to FAP). The format shown in FIG. 1B is also named 1+1 head-to-tail. A FAP-ICOS antibody in 2+1 format (monovalent for the tumor-associated target), wherein the VH and VL domain of FAP are each bound to the C-terminus of each Fc domain is shown in FIG. 1C and in FIGS. 1D and 1E two different types of 2+1 formats are shown wherein the Fab domain comprising the FAP antigen binding domain is fused to a Fab domain of an ICOS IgG (FIG. 1D) or wherein one of ICOS Fab domains is fused to the N-terminus of the FAP Fab domain (inverted, FIG. 1E). Different types of CEA-ICOS bispecific antibodies in 1+1 format are shown in FIG. 1F, FIG. 1G and FIG. 1H.

FIGS. 2A and 2B: Binding of all selected parental lead clones as IgGs vs JMab136 IgG (Molecule 1) to ICOS expressed on CHO-huICOS cells or SR cells. FIG. 2A shows the Dose response curve depicting Median Fluorescence Intensitites (MFI) for the dose dependent binding of ICOS antibodies to human ICOS on recombinant CHO cells. FIG. 2B shows Dose response curve depicting Median Fluorescence Intensitites (MFI) for the dose dependent binding of ICOS antibodies to human ICOS on SR cells. The tested clones are ICOS (009) (Molecule 14), ICOS 1138 (Molecule 18), 1143 (Molecule 20) and 1167 (Molecule 8). Graphs depict mean of technical triplicates, error bars indicate SD.

FIGS. 3A, 3B and 3C: Binding of selected humanization variants of lead clones 009v1, 1143v2 and 1138 to human ICOS, respectively. Shown are dose response curves depicting Median Fluorescence Intensitites (MFI) for the dose dependent binding of humanization variants for three different aICOS molecules to human ICOS on recombinant CHO cells. Graphs depict mean of technical triplicates, error bars indicate SD.

FIG. 4: Dose response curves from the Jurkat-NFAT assay of all selected parental lead clones as IgGs vs JMab136.

FIGS. 5A to 5C: Dose response curves from the Jurkat-NFAT Reporter Assay for humanization variants of lead clones 009v1, 1143v2 and 1138 (as IgGs), respectively. Dose response curves are shown depicting counts per second (CPS) for the dose dependent activation of Jurkat-NFAT cells treated with increasing doses of humanization variants for three different aICOS molecules. Graphs depict mean of technical triplicates, error bars indicate SD.

FIGS. 6A and 6B: Binding of bispecific FAP-ICOS antigen binding molecules to hu ICOS. FIG. 6A shows the Dose response curves depicting Median Fluorescence Intensitites (MFI) for the dose dependent binding of FAP-ICOS molecules to human ICOS on recombinant CHO. Compared are bispecific antibodies in 2+1 format with different ICOS clones 009v1 (Molecule 15), 1138 (Molecule 19), 1143v2 (Molecule 22) and 1167 (Molecule 9). FIG. 6B shows the Dose response curves depicting Median Fluorescence Intensities (MFI) for the dose dependent binding of FAP-ICOS molecules to human ICOS on recombinant CHO cells. Compared are different formats comprising ICOS clone 1167: Molecule 9 (2+1, see FIG. 1C), Molecule 10 (1+1, see FIG. 1A) and Molecule 11 (1+1_HT, see FIG. 1B). Graphs depict mean of technical triplicates, error bars indicate SD.

FIGS. 7A and 7B: Binding of bispecific FAP-ICOS antigen binding molecules to hu FAP (NIH3T3-hFAP). FIG. 7A shows the Dose response curves depicting Median Fluorescence Intensities (MFI) for the dose dependent binding of FAP-ICOS molecules to human FAP on recombinant 3t3-huFAP clone 19 cells. Compared are bispecific antibodies in 2+1 format with different ICOS clones 009v1 (Molecule 15), 1138 (Molecule 19), 1143v2 (Molecule 22) and 1167 (Molecule 9). FIG. 7B shows the Dose response curves depicting Median Fluorescence Intensities (MFI) for the dose dependent binding of FAP-ICOS molecules to human FAP on recombinant 3t3-huFAP clone 19 cells. Compared are different formats comprising ICOS clone 1167: Molecule 9 (2+1, see FIG. 1C), Molecule 10 (1+1, see FIG. 1A) and Molecule 11 (1+1_HT, see FIG. 1B). Graphs depict mean of technical triplicates, error bars indicate SD.

FIGS. 8A and 8B: Binding of bispecific FAP-ICOS antigen binding molecules to cynomolgus ICOS on activated PBMCs. Shown are dose response curves depicting Median Fluorescence Intensitites (MFI) for the dose dependent binding of FAP-ICOS molecules comprising different ICOS clones 009v1 (Molecule 15), 1138 (Molecule 19), 1143v2 (Molecule 22), 1167 (Molecule 9) and JMab136 (Molecule 2). FIGS. 8A and 8B show binding on CD4+ and CD8+ subsets, respectively. Graphs depict mean of technical triplicates, error bars indicate SD.

FIGS. 8C and 8D: Binding of bispecific FAP-ICOS antigen binding molecules to cynomolgus ICOS on activated PBMCs. Shown are dose response curves depicting Median Fluorescence Intensitites (MFI) for the dose dependent binding of FAP-ICOS molecules comprising different formats comprising ICOS clone 1167: Molecule 9 (2+1, see FIG. 1C), Molecule 10 (1+1, see FIG. 1A) and Molecule 11 (1+1_HT). FIGS. 8C and 8D show binding on CD4+ and CD8+ subsets, respectively. Graphs depict mean of technical triplicates, error bars indicate SD.

FIGS. 9A and 9B: Binding of bispecific FAP-ICOS antigen binding molecules to murine ICOS on recombinant CHO cells. In FIG. 9A are shown dose response curves depicting frequency of ICOS+ cells (%) for the dose dependent binding of FAP-ICOS molecules to murine ICOS. FIG. 9B shows the Dose response curves depicting frequency of ICOS+ cells (%) for the dose dependent binding of FAP-ICOS molecules to murine FAP. Graph depict mean of technical triplicates, error bars indicate SD. Provided are the data for FAP-ICOS molecules with different formats comprising ICOS clone 1167: Molecule 9 (2+1, see FIG. 1C), Molecule 10 (1+1, see FIG. 1A) and Molecule 11 (1+1_HT).

FIGS. 10A to 10C: Binding of bispecific FAP-ICOS antigen binding molecules comprising clone 1167 to human ICOS (pre-activated PBMCs) and to human FAP (NIH3T3-hFAP). Shown are the data for the formats of FIG. 1A (Molecule 10), FIG. 1D (Molecule 12) and FIG. 1E (Molecule 13). FIGS. 10A and 10B show the dose dependent binding of FAP-ICOS molecules to human ICOS on activated PBMCs for the CD4+ and CD8+ subsets, respectively. FIG. 10C shows the Dose response curves depicting Median Fluorescence Intensitites (MFI) for the dose dependent binding of the FAP-ICOS molecules to human FAP on recombinant 3t3-huFAP clone 19 cells. Graphs depict mean of technical triplicates, error bars indicate SD.

FIGS. 11A to 11C: Binding of bispecific CEA-ICOS antigen binding molecules to human ICOS (CD4 and CD8 subsets of human PBMCs) and to human CEA (MKN-45). Shown are the data for CEA(A5H1EL1D)-ICOS(1167) 1+1 (Molecule 41), CEA(A5H1EL1D)-ICOS(H009v1_2) (Molecule 42) and CEA(A5H1EL1D)-ICOS(1143v2_1) (Molecule 43). FIGS. 11A and 11B show the Dose response curves depicting Median Fluorescence Intensitites (MFI) for the dose dependent binding of CEA-ICOS molecules to human ICOS on activated PBMCs (CD4+ and CD8+ subsets respectively). FIG. 11C shows the Dose response curves depicting Median Fluorescence Intensitites (MFI) for the dose dependent binding of CEA-ICOS molecules to human CEA on MKN-45 cells. Graphs depict mean of technical triplicates, error bars indicate SD.

FIGS. 12A to 12C: Selection of germlining variants of lead binders in bispecific format. Different germlining variants of clones 009 and 1143 were tested as bispecific FAP-ICOS antibodies in the 2+1 format (FIG. 1C). FIG. 12A shows binding of the molecules to ICOS expressed on SR cells, the dose response curve is depicting Median Fluorescence Intensitites (MFI) for the dose dependent binding of ICOS antibodies to human ICOS on SR cells. FIG. 121B shows binding of the bispecific FAP-ICOS antigen binding molecules to human FAP (NIH3T3-hFAP). Dose response curves are depicting Median Fluorescence Intensitites (MFI) for the dose dependent binding to human FAP on recombinant 3t3-huFAP clone 19 cells. FIG. 12C shows the selection of germlining variants of the lead clones in a primary PMBC assay. Each dot represents an individual donor. Values indicate maximum value of MFI CD69 on CD4+ T-Cells across the concentration range.

FIGS. 13A and 13B: Increased TCB-mediated T-cell activation in the presence of bispecific FAP-ICOS antigen binding molecules. Shown are Median Fluorescence Intensitites (MFI) CD25-positive CD4+ T cells after 48 h of co-incubation of human PBMC effector, MV3 tumor cells at an E:T of 5:1 in the presence of 5 pM MCSP TCB and of increasing concentration of FAP-ICOS. The graphs show the maximal response of three donors for each molecule. FIG. 13A shows the Comparison of different ICOS clones. FIG. 13B shows the Comparison of different formats comprising clone 1167.

FIGS. 14A to 14C: Increased TCB-mediated T-cell activation in presence of bispecific FAP-ICOS antigen binding molecules. Shown are Median Fluorescence Intensitites (MFI) CD69-positive CD4+ T cells after 48 h of co-incubation of human PBMC effector, MKN-45 tumor cells and NIH/3t3-huFAP clone 19 fibroblasts at an E:T of 5:1:1 in presence of 80 pM CEACAM5 TCB in presence of increasing concentration of FAP-ICOS. FIGS. 14A and 14B show the Dose response graphs of two donors. Dots represent mean of technical triplicates, error bars indicate SD. FIG. 14C shows the maximal response of two donors for each molecule. Each dot represents the mean of a technical triplicate.

FIGS. 15A to 15C: Increased TCB-mediated T-cell activation in presence of bispecific CEA-ICOS antigen binding molecules compared to FAP-ICOS. Shown are Median Fluorescence Intensitites (MFI) CD69-positive CD4+ T cells after 48 h of co-incubation of human PBMC effector, MKN-45 tumor cells and NIH/3t3-huFAP clone 19 fibroblasts at an E:T of 5:1:1 in presence of 80 pM CEACAM5 TCB and of increasing concentration of FAP-ICOS. FIGS. 15A and 15B show the Dose response graphs of two donors. Dots represent mean of technical triplicates, error bars indicate SD. FIG. 15C: The graph shows the maximal response of two donors for each molecule. Each dot represents the mean of a technical triplicate.

FIGS. 16A to 16C: Increased TCB-mediated T-cell activation in presence of bispecific CEA-ICOS antigen binding molecules. Shown are Median Fluorescence Intensitites (MFI) CD69-positive CD4+ T cells after 48 h of co-incubation of human PBMC effector, MKN-45 tumor cells and NIH/3t3-huFAP clone 19 fibroblasts at an E:T of 5:1:1 in presence of 80 pM CEACAM5 TCB and of increasing concentration of CEA-ICOS. FIGS. 16A and 16B show the Dose response graphs of two donors. Dots represent mean of technical triplicates, error bars indicate SD. FIG. 16C: The graph shows the maximal response of three donors for each molecule. Each dot represents the mean of a technical triplicate.

FIG. 17: Pharmacokinetic profiles of three bispecific FAP-ICOS antigen binding molecules comprising ICOS clone 1167 (in different formats) after single injection in NSG mice (Example 9.1)

FIG. 18: Study design and treatment groups of the Efficacy study with three bispecific FAP-ICOS antigen binding molecules in combination with CEACAM5 TCB in MKN45 Xenograft in humanized mice (Example 9.2).

FIGS. 19A to 19G: Efficacy study with FAP-ICOS in different formats and CEACAM5 TCB combination in MKN45 Xenograft in humanized mice at the same dose. Shown is the average tumor volume (FIG. 19F) or the growth of tumors in individual mice as plotted on the y-axis (FIGS. 19A to 19E). Tumor weight at day 50 as plotted for individual mice is summarized in FIG. 19G. It can be seen that there is increased TCB-mediated Tumor Regression in the presence of all FAP-ICOS molecules.

FIGS. 20A to 20F: Efficacy study with FAP-ICOS in different formats and CEACAM5 TCB combination in MKN45 Xenograft in humanized mice at the same dose. Shown are the ImmunoPD data in the tumor and spleen.

FIGS. 21A to 21G: Dose Response study with a FAP-ICOS molecule in 1+1 format and CEACAM5 TCB combination in MKN45 Xenograft in humanized mice in different doses. Shown is the average tumor volume (FIG. 21F) or the growth of tumors in individual mice as plotted on the y-axis (FIGS. 21A to 21E). Tumor weight at day 50 as plotted for individual mice is summarized in FIG. 21G. It can be seen that there is increased TCB-mediated Tumor Regression in the presence of the lowest dose of FAP-ICOS.

FIGS. 22A to 22F: Dose Response study with FAP-ICOS with a FAP-ICOS molecule in 1+1 format and CEACAM5 TCB combination in MKN45 Xenograft in humanized mice in different doses. Shown are the ImmunoPD data in the tumor and spleen.

FIG. 23: Cytokine analysis. Intra-tumoral changes in selected chemokine and cytokine expression upon combination therapy with FAP-ICOS in different doses and CEACAM5-TCB in a co-grafting model of MKN45 and 3T3-hFAP cells in humanized NSG mice.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

As used herein, the term “antigen binding domain that binds to a tumor-associated antigen” or “antigen binding domain capable of specific binding to a tumor-associated antigen” or “moiety capable of specific binding to a tumor-associated antigen” refers to a polypeptide molecule that specifically binds to an antigenic determinant. In one aspect, the antigen binding domain is able to activate signaling through its target cell antigen. In a particular aspect, the antigen binding domain is able to direct the entity to which it is attached (e.g. the ICOS agonist) to a target site, for example to a specific type of tumor cell or tumor stroma bearing the antigenic determinant. Antigen binding domains capable of specific binding to a target cell antigen include antibodies and fragments thereof as further defined herein. In addition, antigen binding domains capable of specific binding to a target cell antigen include scaffold antigen binding proteins as further defined herein, e.g. binding domains which are based on designed repeat proteins or designed repeat domains (see e.g. WO 2002/020565).

In relation to an antibody or fragment thereof, the term “antigen binding domain capable of specific binding to a target cell antigen” refers to the part of the molecule that comprises the area which specifically binds to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH). In another aspect, the “antigen binding domain capable of specific binding to a target cell antigen” can also be a Fab fragment or a cross-Fab fragment.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2).

The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can be engineered to include upto 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, a molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

A “tumor-associated antigen” or TAA as used herein refers to an antigenic determinant presented on the surface of a target cell, for example a cell in a tumor such as a cancer cell or a cell of the tumor stroma. In certain embodiments, the target cell antigen is an antigen on the surface of a tumor cell. In one embodiment, TAA is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epidermal growth factor receptor 2 (HER2) and p95HER2. In particular, the tumor-associated antigen is Fibroblast Activation Protein (FAP) or Carcinoembryonic Antigen (CEA).

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:254), or NCBI (www.ncbi.nlm.nih.gov/) RefSeq NP_004451.2. The extracellular domain (ECD) of human FAP extends from amino acid position 26 to 760. The amino acid sequence of a His-tagged human FAP ECD is shown in SEQ ID NO 255. The amino acid sequence of mouse FAP is shown in UniProt accession no. P97321 (version 126, SEQ ID NO:256), or NCBI RefSeq NP_032012.1. The extracellular domain (ECD) of mouse FAP extends from amino acid position 26 to 761. SEQ ID NO 257 shows the amino acid sequence of a His-tagged mouse FAP ECD. SEQ ID NO 258 the amino acid sequence of a His-tagged cynomolgus FAP ECD. Preferably, an anti-FAP binding molecule of the invention binds to the extracellular domain of FAP.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:259). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarström S., Semin Cancer Biol. 9(2):67-81 (1999)), making it inaccessible to antibody in the blood stream. In contrast to normal tissue, CEA tends to be expressed over the entire surface of cancerous cells (Hammarström S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30(a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “FolR1” refers to Folate receptor alpha and has been identified as a potential prognostic and therapeutic target in a number of cancers. It refers to any native FolR1 from any vertebrate source, including mammals such as primates (e.g. humans) non-human primates (e.g. cynomolgus monkeys) and rodents (e.g. mice and rats), unless otherwise indicated. The amino acid sequence of human FolR1 is shown in UniProt accession no. P15328 (SEQ ID NO: 260), murine FolR1 has the amino acid sequence of UniProt accession no. P35846 (SEQ ID NO:261) and cynomolgus FolR1 has the amino acid sequence as shown in UniProt accession no. G7PR14 (SEQ ID NO:262). FolR1 is an N-glycosylated protein expressed on plasma membrane of cells. FolR1 has a high affinity for folic acid and for several reduced folic acid derivatives and mediates delivery of the physiological folate, 5-methyltetrahydrofolate, to the interior of cells. FOLR1 is a desirable target for FOLR1-directed cancer therapy as it is overexpressed in vast majority of ovarian cancers, as well as in many uterine, endometrial, pancreatic, renal, lung, and breast cancers, while the expression of FOLR1 on normal tissues is restricted to the apical membrane of epithelial cells in the kidney proximal tubules, alveolar pneumocytes of the lung, bladder, testes, choroid plexus, and thyroid. Recent studies have identified that FolR1 expression is particularly high in triple negative breast cancers (Necela et al. PloS One 2015, 10(3), e0127133).

The term “Melanoma-associated Chondroitin Sulfate Proteoglycan (MCSP)”, also known as Chondroitin Sulfate Proteoglycan 4 (CSPG4) refers to any native MCSP from any vertebrate source, including mammals such as primates (e.g. humans) non-human primates (e.g. cynomolgus monkeys) and rodents (e.g. mice and rats), unless otherwise indicated. The amino acid sequence of human MCSP is shown in UniProt accession no. Q6UVK1 (version 103, SEQ ID NO:263). MCSP is a highly glycosylated integral membrane chondroitin sulfate proteoglycan consisting of an N-linked 280 kDa glycoprotein component and a 450-kDa chondroitin sulfate proteoglycan component expressed on the cell membrane (Ross et al., Arch. Biochem. Biophys. 1983, 225:370-38). MCSP is more broadly distributed in a number of normal and transformed cells. In particular, MCSP is found in almost all basal cells of the epidermis. MCSP is differentially expressed in melanoma cells, and was found to be expressed in more than 90% of benign nevi and melanoma lesions analyzed. MCSP has also been found to be expressed in tumors of nonmelanocytic origin, including basal cell carcinoma, various tumors of neural crest origin, and in breast carcinomas.

The term “Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533 (version 211, SEQ ID NO:264). The proto-oncogene “HER2”, (human epidermal growth factor receptor 2) encodes a protein tyrosine kinase (p185HER2) that is related to and somewhat homologous to the human epidermal growth factor receptor. HER2 is also known in the field as c-erbB-2, and sometimes by the name of the rat homolog, neu. Amplification and/or overexpression of HER2 is associated with multiple human malignancies and appears to be integrally involved in progression of 25-30% of human breast and ovarian cancers. Furthermore, the extent of amplification is inversely correlated with the observed median patient survival time (Slamon, D. J. et al., Science 244:707-712 (1989)). The amino acid sequence of human HER2 is shown in UniProt accession no. P04626 (version 230, SEQ ID NO:265). The term “p95HER2” as used herein refers to a carboxy terminal fragment (CTF) of the HER2 receptor protein, which is also known as “611-CTF” or “100-115 kDa p95HER2”. The p95HER2 fragment is generated in the cell through initiation of translation of the HER2 mRNA at codon position 611 of the full-length HER2 molecule (Anido et al, EMBO J 25; 3234-44 (2006)). It has a molecular weight of 100 to 115 kDa and is expressed at the cell membrane, where it can form homodimers maintained by intermolecular disulfide bonds (Pedersen et al., Mol Cell Biol 29, 3319-31 (2009)). An exemplary sequence of human p95HER2 is given in SEQ ID NO: 266.

The term “ICOS” (Inducible T cell COStimulator) refers to any Inducible T cell costimulatory protein from any vertebrate source, including mammals such as primates (e.g. humans) non-human primates (e.g. cynomolgus monkeys) and rodents (e.g. mice and rats), unless otherwise indicated. ICOS, also named AILIM or CD278, is a member of the CD28 superfamily (CD28/CTLA-4 cell-surface receptor family) and is specifically expressed on T cells after initial T cell activation. ICOS also plays a role in the development and function of other T cell subsets, including Th1, Th2, and Th17. Notably, ICOS co-stimulates T cell proliferation and cytokine secretion associated with both Th1 and Th2 cells. Accordingly, ICOS KO mice demonstrate impaired development of autoimmune phenotypes in a variety of disease models, including diabetes (Th1), airway inflammation (Th2) and EAE neuro-inflammatory models (Th17). In addition to its role in modulating T effector (Teff) cell function, ICOS also modulates T regulatory cells (Tregs). ICOS is expressed at high levels on Tregs, and has been implicated in Treg homeostasis and function. Upon activation, ICOS, a disulfide-linked homodimer, induces a signal through the PI3K and AKT pathways. Subsequent signaling events result in expression of lineage specific transcription factors (e.g., T-bet, GATA-3) and, in turn, effects on T cell proliferation and survival. The term also encompasses naturally occurring variants of ICOS, e.g., splice variants or allelic variants. The amino acid sequence of human ICOS is shown in UniProt (www.uniprot.org) accession no. Q9Y6W8 (SEQ ID NO:1)

As described herein before, ICOS ligand (ICOS-L; B7-H2; B7RP-1; CD275; GL50), also a member of the B7 superfamily, is the membrane bound natural ligand for ICOS and is expressed on the cell surface of B cells, macrophages and dendritic cells. ICOS-L functions as a non-covalently linked homodimer on the cell surface in its interaction with ICOS. Human ICOS-L has also been reported to bind to human CD28 and CTLA-4 (Yao et al., 2011, Immunity, 34: 729-740). An exemplary amino acid sequence of the ectodomain of huICOS-L is given in SEQ ID NO: 215.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

Kabat et al. defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “CH1 domain” denotes the part of an antibody heavy chain polypeptide that extends approximately from EU position 118 to EU position 215 (EU numbering system according to Kabat). In one aspect, a CH1 domain has the amino acid sequence of ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKV (SEQ ID NO: 267). Usually, a segment having the amino acid sequence of EPKSC (SEQ ID NO:268) is following to link the CH1 domain to the hinge region.

The term “hinge region” denotes the part of an antibody heavy chain polypeptide that joins in a wild-type antibody heavy chain the CH1 domain and the CH2 domain, e. g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083).

The term “Fc domain” or “Fc region” herein is used to define a C-terminal region of an antibody heavy chain that contains at least a portion of the constant region. The term includes native sequence Fc regions and variant Fc regions. In one aspect, a human IgG heavy chain Fc-domain extends from Cys226, or from Pro230, or from Ala231 to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, numbering according to EU index). In one aspect, a heavy chain including an Fc region as specified herein, comprised in an antibody according to the invention, comprises an additional C-terminal glycine residue (G446, numbering according to EU index). An IgG Fc region comprises an IgG CH2 and an IgG CH3 domain.

The “CH2 domain” of a human IgG Fc region usually extends from an amino acid residue at about EU position 231 to an amino acid residue at about EU position 340 (EU numbering system according to Kabat). In one aspect, a CH2 domain has the amino acid sequence of APELLGGPSV FLFPPKPKDT LMISRTPEVT CVWDVSHEDP EVKFNWYVDG VEVHNAKTKP REEQESTYRW SVLTVLHQDW LNGKEYKCKV SNKALPAPIE KTISKAK (SEQ ID NO: 269). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206. In one aspect, a carbohydrate chain is attached to the CH2 domain. The CH2 domain herein may be a native sequence CH2 domain or variant CH2 domain.

The “CH3 domain” comprises the stretch of residues C-terminal to a CH2 domain in an Fc region denotes the part of an antibody heavy chain polypeptide that extends approximately from EU position 341 to EU position 446 (EU numbering system according to Kabat). In one aspect, the CH3 domain has the amino acid sequence of GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSPG (SEQ ID NO: 270). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492, Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is a function mediated by Fc receptor binding and refers to lysis of target cells by an antibody as reported herein in the presence of effector cells. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

An “ectodomain” is the domain of a membrane protein that extends into the extracellular space (i.e. the space outside the target cell). Ectodomains are usually the parts of proteins that initiate contact with surfaces, which leads to signal transduction.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO: 271) GGGGSGGGGS (SEQ ID NO:272), SGGGGSGGGG (SEQ ID NO:273) and GGGGSGGGGSGGGG (SEQ ID NO:274), but also include the sequences GSPGSSSSGS (SEQ ID NO:275), (G4S)₃ (SEQ ID NO:276), (G4S)₄ (SEQ ID NO:277), GSGSGSGS (SEQ ID NO:278), GSGSGNGS (SEQ ID NO:279), GGSGSGSG (SEQ ID NO:280), GGSGSG (SEQ ID NO:281), GGSG (SEQ ID NO:282), GGSGNGSG (SEQ ID NO:283), GGNGSGSG (SEQ ID NO:284) and GGNGSG (SEQ ID NO:285). Peptide linkers of particular interest are (G4S) (SEQ ID NO:271), (G₄S)₂ or GGGGSGGGGS (SEQ ID NO:272), (G4S)₃ (SEQ ID NO:276) and (G4S)₄ (SEQ ID NO:277).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the agonistic ICOS-binding molecules provided herein are contemplated. For example, it may be desirable to improve the binding affinity and/or other biological properties of the agonistic ICOS-binding molecules. Amino acid sequence variants of the agonistic ICOS-binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of insertions include agonistic ICOS-binding molecules with a fusion to the N- or C-terminus to a polypeptide which increases the serum half-life of the agonistic ICOS-binding molecules.

In certain embodiments, the agonistic ICOS-binding molecules provided herein are altered to increase or decrease the extent to which the antibody is glycosylated. Glycosylation variants of the molecules may be conveniently obtained by altering the amino acid sequence such that one or more glycosylation sites is created or removed. Where the agonistic ICOS-binding molecule comprises an Fc domain, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in agonistic ICOS-binding molecules may be made in order to create variants with certain improved properties. In one aspect, variants of agonistic ICOS-binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the agonistic ICOS-binding molecules of the invention include those with bisected oligosaccharides, e.g., in which a biantennary oligosaccharide attached to the Fc region is bisected by GlcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the agonistic ICOS-binding molecules of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the agonistic ICOS-binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed. In another aspect, immunoconjugates of the agonistic ICOS-binding molecules provided herein maybe obtained. An “immunoconjugate” is an antibody conjugated to one or more heterologous molecule(s), including but not limited to a cytotoxic agent.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

Agonistic ICOS-Binding Molecules of the Invention

The invention provides novel bispecific antigen binding molecules with particularly advantageous properties such as producibility, stability, binding affinity, biological activity, targeting efficiency, reduced toxicity, an extended dosage range that can be given to a patient and thereby a possibly enhanced efficacy.

Exemplary Agonistic ICOS-Binding Molecules Comprising at Least One Antigen Binding Domain that Binds to a Tumor-Associated Antigen

In one aspect, the invention provides agonistic ICOS antigen binding molecules comprising at least one antigen binding domain capable of specific binding to a tumor-associated antigen and at least one antigen binding domain capable of specific binding to ICOS comprising
(a) a heavy chain variable region (V_{H}ICOS) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}ICOS) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}ICOS) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}ICOS) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or
(c) a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25, or
(d) a heavy chain variable region (V_{H}ICOS) comprising (i) CDR-H1 comprising the amino acid sequence of SEQ ID NO:28, (ii) CDR-H2 comprising the amino acid sequence of SEQ ID NO:29, and (iii) CDR-H3 comprising the amino acid sequence of SEQ ID NO:30, and a light chain variable region (V_{L}ICOS) comprising (iv) CDR-L1 comprising the amino acid sequence of SEQ ID NO:31, (v) CDR-L2 comprising the amino acid sequence of SEQ ID NO:32, and (vi) CDR-L3 comprising the amino acid sequence of SEQ ID NO:33.

The agonistic ICOS antigen binding molecules are thus characterized by comprising a novel ICOS antigen binding domain with improved properties compared to known ICOS antibodies.

In one aspect, the invention provides such bispecific agonistic ICOS antigen binding molecules, comprising
(a) at least one antigen binding domain capable of specific binding to ICOS, and
(b) at least one antigen binding domain capable of specific binding to a tumor-associated antigen, and
(c) a Fc domain.

In a particular aspect, the agonistic ICOS-binding molecules comprise a Fc domain comprising mutations that reduce or abolish effector function. The use of a Fc domain comprising mutations that reduce or abolish effector function will prevent unspecific agonism by crosslinking via Fc receptors and will prevent ADCC of ICOS⁺ cells.

Thus, provided are agonistic ICOS antigen binding molecules as defined above, further comprising a Fc domain composed of a first and a second subunit capable of stable association which comprises one or more amino acid substitution that reduces the binding affinity of the antigen binding molecule to an Fc receptor and/or effector function. In particular, the agonistic ICOS antigen binding molecule comprises a Fc domain of human IgG1 subclass which comprises the amino acid mutations L234A, L235A and P329G (numbering according to Kabat EU index).

The agonistic ICOS-binding molecules as described herein possess the advantage over conventional antibodies capable of specific binding to ICOS in that they selectively induce immune response at the target cells, which are typically cancer cells or tumor stroma. In one aspect, the tumor-associated antigen is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epidermal growth factor receptor 2 (HER2) and p95HER2. In particular, the tumor-associated antigen is FAP or CEA. In one particular aspect, the tumor-associated antigen is FAP. In another particular aspect, the tumor-associated antigen is CEA.

In one aspect, there is provided an agonistic ICOS-binding molecule comprising at least one antigen binding domain capable of specific binding to a tumor-associated antigen as defined above, wherein the antigen binding domain capable of specific binding to a tumor-associated antigen is an antigen binding domain capable of specific binding to Carcinoembryonic Antigen (CEA). In one aspect, the antigen binding domain capable of specific binding to CEA comprises
(a) a heavy chain variable region (V_{H}CEA) comprising (i) CDR-H1 comprising the amino acid sequence of SEQ ID NO:52, (ii) CDR-H2 comprising the amino acid sequence of SEQ ID NO:53, and (iii) CDR-H3 comprising the amino acid sequence of SEQ ID NO:54, and a light chain variable region (V_{L}CEA) comprising (iv) CDR-L1 comprising the amino acid sequence of SEQ ID NO:55, (v) CDR-L2 comprising the amino acid sequence of SEQ ID NO:56, and (vi) CDR-L3 comprising the amino acid sequence of SEQ ID NO:57, or (b) a heavy chain variable region (V_{H}CEA) comprising (i) CDR-H1 comprising the amino acid sequence of SEQ ID NO:60, (ii) CDR-H2 comprising the amino acid sequence of SEQ ID NO:61, and (iii) CDR-H3 comprising the amino acid sequence of SEQ ID NO:62, and a light chain variable region (V_{L}CEA) comprising (iv) CDR-L1 comprising the amino acid sequence of SEQ ID NO:63, (v) CDR-L2 comprising the amino acid sequence of SEQ ID NO:64, and (vi) CDR-L3 comprising the amino acid sequence of SEQ ID NO:65. In one particular aspect, the antigen binding domain capable of specific binding to CEA comprises a heavy chain variable region (V_{H}CEA) comprising (i) CDR-H1 comprising the amino acid sequence of SEQ ID NO:60, (ii) CDR-H2 comprising the amino acid sequence of SEQ ID NO:61, and (iii) CDR-H3 comprising the amino acid sequence of SEQ ID NO:62, and a light chain variable region (V_{L}CEA) comprising (iv) CDR-L1 comprising the amino acid sequence of SEQ ID NO:63, (v) CDR-L2 comprising the amino acid sequence of SEQ ID NO:64, and (vi) CDR-L3 comprising the amino acid sequence of SEQ ID NO:65.

In another aspect, provided is an agonistic ICOS antigen binding molecule as defined above, wherein the antigen binding domain capable of specific binding to CEA comprises a heavy chain variable region (V_{H}CEA) comprising an amino acid sequence that is at least about 95%, 96%, 97%, 98%, 99% or 100% identical to the amino acid sequence of SEQ ID NO:58, and a light chain variable region (V_{L}CEA) comprising an amino acid sequence that is at least about 95%, 96%, 97%, 98%, 99% or 100% identical to the amino acid sequence of SEQ ID NO:59, or a heavy chain variable region (V_{H}CEA) comprising an amino acid sequence that is at least about 95%, 96%, 97%, 98%, 99% or 100% identical to the amino acid sequence of SEQ ID NO:68, and a light chain variable region (V_{L}CEA) comprising an amino acid sequence that is at least about 95%, 96%, 97%, 98%, 99% or 100% identical to the amino acid sequence of SEQ ID NO:69. In one aspect, the antigen binding domain capable of specific binding to CEA comprises a heavy chain variable region (V_{H}CEA) comprising the amino acid sequence of SEQ ID NO:58, and a light chain variable region (V_{L}CEA) comprising the amino acid sequence of SEQ ID NO:59. In particular, the antigen binding domain capable of specific binding to CEA comprises a heavy chain variable region (V_{H}CEA) comprising the amino acid sequence of SEQ ID NO:68, and a light chain variable region (V_{L}CEA) comprising the amino acid sequence of SEQ ID NO:69.

In a further aspect, there is provided agonistic ICOS antigen binding molecule of any one of claims 1 to 3, wherein the antigen binding domain capable of specific binding to a tumor-associated antigen is an antigen binding domain capable of specific binding to Fibroblast Activation Protein (FAP). In one aspect, the antigen binding domain capable of specific binding to FAP comprises (a) a heavy chain variable region (V_{H}FAP) comprising (i) CDR-H1 comprising the amino acid sequence of SEQ ID NO:36, (ii) CDR-H2 comprising the amino acid sequence of SEQ ID NO:37, and (iii) CDR-H3 comprising the amino acid sequence of SEQ ID NO:38, and a light chain variable region (V_{L}FAP) comprising (iv) CDR-L1 comprising the amino acid sequence of SEQ ID NO:39, (v) CDR-L2 comprising the amino acid sequence of SEQ ID NO:40, and (vi) CDR-L3 comprising the amino acid sequence of SEQ ID NO:41, or
(b) a heavy chain variable region (V_{H}FAP) comprising (i) CDR-H1 comprising the amino acid sequence of SEQ ID NO:44, (ii) CDR-H2 comprising the amino acid sequence of SEQ ID NO:45, and (iii) CDR-H3 comprising the amino acid sequence of SEQ ID NO:46, and a light chain variable region (V_{L}FAP) comprising (iv) CDR-L1 comprising the amino acid sequence of SEQ ID NO:47, (v) CDR-L2 comprising the amino acid sequence of SEQ ID NO:48, and (vi) CDR-L3 comprising the amino acid sequence of SEQ ID NO:49. In one particular aspect, the antigen binding domain capable of specific binding to FAP comprises (a) a heavy chain variable region (V_{H}FAP) comprising (i) CDR-H1 comprising the amino acid sequence of SEQ ID NO:36, (ii) CDR-H2 comprising the amino acid sequence of SEQ ID NO:37, and (iii) CDR-H3 comprising the amino acid sequence of SEQ ID NO:38, and a light chain variable region (V_{L}FAP) comprising (iv) CDR-L1 comprising the amino acid sequence of SEQ ID NO:39, (v) CDR-L2 comprising the amino acid sequence of SEQ ID NO:40, and (vi) CDR-L3 comprising the amino acid sequence of SEQ ID NO:41.

In another aspect, provided is an agonistic ICOS antigen binding molecule comprising at least one antigen binding domain capable of specific binding to FAP, wherein the antigen binding domain capable of specific binding to FAP comprises (a) a heavy chain variable region (V_{H}FAP) comprising an amino acid sequence that is at least about 95%, 96%, 97%, 98%, 99% or 100% identical to the amino acid sequence of SEQ ID NO:42, and a light chain variable region (V_{L}FAP) comprising an amino acid sequence that is at least about 95%, 96%, 97%, 98%, 99% or 100% identical to the amino acid sequence of SEQ ID NO:43, or (b) a heavy chain variable region (V_{H}FAP) comprising an amino acid sequence that is at least about 95%, 96%, 97%, 98%, 99% or 100% identical to the amino acid sequence of SEQ ID NO:50, and a light chain variable region (V_{L}FAP) comprising an amino acid sequence that is at least about 95%, 96%, 97%, 98%, 99% or 100% identical to the amino acid sequence of SEQ ID NO:51. In a particular aspect, the antigen binding domain capable of specific binding to FAP comprises a heavy chain variable region (V_{H}FAP) comprising the amino acid sequence of SEQ ID NO:42, and a light chain variable region (V_{L}FAP) comprising the amino acid sequence of SEQ ID NO:43. In a further aspect, the antigen binding domain capable of specific binding to FAP comprises a heavy chain variable region (V_{H}FAP) comprising the amino acid sequence of SEQ ID NO:50, and a light chain variable region (V_{L}FAP) comprising the amino acid sequence of SEQ ID NO:51.

Furthermore, there is provided an agonistic ICOS-binding molecule comprising at least one antigen binding domain capable of specific binding to a tumor-associated antigen, wherein the antigen binding domain capable of specific binding to ICOS comprises
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

Thus, in one aspect, provided is an agonistic ICOS-binding molecule comprising at least one antigen binding domain capable of specific binding to a tumor-associated antigen and at least one antigen binding domain capable of specific binding to ICOS that originates from mouse immunization, comprising a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11. In particular, an agonistic ICOS-binding molecule comprising at least one antigen binding domain capable of specific binding to a tumor-associated antigen and at least one antigen binding domain capable of specific binding to ICOS that originates from mouse immunization is provided which comprises a heavy chain variable region (V_{H}ICOS) comprising the amino acid sequence of SEQ ID NO:10, and a light chain variable region (V_{L}ICOS) comprising the amino acid sequence of SEQ ID NO:11.

In a particular aspect, an agonistic ICOS-binding molecule comprising at least one antigen binding domain capable of specific binding to a tumor-associated antigen and at least one antigen binding domain capable of specific binding to ICOS that originates from mouse immunization is provided which comprises a heavy chain variable region (V_{H}ICOS) comprising the amino acid sequence of SEQ ID NO:296, and a light chain variable region (V_{L}ICOS) comprising the amino acid sequence of SEQ ID NO:297. In another aspect, a humanized variant thereof is provided, i.e. antigen binding domain capable of specific binding to ICOS which comprises a heavy chain variable region (V_{H}ICOS) comprising the amino acid sequence selected from the group consisting of SEQ ID NO:124, SEQ ID NO:125, SEQ ID NO:126, SEQ ID NO:127, SEQ ID NO:128, SEQ ID NO:129, SEQ ID NO:130 and SEQ ID NO:131, and a light chain variable region (V_{L}ICOS) comprising the amino acid sequence selected from the group consisting of SEQ ID NO:132, SEQ ID NO:133, SEQ ID NO:134 and SEQ ID NO:135.

In another aspect, the invention provides an agonistic ICOS-binding molecule comprising at least one antigen binding domain capable of specific binding to a tumor-associated antigen and at least one antigen binding domain capable of specific binding to ICOS that originates from rabbit immunization, comprising a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%. 98%, 99% or 100% identical to the amino acid sequence of SEQ ID NO:35.

In one aspect, the invention provides an agonistic ICOS-binding molecule comprising at least one antigen binding domain capable of specific binding to a tumor-associated antigen and at least one antigen binding domain capable of specific binding to ICOS that originates from rabbit immunization, comprising a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19. In particular, the antigen binding domain capable of specific binding to ICOS that originates from rabbit immunization comprises a heavy chain variable region (V_{H}ICOS) comprising the amino acid sequence of SEQ ID NO:18, and a light chain variable region (V_{L}ICOS) comprising the amino acid sequence of SEQ ID NO:19.

In a further aspect, the antigen binding domain capable of specific binding to ICOS that originates from rabbit immunization comprises a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35. In particular, the antigen binding domain capable of specific binding to ICOS that originates from rabbit immunization comprises a heavy chain variable region (V_{H}ICOS) comprising the amino acid sequence of SEQ ID NO:34, and a light chain variable region (V_{L}ICOS) comprising the amino acid sequence of SEQ ID NO:35. In one aspect, provided is a humanized variant thereof, comprising a heavy chain variable region (V_{H}ICOS) comprising an amino acid sequence selected from the group consisting of SEQ ID NO:136, SEQ ID NO:137, SEQ ID NO:138, SEQ ID NO:139 and SEQ ID NO:140, and a light chain variable region (V_{L}ICOS) comprising an amino acid sequence selected from the group consisting of SEQ ID NO:141, SEQ ID NO:142 and SEQ ID NO:143.

In a further aspect, the antigen binding domain capable of specific binding to ICOS that originates from rabbit immunization comprises a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27. In one particular aspect, the antigen binding domain capable of specific binding to ICOS that originates from rabbit immunization comprises a heavy chain variable region (V_{H}ICOS) comprising the amino acid sequence of SEQ ID NO:26, and a light chain variable region (V_{L}ICOS) comprising the amino acid sequence of SEQ ID NO:27. In a further aspect, the antigen binding domain capable of specific binding to ICOS that originates from rabbit immunization comprises a heavy chain variable region (V_{H}ICOS) comprising the amino acid sequence of SEQ ID NO:298, and a light chain variable region (V_{L}ICOS) comprising the amino acid sequence of SEQ ID NO:299. In another aspect, the antigen binding domain capable of specific binding to ICOS that originates from rabbit immunization comprises a heavy chain variable region (V_{H}ICOS) comprising the amino acid sequence of SEQ ID NO:300, and a light chain variable region (V_{L}ICOS) comprising the amino acid sequence of SEQ ID NO:301. In one aspect, provided is a humanized variant thereof, comprising a heavy chain variable region (V_{H}ICOS) comprising an amino acid sequence selected from the group consisting of SEQ ID NO:144, SEQ ID NO:145, SEQ ID NO:146, SEQ ID NO:147, SEQ ID NO:148, SEQ ID NO:149, SEQ ID NO:150 and SEQ ID NO:151, and a light chain variable region (V_{L}ICOS) comprising an amino acid sequence selected from the group consisting of SEQ ID NO:152 and SEQ ID NO:153.

In one aspect, the invention provides an agonistic ICOS antigen binding molecule as defined herein before, comprising
(a) one antigen binding domain capable of specific binding to a tumor-associated antigen,
(b) one Fab fragment capable of specific binding to ICOS, and
(c) a Fc domain composed of a first and a second subunit capable of stable association comprising one or more amino acid substitution that reduces the binding affinity of the antigen binding molecule to an Fc receptor and/or effector function. In particular, the agonistic ICOS antigen binding molecule comprises a Fc domain of human IgG1 subclass which comprises the amino acid mutations L234A, L235A and P329G (numbering according to Kabat EU index).

In another aspect, the invention provides an agonistic ICOS antigen binding molecule as defined herein before, comprising
(a) one antigen binding domain capable of specific binding to a tumor-associated antigen,
(b) two Fab fragments capable of specific binding to ICOS, and
(c) a Fc domain composed of a first and a second subunit capable of stable association comprising one or more amino acid substitution that reduces the binding affinity of the antigen binding molecule to an Fc receptor and/or effector function. In particular, the Fc domain of human IgG1 subclass comprises the amino acid mutations L234A, L235A and P329G (numbering according to Kabat EU index).

In particular aspects, the antigen binding domain capable of specific binding to a tumor-associated antigen is a crossFab fragment.

Exemplary Agonistic ICOS-Antibodies of the Invention

In a further aspect, provided is agonistic ICOS antigen binding molecule, in particular an antibody, comprising (a) a heavy chain variable region (V_{H}ICOS) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}ICOS) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}ICOS) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}ICOS) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or
(c) a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25, or
(d) a heavy chain variable region (V_{H}ICOS) comprising (i) CDR-H1 comprising the amino acid sequence of SEQ ID NO:28, (ii) CDR-H2 comprising the amino acid sequence of SEQ ID NO:29, and (iii) CDR-H3 comprising the amino acid sequence of SEQ ID NO:30, and a light chain variable region (V_{L}ICOS) comprising (iv) CDR-L1 comprising the amino acid sequence of SEQ ID NO:31, (v) CDR-L2 comprising the amino acid sequence of SEQ ID NO:32, and (vi) CDR-L3 comprising the amino acid sequence of SEQ ID NO:33.

In one aspect, the agonistic ICOS antigen binding molecule, in particular an antibody, is derived from mouse immunization and comprises a heavy chain variable region (V_{H}ICOS) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}ICOS) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9. In another aspect, the agonistic ICOS antigen binding molecule, in particular an antibody, is derived from rabbit immunization and comprises a heavy chain variable region (V_{H}ICOS) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}ICOS) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25, or a heavy chain variable region (V_{H}ICOS) comprising (i) CDR-H1 comprising the amino acid sequence of SEQ ID NO:28, (ii) CDR-H2 comprising the amino acid sequence of SEQ ID NO:29, and (iii) CDR-H3 comprising the amino acid sequence of SEQ ID NO:30, and a light chain variable region (V_{L}ICOS) comprising (iv) CDR-L1 comprising the amino acid sequence of SEQ ID NO:31, (v) CDR-L2 comprising the amino acid sequence of SEQ ID NO:32, and (vi) CDR-L3 comprising the amino acid sequence of SEQ ID NO:33.

In one aspect, provided is an agonistic ICOS antigen binding molecule, in particular an antibody, which comprises
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

Thus, in one aspect, provided is an agonistic ICOS antigen binding molecule, in particular an antibody, that originates from mouse immunization, comprising a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11. In particular, an agonistic ICOS-binding molecule comprising at least one antigen binding domain capable of specific binding to a tumor-associated antigen and at least one antigen binding domain capable of specific binding to ICOS that originates from mouse immunization is provided which comprises a heavy chain variable region (V_{H}ICOS) comprising the amino acid sequence of SEQ ID NO:10, and a light chain variable region (V_{L}ICOS) comprising the amino acid sequence of SEQ ID NO:11.

In a particular aspect, an agonistic ICOS antigen binding molecule that originates from mouse immunization is provided which comprises a heavy chain variable region (V_{H}ICOS) comprising the amino acid sequence of SEQ ID NO:296, and a light chain variable region (V_{L}ICOS) comprising the amino acid sequence of SEQ ID NO:297. In another aspect, a humanized variant thereof is provided, i.e. antigen binding domain capable of specific binding to ICOS which comprises a heavy chain variable region (V_{H}ICOS) comprising the amino acid sequence selected from the group consisting of SEQ ID NO:124, SEQ ID NO:125, SEQ ID NO:126, SEQ ID NO:127, SEQ ID NO:128, SEQ ID NO:129, SEQ ID NO:130 and SEQ ID NO:131, and a light chain variable region (V_{L}ICOS) comprising the amino acid sequence selected from the group consisting of SEQ ID NO:132, SEQ ID NO:133, SEQ ID NO:134 and SEQ ID NO:135.

In another aspect, the invention provides an agonistic ICOS antigen binding molecule that originates from rabbit immunization, comprising a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

In one aspect, the invention provides an agonistic ICOS antigen binding molecule that originates from rabbit immunization, comprising a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19. In particular, the antigen binding domain capable of specific binding to ICOS that originates from rabbit immunization comprises a heavy chain variable region (V_{H}ICOS) comprising the amino acid sequence of SEQ ID NO:18, and a light chain variable region (V_{L}ICOS) comprising the amino acid sequence of SEQ ID NO:19.

In a further aspect, the agonistic ICOS antigen binding molecule that originates from rabbit immunization comprises a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35. In particular, the antigen binding domain capable of specific binding to ICOS that originates from rabbit immunization comprises a heavy chain variable region (V_{H}ICOS) comprising the amino acid sequence of SEQ ID NO:34, and a light chain variable region (V_{L}ICOS) comprising the amino acid sequence of SEQ ID NO:35. In one aspect, provided is a humanized variant thereof, comprising a heavy chain variable region (V_{H}ICOS) comprising an amino acid sequence selected from the group consisting of SEQ ID NO:136, SEQ ID NO:137, SEQ ID NO:138, SEQ ID NO:139 and SEQ ID NO:140, and a light chain variable region (V_{L}ICOS) comprising an amino acid sequence selected from the group consisting of SEQ ID NO:141, SEQ ID NO:142 and SEQ ID NO:143.

In a further aspect, the agonistic ICOS antigen binding molecule comprises a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27. In one particular aspect, the agonistic ICOS antigen binding molecule that originates from rabbit immunization comprises a heavy chain variable region (V_{H}ICOS) comprising the amino acid sequence of SEQ ID NO:26, and a light chain variable region (V_{L}ICOS) comprising the amino acid sequence of SEQ ID NO:27. In a further aspect, the agonistic ICOS antigen binding molecule that originates from rabbit immunization comprises a heavy chain variable region (V_{H}ICOS) comprising the amino acid sequence of SEQ ID NO:298, and a light chain variable region (V_{L}ICOS) comprising the amino acid sequence of SEQ ID NO:299. In another aspect, the agonistic ICOS antigen binding molecule that originates from rabbit immunization comprises a heavy chain variable region (V_{H}ICOS) comprising the amino acid sequence of SEQ ID NO:300, and a light chain variable region (V_{L}ICOS) comprising the amino acid sequence of SEQ ID NO:301. In one aspect, provided is a humanized variant thereof, comprising a heavy chain variable region (V_{H}ICOS) comprising an amino acid sequence selected from the group consisting of SEQ ID NO:144, SEQ ID NO:145, SEQ ID NO:146, SEQ ID NO:147, SEQ ID NO:148, SEQ ID NO:149, SEQ ID NO:150 and SEQ ID NO:151, and a light chain variable region (V_{L}ICOS) comprising an amino acid sequence selected from the group consisting of SEQ ID NO:152 and SEQ ID NO:153.

In one aspect, the agonistic ICOS antigen binding molecule is full-length antibody. In another aspect, the agonistic ICOS antigen binding molecule is a Fab or crossFab fragment. In a particular aspect, the agonistic ICOS antigen binding molecule is a humanized antibody.

Exemplary Bispecific Agonistic ICOS-Antigen Binding Molecules of the Invention

In one aspect, the invention provides bispecific agonistic ICOS-binding molecules, comprising (a) one antigen binding domain capable of specific binding to ICOS, and (b) one antigen binding domain capable of specific binding to a tumor-associated antigen, and (c) a Fc domain. Thus, in this case the agonistic ICOS-binding molecule is monovalent for the binding to ICOS and monovalent for the binding to the tumor-associated antigen (1+1 format).

In a particular aspect, provided is an agonistic ICOS-binding molecule, wherein said molecule comprises (a) a first Fab fragment capable of specific binding to ICOS, (b) a second Fab fragment capable of specific binding to a tumor-associated antigen, and (c) a Fc domain composed of a first and a second subunit capable of stable association with each other.

In one aspect, provided is an agonistic ICOS-binding molecule, wherein said molecule comprises (a) a first Fab fragment capable of specific binding to ICOS, (b) a second antigen binding domain capable of specific binding to a tumor-associated antigen comprising a VH and VL domain, and (c) a Fc domain composed of a first and a second subunit capable of stable association with each other, and wherein one of the VH and VL domain of the antigen binding domain capable of specific binding to a tumor-associated antigen is fused to the C-terminus of the first subunit of the Fc domain and the other one of VH and VL is fused to the C-terminus of the second subunit of the Fc domain. Such a molecule is termed 1+1 head-to-tail.

In another aspect, the invention provides bispecific agonistic ICOS-binding molecules, comprising (a) two antigen binding domains capable of specific binding to ICOS, and (b) one antigen binding domain capable of specific binding to a tumor-associated antigen, and (c) a Fc domain. Thus, in this case the agonistic ICOS-binding molecule is bivalent for the binding to ICOS and monovalent for the binding to the tumor-associated antigen (2+1 format).

In one aspect, provided is an agonistic ICOS-binding molecule, wherein said molecule comprises (a) two Fab fragments capable of specific binding to ICOS, (b) a second antigen binding domain capable of specific binding to a tumor-associated antigen comprising a VH and VL domain, and (c) a Fc domain composed of a first and a second subunit capable of stable association with each other, and wherein one of the VH and VL domain of the antigen binding domain capable of specific binding to a tumor-associated antigen is fused to the C-terminus of the first subunit of the Fc domain and the other one of VH and VL is fused to the C-terminus of the second subunit of the Fc domain. Such a molecule is termed 2+1.

In another aspect, the invention provides an agonistic ICOS-binding molecule, comprising (a) a first Fab fragment capable of specific binding to ICOS, (b) a second Fab fragment capable of specific binding to a tumor-associated antigen, (c) a third Fab fragment capable of specific binding to ICOS, and (d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, and wherein in the second Fab fragment capable of specific binding to a target cell antigen (i) the variable regions VL and VH of the Fab light chain and Fab heavy chain are replaced by each other.

In yet another aspect, the invention provides an agonistic ICOS-binding molecule, comprising (a) a first Fab fragment capable of specific binding to ICOS, (b) a second Fab fragment capable of specific binding to a tumor-associated antigen, (c) a third Fab fragment capable of specific binding to ICOS, and (d) a Fc domain composed of a first and a second subunit capable of stable association, wherein the first Fab fragment (a) is fused at the C-terminus of the Fab heavy chain to the N-terminus of the Fab heavy chain of the second Fab fragment (b), which is in turn fused at its C-terminus to the N-terminus of the first Fc domain subunit, and the third Fab fragment (c) is fused at the C-terminus of the Fab heavy chain to the N-terminus of the second Fc domain subunit, and wherein in the second Fab fragment capable of specific binding to a target cell antigen (i) the variable regions VL and VH of the Fab light chain and Fab heavy chain are replaced by each other.

Fc Domain Modifications Reducing Fc Receptor Binding and/or Effector Function

The Fc domain of the agonistic ICOS-binding molecules of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

Thus, the agonistic ICOS-binding molecule comprising at least one antigen binding domain that binds to a tumor-associated antigen comprises an IgG Fc domain, specifically an IgG1 Fc domain or an IgG4 Fc domain. More particularly, the agonistic ICOS-binding molecule comprising at least one antigen binding domain that binds to a tumor-associated antigen comprises an IgG1 Fc domain.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular aspects, the Fc domain of the agonistic ICOS-binding molecules of the invention exhibits reduced binding affinity to an Fc receptor and/or reduced effector function, as compared to a native IgG1 Fc domain. In one aspect, the Fc domain does not substantially bind to an Fc receptor and/or does not induce effector function. In a particular aspect, the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc domain of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides an antibody, wherein the Fc domain comprises one or more amino acid substitution that reduces binding to an Fc receptor, in particular towards Fcγ receptor.

In one aspect, the Fc domain of the antibody of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is an antibody according to the invention which comprises an Fc domain with the amino acid substitutions L234A, L235A and P329G (“P329G LALA”, Kabat EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position S228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Antibodies with increased half lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G., Nature 322 (1988) 738-740; U.S. Pat. Nos. 5,648,260; 5,624,821; and WO 94/29351 concerning other examples of Fc region variants.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor. Effector function of an Fc domain, or bispecific antigen binding molecules of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

The following section describes preferred aspects of the agonistic ICOS-binding molecules of the invention comprising Fc domain modifications that reduce Fc receptor binding and/or effector function. In one aspect, the invention relates to the bispecific antigen binding molecule (a) at least one antigen binding domain capable of specific binding to ICOS, (b) at least one antigen binding domain capable of specific binding to a tumor-associated antigen, and (c) a Fc domain composed of a first and a second subunit capable of stable association, wherein the Fc domain comprises one or more amino acid substitution that reduces the binding affinity of the antibody to an Fc receptor, in particular towards Fcγ receptor. In another aspect, the invention relates to the agonistic ICOS-binding molecule comprising (a) at least one antigen binding domain capable of specific binding to ICOS, (b) at least one antigen binding domain capable of specific binding to a target cell antigen, and (c) a Fc domain composed of a first and a second subunit capable of stable association, wherein the Fc domain comprises one or more amino acid substitution that reduces effector function. In particular aspect, the Fc domain is of human IgG1 subclass with the amino acid mutations L234A, L235A and P329G (numbering according to Kabat EU index).

In one aspect of the invention, the Fc region comprises an amino acid substitution at positions D265, and P329. In some aspects, the Fc region comprises the amino acid substitutions D265A and P329G (“DAPG”) in the CH2 domain. In one such embodiment, the Fc region is an IgG1 Fc region, particularly a mouse IgG1 Fc region. DAPG mutations are described e.g. in WO 2016/030350 A1, and can be introduced in CH2 regions of heavy chains to abrogate binding of antigen binding molecules to murine Fc gamma receptors.

Fc Domain Modifications Promoting Heterodimerization

The agonistic ICOS-binding molecules of the invention comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the agonistic ICOS-binding molecules of the invention in recombinant production, it will thus be advantageous to introduce in the Fc domain of the bispecific antigen binding molecules of the invention a modification promoting the association of the desired polypeptides.

Accordingly, in particular aspects the invention relates to agonistic ICOS-binding molecules comprising (a) at least one antigen binding domain capable of specific binding to ICOS, (b) at least one antigen binding domain capable of specific binding to a tumor-associated antigen, and (c) a Fc domain composed of a first and a second subunit capable of stable association with each other, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, the invention relates to the agonistic ICOS-binding molecule comprising (a) at least one antigen binding domain capable of specific binding to ICOS, (b) at least one antigen binding domain capable of specific binding to a tumor-associated antigen, and (c) a Fc domain composed of a first and a second subunit capable of stable association with each other, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the agonistic ICOS-binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in the formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In one aspect, the first subunit of the Fc region comprises aspartic acid residues (D) at positions 392 and 409, and the second subunit of the Fc region comprises lysine residues (K) at positions 356 and 399. In some embodiments, in the first subunit of the Fc region the lysine residues at positions 392 and 409 are replaced with aspartic acid residues (K392D, K409D), and in the second subunit of the Fc region the glutamate residue at position 356 and the aspartic acid residue at position 399 are replaced with lysine residues (E356K, D399K). “DDKK” knob-into-hole technology is described e.g. in WO 2014/131694 A1, and favours the assembly of the heavy chains bearing subunits providing the complementary amino acid residues.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the bispecific antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

Exemplary Agonistic ICOS Antigen Binding Molecules of the Invention

In one aspect, provided is an agonistic ICOS-binding molecule, comprising at least one antigen binding domain capable of specific binding to a tumor-associated antigen comprising a heavy chain variable region (V_{H}FAP) comprising an amino acid sequence of SEQ ID NO:42 and a light chain variable region (V_{L}FAP) comprising an amino acid sequence of SEQ ID NO:43, and at least one antigen binding domain capable of specific binding to ICOS which comprises
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%. 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

More particularly, provided is a bispecific antigen binding molecule, wherein said molecule comprises
(i) a first Fab fragment capable of specific binding to FAP, comprising a heavy chain variable region (V_{H}FAP) comprising an amino acid sequence of SEQ ID NO:42 and a light chain variable region (V_{L}FAP) comprising an amino acid sequence of SEQ ID NO:43 or comprising a heavy chain variable region (V_{H}FAP) comprising an amino acid sequence of SEQ ID NO:50 and a light chain variable region (V_{L}FAP) comprising an amino acid sequence of SEQ ID NO:51, and
(ii) a second Fab fragment capable of specific binding to ICOS, comprising
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%. 96%, 97%. 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

In one aspect, provided is an agonistic ICOS-binding molecule, comprising one antigen binding domain capable of specific binding to a tumor-associated antigen comprising a heavy chain variable region (V_{H}FAP) comprising an amino acid sequence of SEQ ID NO:42 and a light chain variable region (V_{L}FAP) comprising an amino acid sequence of SEQ ID NO:41, and at least one antigen binding domain capable of specific binding to ICOS comprising a heavy chain variable region (V_{H}ICOS) comprising the amino acid sequence of SEQ ID NO:18 and a light chain variable region (V_{L}ICOS) comprising the amino acid sequence of SEQ ID NO:19.

More particularly, provided is a bispecific antigen binding molecule, wherein said molecule comprises (i) a first Fab fragment capable of specific binding to FAP, comprising a heavy chain variable region (V_{H}FAP) comprising an amino acid sequence of SEQ ID NO:42 and a light chain variable region (V_{L}FAP) comprising an amino acid sequence of SEQ ID NO:43 and (ii) a second Fab fragment capable of specific binding to ICOS comprising a heavy chain variable region (V_{H}ICOS) comprising the amino acid sequence of SEQ ID NO:18 and a light chain variable region (V_{L}ICOS) comprising the amino acid sequence of SEQ ID NO:19.

In one aspect, provided is a bispecific antigen binding molecule comprising a first heavy chain (HC1) comprising the amino acid sequence of SEQ ID NO:91, a second heavy chain (HC2) comprising the amino acid sequence of SEQ ID NO:93, a first light chain comprising the amino acid sequence of SEQ ID NO:92 and a second light chain comprising the amino acid sequence of SEQ ID NO:94.

In another aspect, provided is a bispecific antigen binding molecule comprising a first heavy chain (HC1) comprising the amino acid sequence of SEQ ID NO:95, a second heavy chain (HC2) comprising the amino acid sequence of SEQ ID NO:96, and a light chain comprising the amino acid sequence of SEQ ID NO:94.

In a further aspect, the molecule comprises two Fab fragment capable of specific binding to ICOS. In a particular aspect, provided is a molecule, comprising
(i) a first antigen binding domain capable of specific binding to FAP, comprising a heavy chain variable region (V_{H}FAP) comprising an amino acid sequence of SEQ ID NO:42 and a light chain variable region (V_{L}FAP) comprising an amino acid sequence of SEQ ID NO:43 or comprising a heavy chain variable region (V_{H}FAP) comprising an amino acid sequence of SEQ ID NO:50 and a light chain variable region (V_{L}FAP) comprising an amino acid sequence of SEQ ID NO:51, and
(ii) two Fab fragments capable of specific binding to ICOS, each comprising
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

In a particular aspect, provided is a bispecific agonistic ICOS-binding molecule comprising a first heavy chain (HC1) comprising the amino acid sequence of SEQ ID NO:97, a second heavy chain (HC2) comprising the amino acid sequence of SEQ ID NO:96, and two light chains comprising the amino acid sequence of SEQ ID NO:94.

In another particular aspect, provided is a bispecific agonistic ICOS-binding molecule comprising a first heavy chain (HC1) comprising the amino acid sequence of SEQ ID NO:98, a second heavy chain (HC2) comprising the amino acid sequence of SEQ ID NO:99, and two light chains comprising the amino acid sequence of SEQ ID NO:100.

In another particular aspect, provided is a bispecific agonistic ICOS-binding molecule comprising a first heavy chain (HC1) comprising the amino acid sequence of SEQ ID NO:98, a second heavy chain (HC2) comprising the amino acid sequence of SEQ ID NO:101, and two light chains comprising the amino acid sequence of SEQ ID NO:100.

In another particular aspect, provided is a bispecific agonistic ICOS-binding molecule comprising a first heavy chain (HC1) comprising the amino acid sequence of SEQ ID NO:102, a second heavy chain (HC2) comprising the amino acid sequence of SEQ ID NO:103, and two light chains comprising the amino acid sequence of SEQ ID NO:104.

In yet another aspect, provided is a bispecific agonistic ICOS-binding molecule comprising a first heavy chain (HC1) comprising the amino acid sequence of SEQ ID NO:105, a second heavy chain (HC2) comprising the amino acid sequence of SEQ ID NO:106, and two light chains comprising the amino acid sequence of SEQ ID NO:107.

In another aspect, provided is a bispecific agonistic ICOS-binding molecule comprising a first heavy chain (HC1) comprising the amino acid sequence of SEQ ID NO:108, a second heavy chain (HC2) comprising the amino acid sequence of SEQ ID NO:109, and two light chains comprising the amino acid sequence of SEQ ID NO:107.

In another aspect, provided is a bispecific agonistic ICOS-binding molecule comprising a first heavy chain (HC1) comprising the amino acid sequence of SEQ ID NO:110, a second heavy chain (HC2) comprising the amino acid sequence of SEQ ID NO:111, and two light chains comprising the amino acid sequence of SEQ ID NO:107.

In a further aspect, the molecules are provided that comprise two Fab fragments capable of specific binding to ICOS and a Fab fragment capable of specific binding to FAP.

In a further aspect, the molecule comprises two Fab fragment capable of specific binding to ICOS.

In a particular aspect, provided is a molecule, comprising
(i) a first Fab fragment capable of specific binding to FAP, comprising a heavy chain variable region (V_{H}FAP) comprising an amino acid sequence of SEQ ID NO:42 and a light chain variable region (V_{L}FAP) comprising an amino acid sequence of SEQ ID NO:43 or comprising a heavy chain variable region (V_{H}FAP) comprising an amino acid sequence of SEQ ID NO:50 and a light chain variable region (V_{L}FAP) comprising an amino acid sequence of SEQ ID NO:51, and
(ii) two Fab fragments capable of specific binding to ICOS, each comprising
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

In one aspect, provided is a bispecific agonistic ICOS-binding molecule comprising a first heavy chain (HC1) comprising the amino acid sequence of SEQ ID NO:112, a second heavy chain (HC2) comprising the amino acid sequence of SEQ ID NO:114, two first light chain comprising the amino acid sequence of SEQ ID NO:113 and a second light chain comprising the amino acid sequence of SEQ ID NO:115.

In another aspect, provided is a bispecific agonistic ICOS-binding molecule comprising a first heavy chain (HC1) comprising the amino acid sequence of SEQ ID NO:116, a second heavy chain (HC2) comprising the amino acid sequence of SEQ ID NO:118, two first light chain comprising the amino acid sequence of SEQ ID NO:117 and a second light chain comprising the amino acid sequence of SEQ ID NO:119.

In one aspect, provided is an agonistic ICOS-binding molecule, comprising at least one antigen binding domain capable of specific binding to CEA comprising a heavy chain variable region (V_{H}CEA) comprising an amino acid sequence of SEQ ID NO:68 and a light chain variable region (V_{L}CEA) comprising an amino acid sequence of SEQ ID NO:69, and at least one antigen binding domain capable of specific binding to ICOS which comprises
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%. 96%, 97%. 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

More particularly, provided is a bispecific antigen binding molecule, wherein said molecule comprises
(i) a first Fab fragment capable of specific binding to CEA, comprising a heavy chain variable region (V_{H}CEA) comprising an amino acid sequence of SEQ ID NO:68 and a light chain variable region (V_{L}CEA) comprising an amino acid sequence of SEQ ID NO:69, and
(ii) a second Fab fragment capable of specific binding to ICOS, comprising
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

In one aspect, provided is an agonistic ICOS-binding molecule, comprising one antigen binding domain capable of specific binding to a tumor-associated antigen comprising a heavy chain variable region (V_{H}CEA) comprising an amino acid sequence of SEQ ID NO:68 and a light chain variable region (V_{L}CEA) comprising an amino acid sequence of SEQ ID NO:69, and at least one antigen binding domain capable of specific binding to ICOS comprising a heavy chain variable region (V_{H}ICOS) comprising the amino acid sequence of SEQ ID NO:18 and a light chain variable region (V_{L}ICOS) comprising the amino acid sequence of SEQ ID NO:19.

More particularly, provided is a bispecific antigen binding molecule, wherein said molecule comprises (i) a first Fab fragment capable of specific binding to FAP, comprising a heavy chain variable region (V_{H}CEA) comprising an amino acid sequence of SEQ ID NO:68 and a light chain variable region (V_{L}CEA) comprising an amino acid sequence of SEQ ID NO:69 and (ii) a second Fab fragment capable of specific binding to ICOS comprising a heavy chain variable region (V_{H}ICOS) comprising the amino acid sequence of SEQ ID NO:18 and a light chain variable region (V_{L}ICOS) comprising the amino acid sequence of SEQ ID NO:19.

In one aspect, provided is a bispecific antigen binding molecule comprising a first heavy chain (HC1) comprising the amino acid sequence of SEQ ID NO:202, a second heavy chain (HC2) comprising the amino acid sequence of SEQ ID NO:204, a first light chain comprising the amino acid sequence of SEQ ID NO:203 and a second light chain comprising the amino acid sequence of SEQ ID NO:205.

In one aspect, provided is a bispecific antigen binding molecule comprising a first heavy chain (HC1) comprising the amino acid sequence of SEQ ID NO:206, a second heavy chain (HC2) comprising the amino acid sequence of SEQ ID NO:208, a first light chain comprising the amino acid sequence of SEQ ID NO:207 and a second light chain comprising the amino acid sequence of SEQ ID NO:209.

In another aspect, provided is a bispecific antigen binding molecule comprising a first heavy chain (HC1) comprising the amino acid sequence of SEQ ID NO:206, a second heavy chain (HC2) comprising the amino acid sequence of SEQ ID NO:210, a first light chain comprising the amino acid sequence of SEQ ID NO:207 and a second light chain comprising the amino acid sequence of SEQ ID NO:211.

Exemplary Anti-CEA/Anti-CD3 Bispecific Antibodies for Use in the Invention

The present invention relates to anti-CEA/anti-CD3 bispecific antibodies and their use in combination with agonistic ICOS antigen binding molecules, in particular to their use in a method for treating or delaying progression of cancer, more particularly for treating or delaying progression of solid tumors. The anti-CEA/anti-CD3 bispecific antibodies as used herein are bispecific antibodies comprising a first antigen binding domain that binds to CD3, and a second antigen binding domain that binds to CEA.

Thus, the anti-CEA/anti-CD3 bispecific antibody as used herein comprises a first antigen binding domain comprising a heavy chain variable region (V_{H}CD3) and a light chain variable region (V_{L}CD3), and a second antigen binding domain comprising a heavy chain variable region (V_{H}CEA) and a light chain variable region (V_{L}CEA).

In a particular aspect, the anti-CEA/anti-CD3 bispecific antibody for use in the combination comprises a first antigen binding domain comprising a heavy chain variable region (V_{H}CD3) comprising CDR-H1 sequence of SEQ ID NO:218, CDR-H2 sequence of SEQ ID NO:219, and CDR-H3 sequence of SEQ ID NO:220; and/or a light chain variable region (V_{L}CD3) comprising CDR-L1 sequence of SEQ ID NO:221, CDR-L2 sequence of SEQ ID NO:222, and CDR-L3 sequence of SEQ ID NO:223. More particularly, the anti-CEA/anti-CD3 bispecific antibody comprises a first antigen binding domain comprising a heavy chain variable region (V_{H}CD3) that is at least 90%, 95%, 96%, 97%, 98%, or 99% identical to the amino acid sequence of SEQ ID NO:224 and/or a light chain variable region (V_{L}CD3) that is at least 90%, 95%, 96%, 97%, 98%, or 99% identical to the amino acid sequence of SEQ ID NO:225. In a further aspect, the anti-CEA/anti-CD3 bispecific antibody comprises a heavy chain variable region (V_{H}CD3) comprising the amino acid sequence of SEQ ID NO:224 and/or a light chain variable region (V_{L}CD3) comprising the amino acid sequence of SEQ ID NO:225.

In one aspect, the antibody that specifically binds to CD3 is a full-length antibody. In one aspect, the antibody that specifically binds to CD3 is an antibody of the human IgG class, particularly an antibody of the human IgG1 class. In one aspect, the antibody that specifically binds to CD3 is an antibody fragment, particularly a Fab molecule or a scFv molecule, more particularly a Fab molecule. In a particular aspect, the antibody that specifically binds to CD3 is a crossover Fab molecule wherein the variable domains or the constant domains of the Fab heavy and light chain are exchanged (i.e. replaced by each other). In one aspect, the antibody that specifically binds to CD3 is a humanized antibody.

In another aspect, the anti-CEA/anti-CD3 bispecific antibody comprises a second antigen binding domain comprising
(a) a heavy chain variable region (V_{H}CEA) comprising CDR-H1 sequence of SEQ ID NO:226, CDR-H2 sequence of SEQ ID NO:227, and CDR-H3 sequence of SEQ ID NO:228, and/or a light chain variable region (V_{L}CEA) comprising CDR-L1 sequence of SEQ ID NO:229, CDR-L2 sequence of SEQ ID NO:230 and CDR-L3 sequence of SEQ ID NO:231, or
(b) a heavy chain variable region (V_{H}CEA) comprising CDR-H1 sequence of SEQ ID NO:234, CDR-H2 sequence of SEQ ID NO:235, and CDR-H3 sequence of SEQ ID NO:236, and/or a light chain variable region (V_{L}CEA) comprising CDR-L1 sequence of SEQ ID NO:237, CDR-L2 sequence of SEQ ID NO:238, and CDR-L3 sequence of SEQ ID NO:239.

More particularly, the anti-CEA/anti-CD3 bispecific comprises a second antigen binding domain comprising a heavy chain variable region (V_{H}CEA) that is at least 90%, 95%, 96%, 97%, 98%, or 99% identical to the amino acid sequence of SEQ ID NO:232 and/or a light chain variable region (V_{L}CEA) that is at least 90%, 95%, 96%, 97%, 98%, or 99% identical to the amino acid sequence of SEQ ID NO:233. In a further aspect, the anti-CEA/anti-CD3 bispecific comprises a second antigen binding domain comprising a heavy chain variable region (V_{H}CEA) comprising the amino acid sequence of SEQ ID NO:232 and/or a light chain variable region (V_{L}CEA) comprising the amino acid sequence of SEQ ID NO:233. In another aspect, the anti-CEA/anti-CD3 bispecific comprises a second antigen binding domain comprising a heavy chain variable region (V_{H}CEA) that is at least 90%, 95%, 96%, 97%, 98%, or 99% identical to the amino acid sequence of SEQ ID NO:240 and/or a light chain variable region (V_{L}CEA) that is at least 90%, 95%, 96%, 97%, 98%, or 99% identical to the amino acid sequence of SEQ ID NO:241. In a further aspect, the anti-CEA/anti-CD3 bispecific comprises a second antigen binding domain comprising a heavy chain variable region (V_{H}CEA) comprising the amino acid sequence of SEQ ID NO:240 and/or a light chain variable region (V_{L}CEA) comprising the amino acid sequence of SEQ ID NO:241.

In another particular aspect, the anti-CEA/anti-CD3 bispecific antibody comprises a third antigen binding domain that binds to CEA. In particular, the anti-CEA/anti-CD3 bispecific antibody comprises a third antigen binding domain comprising
(a) a heavy chain variable region (V_{H}CEA) comprising CDR-H1 sequence of SEQ ID NO:226, CDR-H2 sequence of SEQ ID NO:227, and CDR-H3 sequence of SEQ ID NO:228, and/or a light chain variable region (V_{L}CEA) comprising CDR-L1 sequence of SEQ ID NO:229, CDR-L2 sequence of SEQ ID NO:230, and CDR-L3 sequence of SEQ ID NO:231, or
(b) a heavy chain variable region (V_{H}CEA) comprising CDR-H1 sequence of SEQ ID NO:234, CDR-H2 sequence of SEQ ID NO:235, and CDR-H3 sequence of SEQ ID NO:236, and/or a light chain variable region (V_{L}CEA) comprising CDR-L1 sequence of SEQ ID NO:237, CDR-L2 sequence of SEQ ID NO:238, and CDR-L3 sequence of SEQ ID NO:239.

More particularly, the anti-CEA/anti-CD3 bispecific comprises a third antigen binding domain comprising a heavy chain variable region (V_{H}CEA) that is at least 90%, 95%, 96%, 97%, 98%, or 99% identical to the amino acid sequence of SEQ ID NO:232 and/or a light chain variable region (V_{L}CEA) that is at least 90%, 95%. 96%, 97%. 98%, or 99% identical to the amino acid sequence of SEQ ID NO:233. In a further aspect, the anti-CEA/anti-CD3 bispecific comprises a third antigen binding domain comprising a heavy chain variable region (V_{H}CEA) comprising the amino acid sequence of SEQ ID NO:232 and/or a light chain variable region (V_{L}CEA) comprising the amino acid sequence of SEQ ID NO:233. In another particular aspect, the anti-CEA/anti-CD3 bispecific comprises a third antigen binding domain comprising a heavy chain variable region (V_{H}CEA) that is at least 90%, 95%, 96%, 97%, 98%, or 99% identical to the amino acid sequence of SEQ ID NO:240 and/or a light chain variable region (V_{L}CEA) that is at least 90%, 95%, 96%, 97%, 98%, or 99% identical to the amino acid sequence of SEQ ID NO:241. In a further aspect, the anti-CEA/anti-CD3 bispecific comprises a third antigen binding domain comprising a heavy chain variable region (V_{H}CEA) comprising the amino acid sequence of SEQ ID NO:240 and/or a light chain variable region (V_{L}CEA) comprising the amino acid sequence of SEQ ID NO:241.

In a further aspect, the anti-CEA/anti-CD3 bispecific antibody is a bispecific antibody, wherein the first antigen binding domain is a cross-Fab molecule wherein the variable domains or the constant domains of the Fab heavy and light chain are exchanged, and the second and third, if present, antigen binding domain is a conventional Fab molecule.

In another aspect, the anti-CEA/anti-CD3 bispecific antibody is bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

The Fab molecules may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers. “n” is generally an integer from 1 to 10, typically from 2 to 4. In one embodiment said peptide linker has a length of at least 5 amino acids, in one embodiment a length of 5 to 100, in a further embodiment of 10 to 50 amino acids. In one embodiment said peptide linker is (GxS)ₙ or (GxS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=2. In one embodiment said peptide linker is (G₄S)₂. A particularly suitable peptide linker for fusing the Fab light chains of the first and the second Fab molecule to each other is (G₄S)₂. An exemplary peptide linker suitable for connecting the Fab heavy chains of the first and the second Fab fragments comprises the sequence (D)-(G₄S)₂. Another suitable such linker comprises the sequence (G₄S)₄. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a further aspect, the anti-CEA/anti-CD3 bispecific antibody comprises an Fc domain comprising one or more amino acid substitutions that reduce binding to an Fc receptor and/or effector function. In particular, the anti-CEA/anti-CD3 bispecific antibody comprises an IgG1 Fc domain comprising the amino aciod substitutions L234A, L235A and P329G.

In a particular aspect, the anti-CEA/anti-CD3 bispecific antibody comprises a polypeptide that is at least 95%, 96%, 97%, 98%, or 99% identical to the sequence of SEQ ID NO: 242, a polypeptide that is at least 95%, 96%, 97%, 98%, or 99% identical to the sequence of SEQ ID NO: 243, a polypeptide that is at least 95%, 96%, 97%, 98%, or 99% identical to the sequence of SEQ ID NO: 244, and a polypeptide that is at least 95%, 96%, 97%, 98%, or 99% identical to the sequence of SEQ ID NO: 245. In a further particular embodiment, the bispecific antibody comprises a polypeptide sequence of SEQ ID NO: 242, a polypeptide sequence of SEQ ID NO: 243, a polypeptide sequence of SEQ ID NO: 244 and a polypeptide sequence of SEQ ID NO: 245 (CEA CD3 TCB).

In a further particular aspect, the anti-CEA/anti-CD3 bispecific antibody comprises a polypeptide that is at least 95%, 96%, 97%, 98%, or 99% identical to the sequence of SEQ ID NO:246, a polypeptide that is at least 95%, 96%, 97%, 98%, or 99% identical to the sequence of SEQ ID NO:247, a polypeptide that is at least 95%, 96%, 97%, 98%, or 99% identical to the sequence of SEQ ID NO:248, and a polypeptide that is at least 95%, 96%, 97%, 98%, or 99% identical to the sequence of SEQ ID NO:249. In a further particular embodiment, the bispecific antibody comprises a polypeptide sequence of SEQ ID NO:246, a polypeptide sequence of SEQ ID NO:247, a polypeptide sequence of SEQ ID NO:248 and a polypeptide sequence of SEQ ID NO:249 (CEACAM5 CD3 TCB).

Particular bispecific antibodies are described in PCT publication no. WO 2014/131712 A1.

In a further aspect, the anti-CEA/anti-CD3 bispecific antibody may also comprise a bispecific T cell engager (BiTE®). In a further aspect, the anti-CEA/anti-CD3 bispecific antibody is a bispecific antibody as described in WO 2007/071426 or WO 2014/131712. In another aspect, the bispecific antibody is MEDI565.

In another aspect, the invention relates to a murine anti-CEA/anti-CD3 bispecific antibody comprising a first antigen binding domain comprising a heavy chain variable region (V_{H}muCD3) and a light chain variable region (V_{L}muCD3), a second antigen binding domain comprising a heavy chain variable region (V_{H}muCEA) and a light chain variable region (V_{L}muCEA) and a third antigen binding domain comprising a heavy chain variable region (V_{H}muCEA) and a light chain variable region (V_{L}muCEA).

In a particular aspect, the murine anti-CEA/anti-CD3 bispecific antibody comprises a polypeptide that is at least 95%, 96%, 97%, 98%, or 99% identical to the sequence of SEQ ID NO:250, a polypeptide that is at least 95%, 96%, 97%, 98%, or 99% identical to the sequence of SEQ ID NO: 251, a polypeptide that is at least 95%, 96%, 97%, 98%, or 99% identical to the sequence of SEQ ID NO:252, a polypeptide that is at least 95%, 96%, 97%, 98%, or 99% identical to the sequence of SEQ ID NO:253. In a further particular aspect, the murine anti-CEA/anti-CD3 bispecific antibody comprises a polypeptide sequence of SEQ ID NO:250, a polypeptide sequence of SEQ ID NO:251, a polypeptide sequence of SEQ ID NO:252 and a polypeptide sequence of SEQ ID NO:253 (mu CEA CD3 TCB).

Agents Blocking PD-L1/PD-1 Interaction for Use in the Invention

In one aspect of the invention, the agonistic ICOS antigen binding molecules are for use in combination with an agent blocking PD-L1/PD-1 interaction. In another aspect, the agonistic ICOS antigen binding molecules are for use in combination with agent blocking PD-L1/PD-1 interaction and a CD3 bispecific antibody. In all these aspects, an agent blocking PD-L1/PD-1 interaction is a PD-L1 binding antagonist or a PD-1 binding antagonist. In particular, the agent blocking PD-L1/PD-1 interaction is an anti-PD-L1 antibody or an anti-PD-1 antibody.

The term “PD-L1”, also known as CD274 or B7-H1, refers to any native PD-L1 from any vertebrate source, including mammals such as primates (e.g. humans) non-human primates (e.g. cynomolgus monkeys) and rodents (e.g. mice and rats), in particular to “human PD-L1”. The amino acid sequence of complete human PD-L1 is shown in UniProt (www.uniprot.org) accession no. Q9NZQ7 (SEQ ID NO:286). The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In particular, a PD-L1 binding antagonist is an anti-PD-L1 antibody. The term “anti-PD-L1 antibody” or “antibody binding to human PD-L1” or “antibody that specifically binds to human PD-L1” or “antagonistic anti-PD-L1” refers to an antibody specifically binding to the human PD-L1 antigen with a binding affinity of KD-value of 1.0×10⁻⁸ mol/l or lower, in one aspect of a KD-value of 1.0×10⁻⁹ mol/l or lower. The binding affinity is determined with a standard binding assay, such as surface plasmon resonance technique (BIAcore®, GE-Healthcare Uppsala, Sweden).

In a particular aspect, the agent blocking PD-L1/PD-1 interaction is an anti-PD-L1 antibody. In a specific aspect, the anti-PD-L1 antibody is selected from the group consisting of atezolizumab (MPDL3280A, RG7446), durvalumab (MEDI4736), avelumab (MSB0010718C) and MDX-1105. In a specific aspect, an anti-PD-L1 antibody is YW243.55.S70 described herein. In another specific aspect, an anti-PD-L1 antibody is MDX-1105 described herein. In still another specific aspect, an anti-PD-L1 antibody is MEDI4736 (durvalumab). In yet a further aspect, an anti-PD-L1 antibody is MSB0010718C (avelumab). More particularly, the agent blocking PD-L1/PD-1 interaction is atezolizumab (MPDL3280A). In another aspect, the agent blocking PD-L1/PD-1 interaction is an anti-PD-L1 antibody comprising a heavy chain variable domain VH(PDL-1) of SEQ ID NO:288 and a light chain variable domain VL(PDL-1) of SEQ ID NO:289. In another aspect, the agent blocking PD-L1/PD-1 interaction is an anti-PD-L1 antibody comprising a heavy chain variable domain VH(PDL-1) of SEQ ID NO:290 and a light chain variable domain VL(PDL-1) of SEQ ID NO:291.

The term “PD-1”, also known as CD279, PD1 or programmed cell death protein 1, refers to any native PD-L1 from any vertebrate source, including mammals such as primates (e.g. humans) non-human primates (e.g. cynomolgus monkeys) and rodents (e.g. mice and rats), in particular to the human protein PD-1 with the amino acid sequence as shown in UniProt (www.uniprot.org) accession no. Q15116 (SEQ ID NO:287). The term “PD-1 binding antagonist” refers to a molecule that inhibits the binding of PD-1 to its ligand binding partners. In some embodiments, the PD-1 binding antagonist inhibits the binding of PD-1 to PD-L1. In some embodiments, the PD-1 binding antagonist inhibits the binding of PD-1 to PD-L2. In some embodiments, the PD-1 binding antagonist inhibits the binding of PD-1 to both PD-L1 and PD-L2. In particular, a PD-L1 binding antagonist is an anti-PD-L1 antibody. The term “anti-PD-1 antibody” or “antibody binding to human PD-1” or “antibody that specifically binds to human PD-1” or “antagonistic anti-PD-1” refers to an antibody specifically binding to the human PD1 antigen with a binding affinity of KD-value of 1.0×10⁻⁸ mol/l or lower, in one aspect of a KD-value of 1.0×10⁻⁹ mol/l or lower. The binding affinity is determined with a standard binding assay, such as surface plasmon resonance technique (BIAcore®, GE-Healthcare Uppsala, Sweden).

In one aspect, the agent blocking PD-L1/PD-1 interaction is an anti-PD-1 antibody. In a specific aspect, the anti-PD-1 antibody is selected from the group consisting of MDX 1106 (nivolumab), MK-3475 (pembrolizumab), CT-011 (pidilizumab), MEDI-0680 (AMP-514), PDR001, REGN2810, and BGB-108, in particular from pembrolizumab and nivolumab. In another aspect, the agent blocking PD-L1/PD-1 interaction is an anti-PD-1 antibody comprising a heavy chain variable domain VH(PD-1) of SEQ ID NO:292 and a light chain variable domain VL(PD-1) of SEQ ID NO:293. In another aspect, the agent blocking PD-L1/PD-1 interaction is an anti-PD-1 antibody comprising a heavy chain variable domain VH(PD-1) of SEQ ID NO:294 and a light chain variable domain VL(PD-1) of SEQ ID NO:295.

Polynucleotides

The invention further provides isolated polynucleotides encoding agonistic ICOS-binding molecule or a T-cell bispecific antibody as described herein or a fragment thereof.

The isolated polynucleotides encoding the bispecific antibodies of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes the entire antigen-binding molecule according to the invention as described herein. In other embodiments, the isolated polynucleotide encodes a polypeptide comprised in the antibody according to the invention as described herein.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

Bispecific antibodies of the invention may be obtained, for example, by solid-state peptide synthesis (e.g. Merrifield solid phase synthesis) or recombinant production. For recombinant production one or more polynucleotide encoding the antibody or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the antibody (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the antibody or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the antibody of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the antibody or polypeptide fragments thereof is desired, DNA encoding a signal sequence may be placed upstream of the nucleic acid an antibody of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a bispecific antibody of the invention or polypeptide fragments thereof.

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) an antibody of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing an agonistic ICOS-binding molecule of the invention or polypeptide fragments thereof is provided, wherein the method comprises culturing a host cell comprising polynucleotides encoding the agonistic ICOS-binding molecule or polypeptide fragments thereof, as provided herein, under conditions suitable for expression of the antibody of the invention or polypeptide fragments thereof, and recovering the antibody of the invention or polypeptide fragments thereof from the host cell (or host cell culture medium).

In certain embodiments the antigen binding domains capable of specific binding to a tumor-associated antigen or antigen binding domains capable of specific binding to ICOS (e.g. Fab fragments or VH and VL) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the antikgne binding domains are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch.14 (Cold Spring Harbor Laboratory, Cold Spring Harbor, N.Y.).

Agonistic ICOS-binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the bispecific antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the Examples. The purity of the bispecific antigen binding molecule or fragments thereof can be determined by any of a variety of well-known analytical methods including gel electrophoresis, high pressure liquid chromatography, and the like. For example, the bispecific antigen binding molecules expressed as described in the Examples were shown to be intact and properly assembled as demonstrated by reducing and non-reducing SDS-PAGE.

Assays

The antigen binding molecules provided herein may be identified, screened for, or characterized for their physical/chemical properties and/or biological activities by various assays known in the art.

1. Affinity Assays

The affinity of the antibody provided herein for ICOS or the tumor-associated antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. The affinity of the bispecific antigen binding molecule for the target cell antigen can also be determined by surface plasmon resonance (SPR), using standard instrumentation such as a BIAcore instrument (GE Healthcare), and receptors or target proteins such as may be obtained by recombinant expression. A specific illustrative and exemplary embodiment for measuring binding affinity is described in Example 9. According to one aspect, K_{D} is measured by surface plasmon resonance using a BIACORE® T100 machine (GE Healthcare) at 25° C.

2. Binding Assays and Other Assays

In one aspect, an antibody as reported herein is tested for its antigen binding activity, e.g., by known methods such as ELISA, Western blot, flow cytometry, etc.

3. Activity Assays

Several cell-based in vitro assays were performed to evaluate the activity of the agonistic ICOS-binding molecules comprising at least one antigen binding domain that binds to a tumor-associated antigen. The assays were designed to show additional agonistic/co-stimulatory activity of the anti-ICOS bispecific molecules in presence of T-cell bispecific-(TCB) mediated activation of T-cells. For example, a Jurkat assay with a reporter cell line with NFAT-regulated expression of luciferase, induced upon engagement of the CD3/TCR and ICOS), wherein ICOS IgG molecules, plate-bound vs. in solution and in absence versus presence of a coated CD3 IgG stimulus were measured, is described in more detail in Example 7.2.

Furthermore, primary human PBMC co-culture assays, wherein FAP-targeted ICOS molecules, cross-linked by simultaneous binding to human ICOS on T-cells and human FAP, expressed on 3T3-hFAP cells (parental cell line ATCC #CCL-92, modified to stably overexpress human FAP), in the presence of a TCB molecule being crosslinked by simultaneous binding to CD3 on T-cells and human CEA on tumor cells were tested and described in Example 7.1.

In certain aspects, an antibody as reported herein is tested for such biological activity.

Pharmaceutical Compositions, Formulations and Routes of Administration

In a further aspect, the invention provides pharmaceutical compositions comprising an agonistic ICOS-binding molecule comprising at least one antigen binding domain capable of specific binding to a tumor-associated antigen and a T-cell activating anti-CD3 bispecific antibody specific for a tumor-associated antigen and pharmaceutically acceptable excipients. In a particular aspect, there is provided a pharmaceutical composition comprising an agonistic ICOS-binding molecule comprising at least one antigen binding domain capable of specific binding to a tumor-associated antigen and a T-cell activating anti-CD3 bispecific antibody specific for a tumor-associated antigen and pharmaceutically acceptable excipients for use in the treatment of cancer, more particularly for the treatment of solid tumors. In one further aspect, provided a pharmaceutical composition comprising an agonistic ICOS-binding molecule comprising at least one antigen binding domain capable of specific binding to a tumor-associated antigen and a T-cell activating anti-CD3 bispecific antibody specific for a tumor-associated antigen, wherein the agonistic ICOS-binding molecule comprising at least one antigen binding domain capable of specific binding to a tumor-associated antigen and the T-cell activating anti-CD3 bispecific antibody specific for a tumor-associated antigen are for administration together in a single composition or for separate administration in two or more different compositions. In another aspect, the agonistic ICOS-binding molecule comprising at least one antigen binding domain capable of specific binding to a tumor-associated antigen is administered concurrently with, prior to, or subsequently to the T-cell activating anti-CD3 bispecific antibody specific for a tumor-associated antigen.

In another aspect, a pharmaceutical composition comprises an agonistic ICOS-binding molecule provided herein and at least one pharmaceutically acceptable excipient. In another aspect, a pharmaceutical composition comprises an agonistic ICOS-binding molecule provided herein and at least one additional therapeutic agent, e.g., as described below.

In yet another aspect, the invention provides a pharmaceutical composition comprising an agonistic ICOS-binding molecule comprising at least one antigen binding domain capable of specific binding to a tumor-associated antigen for use in a method for treating or delaying progression of cancer, wherein the agonistic ICOS-binding molecule comprising at least one antigen binding domain capable of specific binding to a tumor-associated antigen is for use in combination with a T-cell activating anti-CD3 bispecific antibody specific for a tumor-associated antigen or for combination with an agent blocking PD-L1/PD-1 interaction. In another aspect, the agonistic ICOS-binding molecule comprising at least one antigen binding domain capable of specific binding to a tumor-associated antigen is for use in combination with a T-cell activating anti-CD3 bispecific antibody specific for a tumor-associated antigen and in combination with an agent blocking PD-L1/PD-1 interaction. In particular, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab. In another specific aspect, the agent blocking PD-L1/PD-1 interaction is pembrolizumab or nivolumab.

Pharmaceutical compositions of the present invention comprise a therapeutically effective amount of one or more antibodies dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one antibody and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the TNF family ligand trimer-containing antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the agonistic ICOS-binding molecules described herein may also be formulated as a depot preparation. Such long acting formulations may be administered by implantation (for example subcutaneously or intramuscularly) or by intramuscular injection. Thus, for example, the agonistic ICOS-binding molecules may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the agonistic ICOS-binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The agonistic ICOS-binding molecule of the invention may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended. The formulations to be used for in vivo administration are generally sterile. Sterility may be readily accomplished, e.g., by filtration through sterile filtration membranes.

Therapeutic Methods and Compositions

In one aspect, provided is a method for treating or delaying progression of cancer in a subject comprising administering to the subject an effective amount of an agonistic ICOS-binding molecule comprising at least one antigen binding domain capable of specific binding to a tumor-associated antigen and a T-cell activating anti-CD3 bispecific antibody, in particular a anti-CEA/anti-CD3 bispecific antibody.

In one such aspect, the method further comprises administering to the subject an effective amount of at least one additional therapeutic agent. In further embodiments, herein is provided a method for tumor shrinkage comprising administering to the subject an effective amount of an agonistic ICOS-binding molecule comprising at least one antigen binding domain capable of specific binding to a tumor-associated antigen and a T-cell activating anti-CD3 bispecific antibody, in particular an anti-CEA/anti-CD3 bispecific antibody. An “individual” or a “subject” according to any of the above aspects is preferably a human.

In further aspects, a composition for use in cancer immunotherapy is provided comprising an agonistic ICOS-binding molecule comprising at least one antigen binding domain capable of specific binding to a tumor-associated antigen and a T-cell activating anti-CD3 bispecific antibody, in particular a anti-CEA/anti-CD3 bispecific antibody. In certain embodiments, a composition comprising an agonistic ICOS-binding molecule comprising at least one antigen binding domain capable of specific binding to a tumor-associated antigen and a T-cell activating anti-CD3 bispecific antibody, in particular an anti-CEA/anti-CD3 bispecific antibody, for use in a method of cancer immunotherapy is provided.

In a further aspect, herein is provided the use of a composition comprising an agonistic ICOS-binding molecule comprising at least one antigen binding domain capable of specific binding to a tumor-associated antigen and a T-cell activating anti-CD3 bispecific antibody, in particular a anti-CEA/anti-CD3 bispecific antibody, in the manufacture or preparation of a medicament. In one aspect, the medicament is for treatment of cancer. In a further aspect, the medicament is for use in a method of tumor shrinkage comprising administering to an individual having a solid tumor an effective amount of the medicament. In one such aspect, the method further comprises administering to the individual an effective amount of at least one additional therapeutic agent. In a further embodiment, the medicament is for treating solid tumors. In some aspects, the individual has CEA positive cancer. In some aspects, CEA positive cancer is colon cancer, lung cancer, ovarian cancer, gastric cancer, bladder cancer, pancreatic cancer, endometrial cancer, breast cancer, kidney cancer, esophageal cancer, or prostate cancer. In some aspects, the breast cancer is a breast carcinoma or a breast adenocarcinoma. In some aspects, the breast carcinoma is an invasive ductal carcinoma. In some aspects, the lung cancer is a lung adenocarcinoma. In some embodiments, the colon cancer is a colorectal adenocarcinoma. A “subject” or an “individual” according to any of the above embodiments may be a human.

In another aspect, provided is a method for treating or delaying progression of cancer in a subject comprising administering to the subject an effective amount of an agonistic ICOS-binding molecule comprising at least one antigen binding domain capable of specific binding to a tumor-associated antigen and a T-cell activating anti-CD3 bispecific antibody, in particular a anti-CEA/anti-CD3 bispecific antibody, wherein the subject comprises a low ICOS baseline expression on T cells before treatment with the agonistic ICOS-binding molecule.

The combination therapies noted above encompass combined administration (where two or more therapeutic agents are included in the same or separate formulations), and separate administration, in which case, administration of the antibody as reported herein can occur prior to, simultaneously, and/or following, administration of the additional therapeutic agent or agents. In one aspect, administration of a T-cell activating anti-CD3 bispecific antibody, in particular a anti-CEA/anti-CD3 bispecific antibody, and of an agonistic ICOS-binding molecule comprising at least one antigen binding domain capable of specific binding to a tumor-associated antigen and optionally the administration of an additional therapeutic agent occur within about one month, or within about one, two or three weeks, or within about one, two, three, four, five, or six days, of each other.

Both the T-cell activating anti-CD3 bispecific antibody, in particular an anti-CEA/anti-CD3 bispecific antibody, and the agonistic ICOS-binding molecule comprising at least one antigen binding domain capable of specific binding to a tumor-associated antigen as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Both the T-cell activating anti-CD3 bispecific antibody, in particular an anti-CEA/anti-CD3 bispecific antibody, and the agonistic ICOS-binding molecule comprising at least one antigen binding domain capable of specific binding to a tumor-associated antigen as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibodies need not be, but are optionally formulated with one or more agents currently used to prevent or treat the disorder in question. The effective amount of such other agents depends on the amount of antibodies present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

In another aspect, provided is a method for treating or delaying progression of cancer in a subject comprising administering to the subject an effective amount of an agonistic ICOS-binding molecule comprising at least one antigen binding domain capable of specific binding to a tumor-associated antigen.

Other Agents and Treatments

The agonistic ICOS-binding molecules comprising at least one antigen binding domain capable of specific binding to a tumor-associated antigen of the invention may be administered in combination with one or more other agents in therapy. For instance, an agonistic ICOS-binding molecules of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent. In one aspect, the additional therapeutic agent is selected from the group consisting of a chemotherapeutic agent, radiation and other agents for use in cancer immunotherapy. In a further aspect, provided is the agonistic ICOS-binding molecule comprising at least one antigen binding domain capable of specific binding to a tumor-associated antigen as described herein before as described herein for use in the treatment of cancer, wherein the agonistic ICOS-binding molecule comprising at least one antigen binding domain that binds to a tumor-associated antigen is administered in combination with another immunomodulator.

The term “immunomodulator” refers to any substance including a monoclonal antibody that effects the immune system. The molecules of the inventions can be considered immunomodulators. Immunomodulators can be used as anti-neoplastic agents for the treatment of cancer. In one aspect, immunomodulators include, but are not limited to anti-CTLA4 antibodies (e.g. ipilimumab), anti-PD1 antibodies (e.g. nivolumab or pembrolizumab), PD-L1 antibodies (e.g. atezolizumab, avelumab or durvalumab), OX-40 antibodies, LAG3 antibodies, TIM-3 antibodies, 4-1BB antibodies and GITR antibodies.

In a further aspect, provided is the agonistic ICOS-binding molecule comprising at least one antigen binding domain capable of specific binding to a tumor-associated antigen as described herein before as described herein for use in the treatment of cancer, wherein the agonistic ICOS-binding molecule comprising at least one antigen binding domain capable of specific binding to a tumor-associated antigen is administered in combination with an agent blocking PD-L1/PD-1 interaction. In one aspect, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In one specific aspect, the agent blocking PD-L1/PD-1 interaction is atezolizumab. In another aspect, the agent blocking PD-L1/PD-1 interaction is pembrolizumab or nivolumab. Such other agents are suitably present in combination in amounts that are effective for the purpose intended. The effective amount of such other agents depends on the amount of agonistic ICOS-binding molecule used, the type of disorder or treatment, and other factors discussed above. The agonistic ICOS-binding molecules comprising at least one antigen binding domain capable of specific binding to a tumor-associated antigen are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the agonistic ICOS-binding molecules comprising at least one antigen binding domain capable of specific binding to a tumor-associated antigen of the invention can occur prior to, simultaneously, and/or following, administration of the additional therapeutic agent and/or adjuvant.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an agonistic ICOS-binding molecule comprising at least one antigen binding domain capable of specific binding to a tumor-associated antigen of the invention.

The label or package insert indicates that the composition is used for treating the condition of choice. Moreover, the article of manufacture may comprise (a) a first container with a composition contained therein, wherein the composition comprises an agonistic ICOS-binding molecule comprising at least one antigen binding domain capable of specific binding to a tumor-associated antigen of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID NO: | Name | Sequence |
| 1 | human ICOS | UniProt Q9Y6W8: |
| | | MKSGLWYFFL FCLRIKVLTG EINGSANYEM FIFHNGGVQI |
| | | LCKYPDIVQQ FKMQLLKGGQ ILCDLTKTKG SGNTVSIKSL |
| | | KFCHSQLSNN SVSFFLYNLD HSHANYYFCN LSIFDPPPFK |
| | | VTLTGGYLHI YESQLCCQLK FWLPIGCAAF VVVCILGCIL |
| | | ICWLTKKKYS SSVHDPNGEY MFMRAVNTAK KSRLTDVTL |
| 2 | Cynomolgus ICOS | UniProt G7PL89: |
| | | MKSGLWYFFL FCLHMKVLTG EINGSANYEM FIFHNGGVQI |
| | | LCKYPDIVQQ FKMQLLKGGQ ILCDLTKTKG SGNKVSIKSL |
| | | KFCHSQLSNN SVSFFLYNLD RSHANYYFCN LSIFDPPPFK |
| | | VTLTGGYLHI YESQLCCQLK FWLPIGCATF VVVCIFGCIL |
| | | ICWLTKKKYS STVHDPNGEY MFMRAVNTAK KSRLTGTTP |
| 3 | Murine ICOS | UniProt Q9WVS0: |
| | | MKPYFCRVFV FCFLIRLLTG EINGSADHRM FSFHNGGVQI |
| | | SCKYPETVQQ LKMRLFRERE VLCELTKTKG SGNAVSIKNP |
| | | MLCLYHLSNN SVSFFLNNPD SSQGSYYFCS LSIFDPPPFQ |
| | | ERNLSGGYLH IYESQLCCQL KLWLPVGCAA FVVVLLFGCI |
| | | LIIWFSKKKY GSSVHDPNSE YMFMAAVNTN KKSRLAGVTS |
| 4 | ICOS (009) CDR-H1 | GFTFSDYWMN |
| 5 | ICOS (009) CDR-H2 | QIRNKPYNYETYYSDSVKG |
| 6 | ICOS (009) CDR-H3 | PRLRSSDWHFDV |
| 7 | ICOS (009) CDR-L1 | KASQDINKNIA |
| 8 | ICOS (009) CDR-L2 | YTSTLQT |
| 9 | ICOS (009) CDR-L3 | LQFDNLYT |
| 10 | ICOS (009) VH | EVRLDETGGGVVQPGRPMELSCVASGFTFSDYWMNWVRQSPEKG |
| | | LEWVAQIRNKPYNYETYYSDSVKGRFTISRDDSKSRVYLQMNNL |
| | | RAEDMGIYYCTWPRLRSSDWHFDVWGAGTTVTVSS |
| 11 | ICOS (009) VL | AIQMTQSPSSLSASLGGEVTITCKASQDINKNIAWYQHKPGRGP |
| | | RLLIWYTSTLQTGIPSRFSGSGSGRDYSFTISNLEPEDFATYYC |
| | | LQFDNLYTFGSGTKLEIR |
| 12 | ICOS (1167) CDR-H1 | GFTFNTYAVH |
| 13 | ICOS (1167) CDR-H2 | GIGGSGVRTYYADSVKG |
| 14 | ICOS (1167) CDR-H3 | DIYVADFTGYAFDI |
| 15 | ICOS (1167) CDR-L1 | RASQGINNFLA |
| 16 | ICOS (1167) CDR-L2 | DASSLQS |
| 17 | ICOS (1167) CDR-L3 | QQYNFYPLT |
| 18 | ICOS (1167) VH | EVRLLESGGGLVQPGGSLRLSCAASGFTFNTYAVHWVRQAPGKG |
| | | LEWVSGIGGSGVRTYYADSVKGRLTISRDNSKNTLYLQMNSLRA |
| | | EDTAIYFCAKDIYVADFTGYAFDIWGQGTMVTVSS |
| 19 | ICOS (1167) VL | DIQMTQSPSSVSASVGDRVTITCRASQGINNFLAWYQQKPGKAP |
| | | KLLIYDASSLQSGVPSRFAGSGSGTDFTLTISSLQPEDFATYYC |
| | | QQYNFYPLTFGGGTMVE1K |
| 20 | ICOS (1143) CDR-H1 | GFDFSSAYDMC |
| 21 | ICOS (1143) CDR-H2 | CVYYGDGITYYATWAKG |
| 22 | ICOS (1143) CDR-H3 | GAFLGSSYYLSL |
| 23 | ICOS (1143) CDR-L1 | QASENIYNWLA |
| 24 | ICOS (1143) CDR-L2 | DASKLAS |
| 25 | ICOS (1143) CDR-L3 | QQAYTYGNIDNA |
| 26 | ICOS (1143) VH | QSLEESGGDLVKPGASLTLTCKASGFDFSSAYDMCWVRQAPGKG |
| | | LEWIGCVYYGDGITYYATWAKGRFTISKTSSTTVPLQMTSLTAA |
| | | DTATYFCARGAFLGSSYYLSLWGQGTLVTVSS |
| 27 | ICOS (1143) VL | AIDMTQTPASVEAAVGGTVTINCQASENIYNWLAWYQQKPGQPP |
| | | KLLIYDASKLASGVPSRFSASGSGTQFTLTISAVECADAATYYC |
| | | QQAYTYGNIDNAFGGGTEVVVS |
| 28 | ICOS (1138) CDR-H1 | GFDLSSYYYMC |
| 29 | ICOS (1138) CDR-H2 | CIYADIYGGTTHYASWAKG |
| 30 | ICOS (1138) CDR-H3 | EDGSRYGGSGYYNL |
| 31 | ICOS (1138) CDR-L1 | QASQNIYSNLA |
| 32 | ICOS (1138) CDR-L2 | AASYLTS |
| 33 | ICOS (1138) CDR-L3 | QQGHTTDNIDNA |
| 34 | ICOS (1138) VH | QSLEESGGDLVKPGASLTLTCTASGFDLSSYYYMCWVRQAPGKG |
| | | LEWIACIYADIYGGTTHYASWAKGRFTISKTSSTTVTLQMTSLT |
| | | AADTATYFCAREDGSRYGGSGYYNLWGPGTLVTVSS |
| 35 | ICOS (1138) VL | ALVMTQTPSSVSAAVGGTVTINCQASQNIYSNLAWYQQKPGQPP |
| | | KLLIYAASYLTSGVSSRFKGSGAGTQFTLTISGVECADAATYYC |
| | | QQGHTTDNIDNAFGGGTEVVVK |
| 36 | FAP (4B9) CDR-H1 | SYAMS |
| 37 | FAP (4B9) CDR-H2 | AIIGSGASTYYADSVKG |
| 38 | FAP (4B9) CDR-H3 | GWFGGFNY |
| 39 | FAP (4B9) CDR-L1 | RASQSVTSSYLA |
| 40 | FAP (4B9) CDR-L2 | VGSRRAT |
| 41 | FAP (4B9) CDR-L3 | QQGIMLPPT |
| 42 | FAP (4B9) VH | EVQLLESGGGLVQPGGSLRLSCAASGFTFSSYAMSWVRQAPGKG |
| | | LEWVSAIIGSGASTYYADSVKGRFTISRDNSKNTLYLQMNSLRA |
| | | EDTAVYYCAKGWFGGFNYWGQGTLVTVSS |
| 43 | FAP (4B9) VL | EIVLTQSPGTLSLSPGERATLSCRASQSVTSSYLAWYQQKPGQA |
| | | PRLLINVGSRRATGIPDRFSGSGSGTDFTLTISRLEPEDFAVYY |
| | | CQQGIMLPPTFGQGTKVEIK |
| 44 | FAP (28H1) CDR-H1 | SHAMS |
| 45 | FAP (28H1) CDR-H2 | AIWASGEQYYADSVKG |
| 46 | FAP (28H1) CDR-H3 | GWLGNFDY |
| 47 | FAP (28H1) CDR-L1 | RASQSVSRSYLA |
| 48 | FAP (28H1) CDR-L2 | GASTRAT |
| 49 | FAP (28H1) CDR-L3 | QQGQVIPPT |
| 50 | FAP (28H1) VH | EVQLLESGGGLVQPGGSLRLSCAASGFTESSHAMSWVRQAPGKG |
| | | LEWVSAIWASGEQYYADSVKGRFTISRDNSKNTLYLQMNSLRAE |
| | | DTAVYYCAKGWLGNFDYWGQGTLVTVSS |
| 51 | FAP (28H1) VL | EIVLTQSPGTLSLSPGERATLSCRASQSVSRSYLAWYQQKPGQA |
| | | PRLLIIGASTRATGIPDRFSGSGSGTDFTLTISRLEPEDFAVYY |
| | | CQQGQVIPPTFGQGTKVEIK |
| 52 | CEA (MEDI-565)-CDR- | SYWMH |
| | H1 | |
| 53 | CEA (MEDI-565)-CDR- | FIRNKANGGTTEYAAS |
| | H2 | |
| 54 | CEA (MEDI-565)-CDR- | DRGLRFYFDY |
| | H3 | |
| 55 | CEA (MEDI-565)-CDR- | TLRRGINVGAYSIY |
| | L1 | |
| 56 | CEA (MEDI-565)-CDR- | YKSDSDKQQGS |
| | L2 | |
| 57 | CEA (MEDI-565)-CDR- | MIWHSGASAV |
| | L3 | |
| 58 | CEA (MEDI-565) VH | EVQLVESGGGLVQPGRSLRLSCAASGFTVSSYWMHWVRQA |
| | | PGKGLEWVGFIRNKANGGTTEYAASVKGRFTISRDDSKNT |
| | | LYLQMNSLRAEDTAVYYCARDRGLRFYFDYWGQGTTVTVS |
| | | S |
| 59 | CEA (MEDI-565) VL | QAVLTQPASLSASPGASASLTCTLRRGINVGAYSIYWYQQ |
| | | KPGSPPQYLLRYKSDSDKQQGSGVSSRFSASKDASANAGI |
| | | LLISGLQSEDEADYYCMIWHSGASAVFGGGTKLTVL |
| 60 | CEA (A5H1EL1D)-CDR- | DYYMN |
| | H1 | |
| 61 | CEA (A5H1EL1D)-CDR- | FIGNKANAYTTEYSASVKG |
| | H2 | |
| 62 | CEA (A5H1EL1D)-CDR- | DRGLRFYFDY |
| | H3 | |
| 63 | CEA (A5H1EL1D)-CDR- | RASSSVTYIH |
| | L1 | |
| 64 | CEA (A5H1EL1D)-CDR- | ATSNLAS |
| | L2 | |
| 65 | CEA (A5H1EL1D)-CDR- | QHWSSKPPT |
| | L3 | |
| 66 | CEA (A5B7) VH | EVKLVESGGGLVQPGGSLRLSCATSGFTFTDYYMNWVRQP |
| | | PGKALEWLGFIGNKANGYTTEYSASVKGRFTISRDKSQSI |
| | | LYLQMNTLRAEDSATYYCTRDRGLRFYFDYWGQGTTLTVS |
| | | S |
| 67 | CEA (A5B7) VL | QTVLSQSPAILSASPGEKVTMTCRASSSVTYIHWYQQKPG |
| | | SSPKSWIYATSNLASGVPARFSGSGSGTSYSLTISRVEAE |
| | | DAATYYCQHWSSKPPTFGGGTKLEIK |
| 68 | CEA (A5H1EL1D) VH (3- | EVQLLESGGGLVQPGGSLRLSCAASGFTFTDYYMNWVRQA |
| | 23A5-1E) | PGKGLEWLGFIGNKANAYTTEYSASVKGRFTISRDKSKNT |
| | | LYLQMNSLRAEDTATYYCTRDRGLRFYFDYWGQGTTVTVS |
| | | S |
| 69 | CEA (A5H1EL1D) VL | EIVLTQSPATLSLSPGERATLSCRASSSVTYIHWYQQKPG |
| | (A5-L1D) | QAPRSWIYATSNLASGIPARFSGSGSGTDFTLTISSLEPE |
| | | DFAVYYCQHWSSKPPTFGQGTKLEIK |
| 70 | human ICOS antigen Fc | See Table 2 |
| | hole chain (dimeric) | |
| 71 | human ICOS antigen Fc | See Table 2 |
| | knob chain (dimeric) | |
| 72 | human ICOS antigen Fc | See Table 2 |
| | hole chain (monomeric) | |
| 73 | cynomolgus ICOS antigen | See Table 2 |
| | Fc hole chain | |
| 74 | cynomolgus ICOS antigen | See Table 2 |
| | Fc knob chain | |
| 75 | murine ICOS antigen Fc | See Table 2 |
| | hole chain | |
| 76 | murine ICOS antigen Fc | See Table 2 |
| | knob chain | |
| 77 | rbHC.up | AAGCTTGCCACCATGGAGACTGGGCTGCGCTGGCTTC |
| 78 | rbHCf.do | CCATTGGTGAGGGTGCCCGAG |
| 79 | rbLC.up | AAGCTTGCCACCATGGACAYGAGGGCCCCCACTC |
| 80 | rbLC.do | CAGAGTRCTGCTGAGGTTGTAGGTAC |
| 81 | BcPCR_FHLC_Leader.fw | ATGGACATGAGGGTCCCCGC |
| 82 | BcPCR_huCkappa.rev | GATTTCAACTGCTCATCAGATGGC |
| 83 | 1167 light chain (rabbit | See Table 5 |
| | IgG) | |
| 84 | 1167 light chain (rabbit | See Table 5 |
| | IgG) | |
| 85 | 1143 light chain (rabbit | See Table 5 |
| | IgG) | |
| 86 | 1143 light chain (rabbit | See Table 5 |
| | IgG) | |
| 87 | 1138 light chain (rabbit | See Table 5 |
| | IgG) | |
| 88 | 1138 light chain (rabbit | See Table 5 |
| | IgG) | |
| 89 | human ICOS Fc knob Avi- | See Table 7 |
| | tag | |
| 90 | human ICOS Fc hole | See Table 7 |
| 91 | (FAP 4B9) VLCH1-Fc | See Table 11 |
| | hole | |
| 92 | (FAP 4B9) VHCL-Light | See Table 11 |
| | chain 1 | |
| 93 | (1167) VHCH1-Fc knob | See Table 11 |
| 94 | (1167) VLCL-Light chain | See Table 11 |
| | 2 | |
| 95 | Fc hole VH (FAP 4B9) | See Table 13 |
| 96 | (1167) VHCH1 Fc knob | See Table 13 |
| | VL (4B9) | |
| 97 | (ICOS 1167) VHCH1 Fc | See Table 16 |
| | hole VH (FAP 4B9) | |
| 98 | (ICOS 009) VHCH1 Fc | See Table 16 |
| | hole VH (FAP 4B9) | |
| 99 | (ICOS 009) VHCH1 Fc | See Table 16 |
| | knob VL (FAP 4B9) | |
| 100 | (ICOS 009) VLCL-light | See Table 16 |
| | chain | |
| 101 | (ICOS 009v1) VHCH1 Fc | See Table 16 |
| | knob VL (FAP 4B9) | |
| 102 | (ICOS 1138) VHCH1 Fc | See Table 16 |
| | hole VH (FAP 4B9) | |
| 103 | (ICOS 1138)VHCH1 Fc | See Table 16 |
| | knob VL (FAP 4B9) | |
| 104 | (ICOS 1138) VLCL-light | See Table 16 |
| | chain | |
| 105 | (ICOS 1143) VHCH1 Fc | See Table 16 |
| | hole VH (FAP 4B9) | |
| 106 | (ICOS 1143) VHCH1 Fc | See Table 16 |
| | knob VL (4B9) | |
| 107 | (ICOS 1143) VLCL-light | See Table 16 |
| | chain | |
| 108 | (ICOS 1143v1) VHCH1 Fc | See Table 16 |
| | hole VH (FAP 4B9) | |
| 109 | (ICOS 1143v1) VHCH1 Fc | See Table 16 |
| | knob VL (4B9) | |
| 110 | (ICOS 1143v2) VHCH1 Fc | See Table 16 |
| | hole VH (FAP 4B9) | |
| 111 | (ICOS 1143v2) VHCH1 Fc | See Table 16 |
| | knob VL (FAP 4B9) | |
| 112 | (ICOS 1167) VHCH1 Fc | See Table 18 |
| | hole | |
| 113 | (ICOS 1167) VLCL-light | See Table 18 |
| | chain 1 | |
| 114 | (FAP 4B9) VLCH1- | See Table 18 |
| | (ICOS 1167) VHCH1 Fc | |
| | knob | |
| 115 | (FAP 4B9) VHCL-light | See Table 18 |
| | chain 2 | |
| 116 | (ICOS 1167) VHCH1 Fc | See Table 20 |
| | hole | |
| 117 | (ICOS 1167) VLCL-light | See Table 20 |
| | chain 1 | |
| 118 | (ICOS 1167) VHCH1- | See Table 20 |
| | (FAP 4B9) VLCH1 Fc | |
| | knob | |
| 119 | (FAP 4B9) VHCL-light | See Table 20 |
| | chain 2 | |
| 120 | Post-CDR3 from | YYYYYGMDVWGQGTTVTVSS |
| | IGHJ6*01/02 | |
| 121 | Post-CDR3 from | YTFGQGTKLEIK |
| | IGKJ2*01 | |
| 122 | Post-CDR3 from | AEYFQHWGQGTLVTVSS |
| | IGHJ1*01 | |
| 123 | Post-CDR3 from | LTFGGGTKVEIK |
| | IGKJ4*01/02 1 | |
| 124 | ICOS (009)-VHG1a | See Table 28 |
| 125 | ICOS (009)-VHG1b | See Table 28 |
| 126 | ICOS (009)-VHG1c | See Table 28 |
| 127 | ICOS (009)-VHG1d | See Table 28 |
| 128 | ICOS (009)-VHG2a | See Table 28 |
| 129 | ICOS (009)-VHG2b | See Table 28 |
| 130 | ICOS (009)-VHG2c | See Table 28 |
| 131 | ICOS (009)-VHG2d | See Table 28 |
| 132 | ICOS (009)-VLG1a | See Table 28 |
| 133 | ICOS (009)-VLG1b | See Table 28 |
| 134 | ICOS (009)-VLG2a | See Table 28 |
| 135 | ICOS (009)-VLG2b | See Table 28 |
| 136 | ICOS (1138)-VHG1a | See Table 28 |
| 137 | ICOS (1138)-VHG1b | See Table 28 |
| 138 | ICOS (1138)-VHG1c | See Table 28 |
| 139 | ICOS (1138)-VHG1d | See Table 28 |
| 140 | ICOS (1138)-VHG1e | See Table 28 |
| 141 | ICOS (1138)-VLG1a | See Table 28 |
| 142 | ICOS (1138)-VLG1b | See Table 28 |
| 143 | ICOS (1138)-VLG1c | See Table 28 |
| 144 | ICOS (1143)-VHG1a | See Table 28 |
| 145 | ICOS (1143)-VHG1b | See Table 28 |
| 146 | ICOS (1143)-VHG1c | See Table 28 |
| 147 | ICOS (1143)-VHG1d | See Table 28 |
| 148 | ICOS (1143)-VHG1e | See Table 28 |
| 149 | ICOS (1143)-VHG1f | See Table 28 |
| 150 | ICOS (1143)-VHG1g | See Table 28 |
| 151 | ICOS (1143)-VHG1h | See Table 28 |
| 152 | ICOS (1143)-VLG1a | See Table 28 |
| 153 | ICOS (1143)-VLG1b | See Table 28 |
| 154 | Molecule 25 (ICOS | See Table 29 |
| | H009v1_1) VH | |
| 155 | Molecule 25 (ICOS | See Table 29 |
| | H009v1_1) VL | |
| 156 | Molecule 26 (ICOS | See Table 29 |
| | H009v1_2) VH | |
| 157 | Molecule 26 (ICOS | See Table 29 |
| | H009v1_2) VL | |
| 158 | Molecule 27 (ICOS | See Table 29 |
| | H009v1_3) VH | |
| 159 | Molecule 27 (ICOS | See Table 29 |
| | H009v1_3) VL | |
| 160 | Molecule 32 (ICOS 1138) | See Table 29 |
| | VH | |
| 161 | Molecule 32 (ICOS 1138) | See Table 29 |
| | VL | |
| 162 | Molecule 33 (ICOS | See Table 29 |
| | 1138_1) VH | |
| 163 | Molecule 33 (ICOS | See Table 29 |
| | 1138_1) VL | |
| 164 | Molecule 34 (ICOS | See Table 29 |
| | 1138_2) VH | |
| 165 | Molecule 34 (ICOS | See Table 29 |
| | 1138_2) VL | |
| 166 | Molecule 35 (ICOS | See Table 29 |
| | 1138_3) VH | |
| 167 | Molecule 35 (ICOS | See Table 29 |
| | 1138_3) VL | |
| 168 | Molecule 28 (ICOS | See Table 29 |
| | 1143v2) VH | |
| 169 | Molecule 28 (ICOS | See Table 29 |
| | 1143v2) VL | |
| 170 | Molecule 29 (ICOS | See Table 29 |
| | 1143v2_1) VH | |
| 171 | Molecule 29 (ICOS | See Table 29 |
| | 1143v2_1) VL | |
| 172 | Molecule 30 (ICOS | See Table 29 |
| | 1143v2_2) VH | |
| 173 | Molecule 30 (ICOS | See Table 29 |
| | 1143v2_2) VL | |
| 174 | Molecule 31 (ICOS | See Table 29 |
| | 1143v2_3) VH | |
| 175 | Molecule 31 (ICOS | See Table 29 |
| | 1143v2_3) VL | |
| 176 | Murine A5B7 VH | See Table 31 |
| 177 | IGHV3-23-02 | See Table 31 |
| 178 | IGHV3-15*01 | See Table 31 |
| 179 | 3-23A5-1 | See Table 31 |
| 180 | 3-23A5-2 | See Table 31 |
| 181 | 3-23A5-3 | See Table 31 |
| 182 | 3-23A5-4 | See Table 31 |
| 183 | 3-23A5-1A | See Table 31 |
| | (all_backmutations) | |
| 184 | 3-23A5-1C (A93T) | See Table 31 |
| 185 | 3-23A5-1D (K73) | See Table 31 |
| 186 | 3-15A5-1 | See Table 31 |
| 187 | 3-15A5-2 | See Table 31 |
| 188 | 3-15A5-3 | See Table 31 |
| 189 | Murine A5B7 VL | See Table 32 |
| 190 | IGKV3-11 | See Table 32 |
| 191 | A5-L1 | See Table 32 |
| 192 | A5-L2 | See Table 32 |
| 193 | A5-L3 | See Table 32 |
| 194 | A5-L4 | See Table 32 |
| 195 | A5-L1A | See Table 32 |
| | (all_backmutations) | |
| 196 | A5-L1B (Q1T2) | See Table 32 |
| 197 | A5-L1C (FR2) | See Table 32 |
| 198 | ICOS (JMAb136) VHCH1- | See Table 35 |
| | Fc hole | |
| 199 | ICOS (JMAb136) VLCL- | See Table 35 |
| | Light chain 1 | |
| 200 | CEA (MEDI-565) VLCH1- | See Table 35 |
| | Fc knob | |
| 201 | CEA (MEDI-565) VHCL- | See Table 35 |
| | Light chain 2 | |
| 202 | CEA (A5H1EL1D) VLCH1- | See Table 35 |
| | Fc hole | |
| 203 | CEA (A5H1EL1D) VHCL- | See Table 35 |
| | Light chain 1 | |
| 204 | ICOS (1167) VHCH1-Fc | See Table 35 |
| | knob | |
| 205 | ICOS (1167) VLCL-Light | See Table 35 |
| | chain 2 | |
| 206 | CEA (A5H1EL1D) VHCH1- | See Table 35 |
| | Fc knob | |
| 207 | CEA (A5H1EL1D) VLCL- | See Table 35 |
| | Light chain 1 | |
| 208 | ICOS (H009v1_2) VLCH1- | See Table 35 |
| | Fc hole | |
| 209 | ICOS (H009v1_2) VHCL- | See Table 35 |
| | Light chain 2 | |
| 210 | ICOS (H1143v2_1) VLCH1- | See Table 35 |
| | Fc hole | |
| 211 | ICOS (H1143v2_1) VHCL- | See Table 35 |
| | Light chain 2 | |
| 212 | ICOS (JMAb136) VHCH1 | See Table 37 |
| | Fc hole VH CEA (MEDI- | |
| | 565) | |
| 213 | ICOS (JMAb136) VHCH1 | See Table 37 |
| | Fc knob VL CEA (MEDI- | |
| | 565) | |
| 214 | ICOS (JMAb136) VLCL- | See Table 37 |
| | light chain | |
| 215 | Human ICOS ligand | MRLGSPGLLF LLFSSLRADT QEKEVRAMVG SDVELSCACP |
| | UniProt O75144 | EGSRFDLNDV YVYWQTSESK TVVTYHIPQN SSLENVDSRY |
| | | RNRALMSPAG MLRGDFSLRL FNVTPQDEQK FHCLVLSQSL |
| | | GFQEVLSVEV TLHVAANFSV PVVSAPHSPS QDELTFTCTS |
| | | INGYPRPNVY WINKTDNSLL DQALQNDTVF LNMRGLYDVV |
| | | SVLRIARTPS VNIGCCIENV LLQQNLTVGS QTGNDIGERD |
| | | KITENPVSTG EKNAATWSIL AVLCLLVVVA VAIGWVCRDR |
| | | CLQHSYAGAW AVSPETELTG HV |
| 216 | ICOS (JMab 136) VH | QVQLVQSGAEVKKPGASVKVSCKASGYTFTGYYMHWVRQAPGQG |
| | | LEWMGWINPHSGGTNYAQKFQGRVTMTRDTSISTAYMELSRLRS |
| | | DDTAVYYCARTYYYDSSGYYHDAFDIWGQGTMVTVSS |
| 217 | ICOS (JMab136) VL | DIQMTQSPSSVSASVGDRVTITCRASQGISRLLAWYQQKPGKAP |
| | | KLLIYVASSLQSGVPSRFSGSGSGTDFTLTISSLQPEDFATYYC |
| | | QQANSFPWTFGQGTKVEIK |
| 218 | CD3 CDR-H1 | TYAMN |
| 219 | CD3 CDR-H2 | RIRSKYNNYATYYADSVKG |
| 220 | CD3 CDR-H3 | HGNFGNSYVSWFAY |
| 221 | CD3 CDR-L1 | GSSTGAVTTSNYAN |
| 222 | CD3 CDR-L2 | GTNKRAP |
| 223 | CD3 CDR-L3 | ALWYSNLWV |
| 224 | CD3 VH | EVQLLESGGGLVQPGGSLRLSCAASGFTESTYAMNWVRQAPGKG |
| | | LEWVSRIRSKYNNYATYYADSVKGRFTISRDDSKNTLYLQMNSL |
| | | RAEDTAVYYCVRHGNFGNSYVSWFAYWGQGTLVTVSS |
| 225 | CD3 VL | QAVVTQEPSLTVSPGGTVTLTCGSSTGAVTTSNYANWVQEKPGQ |
| | | AFRGLIGGTNKRAPGTPARFSGSLLGGKAALTLSGAQPEDEAEY |
| | | YCALWYSNLWVFGGGTKLTVL |
| 226 | CEA CDR-H1 | EFGMN |
| 227 | CEA CDR-H2 | WINTKTGEATYVEEFKG |
| 228 | CEA CDR-H3 | WDFAYYVEAMDY |
| 229 | CEA CDR-L1 | KASAAVGTYVA |
| 230 | CEA CDR-L2 | SASYRKR |
| 231 | CEA CDR-L3 | HQYYTYPLFT |
| 232 | CEA VH | QVQLVQSGAEVKKPGASVKVSCKASGYTFTEFGMNWVRQAPGQG |
| | | LEWMGWINTKTGEATYVEEFKGRVTFTTDTSTSTAYMELRSLRS |
| | | DDTAVYYCARWDFAYYVEAMDYWGQGTTVTVSS |
| 233 | CEA VL | DIQMTQSPSSLSASVGDRVTITCKASAAVGTYVAWYQQKPGKAP |
| | | KLLIYSASYRKRGVPSRFSGSGSGTDFTLTISSLQPEDFATYYC |
| | | HQYYTYPLFTFGQGTKLEIK |
| 234 | CEA CDR-H1 | DTYMH |
| | (CEACAM5) | |
| 235 | CEA CDR-H2 | RIDPANGNSKYVPKFQG |
| | (CEACAM5) | |
| 236 | CEA CDR-H3 | FGYYVSDYAMAY |
| | (CEACAM5) | |
| 237 | CEA CDR-L1 | RAGESVDIFGVGFLH |
| | (CEACAM5) | |
| 238 | CEA CDR-L2 | RASNRAT |
| | (CEACAM5) | |
| 239 | CEA-CDR-L3 | QQTNEDPYT |
| | (CEACAM5) | |
| 240 | CEA (CEACAM5) | QVQLVQSGAEVKKPGSSVKVSCKASGENIKDTYMHWVRQAPGQG |
| | | LEWMGRIDPANGNSKYVPKFQGRVTITADTSTSTAYMELSSLRS |
| | | EDTAVYYCAPFGYYVSDYAMAYWGQGTLVTVSS |
| 241 | CEA VL (CEACAM5) | EIVLTQSPATLSLSPGERATLSCRAGESVDIFGVGFLHWYQQKP |
| | | GQAPRLLIYRASNRATGIPARESGSGSGTDFTLTISSLEPEDFA |
| | | VYYCQQTNEDPYTFGQGTKLEIK |
| 242 | Light chain | DIQMTQSPSSLSASVGDRVTITCKASAAVGTYVAWYQQKPGKAP |
| | _{”}CEA _{2F1}” | KLLIYSASYRKRGVPSRFSGSGSGTDFTLTISSLQPEDFATYYC |
| | (CEA TCB) | HQYYTYPLFTFGQGTKLEIKRTVAAPSVFIFPPSDEQLKSGTAS |
| | | VVCLLNNFYPREAKVQWKVDNALQSGNSQESVTEQDSKDSTYSL |
| | | SSTLTLSKADYEKHKVYACEVTHQGLSSPVTKSFNRGEC |
| 243 | Light Chain humanized | QAVVTQEPSLTVSPGGTVTLTCGSSTGAVTTSNYANWVQEKPGQ |
| | CD3_{CH2527} (Crossfab, VL- | AFRGLIGGTNKRAPGTPARFSGSLLGGKAALTLSGAQPEDEAEY |
| | CH1) | YCALWYSNLWVFGGGTKLTVLSSASTKGPSVFPLAPSSKSTSGG |
| | (CEA TCB) | TAALGCLVKDYFPEPVTVSWNSGALTSGVHTFPAVLQSSGLYSL |
| | | SSVVTVPSSSLGTQTYICNVNHKPSNTKVDKKVEPKSC |
| 244 | CEA_{CH1A1A 98/99}- | QVQLVQSGAEVKKPGASVKVSCKASGYTFTEFGMNWVRQAPGQG |
| | humanized CD3_{CH2527} | LEWMGWINTKTGEATYVEEFKGRVTFTTDTSTSTAYMELRSLRS |
| | (Crossfab VH-Ck)- | DDTAVYYCARWDFAYYVEAMDYWGQGTTVTVSSASTKGPSVFPL |
| | Fc(knob) P329GLALA | APSSKSTSGGTAALGCLVKDYFPEPVTVSWNSGALTSGVHTFPA |
| | (CEA TCB) | VLQSSGLYSLSSVVTVPSSSLGTQTYICNVNHKPSNTKVDKKVE |
| | | PKSCDGGGGSGGGGSEVQLLESGGGLVQPGGSLRLSCAASGFTF |
| | | STYAMNWVRQAPGKGLEWVSRIRSKYNNYATYYADSVKGRFTIS |
| | | RDDSKNTLYLQMNSLRAEDTAVYYCVRHGNFGNSYVSWFAYWGQ |
| | | GTLVTVSSASVAAPSVFIFPPSDEQLKSGTASVVCLLNNFYPRE |
| | | AKVQWKVDNALQSGNSQESVTEQDSKDSTYSLSSTLTLSKADYE |
| | | KHKVYACEVTHQGLSSPVTKSFNRGECDKTHTCPPCPAPEAAGG |
| | | PSVFLFPPKPKDTLMISRTPEVTCVVVDVSHEDPEVKFNWYVDG |
| | | VEVHNAKTKPREEQYNSTYRVVSVLTVLHQDWLNGKEYKCKVSN |
| | | KALGAPIEKTISKAKGQPREPQVYTLPPCRDELTKNQVSLWCLV |
| | | KGFYPSDIAVEWESNGQPENNYKTTPPVLDSDGSFFLYSKLTVD |
| | | KSRWQQGNVFSCSVMHEALHNHYTQKSLSLSPGK |
| 245 | CEA_{CH1A1A 98/99} (VH-CH1)- | QVQLVQSGAEVKKPGASVKVSCKASGYTFTEFGMNWVRQAPGQG |
| | Fc(hole) P329GLALA | LEWMGWINTKTGEATYVEEFKGRVTFTTDTSTSTAYMELRSLRS |
| | (CEA TCB) | DDTAVYYCARWDFAYYVEAMDYWGQGTTVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPEAAGGPSVFLFPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFELVSKLTVDKSRWQQGNVESCSVMHEALHNHY |
| | | TQKSLSLSPGK |
| 246 | CD3 VH-CL (CEACAM5 | EVQLLESGGGLVQPGGSLRLSCAASGFTESTYAMNWVRQAPGKG |
| | TCB) | LEWVSRIRSKYNNYATYYADSVKGRFTISRDDSKNTLYLQMNSL |
| | | RAEDTAVYYCVRHGNFGNSYVSWFAYWGQGTLVTVSSASVAAPS |
| | | VFIFPPSDEQLKSGTASVVCLLNNFYPREAKVQWKVDNALQSGN |
| | | SQESVTEQDSKDSTYSLSSTLTLSKADYEKHKVYACEVTHQGLS |
| | | SPVTKSFNRGEC |
| 247 | humanized CEA VH- | QVQLVQSGAEVKKPGSSVKVSCKASGFNIKDTYMHWVRQAPGQG |
| | CH1(EE)-Fc (hole, P329G | LEWMGRIDPANGNSKYVPKFQGRVTITADTSTSTAYMELSSLRS |
| | LALA) | EDTAVYYCAPFGYYVSDYAMAYWGQGTLVTVSSASTKGPSVFPL |
| | (CEACAM5 TCB) | APSSKSTSGGTAALGCLVEDYFPEPVTVSWNSGALTSGVHTFPA |
| | | VLQSSGLYSLSSVVTVPSSSLGTQTYICNVNHKPSNTKVDEKVE |
| | | PKSCDKTHTCPPCPAPEAAGGPSVFLFPPKPKDTLMISRTPEVT |
| | | CVVVDVSHEDPEVKFNWYVDGVEVHNAKTKPREEQYNSTYRVVS |
| | | VLTVLHQDWLNGKEYKCKVSNKALGAPIEKTISKAKGQPREPQV |
| | | CTLPPSRDELTKNQVSLSCAVKGFYPSDIAVEWESNGQPENNYK |
| | | TTPPVLDSDGSFFLVSKLTVDKSRWQQGNVFSCSVMHEALHNHY |
| | | TQKSLSLSP |
| 248 | humanized CEA VH- | QVQLVQSGAEVKKPGSSVKVSCKASGFNIKDTYMHWVRQAPGQG |
| | CH1(EE)-CD3 VL-CH1-Fc | LEWMGRIDPANGNSKYVPKFQGRVTITADTSTSTAYMELSSLRS |
| | (knob, P329G LALA) | EDTAVYYCAPFGYYVSDYAMAYWGQGTLVTVSSASTKGPSVFPL |
| | (CEACAM5 TCB) | APSSKSTSGGTAALGCLVEDYFPEPVTVSWNSGALTSGVHTFPA |
| | | VLQSSGLYSLSSVVTVPSSSLGTQTYICNVNHKPSNTKVDEKVE |
| | | PKSCDGGGGSGGGGSQAVVTQEPSLTVSPGGTVTLTCGSSTGAV |
| | | TTSNYANWVQEKPGQAFRGLIGGTNKRAPGTPARFSGSLLGGKA |
| | | ALTLSGAQPEDEAEYYCALWYSNLWVFGGGTKLTVLSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPEAAGGPSVFLFPPKPKDTLMISR |
| | | TPEVTCVVVDVSHEDPEVKFNWYVDGVEVHNAKTKPREEQYNST |
| | | YRVVSVLTVLHQDWLNGKEYKCKVSNKALGAPIEKTISKAKGQP |
| | | REPQVYTLPPCRDELTKNQVSLWCLVKGFYPSDIAVEWESNGQP |
| | | ENNYKTTPPVLDSDGSFFLYSKLTVDKSRWQQGNVFSCSVMHEA |
| | | LHNHYTQKSLSLSP |
| 249 | humanized CEA VL-CL(RK) | EIVLTQSPATLSLSPGERATLSCRAGESVDIFGVGFLHWYQQKP |
| | (CEACAM5 TCB) | GQAPRLLIYRASNRATGIPARFSGSGSGTDFTLTISSLEPEDFA |
| | | VYYCQQTNEDPYTFGQGTKLEIKRTVAAPSVFIFPPSDRKLKSG |
| | | TASVVCLLNNFYPREAKVQWKVDNALQSGNSQESVTEQDSKDST |
| | | YSLSSTLTLSKADYEKHKVYACEVTHQGLSSPVTKSFNRGEC |
| 250 | VHCH1 (CH1A1A 98/99 | QVQLVQSGAEVKKPGASVKVSCKASGYTFTEFGMNWVRQAPGQG |
| | 2F1)-Fc(KK) DAPG chain | LEWMGWINTKTGEATYVEEFKGRVTFTTDTSTSTAYMELRSLRS |
| | | DDTAVYYCARWDFAYYVEAMDYWGQGTTVTVSSAKTTPPSVYPL |
| | | APGSAAQTNSMVTLGCLVKGYFPEPVTVTWNSGSLSSGVHTFPA |
| | | VLQSDLYTLSSSVTVPSSTWPSQTVTCNVAHPASSTKVDKKIVP |
| | | RDCGCKPCICTVPEVSSVFIFPPKPKDVLTITLTPKVTCVVVAI |
| | | SKDDPEVQFSWFVDDVEVHTAQTKPREEQINSTFRSVSELPIMH |
| | | QDWLNGKEFKCRVNSAAFGAPIEKTISKTKGRPKAPQVYTIPPP |
| | | KKQMAKDKVSLTCMITNFFPEDITVEWQWNGQPAENYKNTQPIM |
| | | KTDGSYFVYSKLNVQKSNWEAGNTFTCSVLHEGLHNHHTEKSLS |
| | | HSPGK |
| 251 | VLCL (CH1A1A 98/99 | DIQMTQSPSSLSASVGDRVTITCKASAAVGTYVAWYQQKPGKAP |
| | 2F1) Light chain | KLLIYSASYRKRGVPSRFSGSGSGTDFTLTISSLQPEDFATYYC |
| | | HQYYTYPLFTFGQGTKLEIKRADAAPTVSIFPPSSEQLTSGGAS |
| | | VVCFLNNFYPKDINVKWKIDGSERQNGVLNSWTDQDSKDSTYSM |
| | | SSTLTLTKDEYERHNSYTCEATHKTSTSPIVKSFNRNEC |
| 252 | VHCL VHCH1 (2C11- | EVQLVESGGGLVQPGKSLKLSCEASGFTFSGYGMHWVRQAPGRG |
| | CH1A1A 98/99 2F1)- | LESVAYITSSSINIKYADAVKGRFTVSRDNAKNLLFLQMNILKS |
| | Fc(DD) DAPG chain | EDTAMYYCARFDWDKNYWGQGTMVTVSSASDAAPTVSIFPPSSE |
| | | QLTSGGASVVCFLNNFYPKDINVKWKIDGSERQNGVLNSWTDQD |
| | | SKDSTYSMSSTLTLTKDEYERHNSYTCEATHKTSTSPIVKSFNR |
| | | NECGGGGSGGGGSQVQLVQSGAEVKKPGASVKVSCKASGYTFTE |
| | | FGMNWVRQAPGQGLEWMGWINTKTGEATYVEEFKGRVTFTTDTS |
| | | TSTAYMELRSLRSDDTAVYYCARWDFAYYVEAMDYWGQGTTVTV |
| | | SSAKTTPPSVYPLAPGSAAQTNSMVTLGCLVKGYFPEPVTVTWN |
| | | SGSLSSGVHTFPAVLQSDLYTLSSSVTVPSSTWPSQTVTCNVAH |
| | | PASSTKVDKKIVPRDCGCKPCICTVPEVSSVFIFPPKPKDVLTI |
| | | TLTPKVTCVVVAISKDDPEVQFSWFVDDVEVHTAQTKPREEQIN |
| | | STERSVSELPIMHQDWLNGKEEKCRVNSAAFGAPIEKTISKTKG |
| | | RPKAPQVYTIPPPKEQMAKDKVSLTCMITNFFPEDITVEWQWNG |
| | | QPAENYDNTQPIMDTDGSYFVYSDLNVQKSNWEAGNTFTCSVLH |
| | | EGLHNHHTEKSLSHSPGK |
| 253 | VLCH1 (2C11) | DIQMTQSPSSLPASLGDRVTINCQASQDISNYLNWYQQKPGKAP |
| | Light chain | KLLIYYTNKLADGVPSRFSGSGSGRDSSFTISSLESEDIGSYYC |
| | | QQYYNYPWTFGPGTKLEIKSSAKTTPPSVYPLAPGSAAQTNSMV |
| | | TLGCLVKGYFPEPVTVTWNSGSLSSGVHTFPAVLQSDLYTLSSS |
| | | VTVPSSTWPSQTVTCNVAHPASSTKVDKKIVPRDC |
| 254 | human FAP | UniProt accession no. Q12884 |
| | | MKTWVKIVFG VATSAVLALL VMCIVLRPSR VHNSEENTMR |
| | | ALTLKDILNG TFSYKTFFPN WISGQEYLHQ SADNNIVLYN |
| | | IETGQSYTIL SNRTMKSVNA SNYGLSPDRQ FVYLESDYSK |
| | | LWRYSYTATY YIYDLSNGEF VRGNELPRPI QYLCWSPVGS |
| | | KLAYVYQNNI YLKQRPGDPP FQITENGREN KIFNGIPDWV |
| | | YEEEMLATKY ALWWSPNGKF LAYAEFNDTD IPVIAYSYYG |
| | | DEQYPRTINI PYPKAGAKNP VVRIFIIDTT YPAYVGPQEV |
| | | PVPAMIASSD YYFSWLTWVT DERVCLQWLK RVQNVSVLSI |
| | | CDFREDWQTW DCPKTQEHIE ESRTGWAGGF FVSTPVFSYD |
| | | AISYYKIFSD KDGYKHIHYI KDTVENAIQI TSGKWEAINI |
| | | FRVTQDSLFY SSNEFEEYPG RRNIYRISIG SYPPSKKCVT |
| | | CHLRKERCQY YTASFSDYAK YYALVCYGPG IPISTLHDGR |
| | | TDQEIKILEE NKELENALKN IQLPKEEIKK LEVDEITLWY |
| | | KMILPPQFDR SKKYPLLIQV YGGPCSQSVR SVFAVNWISY |
| | | LASKEGMVIA LVDGRGTAFQ GDKLLYAVYR KLGVYEVEDQ |
| | | ITAVRKFIEM GFIDEKRIAI WGWSYGGYVS SLALASGTGL |
| | | FKCGIAVAPV SSWEYYASVY TERFMGLPTK DDNLEHYKNS |
| | | TVMARAEYFR NVDYLLIHGT ADDNVHFQNS AQIAKALVNA |
| | | QVDFQAMWYS DQNHGLSGLS TNHLYTHMTH FLKQCFSLSD |
| 255 | His-tagged human FAP | RPSRVHNSEENTMRALTLKDILNGTFSYKTFFPNWISGQEYLHQ |
| | FED | SADNNIVLYNIETGQSYTILSNRTMKSVNASNYGLSPDRQFVYL |
| | | ESDYSKLWRYSYTATYYIYDLSNGEFVRGNELPRPIQYLCWSPV |
| | | GSKLAYVYQNNIYLKQRPGDPPFQITENGRENKIENGIPDWVYE |
| | | EEMLATKYALWWSPNGKFLAYAEFNDTDIPVIAYSYYGDEQYPR |
| | | TINIPYPKAGAKNPVVRIFIIDTTYPAYVGPQEVPVPAMIASSD |
| | | YYFSWLTWVTDERVCLQWLKRVQNVSVLSICDFREDWQTWDCPK |
| | | TQEHIEESRTGWAGGFEVSTPVESYDAISYYKIFSDKDGYKHIH |
| | | YIKDTVENAIQITSGKWEAINIERVTQDSLEYSSNEFEEYPGRR |
| | | NIYRISIGSYPPSKKCVTCHLRKERCQYYTASFSDYAKYYALVC |
| | | YGPGIPISTLHDGRTDQEIKILEENKELENALKNIQLPKEEIKK |
| | | LEVDEITLWYKMILPPQFDRSKKYPLLIQVYGGPCSQSVRSVFA |
| | | VNWISYLASKEGMVIALVDGRGTAFQGDKLLYAVYRKLGVYEVE |
| | | DQITAVRKFIEMGFIDEKRIAIWGWSYGGYVSSLALASGTGLFK |
| | | CGIAVAPVSSWEYYASVYTERFMGLPTKDDNLEHYKNSTVMARA |
| | | EYFRNVDYLLIHGTADDNVHFQNSAQIAKALVNAQVDFQAMWYS |
| | | DQNHGLSGLSTNHLYTHMTHFLKQCFSLSDGKKKKKKGHHHHHH |
| 256 | mouse FAP | UniProt accession no. P97321 |
| | | MKTWLKTVFG VTTLAALALV VICIVLRPSR VYKPEGNTKR |
| | | ALTLKDILNG TFSYKTYFPN WISEQEYLHQ SEDDNIVFYN |
| | | IETRESYIIL SNSTMKSVNA TDYGLSPDRQ FVYLESDYSK |
| | | LWRYSYTATY YIYDLQNGEF VRGYELPRPI QYLCWSPVGS |
| | | KLAYVYQNNI YLKQRPGDPP FQITYTGREN RIFNGIPDWV |
| | | YEEEMLATKY ALWWSPDGKF LAYVEFNDSD IPIIAYSYYG |
| | | DGQYPRTINI PYPKAGAKNP VVRVFIVDTT YPHHVGPMEV |
| | | PVPEMIASSD YYFSWLTWVS SERVCLQWLK RVQNVSVLSI |
| | | CDFREDWHAW ECPKNQEHVE ESRTGWAGGF FVSTPAFSQD |
| | | ATSYYKIFSD KDGYKHIHYI KDTVENAIQI TSGKWEAIYI |
| | | FRVTQDSLFY SSNEFEGYPG RRNIYRISIG NSPPSKKCVT |
| | | CHLRKERCQY YTASFSYKAK YYALVCYGPG LPISTLHDGR |
| | | TDQEIQVLEE NKELENSLRN IQLPKVEIKK LKDGGLTFWY |
| | | KMILPPQFDR SKKYPLLIQV YGGPCSQSVK SVFAVNWITY |
| | | LASKEGIVIA LVDGRGTAFQ GDKFLHAVYR KLGVYEVEDQ |
| | | LTAVRKFIEM GFIDEERIAI WGWSYGGYVS SLALASGTGL |
| | | FKCGIAVAPV SSWEYYASIY SERFMGLPTK DDNLEHYKNS |
| | | TVMARAEYFR NVDYLLIHGT ADDNVHFQNS AQIAKALVNA |
| | | QVDFQAMWYS DQNHGISSGR SQNHLYTHMT HFLKQCFSLS |
| | | D |
| 257 | His-tagged mouse FAP | RPSRVYKPEGNTKRALTLKDILNGTFSYKTYFPNWISEQEYLHQ |
| | FED | SEDDNIVFYNIETRESYIILSNSTMKSVNATDYGLSPDRQFVYL |
| | | ESDYSKLWRYSYTATYYIYDLQNGEFVRGYELPRPIQYLCWSPV |
| | | GSKLAYVYQNNIYLKQRPGDPPFQITYTGRENRIENGIPDWVYE |
| | | EEMLATKYALWWSPDGKFLAYVEFNDSDIPIIAYSYYGDGQYPR |
| | | TINIPYPKAGAKNPVVRVFIVDTTYPHHVGPMEVPVPEMIASSD |
| | | YYFSWLTWVSSERVCLQWLKRVQNVSVLSICDFREDWHAWECPK |
| | | NQEHVEESRTGWAGGFFVSTPAFSQDATSYYKIFSDKDGYKHIH |
| | | YIKDTVENAIQITSGKWEAIYIERVTQDSLEYSSNEFEGYPGRR |
| | | NIYRISIGNSPPSKKCVTCHLRKERCQYYTASFSYKAKYYALVC |
| | | YGPGLPISTLHDGRTDQEIQVLEENKELENSLRNIQLPKVEIKK |
| | | LKDGGLTFWYKMILPPQFDRSKKYPLLIQVYGGPCSQSVKSVFA |
| | | VNWITYLASKEGIVIALVDGRGTAFQGDKFLHAVYRKLGVYEVE |
| | | DQLTAVRKFIEMGFIDEERIAIWGWSYGGYVSSLALASGTGLFK |
| | | CGIAVAPVSSWEYYASIYSERFMGLPTKDDNLEHYKNSTVMARA |
| | | EYFRNVDYLLIHGTADDNVHFQNSAQIAKALVNAQVDFQAMWYS |
| | | DQNHGILSGRSQNHLYTHMTHFLKQCFSLSDGKKKKKKGHHHHH |
| | | H |
| 258 | His-tagged cynomolgus | RPPRVHNSEENTMRALTLKDILNGTFSYKTFFPNWISGQEYLHQ |
| | FAP ECD | SADNNIVLYNIETGQSYTILSNRTMKSVNASNYGLSPDRQFVYL |
| | | ESDYSKLWRYSYTATYYIYDLSNGEFVRGNELPRPIQYLCWSPV |
| | | GSKLAYVYQNNIYLKQRPGDPPFQITENGRENKIENGIPDWVYE |
| | | EEMLATKYALWWSPNGKFLAYAEFNDTDIPVIAYSYYGDEQYPR |
| | | TINIPYPKAGAKNPFVRIFIIDTTYPAYVGPQEVPVPAMIASSD |
| | | YYFSWLTWVTDERVCLQWLKRVQNVSVLSICDFREDWQTWDCPK |
| | | TQEHIEESRTGWAGGFEVSTPVESYDAISYYKIFSDKDGYKHIH |
| | | YIKDTVENAIQITSGKWEAINIERVTQDSLEYSSNEFEDYPGRR |
| | | NIYRISIGSYPPSKKCVTCHLRKERCQYYTASFSDYAKYYALVC |
| | | YGPGIPISTLHDGRTDQEIKILEENKELENALKNIQLPKEEIKK |
| | | LEVDEITLWYKMILPPQFDRSKKYPLLIQVYGGPCSQSVRSVFA |
| | | VNWISYLASKEGMVIALVDGRGTAFQGDKLLYAVYRKLGVYEVE |
| | | DQITAVRKFIEMGFIDEKRIAIWGWSYGGYVSSLALASGTGLFK |
| | | CGIAVAPVSSWEYYASVYTERFMGLPTKDDNLEHYKNSTVMARA |
| | | EYFRNVDYLLIHGTADDNVHFQNSAQIAKALVNAQVDFQAMWYS |
| | | DQNHGLSGLSTNHLYTHMTHFLKQCFSLSDGKKKKKKGHHHHHH |
| 259 | human CEA | UniProt accession no. P06731 |
| | | MESPSAPPHR WCIPWQRLLL TASLLTFWNP PTTAKLTIES |
| | | TPFNVAEGKE VLLLVHNLPQ HLFGYSWYKG ERVDGNRQII |
| | | GYVIGTQQAT PGPAYSGREI IYPNASLLIQ NIIQNDTGFY |
| | | TLHVIKSDLV NEEATGQFRV YPELPKPSIS SNNSKPVEDK |
| | | DAVAFTCEPE TQDATYLWWV NNQSLPVSPR LQLSNGNRTL |
| | | TLFNVTRNDT ASYKCETQNP VSARRSDSVI LNVLYGPDAP |
| | | TISPLNTSYR SGENLNLSCH AASNPPAQYS WFVNGTFQQS |
| | | TQELFIPNIT VNNSGSYTCQ AHNSDTGLNR TTVTTITVYA |
| | | EPPKPFITSN NSNPVEDEDA VALTCEPEIQ NTTYLWWVNN |
| | | QSLPVSPRLQ LSNDNRTLTL LSVTRNDVGP YECGIQNKLS |
| | | VDHSDPVILN VLYGPDDPTI SPSYTYYRPG VNLSLSCHAA |
| | | SNPPAQYSWL IDGNIQQHTQ ELFISNITEK NSGLYTCQAN |
| | | NSASGHSRTT VKTITVSAEL PKPSISSNNS KPVEDKDAVA |
| | | FTCEPEAQNT TYLWWVNGQS LPVSPRLQLS NGNRTLTLFN |
| | | VTRNDARAYV CGIQNSVSAN RSDPVTLDVL YGPDTPIISP |
| | | PDSSYLSGAN LNLSCHSASN PSPQYSWRIN GIPQQHTQVL |
| | | FIAKITPNNN GTYACFVSNL ATGRNNSIVK SITVSASGTS |
| | | PGLSAGATVG IMIGVLVGVA LI |
| 260 | human FolR1 | UniProt accession no. P15328: |
| | | MAQRMTTQLL LLLVWVAVVG EAQTRIAWAR TELLNVCMNA |
| | | KHHKEKPGPE DKLHEQCRPW RKNACCSTNT SQEAHKDVSY |
| | | LYRFNWNHCG EMAPACKRHF IQDTCLYECS PNLGPWIQQV |
| | | DQSWRKERVL NVPLCKEDCE QWWEDCRTSY TCKSNWHKGW |
| | | NWTSGFNKCA VGAACQPFHF YFPTPTVLCN EIWTHSYKVS |
| | | NYSRGSGRCI QMWFDPAQGN PNEEVARFYA AAMSGAGPWA |
| | | AWPFLLSLAL MLLWLLS |
| 261 | murine FolR1 | UniProt accession no. P35846: |
| | | MAHLMTVQLL LLVMWMAECA QSRATRARTE LLNVCMDAKH |
| | | HKEKPGPEDN LHDQCSPWKT NSCCSTNTSQ EAHKDISYLY |
| | | RFNWNHCGTM TSECKRHFIQ DTCLYECSPN LGPWIQQVDQ |
| | | SWRKERILDV PLCKEDCQQW WEDCQSSFTC KSNWHKGWNW |
| | | SSGHNECPVG ASCHPFTFYF PTSAALCEEI WSHSYKLSNY |
| | | SRGSGRCIQM WFDPAQGNPN EEVARFYAEA MSGAGFHGTW |
| | | PLLCSLSLVL LWVIS |
| 262 | cynomolgus FolR1 | UniProt accession no. G7PR14: |
| | | MAQRMTTQLL LLLVWVAVVG EAQTRTARAR TELLNVCMNA |
| | | KHHKEKPGPE DKLHEQCRPW KKNACCSTNT SQEAHKDVSY |
| | | LYRFNWNHCG EMAPACKRHF IQDTCLYECS PNLGPWIQQV |
| | | DQSWRKERVL NVPLCKEDCE RWWEDCRTSY TCKSNWHKGW |
| | | NWTSGFNKCP VGAACQPFHF YFPTPTVLCN EIWTYSYKVS |
| | | NYSRGSGRCI QMWFDPAQGN PNEEVARFYA AAMSGAGPWA |
| | | AWPLLLSLAL TLLWLLS |
| 263 | human MCSP | UniProt accession no. Q6UVK1: |
| | | MQSGPRPPLP APGLALALTL TMLARLASAA SFFGENHLEV |
| | | PVATALTDID LQLQFSTSQP EALLLLAAGP ADHLLLQLYS |
| | | GRLQVRLVLG QEELRLQTPA ETLLSDSIPH TVVLTVVEGW |
| | | ATLSVDGFLN ASSAVPGAPL EVPYGLFVGG TGTLGLPYLR |
| | | GTSRPLRGCL HAATLNGRSL LRPLTPDVHE GCAEEFSASD |
| | | DVALGFSGPH SLAAFPAWGT QDEGTLEFTL TTQSRQAPLA |
| | | FQAGGRRGDF IYVDIFEGHL RAVVEKGQGT VLLHNSVPVA |
| | | DGQPHEVSVH INAHRLEISV DQYPTHTSNR GVLSYLEPRG |
| | | SLLLGGLDAE ASRHLQEHRL GLTPEATNAS LLGCMEDLSV |
| | | NGQRRGLREA LLTRNMAAGC RLEEEEYEDD AYGHYEAFST |
| | | LAPEAWPAME LPEPCVPEPG LPPVFANFTQ LLTISPLVVA |
| | | EGGTAWLEWR HVQPTLDLME AELRKSQVLF SVTRGARHGE |
| | | LELDIPGAQA RKMFTLLDVV NRKARFIHDG SEDTSDQLVL |
| | | EVSVTARVPM PSCLRRGQTY LLPIQVNPVN DPPHIIFPHG |
| | | SLMVILEHTQ KPLGPEVFQA YDPDSACEGL TFQVLGTSSG |
| | | LPVERRDQPG EPATEFSCRE LEAGSLVYVH RGGPAQDLTF |
| | | RVSDGLQASP PATLKVVAIR PAIQIHRSTG LRLAQGSAMP |
| | | ILPANLSVET NAVGQDVSVL FRVTGALQFG ELQKQGAGGV |
| | | EGAEWWATQA FHQRDVEQGR VRYLSTDPQH HAYDTVENLA |
| | | LEVQVGQEIL SNLSFPVTIQ RATVWMLRLE PLHTQNTQQE |
| | | TLTTAHLEAT LEEAGPSPPT FHYEVVQAPR KGNLQLQGTR |
| | | LSDGQGFTQD DIQAGRVTYG ATARASEAVE DTERFRVTAP |
| | | PYFSPLYTFP IHIGGDPDAP VLTNVLLVVP EGGEGVLSAD |
| | | HLFVKSLNSA SYLYEVMERP RHGRLAWRGT QDKTTMVTSF |
| | | TNEDLLRGRL VYQHDDSETT EDDIPFVATR QGESSGDMAW |
| | | EEVRGVFRVA IQPVNDHAPV QTISRIFHVA RGGRRLLTTD |
| | | DVAFSDADSG FADAQLVLTR KDLLFGSIVA VDEPTRPIYR |
| | | FTQEDLRKRR VLFVHSGADR GWIQLQVSDG QHQATALLEV |
| | | QASEPYLRVA NGSSLVVPQG GQGTIDTAVL HLDTNLDIRS |
| | | GDEVHYHVTA GPRWGQLVRA GQPATAFSQQ DLLDGAVLYS |
| | | HNGSLSPRDT MAFSVEAGPV HTDATLQVTI ALEGPLAPLK |
| | | LVRHKKIYVF QGEAAEIRRD QLEAAQEAVP PADIVFSVKS |
| | | PPSAGYLVMV SRGALADEPP SLDPVQSFSQ EAVDTGRVLY |
| | | LHSRPEAWSD AFSLDVASGL GAPLEGVLVE LEVLPAAIPL |
| | | EAQNFSVPEG GSLTLAPPLL RVSGPYFPTL LGLSLQVLEP |
| | | PQHGALQKED GPQARTLSAF SWRMVEEQLI RYVHDGSETL |
| | | TDSFVLMANA SEMDRQSHPV AFTVTVLPVN DQPPILTTNT |
| | | GLQMWEGATA PIPAEALRST DGDSGSEDLV YTIEQPSNGR |
| | | VVLRGAPGTE VRSFTQAQLD GGLVLFSHRG TLDGGFRERL |
| | | SDGEHTSPGH FFRVTAQKQV LLSLKGSQTL TVCPGSVQPL |
| | | SSQTLRASSS AGTDPQLLLY RVVRGPQLGR LFHAQQDSTG |
| | | EALVNFTQAE VYAGNILYEH EMPPEPFWEA HDTLELQLSS |
| | | PPARDVAATL AVAVSFEAAC PQRPSHLWKN KGLWVPEGQR |
| | | ARITVAALDA SNLLASVPSP QRSEHDVLFQ VTQFPSRGQL |
| | | LVSEEPLHAG QPHFLQSQLA AGQLVYAHGG GGTQQDGFHF |
| | | RAHLQGPAGA SVAGPQTSEA FAITVRDVNE RPPQPQASVP |
| | | LRLTRGSRAP ISRAQLSVVD PDSAPGEIEY EVQRAPHNGF |
| | | LSLVGGGLGP VTRFTQADVD SGRLAFVANG SSVAGIFQLS |
| | | MSDGASPPLP MSLAVDILPS AIEVQLRAPL EVPQALGRSS |
| | | LSQQQLRVVS DREEPEAAYR LIQGPQYGHL LVGGRPTSAF |
| | | SQFQIDQGEV VFAFTNFSSS HDHFRVLALA RGVNASAVVN |
| | | VTVRALLHVW AGGPWPQGAT LRLDPTVLDA GELANRTGSV |
| | | PRFRLLEGPR HGRVVRVPRA RTEPGGSQLV EQFTQQDLED |
| | | GRLGLEVGRP EGRAPGPAGD SLTLELWAQG VPPAVASLDF |
| | | ATEPYNAARP YSVALLSVPE AARTEAGKPE SSTPTGEPGP |
| | | MASSPEPAVA KGGFLSFLEA NMFSVIIPMC LVLLLLALIL |
| | | PLLFYLRKRN KTGKHDVQVL TAKPRNGLAG DTETFRKVEP |
| | | GQAIPLTAVP GQGPPPGGQP DPELLQFCRT PNPALKNGQY |
| | | WV |
| 264 | human EGFR | UniProt accession no. P00533: |
| | | MRPSGTAGAA LLALLAALCP ASRALEEKKV CQGTSNKLTQ |
| | | LGTFEDHFLS LQRMFNNCEV VLGNLEITYV QRNYDLSFLK |
| | | TIQEVAGYVL IALNTVERIP LENLQIIRGN MYYENSYALA |
| | | VLSNYDANKT GLKELPMRNL QEILHGAVRF SNNPALCNVE |
| | | SIQWRDIVSS DFLSNMSMDF QNHLGSCQKC DPSCPNGSCW |
| | | GAGEENCQKL TKIICAQQCS GRCRGKSPSD CCHNQCAAGC |
| | | TGPRESDCLV CRKFRDEATC KDTCPPLMLY NPTTYQMDVN |
| | | PEGKYSFGAT CVKKCPRNYV VTDHGSCVRA CGADSYEMEE |
| | | DGVRKCKKCE GPCRKVCNGI GIGEFKDSLS INATNIKHFK |
| | | NCTSISGDLH ILPVAFRGDS FTHTPPLDPQ ELDILKTVKE |
| | | ITGFLLIQAW PENRTDLHAF ENLEIIRGRT KQHGQFSLAV |
| | | VSLNITSLGL RSLKEISDGD VIISGNKNLC YANTINWKKL |
| | | FGTSGQKTKI ISNRGENSCK ATGQVCHALC SPEGCWGPEP |
| | | RDCVSCRNVS RGRECVDKCN LLEGEPREFV ENSECIQCHP |
| | | ECLPQAMNIT CTGRGPDNCI QCAHYIDGPH CVKTCPAGVM |
| | | GENNTLVWKY ADAGHVCHLC HPNCTYGCTG PGLEGCPTNG |
| | | PKIPSIATGM VGALLLLLVV ALGIGLFMRR RHIVRKRTLR |
| | | RLLQERELVE PLTPSGEAPN QALLRILKET EFKKIKVLGS |
| | | GAFGTVYKGL WIPEGEKVKI PVAIKELREA TSPKANKEIL |
| | | DEAYVMASVD NPHVCRLLGI CLTSTVQLIT QLMPFGCLLD |
| | | YVREHKDNIG SQYLLNWCVQ IAKGMNYLED RRLVHRDLAA |
| | | RNVLVKTPQH VKITDFGLAK LLGAEEKEYH AEGGKVPIKW |
| | | MALESILHRI YTHQSDVWSY GVTVWELMTF GSKPYDGIPA |
| | | SEISSILEKG ERLPQPPICT IDVYMIMVKC WMIDADSRPK |
| | | FRELIIEFSK MARDPQRYLV IQGDERMHLP SPTDSNFYRA |
| | | LMDEEDMDDV VDADEYLIPQ QGFFSSPSTS RTPLLSSLSA |
| | | TSNNSTVACI DRNGLQSCPI KEDSFLQRYS SDPTGALTED |
| | | SIDDTFLPVP EYINQSVPKR PAGSVQNPVY HNQPLNPAPS |
| | | RDPHYQDPHS TAVGNPEYLN TVQPTCVNST FDSPAHWAQK |
| | | GSHQISLDNP DYQQDFFPKE AKPNGIFKGS TAENAEYLRV |
| | | APQSSEFIGA |
| 265 | human HER2 | Uniprot accession no. P04626: |
| | | MELAALCRWG LLLALLPPGA ASTQVCTGTD MKLRLPASPE |
| | | THLDMLRHLY QGCQVVQGNL ELTYLPTNAS LSFLQDIQEV |
| | | QGYVLIAHNQ VRQVPLQRLR IVRGTQLFED NYALAVLDNG |
| | | DPLNNTTPVT GASPGGLREL QLRSLTEILK GGVLIQRNPQ |
| | | LCYQDTILWK DIFHKNNQLA LTLIDTNRSR ACHPCSPMCK |
| | | GSRCWGESSE DCQSLTRTVC AGGCARCKGP LPTDCCHEQC |
| | | AAGCTGPKHS DCLACLHFNH SGICELHCPA LVTYNTDTFE |
| | | SMPNPEGRYT FGASCVTACP YNYLSTDVGS CTLVCPLHNQ |
| | | EVTAEDGTQR CEKCSKPCAR VCYGLGMEHL REVRAVTSAN |
| | | IQEFAGCKKI FGSLAFLPES FDGDPASNTA PLQPEQLQVF |
| | | ETLEEITGYL YISAWPDSLP DLSVFQNLQV IRGRILHNGA |
| | | YSLTLQGLGI SWLGLRSLRE LGSGLALIHH NTHLCFVHTV |
| | | PWDQLFRNPH QALLHTANRP EDECVGEGLA CHQLCARGHC |
| | | WGPGPTQCVN CSQFLRGQEC VEECRVLQGL PREYVNARHC |
| | | LPCHPECQPQ NGSVTCFGPE ADQCVACAHY KDPPFCVARC |
| | | PSGVKPDLSY MPIWKFPDEE GACQPCPINC THSCVDLDDK |
| | | GCPAEQRASP LTSIISAVVG ILLVVVLGVV FGILIKRRQQ |
| | | KIRKYTMRRL LQETELVEPL TPSGAMPNQA QMRILKETEL |
| | | RKVKVLGSGA FGTVYKGIWI PDGENVKIPV AIKVLRENTS |
| | | PKANKEILDE AYVMAGVGSP YVSRLLGICL TSTVQLVTQL |
| | | MPYGCLLDHV RENRGRLGSQ DLLNWCMQIA KGMSYLEDVR |
| | | LVHRDLAARN VLVKSPNHVK ITDFGLARLL DIDETEYHAD |
| | | GGKVPIKWMA LESILRRRFT HQSDVWSYGV TVWELMTFGA |
| | | KPYDGIPARE IPDLLEKGER LPQPPICTID VYMIMVKCWM |
| | | IDSECRPRFR ELVSEFSRMA RDPQRFVVIQ NEDLGPASPL |
| | | DSTFYRSLLE DDDMGDLVDA EEYLVPQQGF FCPDPAPGAG |
| | | GMVHHRHRSS STRSGGGDLT LGLEPSEEEA PRSPLAPSEG |
| | | AGSDVFDGDL GMGAAKGLQS LPTHDPSPLQ RYSEDPTVPL |
| | | PSETDGYVAP LTCSPQPEYV NQPDVRPQPP SPREGPLPAA |
| | | RPAGATLERP KTLSPGKNGV VKDVFAFGGA VENPEYLTPQ |
| | | GGAAPQPHPP PAFSPAFDNL YYWDQDPPER GAPPSTFKGT |
| | | PTAENPEYLG LDVPV |
| 266 | p95 HER2 | MPIWKFPDEEGACQPCPINCTHSCVDLDDKGCPAEQRASPLTSI |
| | | ISAVVGILLVVVLGVVFGILIKRRQQKIRKYTMRRLLQETELVE |
| | | PLTPSGAMPNQAQMRILKETELRKVKVLGSGAFGTVYKGIWIPD |
| | | GENVKIPVAIKVLRENTSPKANKEILDEAYVMAGVGSPYVSRLL |
| | | GICLTSTVQLVTQLMPYGCLLDHVRENRGRLGSQDLLNWCMQIA |
| | | KGMSYLEDVRLVHRDLAARNVLVKSPNHVKITDFGLARLLDIDE |
| | | TEYHADGGKVPIKWMALESILRRRFTHQSDVWSYGVTVWELMTF |
| | | GAKPYDGIPAREIPDLLEKGERLPQPPICTIDVYMIMVKCWMID |
| | | SECRPRFRELVSEFSRMARDPQRFVVIQNEDLGPASPLDSTFYR |
| | | SLLEDDDMGDLVDAEEYLVPQQGFFCPDPAPGAGGMVHHRHRSS |
| | | STRSGGGDLTLGLEPSEEEAPRSPLAPSEGAGSDVFDGDLGMGA |
| | | AKGLQSLPTHDPSPLQRYSEDPTVPLPSETDGYVAPLTCSPQPE |
| | | YVNQPDVRPQPPSPREGPLPAARPAGATLERPKTLSPGKNGVVK |
| | | DVFAFGGAVENPEYLTPQGGAAPQPHPPPAFSPAFDNLYYWDQD |
| | | PPERGAPPSTFKGTPTAENPEYLGLDVPV |
| 267 | CH1 domain | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKV |
| 268 | CH1 to hinge | EPKSC |
| 269 | CH2 domain | APELLGGPSV FLFPPKPKDT LMISRTPEVT CVWDVSHEDP |
| | | EVKFNWYVDG VEVHNAKTKP REEQESTYRW SVLTVLHQDW |
| | | LNGKEYKCKV SNKALPAPIE KTISKAK |
| 270 | CH3 domain | GQPREPQVYT LPPSRDELTK NQVSLTCLVK GFYPSDIAVE |
| | | WESNGQPENN YKTTPPVLDS DGSFFLYSKL TVDKSRWQQG |
| | | NVFSCSVMHE ALHNHYTQKS LSLSPG |
| 271 | Peptide linker (G4S) | GGGGS |
| 272 | Peptide linker (G4S)₂ | GGGGSGGGGS |
| 273 | Peptide linker (SG4)₂ | SGGGGSGGGG |
| 274 | Peptide linker G4(SG4)₂ | GGGGSGGGGSGGGG |
| 275 | peptide linker | GSPGSSSSGS |
| 276 | (G4S)₃ peptide linker | GGGGSGGGGSGGGGS₃ |
| 277 | (G4S)₄ peptide linker | GGGGSGGGGSGGGGSGGGGS |
| 278 | peptide linker | GSGSGSGS |
| 279 | peptide linker | GSGSGNGS |
| 280 | peptide linker | GGSGSGSG |
| 281 | peptide linker | GGSGSG |
| 282 | peptide linker | GGSG |
| 283 | peptide linker | GGSGNGSG |
| 284 | peptide linker | GGNGSGSG |
| 285 | peptide linker | GGNGSG |
| 286 | human PD-L1 (Uniprot | MRIFAVFIFMTYWHLLNAFTVTVPKDLYVVEYGSNMTIECKFPV |
| | Q9NZQ7) | EKQLDLAALIVYWEMEDKNIIQFVHGEEDLKVQHSSYRQRARLL |
| | | KDQLSLGNAALQITDVKLQDAGVYRCMISYGGADYKRITVKVNA |
| | | PYNKINQRILVVDPVTSEHELTCQAEGYPKAEVIWTSSDHQVLS |
| | | GKTTTTNSKREEKLFNVTSTLRINTTTNEIFYCTFRRLDPEENH |
| | | TAELVIPELPLAHPPNERTHLVILGAILLCLGVALTFIFRLRKG |
| | | RMMDVKKCGIQDTNSKKQSDTHLEET |
| 287 | human PD-1 (Uniprot | MQIPQAPWPVVWAVLQLGWRPGWFLDSPDRPWNPPTFSPALLVV |
| | Q15116) | TEGDNATFTCSFSNTSESFVLNWYRMSPSNQTDKLAAFPEDRSQ |
| | | PGQDCRFRVTQLPNGRDFHMSVVRARRNDSGTYLCGAISLAPKA |
| | | QIKESLRAELRVTERRAEVPTAHPSPSPRPAGQFQTLVVGVVGG |
| | | LLGSLVLLVWVLAVICSRAARGTIGARRTGQPLKEDPSAVPVFS |
| | | VDYGELDFQWREKTPEPPVPCVPEQTEYATIVFPSGMGTSSPAR |
| | | RGSADGPRSAQPLRPEDGHCSWPL |
| 288 | VH (PD-L1) | EVQLVESGGGLVQPGGSLRLSCAASGFTFSDSWIHWVRQAPGKG |
| | | LEWVAWISPYGGSTYYADSVKGRFTISADTSKNTAYLQMNSLRA |
| | | EDTAVYYCARRHWPGGFDYWGQGTLVTVSS |
| 289 | VL (PD-L1) | DIQMTQSPSSLSASVGDRVTITCRASQDVSTAVAWYQQKPGKAP |
| | | KLLIYSASFLYSGVPSRFSGSGSGTDFTLTISSLQPEDFATYYC |
| | | QQYLYHPATFGQGTKVEIK |
| 290 | VH (PD-L1) | EVQLVESGGGLVQPGGSLRLSCAASGFTFSRYWMSWVRQAPGKG |
| | | LEWVANIKQDGSEKYYVDSVKGRFTISRDNAKNSLYLQMNSLRA |
| | | EDTAVYYCAREGGWFGELAFDYWGQGTLVTVSS |
| 291 | VL (PD-L1) | EIVLTQSPGTLSLSPGERATLSCRASQRVSSSYLAWYQQKPGQA |
| | | PRLLIYDASSRATGIPDRFSGSGSGTDFTLTISRLEPEDFAVYY |
| | | CQQYGSLPWTFGQGTKVEIK |
| 292 | VH (PD-1) | QVQLVQSGVEVKKPGASVKVSCKASGYTFTNYYMYWVRQAPGQG |
| | | LEWMGGINPSNGGTNENEKEKNRVTLTTDSSTTTAYMELKSLQF |
| | | DDTAVYYCARRDYREDMGEDYWGQGTTVTVSS |
| 293 | VL (PD-1) | EIVLTQSPATLSLSPGERATLSCRASKGVSTSGYSYLHWYQQKP |
| | | GQAPRLLIYLASYLESGVPARFSGSGSGTDFTLTISSLEPEDFA |
| | | VYYCQHSRDLPLTFGGGTKVEIK |
| 294 | VH (PD-1) | QVQLVESGGGVVQPGRSLRLDCKASGITFSNSGMHWVRQAPGKG |
| | | LEWVAVIWYDGSKRYYADSVKGRFTISRDNSKNTLFLQMNSLRA |
| | | EDTAVYYCATNDDYWGQGTLVTVSS |
| 295 | VL (PD-1) | EIVLTQSPATLSLSPGERATLSCRASQSVSSYLAWYQQKPGQAP |
| | | RLLIYDASNRATGIPARESGSGSGTDFTLTISSLEPEDFAVYYC |
| | | QQSSNWPRTFGQGTKVEIK |
| 296 | ICOS (009v1) VH | EVRLDETGGGVVQPGRPMELSCVASGFTESDYWNINWVRQSPKGL |
| | | EWVAQIRNKPYNYETYYSDSVKGRFTISRDDSKSRVYLQMNNLR |
| | | AEDMGIYYCTWPRLRSSDWHFDVWGAGTTVTVSS |
| 297 | ICOS (009v1) VL | AIQMTQSPSSLSASLGGEVTITCKASQDINKNIAWYQHKP |
| | | GRGPRLLIWYTSTLQTGIPSRFSGSGSGRDYSFTISNLEP |
| | | EDFATYYCLQFDNLYTFGSGTKLEIR |
| 298 | ICOS (1143v1) VH | QSLEESGGDLVKPGASLTLTCKASGFDFSSAYDMSWVRQAPGKG |
| | | LEWIGVVYYGDGITYYATWAKGRFTISKTSSTTVPLQMTSLTAA |
| | | DTATYFCARGAFLGSSYYLSLWGQGTLVTVSS |
| 299 | ICOS (1143v1) VL | AIDMTQTPASVEAAVGGTVTINCQASENIYNWLAWYQQKPGQPP |
| | | KLLIYDASKLASGVPSRFSASGSGTQFTLTISAVECADAATYYC |
| | | QQAYTYGNIDNAFGGGTEVVVS |
| 300 | ICOS (1143v2) VH | QSLEESGGDLVKPGASLTLTCKASGFDFSSAYDMSWVRQAPGKG |
| | | LEWIGVIYYGDGITYYATSVKGRFTISKTSSTTVPLQMTSLTAA |
| | | DTATYFCARGAFLGSSYYLSLWGQGTLVTVSS |
| 301 | ICOS (1143v2) VL | AIDMTQTPASVEAAVGGTVTINCQASENIYNWLAWYQQKPGQPP |
| | | KLLIYDASKLASGVPSRFSASGSGTQFTLTISAVECADAATYYC |
| | | QQAYTYGNIDNAFGGGTEVVVS |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the numbering systems according to Kabat (Kabat, E. A., et al., Sequences of Proteins of Immunological Interest, 5th ed., Public Health Service, National Institutes of Health, Bethesda, Md. (1991)) as defined above.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Protein Purification

Proteins were purified from filtered cell culture supernatants referring to standard protocols. In brief, antigen binding molecules were applied to a Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. Aggregated protein was separated from monomeric antibodies by size exclusion chromatography (Superdex 200, GE Healthcare) in PBS or in 20 mM Histidine, 140 mM NaCl at pH 6.0. Monomeric antigen binding molecule fractions can be pooled, concentrated (if required) using e.g., a MILLIPORE Amicon Ultra (30 MWCO) centrifugal concentrator, frozen and stored at −20° C. or −80° C. Part of the samples can be provided for subsequent protein analytics and analytical characterization e.g. by SDS-PAGE, size exclusion chromatography (SEC) or mass spectrometry.

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

Mass Spectrometry

This section describes the characterization of the multispecific antibodies with VH/VL exchange (VH/VL CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs.

The VH/VL CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 μg deglycosylated VH/VL CrossMabs in a Tris buffer pH 8 at room temperature for 120 hours and at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

Determination of Binding and Binding Affinity of Multispecific Antibodies to the Respective Antigens Using Surface Plasmon Resonance (SPR) (BIACORE)

Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CM5 chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3-10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

### Generation of ICOS Antibodies

### 1.1 Preparation, Purification and Characterization of Antigens and Screening Tools for the Generation of Novel ICOS Binders by Immunization

### 1.1.1 Preparation, Purification and Characterization of Monomeric Und Dimeric ICOS Antigen Fc(kih) Fusion Molecules

DNA sequences encoding the ectodomains of human, cynomolgus or mouse or 4-1BB (Table 1) were subcloned in frame with the human IgG1 heavy chain CH2 and CH3 domains on the knob for monomeric and on the hole and knob for dimeric ICOS antigen Fc fusion molecules (Merchant et al., 1998). An Avi tag for directed biotinylation was introduced at the C-terminus of the antigen-Fc knob. Combination of the antigen-Fc knob chain containing the S354C/T366W mutations, with a Fc hole chain containing the Y349C/T366S/L368A/Y407V mutations allows generation of a ICOS heterodimer which includes a single copy or a homodimer which includes two copies of the ectodomain containing chain, thus creating a monomeric or dimeric form of Fc-linked antigen. Table 2 shows the amino acid sequences of the antigen Fc-fusion constructs.

TABLE 1
Amino acid numbering of antigen ectodomains (ECD)
and their origin

| SEQ | | | |
|---|---|---|---|
| ID | | | |
| NO: | Organism | Origin | ECD |
| 1 | human ICOS | Synthetized according | aa 21-140 |
| | | to Q9Y6W8 | |
| 2 | cynomolgus ICOS | Synthetized according | aa 21-140 |
| | | to G7PL89 | |
| 3 | murine ICOS | Synthetized according | aa 21-144 |
| | | to Q9WVS0 | |

TABLE 2
cDNA and amino acid sequences of dimeric antigen Fc(kih) fusion molecules

| SEQ ID | | |
|---|---|---|
| NO: | Antigen | Sequence |
| 70 | human ICOS | EINGSANYEMFIFHNGGVQILCKYPDIVQQFKMQLLKGGQILCDLTKT |
| | antigen Fc hole | KGSGNTVSIKSLKFCHSQLSNNSVSFFLYNLDHSHANYYFCNLSIFDP |
| | chain (dimeric) | PPFKVTLTGGYLHIYESQLCCQLKSADVDDKTHTCPPCPAPEAAGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALGAPIEKT |
| | | ISKAKGQPREPQVCTLPPSRDELTKNQVSLSCAVKGFYPSDIAVEWES |
| | | NGQPENNYKTTPPVLDSDGSFFLVSKLTVDKSRWQQGNVFSCSVMHEA |
| | | LHNRFTQKSLSLSPGK |
| 71 | human ICOS | EINGSANYEMFIFHNGGVQILCKYPDIVQQFKMQLLKGGQILCDLTKT |
| | antigen Fc knob | KGSGNTVSIKSLKFCHSQLSNNSVSFFLYNLDHSHANYYFCNLSIFDP |
| | chain (dimeric) | PPFKVTLTGGYLHIYESQLCCQLKSADVDASGGSPTPPTPGGGSADKT |
| | | HTCPPCPAPEAAGGPSVFLFPPKPKDTLMISRTPEVTCVVVDVSHEDP |
| | | EVKFNWYVDGVEVHNAKTKPREEQYNSTYRVVSVLTVLHQDWLNGKEY |
| | | KCKVSNKALGAPIEKTISKAKGQPREPQVYTLPPCRDELTKNQVSLWC |
| | | LVKGFYPSDIAVEWESNGQPENNYKTTPPVLDSDGSFFLYSKLTVDKS |
| | | RWQQGNVFSCSVMHEALHNHYTQKSLSLSPGKSGGLNDIFEAQKIEWH |
| | | E |
| 72 | human ICOS | DKTHTCPPCPAPEAAGGPSVFLFPPKPKDTLMISRTPEVTCVVVDVSH |
| | antigen Fc hole | EDPEVKFNWYVDGVEVHNAKTKPREEQYNSTYRVVSVLTVLHQDWLNG |
| | chain | KEYKCKVSNKALGAPIEKTISKAKGQPREPQVCTLPPSRDELTKNQVS |
| | (monomeric) | LSCAVKGFYPSDIAVEWESNGQPENNYKTTPPVLDSDGSFFLVSKLTV |
| | | DKSRWQQGNVFSCSVMHEALHNRFTQKSLSLSPGK |
| 71 | human ICOS | See human ICOS antigen Fc knob chain (dimeric) |
| | antigen Fc knob | |
| | chain | |
| | (monomeric) | |
| 73 | cynomolgus | EINGSANYEMFIFHNGGVQILCKYPDIVQQFKMQLLKGGQILCDLTKT |
| | ICOS antigen Fc | KGSGNKVSIKSLKFCHSQLSNNSVSFFLYNLDRSHANYYFCNLSIFDP |
| | hole chain | PPFKVTLTGGYLHIYESQLCCQLKSADVDDKTHTCPPCPAPEAAGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALGAPIEKT |
| | | ISKAKGQPREPQVCTLPPSRDELTKNQVSLSCAVKGFYPSDIAVEWES |
| | | NGQPENNYKTTPPVLDSDGSFELVSKLTVDKSRWQQGNVESCSVMHEA |
| | | LHNRFTQKSLSLSPGK |
| 74 | cynomolgus | EINGSANYEMFIFHNGGVQILCKYPDIVQQFKMQLLKGGQILCDLTKT |
| | ICOS antigen Fc | KGSGNKVSIKSLKFCHSQLSNNSVSFFLYNLDRSHANYYFCNLSIFDP |
| | knob chain | PPFKVTLTGGYLHIYESQLCCQLKSADVDASGGSPTPPTPGGGSADKT |
| | | HTCPPCPAPEAAGGPSVFLFPPKPKDTLMISRTPEVTCVVVDVSHEDP |
| | | EVKFNWYVDGVEVHNAKTKPREEQYNSTYRVVSVLTVLHQDWLNGKEY |
| | | KCKVSNKALGAPIEKTISKAKGQPREPQVYTLPPCRDELTKNQVSLWC |
| | | LVKGFYPSDIAVEWESNGQPENNYKTTPPVLDSDGSFFLYSKLTVDKS |
| | | RWQQGNVFSCSVMHEALHNHYTQKSLSLSPGKSGGLNDIFEAQKIEWH |
| | | E |
| 75 | murine ICOS | EINGSADHRMFSFHNGGVQISCKYPETVQQLKMRLFREREVLCELTKT |
| | antigen Fc hole | KGSGNAVSIKNPMLCLYHLSNNSVSFFLNNPDSSQGSYYFCSLSIFDP |
| | chain | PPFQERNLSGGYLHIYESQLCCQLKLWLSADVDDKTHTCPPCPAPEAA |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALGAP |
| | | IEKTISKAKGQPREPQVCTLPPSRDELTKNQVSLSCAVKGFYPSDIAV |
| | | EWESNGQPENNYKTTPPVLDSDGSFELVSKLTVDKSRWQQGNVESCSV |
| | | MHEALHNRFTQKSLSLSPGK |
| 76 | murine ICOS | EINGSADHRMFSFHNGGVQISCKYPETVQQLKMRLFREREVLCELTKT |
| | antigen Fc knob | KGSGNAVSIKNPMLCLYHLSNNSVSFFLNNPDSSQGSYYFCSLSIFDP |
| | chain | PPFQERNLSGGYLHIYESQLCCQLKLWLSADVDASGGSPTPPTPGGGS |
| | | ADKTHTCPPCPAPEAAGGPSVFLFPPKPKDTLMISRTPEVTCVVVDVS |
| | | HEDPEVKFNWYVDGVEVHNAKTKPREEQYNSTYRVVSVLTVLHQDWLN |
| | | GKEYKCKVSNKALGAPIEKTISKAKGQPREPQVYTLPPCRDELTKNQV |
| | | SLWCLVKGFYPSDIAVEWESNGQPENNYKTTPPVLDSDGSFFLYSKLT |
| | | VDKSRWQQGNVFSCSVMHEALHNHYTQKSLSLSPGKSGGLNDIFEAQK |
| | | IEWHE |

All ICOS-Fc-fusion encoding sequences were cloned into a plasmid vector driving expression of the insert from an chimeric MPSV promoter and containing a synthetic polyA signal sequence located at the 3′ end of the CDS. In addition, the vector contained an EBV OriP sequence for episomal maintenance of the plasmid.

For preparation of the biotinylated antigen/Fc fusion molecules, exponentially growing suspension HEK293 EBNA cells were co-transfected with three vectors encoding the two components of fusion protein (knob and hole chains) as well as BirA, an enzyme necessary for the biotinylation reaction. The corresponding vectors were used at a 1:1:0.05 ratio (“Fc knob”:“Fc hole”:“BirA”).

For protein production in 500 ml shake flasks, 400 million HEK293 EBNA cells were seeded 24 hours before transfection. For transfection cells were centrifuged for 5 minutes at 210 g, and supernatant was replaced by pre-warmed CD CHO medium. Expression vectors were resuspended in 20 mL of CD CHO medium containing 200 μg of vector DNA. After addition of 540 μL of polyethylenimine (PEI), the solution was vortexed for 15 seconds and incubated for 10 minutes at room temperature. Afterwards, cells were mixed with the DNA/PEI solution, transferred to a 500 mL shake flask and incubated for 3 hours at 37° C. in an incubator with a 5% CO₂ atmosphere. After the incubation, 160 mL of F17 medium was added and cells were cultured for 24 hours. One day after transfection, 1 mM valproic acid and 7% Feed were added to the culture. After 7 days of culturing, the cell supernatant was collected by spinning down cells for 15 min at 210 g. The solution was sterile filtered (0.22 μm filter), supplemented with sodium azide to a final concentration of 0.01% (w/v), and kept at 4° C.

Secreted proteins were purified from cell culture supernatants by affinity chromatography using Protein A, followed by size exclusion chromatography. For affinity chromatography, the supernatant was loaded on a HiTrap ProteinA HP column (CV=5 mL, GE Healthcare) equilibrated with 40 mL 20 mM sodium phosphate, 20 mM sodium citrate pH 7.5. Unbound protein was removed by washing with at least 10 column volumes of a buffer containing 20 mM sodium phosphate, 20 mM sodium citrate and 0.5 M sodium chloride (pH 7.5). The bound protein was eluted using a linear pH-gradient of sodium chloride (from 0 to 500 mM) created over 20 column volumes of 20 mM sodium citrate, 0.01% (v/v) Tween-20, pH 3.0. The column was then washed with 10 column volumes of a solution containing 20 mM sodium citrate, 500 mM sodium chloride and 0.01% (v/v) Tween-20, pH 3.0.

The pH of the collected fractions was adjusted by adding 1/40 (v/v) of 2M Tris, pH8.0. The protein was concentrated and filtered prior to loading on a HiLoad Superdex 200 column (GE Healthcare) equilibrated with 2 mM MOPS, 150 mM sodium chloride, 0.02% (w/v) sodium azide solution of pH 7.4.

### 1.1.2 Generation and Characterisation of Stable Cell Lines Expressing Recombinant ICOS

Full-length cDNAs encoding human or murine ICOS were subcloned into mammalian expression vector. Plasmids were transfected into CHO-K1 (ATCC, CCL-61) cells using Lipofectamine LTX Reagent (Invitrogen, #15338100) according to the manufacturer's protocol. Stably transfected ICOS-positive CHO-K1 cells were maintained in DMEM/F-12 (Gibco, #11320033) supplemented with 10% fetal bovine serum (Gibco, #16140063) and 1% GlutaMAX Supplement (Gibco; #31331-028). Two days after transfection, puromycin (Invivogen; #ant-pr-1) was added to 6 μg/mL. After initial selection, the cells with the highest cell surface expression of ICOS were sorted using BD FACSAria III cell sorter (BD Biosciences) and cultured to establish stable cell clones. The expression level and stability was confirmed by FACS analysis using PE anti-human/mouse/rat CD278 antibody (BioLegend; #313508) over a period of 4 weeks.

### 1.1.3 Generation of an ICOS Expression Vector for DNA Immunization

Full-length cDNAs encoding human ICOS was subcloned into standard mammalian expression vector. The plasmid DNA was purified from transformed bacteria and concentration determined by UV spectroscopy. The DNA sequence of the subcloned gene fragments was confirmed by DNA sequencing.

### 1.2 Generation of ICOS-Specific 009, 1167, 1143 and 1138 Antibodies by Rabbit and Mouse Immunization

### 1.2.1 Immunization Campaigns

For immunization, NMRI mice and New Zealand White rabbits (NZW) obtained from Charles River Laboratories International, Inc. as well as Roche proprietary transgenic rabbits, expressing a humanized antibody repertoire, upon immunization with ICOS-derived antigens, were used. Transgenic rabbits comprising a human immunoglobulin locus are reported in WO 2000/46251, WO 2002/12437, WO 2005/007696, WO 2006/047367, US 2007/0033661, and WO 2008/027986. The animals were housed according to the Appendix A “Guidelines for accommodation and care of animals” in an AAALAC-accredited animal facility. All animal immunization protocols and experiments were approved by the Government of Upper Bavaria (permit number 55.2-1-54-2532-66-16 and 55.2-1-54-2532-90-14) and performed according to the German Animal Welfare Act and the Directive 2010/63 of the European Parliament and Council.

Generation of ICOS Antibody 009

NMRI mice (n=5), 6-8 weeks old, received three immunizations with a recombinant Fc-fused human ICOS ECD molecule (see Example 1.1.1) over a course of 1.5 months. For the first immunization, 100 μg protein dissolved in 20 mM His/HisCl, 140 mM NaCl, pH 6.0, was mixed with an equal volume of complete Freund's adjuvant (BD Difco, #263810) and administered intraperitoneally. Booster immunizations were given on days 21 and 42 in a similar fashion, except that incomplete Freund's adjuvant (BD Difco, #DIFC263910) was used. Four to five weeks after the final immunization, mice received approximately 50 μg of the immunogen intraperitoneally in sterile PBS and one day later 25 μg of the immunogen intravenously in sterile PBS. 48 h later, spleens were aseptically harvested and prepared for hybridoma generation. Serum was tested for recombinant Fc-fused human ICOS ECD (see Example 1.1.1) by ELISA after the third immunization.

Generation of ICOS Antibodies 1183, 1143 (NZW Rabbits) and 1167 (Tg Rabbits)

Rabbits (NZW: n=2, trangenic rabbits: n=2), 12-16 weeks old, were genetically immunized with a plasmid expression vector encoding for full-length human ICOS (see Example 1.1.3) and human ICOS expressing cells in an alternating regime.

All animals received 400 μg vector DNA by intradermal application with concomitant electroporation (5 square pulses of 750 V/cm, duration 10 ms, interval 1 s) at weeks 0, 4 and 12. In addition 3-5×10⁷ human ICOS expressing SR cells (ATCC; CRL-2262) or activated human primary T cells emulsified in complete Freund's adjuvant (CFA; BD Difco, #263810) or mixed with a combination of TLR agonists were injected intradermal at week 2, intramuscular at week 8 and subcutaneous at week 16. Booster immunizations were given on days 28 (DNA), 42 (T cells), 56 (DNA) and 70 (T cells) in a similar fashion, except that CFA was used as adjuvant for cell immunizations.

Blood (10% of estimated total blood volume) was retrieved at days 6 to 8 post immunizations, starting from the 3rd immunization onwards. Serum was prepared, which was used for antigen-specific titer determination by ELISA, and peripheral mononuclear cells were isolated, which were used as a source of antigen-specific B cells in the B cell cloning process (see Example 1.2.2).

1.2.2 B-Cell Cloning from Rabbits

Blood samples were taken of immunized wild type rabbits or rabbits transgenic for human IgGs. EDTA containing whole blood was diluted twofold with 1×PBS before density centrifugation using lympholyte mammal (Cedarlane Laboratories) according to the specifications of the manufacturer. The PBMCs were washed twice with 1×PBS.

EL-4 B5 Medium

RPMI 1640 medium supplemented with 10% FCS, 2 mM Glutamin, 1% penicillin/streptomycin solution, 2 mM sodium pyruvate, 10 mM HEPES and 0.05 mM b-mercaptoethanole was used.

Coating of Plates

Sterile 6-well plates (cell culture grade) were used for coating with antigen.

Coating 1, Protein: The human ICOS protein antigen (ID 1486) was diluted with carbonate coating buffer (0.1 M sodium bicarbonate, 34 mM Disodiumhydrogencarbonate, pH 9.55) to a final concentration of 2 μg/ml. 3 ml of this solution were added to each well of a 6-well plate and incubated over night at room temperature. Prior to use the supernatant was removed and the wells were washed 3× with PBS.

Coating 2, Cells: The parental CHO-K1 cell line (Coating 2a) or CHO cells expressing murine ICOS (Coating 2b) were seeded in 6-well plates and incubated at 37° C. in the incubator until confluent growth was observed.

Removal of Macrophages/Monocytes from PBMCs

The PBMCs were either seeded on plain sterile 6-well plates (cell culture grade) or on 6-well plates already containing a cell layer with CHO cells to deplete macrophages and monocytes through unspecific adhesion.

Each well was filled at maximum with 4 ml medium and up to 6×10e6 PBMCs from the immunized rabbit and were allowed to bind for 1 h at 37° C. in the incubator. The cells in the supernatant (peripheral blood lymphocytes (PBLs)) were used for the antigen panning step and were therefore concentrated by centrifugation at 800×g for 10 min. The pellet was resuspended in medium.

Enrichment of Antigen-Specific B-Cells

The PBLs of the blood sample were adjusted to a cell density of 2×10e6 cells/ml and 3 ml are added to each well (up to 6×10⁶ cells per 3-4 ml medium) of a 6-well plate coated either with Coating 1 or 2. The plate was incubated for 60 to 90 min at 37° C. in the incubator. The supernatant was removed and non-adherent cells were removed by carefully washing the wells 1-4 times with 1×PBS. For retrieval of the sticky antigen-specific B cells, 1 ml of a trypsin/EDTA-solution was added to the wells of the 6 well plate and incubated for 5 to 10 min at 37° C. The incubation was stopped by addition of medium and the supernatant was transferred to a centrifugation vial. The wells were washed twice with PBS and the supernatants were combined with the other supernatants. The cells were pelleted by centrifugation for 10 min at 800×g and were kept on ice until the immune fluorescence staining.

Immune Fluorescence Staining and Flow Cytometry

The anti-IgG FITC (AbD Serotec) and the anti-huCk PE (Dianova) antibody was used for single cell sorting. For surface staining, cells from the depletion and enrichment step were incubated with the anti-IgG FITC and the anti-huCk PE antibody in PBS for 45 min in the dark at 4° C. After staining the PBMCs were washed two fold with ice cold PBS. Finally, the PBMCs were resuspended in ice cold PBS and immediately subjected to the FACS analyses. Propidium iodide in a concentration of 5 μg/ml (BD Pharmingen) was added prior to the FACS analyses to discriminate between dead and live cells.

A Becton Dickinson FACSAria equipped with a computer and the FACSDiva software (BD Biosciences) were used for single cell sort.

B-Cell Cultivation

The cultivation of the rabbit B cells was performed by a method described by Seeber et al., PLoS One 2014, 9(2), e86184. Briefly, single-cell sorted rabbit B cells were incubated in 96-well plates with 200 μl/well EL-4 B5 medium containing Pansorbin Cells (1:100000) (Calbiochem), 5% rabbit thymocyte supernatant (MicroCoat) and gamma-irradiated murine EL-4 B5 thymoma cells (5×10e5 cells/well) for 7 days at 37° C. in the incubator. The supernatants of the B-cell cultivation were removed for screening and the remaining cells were harvested immediately and were frozen at −80° C. in 100 μl RLT buffer (Qiagen).

### 1.2.3 PCR Amplification of V-Domains

Total RNA was prepared from B cells lysate (resuspended in RLT buffer, Qiagen—Cat. N^{o} 79216) using the NucleoSpin 8/96 RNA kit (Macherey&Nagel; 740709.4, 740698) according to manufacturer's protocol. RNA was eluted with 60 μl RNase free water. 6 μl of RNA was used to generate cDNA by reverse transcriptase reaction using the Superscript III First-Strand Synthesis SuperMix (Invitrogen 18080-400) and an oligo dT-primer according to the manufacture's instructions. All steps were performed on a Hamilton ML Star System. 4 μl of cDNA were used to amplify the immunoglobulin heavy and light chain variable regions (VH and VL) with the AccuPrime Supermix (Invitrogen 12344-040) in a final volume of 50 μl using the primers rbHC.up and rbHC.do for the heavy chain, rbLC.up and rbLC.do for the light chain of Wild Type Rabbit B cells and BcPCR_FHLC_leader.fw and BcPCR_huCkappa.rev for the light chain of transgenic rabbit B cells as described in WO 2015/101588 (see Table 3). All forward primers were specific for the signal peptide (of respectively VH and VL) whereas the reverse primers were specific for the constant regions (of respectively VH and VL). The PCR conditions for the RbVH+RbVL were as follows: Hot start at 94° C. for 5 min; 35 cycles of 20 s at 94° C., 20 s at 70° C., 45 s at 68° C., and a final extension at 68° C. for 7 min. The PCR conditions for the HuVL were as follows: Hot start at 94° C. for 5 min; 40 cycles of 20 s at 94° C., 20 s at 52° C., 45 s at 68° C., and a final extension at 68° C. for 7 min. 8 μl of 50 μl PCR solution were loaded on a 48 E-Gel 2% (Invitrogen G8008-02). Positive PCR reactions were cleaned using the NucleoSpin Extract II kit (Macherey&Nagel; 740609250) according to manufacturer's protocol and eluted in 50 μl elution buffer. All cleaning steps were performed on a Hamilton ML Starlet System.

TABLE 3
Nucleotide sequences PCR primers

| SEQ ID NO: | | |
|---|---|---|
| 77 | rbHC.up | AAGCTTGCCACCATGGAGACTGGGCTGCGCTGGCTTC |
| 78 | rbHCf.do | CCATTGGTGAGGGTGCCCGAG |
| 79 | rbLC.up | AAGCTTGCCACCATGGACAYGAGGGCCCCCACTC |
| 80 | rbLC.do | CAGAGTRCTGCTGAGGTTGTAGGTAC |
| 81 | BcPCR_FHLC_leader.fw | ATGGACATGAGGGTCCCCGC |
| 82 | BcPCR_huCkappa.rev | GATTTCAACTGCTCATCAGATGGC |

### 1.2.4 Generation of Hybridoma

Prepared spleens were disrupted mechanically. Cells were washed, harvested by centrifugation and re-suspended in 10 ml lysis buffer. After 5 min lysis at 4° C., 40 ml cold medium (RPMI 1640) was added, cells were washed and re-suspended in 50 ml cold RPMI 1640. After determination of the lymphocyte cell number, P3x63-Ag8.653 cells (washed and re-suspended in RPMI 1640 medium) were added. The ratio of lymphocytes to myeloma cells was chosen as 2:1. Cells were harvested by centrifugation, the medium was removed and the fusion of both cell types was started by addition of PEG 1500 (37° C.; 1.5 ml PEG per 108 lymphocytes). After incubation for 1 min, RPMI 1640 medium was added in three consecutive steps (1, 3 and 16 ml). Cells were harvested by centrifugation, re-suspended in 1 ml RPMI 1640 and plated on semi-solid medium in 6 well plates. Addition of HAT (Hypoxanthine/Aminopterine/Thymidine) was used to select for fused hybridoma cells. Clones were picked after 9-13 days of incubation at 37° C.

Isolated clones were transferred to 96 well plates and incubated for 72 hrs. The supernatants are used for primary screening and identification of GITR specific antibodies. For secondary screening the cells from selected hits were transferred to 24 well plates, split and expanded. For μ-purification of IgGs the supernatants were transferred to 2 ml 96 deep well plates while the cells were stored at −150° C. until further evaluation.

1.2.5 Antibody Sequencing from Hybridoma Cells

mRNA was extracted and purified from a hybridoma cell pellet using QIAGEN® RNAeasy® Mini kit. Purified mRNA was next transcribed into cDNA using the CLONETECH SMARTer RACE 5′/3′ kit according to the manufactures instructions. Nucleic acid sequences coding for the Clone 009 heavy and light chain variable regions were amplified from the cDNA by PCR, using degenerate VH and VL sense primers and a gene-specific (CH/CL) anti-sense primer. The PCR products were gel-purified and cloned into a vector using the In-Fusion® HD Cloning Kit, and then sequenced. Sequences were analysed to have antibody variable regions of light or heavy chains. Positive sequences were cloned into an antibody expression vector and screened for antigen specificity.

Clones 009, 1167, 1143, and 1138 were identified as human ICOS-specific binders through the procedures described above. The amino acid sequences of their variable regions are shown in Table 4 below.

TABLE 4
Amino acid sequences of the Variable domains of immunization-derived ICOS
antibodies. Underlined are the complementarity determining regions (CDRs).

| | SEQ ID | |
|---|---|---|
| Clone | NO: | Sequence |
| 009 | 11 (VL) | AIQMTQSPSSLSASLGGEVTITCKASQDINKNIAWYQHKPGRGPRLLIWYTST |
| | | LQTGIPSRFSGSGSGRDYSFTISNLEPEDFATYYCLQFDNLYTFGSGTKLEIR |
| | 10 (VH) | EVRLDETGGGVVQPGRPMELSCVASGFTFSDYWMNWVRQSPEKGLEWVAQIRN |
| | | KPYNYETYYSDSVKGRFTISRDDSKSRVYLQMNNLRAEDMGIYYCTWPRLRSS |
| | | DWHFDVWGAGTTVTVSS |
| 1167 | 19 (VL) | DIQMTQSPSSVSASVGDRVTITCRASQGINNFLAWYQQKPGKAPKLLIYDASS |
| | | LQSGVPSRFAGSGSGTDFTLTISSLQPEDFATYYCQQYNFYPLTFGGGTMVEI |
| | | K |
| | 18 (VH) | EVRLLESGGGLVQPGGSLRLSCAASGFTFNTYAVHWVRQAPGKGLEWVSGIGG |
| | | SGVRTYYADSVKGRLTISRDNSKNTLYLQMNSLRAEDTAIYFCAKDIYVADFT |
| | | GYAFDIWGQGTMVTVSS |
| 1143 | 27 (VL) | AIDMTQTPASVEAAVGGTVTINCQASENIYNWLAWYQQKPGQPPKLLIYDASK |
| | | LASGVPSRFSASGSGTQFTLTISAVECADAATYYCQQAYTYGNIDNAFGGGTE |
| | | VWS |
| | 26 (VH) | QSLEESGGDLVKPGASLTLTCKASGFDFSSAYDMCWVRQAPGKGLEWIGCVYY |
| | | GDGITYYATWAKGRFTISKTSSTTVPLQMTSLTAADTATYFCARGAFLGSSYY |
| | | LSLWGQGTLVTVSS |
| 1138 | 35 (VL) | ALVMTQTPSSVSAAVGGTVTINCQASQNIYSNLAWYQQKPGQPPKLLIYAASY |
| | | LTSGVSSRFKGSGAGTQFTLTISGVECADAATYYCQQGHTTDNIDNAFGGGTE |
| | | VVVK |
| | 34 (VH) | QSLEESGGDLVKPGASLTLTCTASGFDLSSYYYMCWVRQAPGKGLEWIACIYA |
| | | DIYGGTTHYASWAKGRFTISKTSSTTVTLQMTSLTAADTATYFCAREDGSRYG |
| | | GSGYYNLWGPGTLVTVSS |

### 1.3 Preparation, Purification and Characterization of Anti-ICOS Rabbit IgG and Mouse Hybridoma IgG Antibodies

### 1.3.1 Cloning and Expression of Anti-ICOS Rabbit IgG Antibodies

For recombinant expression of rabbit monoclonal bivalent antibodies, PCR-products coding for VH or VL were cloned as cDNA into expression vectors by the overhang cloning method (R S Haun et al., Biotechniques (1992) 13, 515-518; M Z Li et al., Nature Methods (2007) 4, 251-256). The expression vectors contained an expression cassette consisting of a 5′ CMV promoter including intron A, and a 3′ BGH poly adenylation sequence. In addition to the expression cassette, the plasmids contained a pUC18-derived origin of replication and a beta-lactamase gene conferring ampicillin resistance for plasmid amplification in E. coli. Three variants of the basic plasmid were used: one plasmid containing the rabbit IgG constant region designed to accept the VH regions while two additional plasmids containing rabbit or human kappa LC constant region to accept the VL regions.

Linearized expression plasmids coding for the kappa or gamma constant region and for the VL/VH inserts were amplified by PCR using overlapping primers. Purified PCR products were incubated with T4 DNA-polymerase which generated single-strand overhangs. The reaction was stopped by dCTP addition. Plasmid and insert were combined and incubated with recA which induced site specific recombination. The recombined plasmids were transformed into E. coli. The next day, the grown colonies were picked and tested for correct recombined plasmid by plasmid preparation, restriction analysis and DNA-sequencing. The amino acid sequences of the anti-ICOS clones are shown in Table 5.

TABLE 5
Amino acid sequences of anti-ICOS clones in rabbit IgG format

| | SEQ ID | |
|---|---|---|
| Molecule | No. | Sequence |
| 8 | 83 | DIQMTQSPSSVSASVGDRVTITCRASQGINNFLAWYQQKPGKAPKLLIY |
| | (1167 light | DASSLQSGVPSRFAGSGSGTDFTLTISSLQPEDFATYYCQQYNFYPLTF |
| | chain) | GGGTMVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| | 84 | EVRLLESGGGLVQPGGSLRLSCAASGFTFNTYAVHWVRQAPGKGLEWVS |
| | (1167 heavy | GIGGSGVRTYYADSVKGRLTISRDNSKNTLYLQMNSLRAEDTAIYFCAK |
| | chain) | DIYVADFTGYAFDIWGQGTMVTVSSGQPKAPSVFPLAPCCGDTPSSTVT |
| | | LGCLVKGYLPEPVTVTWNSGTLTNGVRTFPSVRQSSGLYSLSSVVSVTS |
| | | SSQPVTCNVAHPATNTKVDKTVAPSTCSKPTCPPPELLGGPSVFIFPPK |
| | | PKDTLMISRTPEVTCVVVDVSQDDPEVQFTWYINNEQVRTARPPLREQQ |
| | | FNSTIRVVSTLPIAHQDWLRGKEFKCKVHNKALPAPIEKTISKARGQPL |
| | | EPKVYTMGPPREELSSRSVSLTCMINGFYPSDISVEWEKNGKAEDNYKT |
| | | TPAVLDSDGSYFLYNKLSVPTSEWQRGDVFTCSVMHEALHNHYTQKSIS |
| | | RSPGK |
| 20 | 85 | AIDMTQTPASVEAAVGGTVTINCQASENIYNWLAWYQQKPGQPPKLLIY |
| | (1143 light | DASKLASGVPSRFSASGSGTQFTLTISAVECADAATYYCQQAYTYGNID |
| | chain) | NAFGGGTEVVVSGDPVAPTVLIFPPAADQVATGTVTIVCVANKYFPDVT |
| | | VTWEVDGTTQTTGIENSKTPQNSADCTYNLSSTLTLTSTQYNSHKEYTC |
| | | KVTQGTTSVVQSFNRGDC |
| | 86 | QSLEESGGDLVKPGASLTLTCKASGFDFSSAYDMCWVRQAPGKGLEWIG |
| | (1143 heavy | CVYYGDGITYYATWAKGRFTISKTSSTTVPLQMTSLTAADTATYFCARG |
| | chain) | AFLGSSYYLSLWGQGTLVTVSSGQPKAPSVFPLAPCCGDTPSSTVTLGC |
| | | LVKGYLPEPVTVTWNSGTLTNGVRTFPSVRQSSGLYSLSSVVSVTSSSQ |
| | | PVTCNVAHPATNTKVDKTVAPSTCSKPTCPPPELLGGPSVFIFPPKPKD |
| | | TLMISRTPEVTCVVVDVSQDDPEVQFTWYINNEQVRTARPPLREQQFNS |
| | | TIRVVSTLPIAHQDWLRGKEFKCKVHNKALPAPIEKTISKARGQPLEPK |
| | | VYTMGPPREELSSRSVSLTCMINGFYPSDISVEWEKNGKAEDNYKTTPA |
| | | VLDSDGSYFLYNKLSVPTSEWQRGDVFTCSVMHEALHNHYTQKSISRSP |
| | | GK |
| 18 | 87 | ALVMTQTPSSVSAAVGGTVTINCQASQNIYSNLAWYQQKPGQPPKLLIY |
| | (1138 light | AASYLTSGVSSRFKGSGAGTQFTLTISGVECADAATYYCQQGHTTDNID |
| | chain) | NAFGGGTEVVVKGDPVAPTVLIFPPAADQVATGTVTIVCVANKYFPDVT |
| | | VTWEVDGTTQTTGIENSKTPQNSADCTYNLSSTLTLTSTQYNSHKEYTC |
| | | KVTQGTTSVVQSFNRGDC |
| | 88 | QSLEESGGDLVKPGASLTLTCTASGFDLSSYYYMCWVRQAPGKGLEWIA |
| | (1138 heavy | CIYADIYGGTTHYASWAKGRFTISKTSSTTVTLQMTSLTAADTATYFCA |
| | chain) | REDGSRYGGSGYYNLWGPGTLVTVSSGQPKAPSVFPLAPCCGDTPSSTV |
| | | TLGCLVKGYLPEPVTVTWNSGTLTNGVRTFPSVRQSSGLYSLSSVVSVT |
| | | SSSQPVTCNVAHPATNTKVDKTVAPSTCSKPTCPPPELLGGPSVFIFPP |
| | | KPKDTLMISRTPEVTCVVVDVSQDDPEVQFTWYINNEQVRTARPPLREQ |
| | | QFNSTIRVVSTLPIAHQDWLRGKEFKCKVHNKALPAPIEKTISKARGQP |
| | | LEPKVYTMGPPREELSSRSVSLTCMINGFYPSDISVEWEKNGKAEDNYK |
| | | TTPAVLDSDGSYFLYNKLSVPTSEWQRGDVFTCSVMHEALHNHYTQKSI |
| | | SRSPGK |

For antibody expression, HEK293F culture was expanded to a volume of 1 L (Freestyle F17 with 1% Penicillin/Streptomycin, 2 mM L-Glutamine and 0.1% Pluronic) in a 3 L Erlenmeyer flask (Corning, 15 L working volume, 37° C., 8% v/v CO₂, 80 rpm, 50 mm amplitude). The culture was diluted one day before transfection and cell number adjusted to 10⁶ cells/ml in 1 L medium.

Transient expression was performed by co-transfection of the isolated HC and LC plasmids. A MasterMix of DNA/FectoPro (FectoPro, PolyPlus) was prepared in pure F17 Medium and incubated for 10 minutes (according to PolyPlus protocol). This transfection mix was added to the cell suspension dropwise and the Booster was added immediately. 18 hrs after transfection the culture was fed with 3 g/L Glucose. Supernatants were harvested after 1 week and cleared by centrifugation at 4000×g. 1 M Glycine and 300 mM NaCl was added to the cleared supernatant and used for purification by affinity chromatography.

The initial capture step was performed at room temperature by loading 1 L supernatant at a flow rate of 0.7 mL/min onto 25 mL MabSelectSure columns (GE Healthcare), equilibrated in 1×PBS pH 7.4 connected to an ÄKTA prime system. The columns were washed with 1×PBS pH 7.4 at a flow rate of 3 mL/min until UV-absorption at 280 nm reached a stable baseline. The protein bound was eluted with 50 mM Acetate/NaOH pH 3.2 at a flow rate of 3 mL/min as 3 mL fractions in tubes containing 1.2 mL 0.5M Histidine/HCl pH 6.

The pooled fractions were concentrated and applied to a Superdex200 16/60 or Superdex 100 10/300 increase column, equilibrated in 20 mM Histidine/HCl pH 6.0, 140 mM NaCl at a flow rate of 0.5 or 1 mL/min, respectively. Analysis of protein aggregation was performed by size-exclusion chromatography on a Dionex UltiMate 3000 series HPLC system equipped with a Tosoh TSKgel G5000PWXL 10 μm 7.8×300 mm column at a flow rate of 0.75 mL/min. Purity and molecular weight of the antibodies were analyzed by SDS-PAGE or via microfluidic chip capillary electrophoresis (LabChip GX) using buffers with or without DTT.

1.3.2 Preparation of Monoclonal Antibodies from Hybridoma

For the preparation of monoclonal antibodies from hybridoma cultures, cells were seeded at 2×10⁵ cells/mL and cultured for 7 days in 500 mL culture medium. The hybridoma supernatants were steril filtered and purified via protein A affinity chromatography and size exclusion chromatography. Fractions containing monomer Fc-fusion protein from the size exclusion chromatography were pooled and the protein concentration was determined by a UV method using the NanoDrop System (PeqLab ND-1000) based on the calculated extinction coefficient at 280 nm. Analysis of protein aggregation was performed by size-exclusion chromatography on a Dionex UltiMate 3000 series HPLC system equipped with a Tosoh TSKgel G5000PWXL 10 μm 7.8×300 mm column at a flow rate of 0.75 mL/min. Purity and molecular weight of the antibodies were analyzed by SDS-PAGE or via microfluidic chip capillary electrophoresis (LabChip GX) using buffers with or without DTT.

Table 6 summarizes the yield and final content of the anti-ICOS IgG1 antibodies.

TABLE 6
Biochemical analysis of anti-ICOS rabbit and
mouse IgG clones

| | | Monomer | Purity |
|---|---|---|---|
| | Yield | [%] | [%] |
| Molecule | [mg/l] | aSEC | CE-SDS |
| 14 (muIgG of 009) | 1.08 | >99 | >98 |
| 8 (rbIgG of 1167) | 1.07 | >99 | >98 |
| 20 (rbIgG of 1143) | 1.01 | >99 | >97 |
| 18 (rbIgG of 1138) | 1.02 | >99 | >96 |

### Example 2

### Characterization of Anti-ICOS Antibodies

### 2.1 Binding on Human ICOS

### 2.1.1 Surface Plasmon Resonance (Avidity+Affinity)

Binding of immunization-derived ICOS-specific antibodies to the recombinant monomeric ICOS Fc(kih) was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany).

Kinetic constants were derived using the Biacore T200 Evaluation Software (vAA, Biacore AB, Uppsala/Sweden), to fit rate equations for 1:1 Langmuir binding by numerical integration and used to estimate qualitatively the avidity.

In the same experiment, the affinities of the interaction between immunization-derived ICOS-specific antibodies molecule 8, molecule 14, molecule 18 and molecule 20 to recombinant human ICOS were determined. For this purpose, the ectodomain of human ICOS was subcloned in frame with an avi (GLNDIFEAQKIEWHE) tag (for the sequences see Table 7).

TABLE 7
Amino acid sequences of monomeric human ICOS Fc(kih) Avi tag

| SEQ ID | | |
|---|---|---|
| NO: | Antigen | Sequence |
| 89 | human ICOS Fc | EINGSANYEMFIFHNGGVQILCKYPDIVQQFKMQLLKGGQILCDLT |
| | knob Avi-tag | KTKGSGNTVSIKSLKFCHSQLSNNSVSFFLYNLDHSHANYYFCNLS |
| | | IFDPPPFKVTLTGGYLHIYESQLCCQLKSADVDASGGSPTPPTPGG |
| | | GSADKTHTCPPCPAPEAAGGPSVFLFPPKPKDTLMISRTPEVTCVV |
| | | VDVSHEDPEVKFNWYVDGVEVHNAKTKPREEQYNSTYRVVSVLTVL |
| | | HQDWLNGKEYKCKVSNKALGAPIEKTISKAKGQPREPQVYTLPPCR |
| | | DELTKNQVSLWCLVKGFYPSDIAVEWESNGQPENNYKTTPPVLDSD |
| | | GSFFLYSKLTVDKSRWQQGNVESCSVMHEALHNHYTQKSLSLSPGK |
| | | SGGLNDIFEAQKIEWHE |
| 90 | human ICOS Fc | DKTHTCPPCPAPEAAGGPSVFLFPPKPKDTLMISRTPEVTCVVVDV |
| | hole | SHEDPEVKFNWYVDGVEVHNAKTKPREEQYNSTYRVVSVLTVLHQD |
| | | WLNGKEYKCKVSNKALGAPIEKTISKAKGQPREPQVCTLPPSRDEL |
| | | TKNQVSLSCAVKGFYPSDIAVEWESNGQPENNYKTTPPVLDSDGSF |
| | | FLVSKLTVDKSRWQQGNVFSCSVMHEALHNRFTQKSLSLSPGK |

Protein production was performed as described above for the Fc fusion protein. Secreted proteins were purified from cell culture supernatants by chelating chromatography, followed by size exclusion chromatography.

The first chromatographic step was performed on a NiNTA Superflow Cartridge (5 ml, Qiagen) equilibrated in 20 mM sodium phosphate, 500 nM sodium chloride, pH7.4. Elution was performed by applying a gradient over 12 column volume from 5% to 45% of elution buffer (20 mM sodium phosphate, 500 nM sodium chloride, 500 mM Imidazole, pH7.4).

The protein was concentrated and filtered prior to loading on a HiLoad Superdex 75 column (GE Healthcare) equilibrated with 2 mM MOPS, 150 mM sodium chloride, 0.02% (w/v) sodium azide solution of pH 7.4.

Affinity determination was performed using two setups.

Setup 1: Anti-rabbit Fc antibody (Jackson ImmunoResearch, Cambridgeshire/UK) was directly coupled on a CM5 chip at pH 4.5 using the standard amine coupling kit (Biacore, Freiburg/Germany). The immobilization level was approximately 9000 RU. Rabbit immunization-derived antibodies to ICOS (molecule 8, molecule 18, molecule 20) were captured for 30 seconds at a concentration of 5.0 nM. Recombinant human ICOS Fc(kih) was passed at a concentration range from 7.5 to 600 nM with a flow of 60 μL/minutes through the flow cells over 120 seconds. The dissociation was monitored for 720 seconds. Bulk refractive index differences were corrected for by subtracting the response obtained on reference flow cell. Here, the antigens were flown over a surface with immobilized anti-rabbit Fc antibody but on which HBS-EP has been injected rather than the antibodies.

Setup 2: Anti-mouse IgG antibody (GE Healthcare, Chicago/US) was directly coupled on a CM5 chip at pH 5.0 using the standard amine coupling kit (Biacore, Freiburg/Germany). The immobilization level was approximately 5000 RU. Mouse immunization derived antibody to ICOS (molecule 14) was captured for 30 seconds at a concentration of 5.0 nM. Recombinant human ICOS Fc(kih) was passed at a concentration range from 7.5 to 600 nM with a flow of 60 μL/minutes through the flow cells over 120 seconds. The dissociation was monitored for 720 seconds. Bulk refractive index differences were corrected for by subtracting the response obtained on reference flow cell. Here, the antigens were flown over a surface with immobilized anti-mouse IgG antibody but on which HBS-EP has been injected rather than the antibodies.

Affinity constants for the interaction between anti-ICOS Antibodies and human ICOS Fc(kih) were determined by fitting to a 1:1 Langmuir binding using the BIAeval software (GE Healthcare). It was shown that molecule 8, molecule 14, molecule 18 and molecule 20 binds human ICOS (Table 8).

TABLE 8
Binding of anti-ICOS antibodies to
recombinant human ICOS

| | Recombinant human ICOS Fc (kih) | | | |
|---|---|---|---|---|
| | ka | kd | t½ | K_{D} |
| Molecule | (1/Ms) | (1/s) | (min) | (nM) |
| 14 | 6.1E+04 | 1.4E−04 | 81.6 | 2 |
| 8 | 8.1E+04 | 6.1E−03 | 1.9 | 76 |
| 20 | 1.7E+05 | 2.3E−05 | 498.3 | 0.1 |
| 18 | 4.0E+05 | 2.6E−04 | 43.7 | 0.7 |

### 2.2 Ligand Blocking Property

Cell-based receptor ligand binding assays were performed to determine the ability of the anti-ICOS antibodies to block the binding of ICOS to its ligand ICOSLG.

Biotinylated recombinant human ICOS protein was prepared as described for recombinant human ICOS Fc(kih) in Example 2.1.

Full-length cDNA encoding human ICOS ligand was subcloned into mammalian expression vector and transfected into CHO-K1 (ATCC, CCL-61) to generate recombinant ICOS ligand expressing cells (CHO-ICOSLG). Plasmids were transfected using Lipofectamine LTX Reagent (Invitrogen, #15338100) according to the manufacturer's protocol. Stably transfected ICOSLG-positive CHO-K1 cells were maintained in DMEM/F-12 (Gibco, #11320033) supplemented with 10% fetal bovine serum (Gibco, #16140063) and 1% GlutaMAX Supplement (Gibco; #31331-028). Two days after transfection, puromycin (Invivogen; #ant-pr-1) was added to 6 μg/mL. After initial selection, the cells with the highest cell surface expression of ICOSLG were sorted using BD FACSAria III cell sorter (BD Biosciences) and cultured to establish stable cell clones. The expression level and stability was confirmed by FACS analysis using APC anti-human CD275 antibody (BioLegend; #309407) over a period of 4 weeks.

384-well poly-D-lysin plates (Corning, #356662) were coated with 25 μl/1×10⁴ CHO-ICOSLG cells per well, sealed and incubated at 37° C. overnight. Biotinylated human ICOS Fc(kih) at a final assay concentration of 150 ng/ml was pre-incubated with the respective anti-ICOS antibody (14 dilution steps 1:2, starting concentration in assay 4 μg/ml) and incubated for 1 h at room temperature.

After centrifugation of cell-coated plates, 25 μl/well of the pre-incubated samples were added to the cells and incubated for 2 h at 4° C. After washing 3×90 μl/well with PBST-buffer (DPBS, PAN, P04-36500+0.1% Tween 20), each well was incubated with 0.05% Glutaraldehyde in 1×PBS (50 μl/well, Sigma Cat. No: G5882) for 10 min at room temperature to fix the cell-sample mixtures.

After washing 3×90 μl/well with PBST-buffer, human ICOS interacting with human ICOS-L on the cell surface was detected via addition of Streptavidin-POD conjugate (Roche, #11089153001, 1:4000) and incubation for 1 h at RT. After additionally washing 3×90 μl/well with PBST-buffer, 25 μl/well TMB substrate (Roche Diagnostics GmbH, #11835033001) was added for 5 min. Measurement took place at 370/492 nm (Table 9).

TABLE 9
Ligand binding property of the anti-ICOS
clones determined by enzyme-linked
immunosorbent assay

| | | Ligand |
|---|---|---|
| Molecule | Origin | blocking |
| 14 | Mouse immunization | YES |
| 8 | Rabbit immunization | YES |
| 20 | Rabbit immunization | YES |
| 18 | Rabbit immunization | YES |

### 2.3 Epitope Characterization

The epitope recognized by the immunization-derived anti-ICOS antibodies was characterized by surface plasmon resonance.

### 2.3.1 Competition Binding (Surface Plasmon Resonance)

To analyze competitive binding for the human receptor of the anti-ICOS antibodies, biotinylated human ICOS Fc(kih) was directly coupled to different flow cells of a streptavidin (SA) sensor chip. Immobilization levels up to 600 resonance units (RU) were used. Immunization-derived anti-ICOS clones Molecule 8, Molecule 14, Molecule 18 and Molecule 20 were passed at a concentration range from 2 to 500 nM (3-fold dilution) with a flow of 30 μL/minute through the flow cells over 120 seconds. The dissociation was omitted and a second anti-ICOS antibody was passed at a concentration of 100 nM with a flow of 30 μL/min over 90 seconds. Bulk refractive index differences were corrected for by subtracting the response obtained on reference flow cell.

The SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). The competition binding experiment showed that the immunization-derived anti-ICOS clones molecule 8, molecule 14 and molecule 20 shares a different epitope bin as molecule 18, since the two antibodies can bind simultaneously to human ICOS Fc(kih) (Table 10).

TABLE 10
Summary of competition binding experiments

| Immobilized | First | Second injection | | | |
|---|---|---|---|---|---|
| on chip | injection | 14 | 18 | 20 | 8 |
| 14 | human | X | 1 | 0 | 0 |
| | ICOS | | | | |
| | Fc(kih) | | | | |
| 18 | human | 1 | X | 1 | 1 |
| | ICOS | | | | |
| | Fc(kih) | | | | |
| 20 | human | 0 | 1 | X | 0 |
| | ICOS | | | | |
| | Fc(kih) | | | | |
| 8 | human | 0 | 1 | 0 | X |
| | ICOS | | | | |
| | Fc(kih) | | | | |

0 = no binding; 1 = binding; X = not determined since the second injection contains the same antibody as the one immobilized on the chip

### Example 3

### Generation of Bispecific Constructs Targeting ICOS and Fibroblast Activation Protein (FAP)

### 3.1 Generation of Bispecific Monovalent Antigen Binding Molecules Targeting ICOS and Fibroblast Activation Protein (FAP) (1+1 Format)

Bispecific agonistic ICOS antibodies with monovalent binding for ICOS and for FAP were prepared by applying the knob-into-hole technology to allow the assembling of two different heavy chains. The crossmab technology was applied to reduce the formation of wrongly paired light chains as described in International patent application No. WO 2010/145792 A1.

The generation and preparation of the FAP binder (4B9) is described in WO 2012/020006 A2, which is incorporated herein by reference.

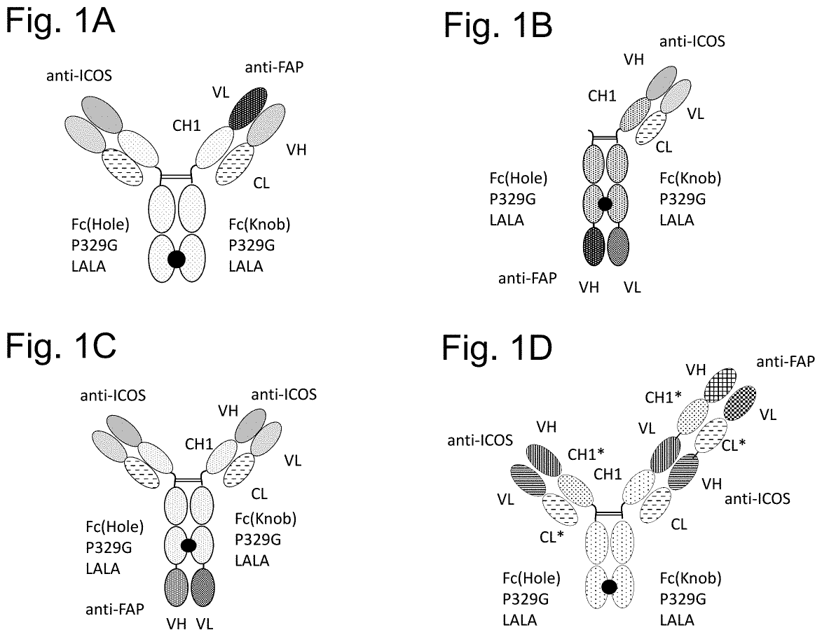

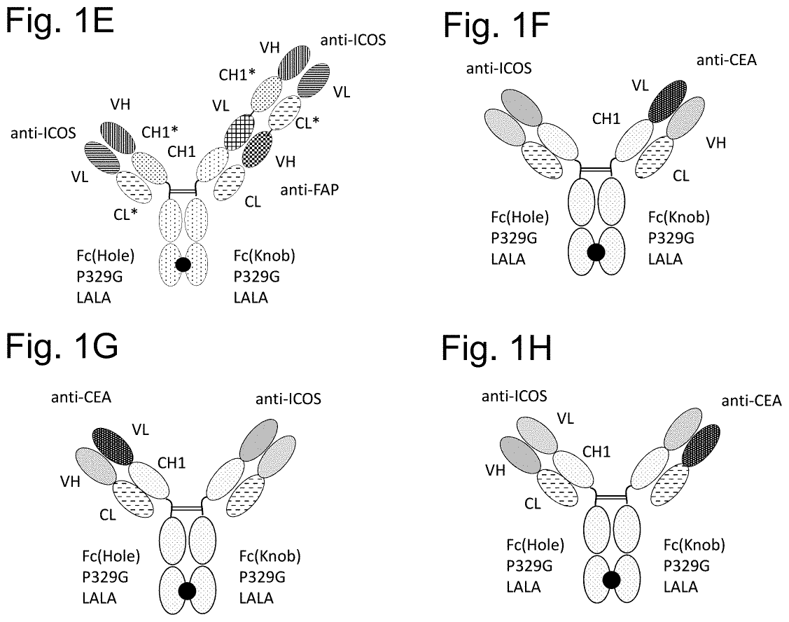

The bispecific construct binds monovalently to ICOS and to FAP (FIG. 1A). It contains a crossed Fab unit (VLCH1) of the FAP antigen binding domain fused to the hole heavy chain of an anti-ICOS huIgG1 (containing the Y349C/T366S/L368A/Y407V mutations). The Fc knob heavy chain (containing the S354C/T366W mutations) is fused to a Fab comprising the anti-ICOS antigen binding domain. Combination of the targeted anti-FAP-Fc hole with the anti-ICOS-Fc knob chain allows generation of a heterodimer, which includes a Fab that specifically binds to FAP and a Fab that specifically binds to ICOS.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831 A1.

The resulting bispecific, bivalent construct is analogous to the one depicted in FIG. 1A. The amino acid sequences of a mature bispecific monovalent anti-ICOS (1167)/anti-FAP (4B9) huIgG1 P329GLALA kih antibody (1+1 format) are shown in Table 11.

TABLE 11
Amino acid sequences of mature bispecific monovalent anti-ICOS (1167)/anti-
FAP(4B9) huIgG1 P329GLALA kih antibody (Molecule 10)

| | SEQ | | |
|---|---|---|---|
| Molecule | ID NO: | Name | Sequence |
| 10 | 91 | (FAP 4B9) | EIVLTQSPGTLSLSPGERATLSCRASQSVTSSYLAWYQ |
| | | VLCH1-Fc hole | QKPGQAPRLLINVGSRRATGIPDRFSGSGSGTDFTLTI |
| | | | SRLEPEDFAVYYCQQGIMLPPTFGQGTKVEIKSSASTK |
| | | | GPSVFPLAPSSKSTSGGTAALGCLVKDYFPEPVTVSWN |
| | | | SGALTSGVHTFPAVLQSSGLYSLSSVVTVPSSSLGTQT |
| | | | YICNVNHKPSNTKVDKKVEPKSCDKTHTCPPCPAPEAA |
| | | | GGPSVFLFPPKPKDTLMISRTPEVTCVVVDVSHEDPEV |
| | | | KFNWYVDGVEVHNAKTKPREEQYNSTYRVVSVLTVLHQ |
| | | | DWLNGKEYKCKVSNKALGAPIEKTISKAKGQPREPQVC |
| | | | TLPPSRDELTKNQVSLSCAVKGFYPSDIAVEWESNGQP |
| | | | ENNYKTTPPVLDSDGSFFLVSKLTVDKSRWQQGNVFSC |
| | | | SVMHEALHNHYTQKSLSLSP |
| | 92 | (FAP 4B9) VHCL- | EVQLLESGGGLVQPGGSLRLSCAASGFTFSSYAMSWVR |
| | | Light chain 1 | QAPGKGLEWVSAIIGSGASTYYADSVKGRFTISRDNSK |
| | | | NTLYLQMNSLRAEDTAVYYCAKGWFGGFNYWGQGTLVT |
| | | | VSSASVAAPSVFIFPPSDEQLKSGTASVVCLLNNFYPR |
| | | | EAKVQWKVDNALQSGNSQESVTEQDSKDSTYSLSSTLT |
| | | | LSKADYEKHKVYACEVTHQGLSSPVTKSFNRGEC |
| | 93 | (1167) VHCH1-Fc | EVRLLESGGGLVQPGGSLRLSCAASGFTFNTYAVHWVR |
| | | knob | QAPGKGLEWVSGIGGSGVRTYYADSVKGRLTISRDNSK |
| | | | NTLYLQMNSLRAEDTAIYFCAKDIYVADFTGYAFDIWG |
| | | | QGTMVTVSSASTKGPSVFPLAPSSKSTSGGTAALGCLV |
| | | | EDYFPEPVTVSWNSGALTSGVHTFPAVLQSSGLYSLSS |
| | | | VVTVPSSSLGTQTYICNVNHKPSNTKVDEKVEPKSCDK |
| | | | THTCPPCPAPEAAGGPSVFLFPPKPKDTLMISRTPEVT |
| | | | CVVVDVSHEDPEVKFNWYVDGVEVHNAKTKPREEQYNS |
| | | | TYRVVSVLTVLHQDWLNGKEYKCKVSNKALGAPIEKTI |
| | | | SKAKGQPREPQVYTLPPCRDELTKNQVSLWCLVKGFYP |
| | | | SDIAVEWESNGQPENNYKTTPPVLDSDGSFFLYSKLTV |
| | | | DKSRWQQGNVFSCSVMHEALHNHYTQKSLSLSP |
| | 94 | (1167) VLCL-Light | DIQMTQSPSSVSASVGDRVTITCRASQGINNFLAWYQ4 |
| | | chain 2 | KPGKAPKLLIYDASSLQSGVPSRFAGSGSGTDFTLTIS |
| | | | SLQPEDFATYYCQQYNFYPLTFGGGTMVEIKRTVAAPS |
| | | | VFIFPPSDRKLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |

The bispecific monovalent anti-ICOS and anti-FAP huIgG1 P329GLALA was produced by co-transfecting HEK293F cells with the mammalian expression vectors using FectoPro (PolyPlus, US). The cells were transfected with the corresponding expression vectors in a 1:1:1:1 ratio (“vector knob heavy chain”:“vector light chain1”:“vector hole heavy chain”:“vector light chain2”).

For production in 1 L shake flasks, 10⁶ cells/mL HEK293F cells were seeded 24 hours before transfection. A transient transfection was performed with the plasmids encoding the target protein of interest. A MasterMix of DNA/FectoPro was prepared in pure F17 Medium and incubated for 10 minutes. This transfection mix was added to the cell suspension dropwise and the Booster was added immediately. 18 hours after transfection the culture was fed with 3 g/L Glucose.

After culturing for 7 days, the cell supernatant was collected by centrifugation for 15 minutes at 210×g. The solution was sterile filtered (0.22 μm filter), supplemented with sodium azide to a final concentration of 0.01% (w/v), and kept at 4° C.

The recombinant antibodies contained therein were purified from the supernatant in two steps by affinity chromatography using protein A-Sepharose™ affinity chromatography (GE Healthcare, Sweden) and Superdex200 size exclusion chromatography. Briefly, the antibody containing clarified culture supernatants were applied on a MabSelectSuRe Protein A (5-50 ml) column equilibrated with PBS buffer (10 mM Na2HPO4, 1 mM KH2PO4, 137 mM NaCl and 2.7 mM KCl, pH 7.4). Unbound proteins were washed out with equilibration buffer. The antibodies were eluted with 50 mM citrate buffer, pH 3.0. The protein containing fractions were neutralized with 2 M Tris buffer, pH 9.0. Then, the eluted protein fractions were pooled, concentrated with an Amicon Ultra centrifugal filter device (MWCO: 30 K, Millipore) and loaded on a Superdex200 HiLoad 26/60 gel filtration column (GE Healthcare, Sweden) equilibrated with 20 mM histidine, 140 mM NaCl, at pH 6.0. The protein concentration of the various fractions was determined by determining the optical density (OD) at 280 nm with the OD at 320 nm as the background correction, using the molar extinction coefficient calculated on the basis of the amino acid sequence according to Pace et. al., Protein Science 4 (1995) 2411-2423. Monomeric antibody fractions were pooled, snap-frozen and stored at −80° C. Part of the samples was provided for subsequent protein analytics and characterization.

Purified proteins were quantified using a Nanodrop spectrophotometer (ThermoFisher) and analyzed by CE-SDS under denaturing and reducing conditions (LabChip GX, Perkin Elmer) and analytical SEC (UP-SW3000, Tosho Bioscience). Under reducing conditions, polypeptide chains related to the IgG were identified with the Lab Chip device by comparison of the apparent molecular sizes to a molecular weight standard. Determination of molecular identity was done via a state of the art electrospray-quadrupole-time-of-flight (ESI-Q-ToF) mass spectrometer (Bruker maXis) coupled to an ultra-performance liquid chromatography system (UPLC).

Expression levels of all constructs were analyzed by protein A. Average protein yields were between 25 mg and 86 mg of purified protein per liter of cell-culture supernatant in such non-optimized transient expression experiments (see Tables 12, 14, 16, 18, 21, 31 and 33).

TABLE 12
Biochemical analysis of bispecific monovalent
anti-ICOS/anti-FAP IgG1 P329G LALA
antigen binding molecule (Molecule 10)

| | | CE-SDS | |
|---|---|---|---|
| | Monomer | (non-reduced) | Yield |
| Molecule | [%] | [%] | [mg/l] |
| 10 | 98 | 96 | 2.9 |

### 3.2 Generation of Bispecific Monovalent Antigen Binding Molecules Targeting ICOS and Fibroblast Activation Protein (FAP) (1+1 Head-To-Tail Format)

Bispecific agonistic 4-1BB antibodies with monovalent binding for ICOS and monovalent binding for FAP, also termed 1+1 head-to-tail format, have been prepared as depicted in FIG. 1B.

In this example, the first heavy chain HC1 of the construct was comprised of the following components: VHCH1 of anti-ICOS binder, followed by Fc knob, at which C-terminus a VL of anti-FAP binder was fused. The second heavy chain HC2 was comprised of Fc hole, at which C-terminus a VH of anti-FAP binder was fused. The generation and preparation of FAP binder 4B9 is described in WO 2012/020006 A2, which is incorporated herein by reference. The binder against ICOS (1167), was generated as described in Example 1.

The Pro329Gly, Leu234Ala and Leu235Ala mutations have been introduced in the constant region of the knob and hole heavy chains to abrogate binding to Fcgamma receptors according to the method described in International Patent Appl. Publ. No. WO2012/130831A1.

The bispecific 1+1 anti-ICOS anti-FAP huIgG1 P329GLALA antibody was produced by co-transfecting HEK293F cells with the mammalian expression vectors using FectoPro (PolyPlus, US). The cells were transfected with the corresponding expression vectors in a 1:1:1 ratio (“vector knob heavy chain”:“vector light chain”:“vector hole heavy chain”). The constructs were produced and purified as described for the bispecific monovalent anti-ICOS and anti-FAP huIgG1 P329GLALA antibody (see Example 3.1).

The amino acid sequences for the 1+1 head-to-tail anti-ICOS, anti-FAP construct can be found in Table 13.

TABLE 13
Amino acid sequences of mature bispecific 1 + 1 head-to-tail anti-ICOS (1167)/
anti-FAP(4B9) huIgG1 P329GLALA kih antibody (Molecule 11)

| | SEQ | | |
|---|---|---|---|
| Molecule | ID NO: | Name | Sequence |
| 11 | 95 | Fc hole VH (FAP 4B9) | DKTHTCPPCPAPEAAGGPSVFLFPPKPKDTLMISRTP |
| | | | EVTCVVVDVSHEDPEVKFNWYVDGVEVHNAKTKPREE |
| | | | QYNSTYRVVSVLTVLHQDWLNGKEYKCKVSNKALGAP |
| | | | IEKTISKAKGQPREPQVCTLPPSRDELTKNQVSLSCA |
| | | | VKGFYPSDIAVEWESNGQPENNYKTTPPVLDSDGSFF |
| | | | LVSKLTVDKSRWQQGNVFSCSVMHEALHNHYTQKSLS |
| | | | LSPGGGGGSGGGGSGGGGSGGGGSEIVLTQSPGTLSL |
| | | | SPGERATLSCRASQSVTSSYLAWYQQKPGQAPRLLIN |
| | | | VGSRRATGIPDRFSGSGSGTDFTLTISRLEPEDFAVY |
| | | | YCQQGIMLPPTFGQGTKVEIK |
| | 96 | (1167)VHCH1 Fc knob | EVRLLESGGGLVQPGGSLRLSCAASGFTFNTYAVHWV |
| | | VL (4B9) | RQAPGKGLEWVSGIGGSGVRTYYADSVKGRLTISRDN |
| | | | SKNTLYLQMNSLRAEDTAIYFCAKDIYVADFTGYAFD |
| | | | IWGQGTMVTVSSASTKGPSVFPLAPSSKSTSGGTAAL |
| | | | GCLVKDYFPEPVTVSWNSGALTSGVHTFPAVLQSSGL |
| | | | YSLSSVVTVPSSSLGTQTYICNVNHKPSNTKVDKKVE |
| | | | PKSCDKTHTCPPCPAPEAAGGPSVFLFPPKPKDTLMI |
| | | | SRTPEVTCVVVDVSHEDPEVKFNWYVDGVEVHNAKTK |
| | | | PREEQYNSTYRVVSVLTVLHQDWLNGKEYKCKVSNKA |
| | | | LGAPIEKTISKAKGQPREPQVYTLPPCRDELTKNQVS |
| | | | LWCLVKGFYPSDIAVEWESNGQPENNYKTTPPVLDSD |
| | | | GSFFLYSKLTVDKSRWQQGNVFSCSVMHEALHNHYTQ |
| | | | KSLSLSPGGGGGSGGGGSGGGGSGGGGSEVQLLESGG |
| | | | GLVQPGGSLRLSCAASGFTFSSYAMSWVRQAPGKGLE |
| | | | WVSATIGSGASTYYADSVKGRETISRDNSKNTLYLQM |
| | | | NSLRAEDTAVYYCAKGWFGGFNYWGQGTLVTVSS |
| | 94 | (1167) VLCL-light | See Table 11 |
| | | chain | |

TABLE 14
Biochemical analysis of bispecific 1 + 1 head-to-tail
anti-ICOS (1167)/anti-FAP (4B9) IgG1 P329G LALA
antigen binding molecule (Molecule 11)

| | | CE-SDS | |
|---|---|---|---|
| | Monomer | (non-reduced) | Yield |
| Molecule | [%] | [%] | [mg/l] |
| 11 | 91.3 | 96 | 1.7 |

3.3 Generation of Bispecific Antigen Binding Molecules Targeting ICOS and Fibroblast Activation Protein (FAP) that are Bivalent for ICOS and Monovalent for FAP (2+1 Format)

Bispecific agonistic ICOS antibodies with bivalent binding for ICOS and monovalent binding for FAP, also termed 2+1, have been prepared as depicted in FIG. 1C.

In this example, the first heavy chain HC1 of the construct was comprised of the following components: VHCH1 of anti-ICOS binder, followed by Fc knob, at which C-terminus a VL of anti-FAP binder was fused. The second heavy chain HC2 was comprised of VHCH1 of anti-ICOS followed by Fc hole, at which C-terminus a VH of anti-FAP binder was fused. Binders against ICOS (009, 1138, 1143 and 1167) were generated as described in Example 1.

Homology modeling of the rabbit antibodies molecule 20 and molecule 18 suggested that the two cysteines at VH positions 199 and 251 (Kabat 35A and 50) are forming a disulfide bridge between CDR-H1 and CDR-H2, while the cysteine at VL framework position 726 (Kabat 80) is free and exposed to the solvent. In both cases, we went for the most conservative option, which is substituting all undesired cysteines by serine, i.e. C199S, C251S, and C726S. As we anticipated that antibody molecule 20 would need to be humanized, we added two additional variants where the substitutions are made in accord with the closest matching human germline IGHV3-23*01, i.e. C199S and C251V. Positions 252, 296 and 297 (Kabat 51, 62 and 63) were changed accordingly to evaluate if these substitutions in CDR-H2 would be tolerated without loss of binding affinity, with the added benefit of increased humanness. If not explicitly stated, residue indices are given in WolfGuy numbering. Table 15 summarizes the variations in the amino acid sequences of molecule 20 and molecule 18 and the numbers of the variant molecules.

TABLE 15
Amino acid variants of Molecule 18 and Molecule 20

| | Parental | |
|---|---|---|
| | Antibody | |
| Molecule | [Molecule] | Mutation |
| 44 | 20 | VH: C199S_C251S |
| | | VL: C726S |
| 21 | 20 | VH: C199S_C251V |
| | | VL: C726S |
| 22 | 20 | VH: C199S_C251V_V2521_W296S_A297V |
| | | VL: C726S |
| 19 | 18 | VH: C199S_C251S |
| | | VL: C726S |

In order to validate the impact of a framework mutation in molecule 14 two variants in a 2+1 format were generated (molecule 15 and molecule 40).

The generation and preparation of the FAP binder (4B9) is described in WO 2012/020006 A2, which is incorporated herein by reference. The Pro329Gly, Leu234Ala and Leu235Ala mutations have been introduced in the constant region of the knob and hole heavy chains to abrogate binding to Fcgamma receptors according to the method described in International Patent Appl. Publ. No. WO2012/130831A1.

The bispecific 2+1 anti-ICOS, anti-FAP huIgG1 P329GLALA antibodies were produced by co-transfecting HEK293F cells with the mammalian expression vectors using FectoPro (PolyPlus, US). The cells were transfected with the corresponding expression vectors in a 1:2:1 ratio (“vector knob heavy chain”:“vector light chain”:“vector hole heavy chain”). The constructs were produced and purified as described for the bispecific monovalent anti-ICOS and anti-FAP huIgG1 P329GLALA antibody (see Example 3.1).

The amino acid sequences for 2+1 anti-ICOS, anti-FAP constructs can be found in Table 16.

TABLE 16
Amino acid sequences of mature bispecific 2 + 1 anti-ICOS/anti-FAP(4B9)
huIgG1 P329GLALA kih antibody (Molecule 11)

| | SEQ ID | | |
|---|---|---|---|
| Molecule | NO: | Name | Sequence |
| 9 | 97 | (ICOS 1167) | EVRLLESGGGLVQPGGSLRLSCAASGFTFNTYAVH |
| | | VHCH1 Fc hole VH | WVRQAPGKGLEWVSGIGGSGVRTYYADSVKGRLTI |
| | | (FAP 4B9) | SRDNSKNTLYLQMNSLRAEDTAIYFCAKDIYVADF |
| | | | TGYAFDIWGQGTMVTVSSASTKGPSVFPLAPSSKS |
| | | | TSGGTAALGCLVKDYFPEPVTVSWNSGALTSGVHT |
| | | | FPAVLQSSGLYSLSSVVTVPSSSLGTQTYICNVNH |
| | | | KPSNTKVDKKVEPKSCDKTHTCPPCPAPEAAGGPS |
| | | | VFLFPPKPKDTLMISRTPEVTCVVVDVSHEDPEVK |
| | | | FNWYVDGVEVHNAKTKPREEQYNSTYRVVSVLTVL |
| | | | HQDWLNGKEYKCKVSNKALGAPIEKTISKAKGQPR |
| | | | EPQVCTLPPSRDELTKNQVSLSCAVKGFYPSDIAV |
| | | | EWESNGQPENNYKTTPPVLDSDGSFFLVSKLTVDK |
| | | | SRWQQGNVFSCSVMHEALHNHYTQKSLSLSPGGGG |
| | | | GSGGGGSGGGGSGGGGSEIVLTQSPGTLSLSPGER |
| | | | ATLSCRASQSVTSSYLAWYQQKPGQAPRLLINVGS |
| | | | RRATGIPDRFSGSGSGTDFTLTISRLEPEDFAVYY |
| | | | CQQGIMLPPTFGQGTKVEIK |
| | 96 | (ICOS 1167)VHCH1 | See Table 13 |
| | | Fc knob VL (4B9) | |
| | 94 | (ICOS 1167) VLCL- | See Table 13 |
| | | light chain | |
| 40 | 98 | (ICOS 009) VHCH1 | EVRLDETGGGVVQPGRPMELSCVASGFTFSDYWMN |
| | | Fc hole VH (FAP | WVRQSPEKGLEWVAQIRNKPYNYETYYSDSVKGRF |
| | | 4B9) | TISRDDSKSRVYLQMNNLRAEDMGIYYCTWPRLRS |
| | | | SDWHFDVWGAGTTVTVSSASTKGPSVFPLAPSSKS |
| | | | TSGGTAALGCLVKDYFPEPVTVSWNSGALTSGVHT |
| | | | FPAVLQSSGLYSLSSVVTVPSSSLGTQTYICNVNH |
| | | | KPSNTKVDKKVEPKSCDKTHTCPPCPAPEAAGGPS |
| | | | VFLFPPKPKDTLMISRTPEVTCVVVDVSHEDPEVK |
| | | | FNWYVDGVEVHNAKTKPREEQYNSTYRVVSVLTVL |
| | | | HQDWLNGKEYKCKVSNKALGAPIEKTISKAKGQPR |
| | | | EPQVCTLPPSRDELTKNQVSLSCAVKGFYPSDIAV |
| | | | EWESNGQPENNYKTTPPVLDSDGSFFLVSKLTVDK |
| | | | SRWQQGNVFSCSVMHEALHNHYTQKSLSLSPGGGG |
| | | | GSGGGGSGGGGSGGGGSEIVLTQSPGTLSLSPGER |
| | | | ATLSCRASQSVTSSYLAWYQQKPGQAPRLLINVGS |
| | | | RRATGIPDRFSGSGSGTDFTLTISRLEPEDFAVYY |
| | | | CQQGIMLPPTFGQGTKVEIK |
| | 99 | (ICOS 009) VHCH1 | EVRLDETGGGVVQPGRPMELSCVASGFTFSDYWMN |
| | | Fc knob VL (FAP | WVRQSPEKGLEWVAQIRNKPYNYETYYSDSVKGRF |
| | | 4B9) | TISRDDSKSRVYLQMNNLRAEDMGIYYCTWPRLRS |
| | | | SDWHFDVWGAGTTVTVSSASTKGPSVFPLAPSSKS |
| | | | TSGGTAALGCLVKDYFPEPVTVSWNSGALTSGVHT |
| | | | FPAVLQSSGLYSLSSVVTVPSSSLGTQTYICNVNH |
| | | | KPSNTKVDKKVEPKSCDKTHTCPPCPAPEAAGGPS |
| | | | VFLFPPKPKDTLMISRTPEVTCVVVDVSHEDPEVK |
| | | | FNWYVDGVEVHNAKTKPREEQYNSTYRVVSVLTVL |
| | | | HQDWLNGKEYKCKVSNKALGAPIEKTISKAKGQPR |
| | | | EPQVYTLPPCRDELTKNQVSLWCLVKGFYPSDIAV |
| | | | EWESNGQPENNYKTTPPVLDSDGSFFLYSKLTVDK |
| | | | SRWQQGNVFSCSVMHEALHNHYTQKSLSLSPGGGG |
| | | | GSGGGGSGGGGSGGGGSEVQLLESGGGLVQPGGSL |
| | | | RLSCAASGFTFSSYAMSWVRQAPGKGLEWVSAIIG |
| | | | SGASTYYADSVKGRFTISRDNSKNTLYLQMNSLRA |
| | | | EDTAVYYCAKGWFGGFNYWGQGTLVTVSS |
| | 100 | (ICOS 009) VLCL- | AIQMTQSPSSLSASLGGEVTITCKASQDINKNIAW |
| | | light chain | YQHKPGRGPRLLIWYTSTLQTGIPSRFSGSGSGRD |
| | | | YSFTISNLEPEDFATYYCLQFDNLYTFGSGTKLEI |
| | | | RRTVAAPSVFIFPPSDEQLKSGTASVVCLLNNFYP |
| | | | REAKVQWKVDNALQSGNSQESVTEQDSKDSTYSLS |
| | | | STLTLSKADYEKHKVYACEVTHQGLSSPVTKSFNR |
| | | | GEC |
| 15 | 98 | (ICOS 009v1) VHCH1 | See above, corresponds to ICOS 009 |
| | | Fc hole VH (FAP 4B9) | |
| | 101 | (ICOS 009v1) VHCH1 | EVRLDETGGGVVQPGRPMELSCVASGFTFSDYWMN |
| | | Fc knob VL (FAP | WVRQSPKGLEWVAQIRNKPYNYETYYSDSVKGRFT |
| | | 4B9) | ISRDDSKSRVYLQMNNLRAEDMGIYYCTWPRLRSS |
| | | | DWHFDVWGAGTTVTVSSASTKGPSVFPLAPSSKST |
| | | | SGGTAALGCLVKDYFPEPVTVSWNSGALTSGVHTF |
| | | | PAVLQSSGLYSLSSVVTVPSSSLGTQTYICNVNHK |
| | | | PSNTKVDKKVEPKSCDKTHTCPPCPAPEAAGGPSV |
| | | | FLFPPKPKDTLMISRTPEVTCVVVDVSHEDPEVKF |
| | | | NWYVDGVEVHNAKTKPREEQYNSTYRVVSVLTVLH |
| | | | QDWLNGKEYKCKVSNKALGAPIEKTISKAKGQPRE |
| | | | PQVYTLPPCRDELTKNQVSLWCLVKGFYPSDIAVE |
| | | | WESNGQPENNYKTTPPVLDSDGSFFLYSKLTVDKS |
| | | | RWQQGNVFSCSVMHEALHNHYTQKSLSLSPGGGGG |
| | | | SGGGGSGGGGSGGGGSEVQLLESGGGLVQPGGSLR |
| | | | LSCAASGFTFSSYAMSWVRQAPGKGLEWVSAIIGS |
| | | | GASTYYADSVKGRFTISRDNSKNTLYLQMNSLRAE |
| | | | DTAVYYCAKGWFGGFNYWGQGTLVTVSS |
| | 100 | (ICOS 009v1) VLCL- | See above, corresponds to 009 |
| | | light chain | |
| 19 | 102 | (ICOS 1138) VHCH1 | QSLEESGGDLVKPGASLTLTCTASGFDLSSYYYMS |
| | | Fc hole VH (FAP 4B9) | WVRQAPGKGLEWIASIYADIYGGTTHYASWAKGRF |
| | | | TISKTSSTTVTLQMTSLTAADTATYFCAREDGSRY |
| | | | GGSGYYNLWGPGTLVTVSSASTKGPSVFPLAPSSK |
| | | | STSGGTAALGCLVKDYFPEPVTVSWNSGALTSGVH |
| | | | TFPAVLQSSGLYSLSSVVTVPSSSLGTQTYICNVN |
| | | | HKPSNTKVDKKVEPKSCDKTHTCPPCPAPEAAGGP |
| | | | SVFLFPPKPKDTLMISRTPEVTCVVVDVSHEDPEV |
| | | | KFNWYVDGVEVHNAKTKPREEQYNSTYRVVSVLTV |
| | | | LHQDWLNGKEYKCKVSNKALGAPIEKTISKAKGQP |
| | | | REPQVCTLPPSRDELTKNQVSLSCAVKGFYPSDIA |
| | | | VEWESNGQPENNYKTTPPVLDSDGSFFLVSKLTVD |
| | | | KSRWQQGNVFSCSVMHEALHNHYTQKSLSLSPGGG |
| | | | GGSGGGGSGGGGSGGGGSEIVLTQSPGTLSLSPGE |
| | | | RATLSCRASQSVTSSYLAWYQQKPGQAPRLLINVG |
| | | | SRRATGIPDRFSGSGSGTDFTLTISRLEPEDFAVY |
| | | | YCQQGIMLPPTFGQGTKVEIK |
| | 103 | (ICOS 1138) VHCH1 | QSLEESGGDLVKPGASLTLTCTASGFDLSSYYYMS |
| | | Fc knob VL (FAP | WVRQAPGKGLEWIASIYADIYGGTTHYASWAKGRF |
| | | 4B9) | TISKTSSTTVTLQMTSLTAADTATYFCAREDGSRY |
| | | | GGSGYYNLWGPGTLVTVSSASTKGPSVFPLAPSSK |
| | | | STSGGTAALGCLVKDYFPEPVTVSWNSGALTSGVH |
| | | | TFPAVLQSSGLYSLSSVVTVPSSSLGTQTYICNVN |
| | | | HKPSNTKVDKKVEPKSCDKTHTCPPCPAPEAAGGP |
| | | | SVFLFPPKPKDTLMISRTPEVTCVVVDVSHEDPEV |
| | | | KFNWYVDGVEVHNAKTKPREEQYNSTYRVVSVLTV |
| | | | LHQDWLNGKEYKCKVSNKALGAPIEKTISKAKGQP |
| | | | REPQVYTLPPCRDELTKNQVSLWCLVKGFYPSDIA |
| | | | VEWESNGQPENNYKTTPPVLDSDGSFFLYSKLTVD |
| | | | KSRWQQGNVFSCSVMHEALHNHYTQKSLSLSPGGG |
| | | | GGSGGGGSGGGGSGGGGSEVQLLESGGGLVQPGGS |
| | | | LRLSCAASGETFSSYAMSWVRQAPGKGLEWVSAII |
| | | | GSGASTYYADSVKGRFTISRDNSKNTLYLQMNSLR |
| | | | AEDTAVYYCAKGWFGGFNYWGQGTLVTVSS |
| | 104 | (ICOS 1138) VLCL- | ALVMTQTPSSVSAAVGGTVTINCQASQNIYSNLAW |
| | | light chain | YQQKPGQPPKLLIYAASYLTSGVSSRFKGSGAGTQ |
| | | | FTLTISGVESADAATYYCQQGHTTDNIDNAFGGGT |
| | | | EVVVKRTVAAPSVFIFPPSDEQLKSGTASVVCLLN |
| | | | NFYPREAKVQWKVDNALQSGNSQESVTEQDSKDST |
| | | | YSLSSTLTLSKADYEKHKVYACEVTHQGLSSPVTK |
| | | | SFNRGEC |
| 44 | 105 | (ICOS 1143) VHCH1 | QSLEESGGDLVKPGASLTLTCKASGFDFSSAYDMS |
| | | Fc hole VH (FAP 4B9) | WVRQAPGKGLEWIGSVYYGDGITYYATWAKGRFTI |
| | | | SKTSSTTVPLQMTSLTAADTATYFCARGAFLGSSY |
| | | | YLSLWGQGTLVTVSSASTKGPSVFPLAPSSKSTSG |
| | | | GTAALGCLVKDYFPEPVTVSWNSGALTSGVHTFPA |
| | | | VLQSSGLYSLSSVVTVPSSSLGTQTYICNVNHKPS |
| | | | NTKVDKKVEPKSCDKTHTCPPCPAPEAAGGPSVFL |
| | | | FPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALGAPIEKTISKAKGQPREPQ |
| | | | VCTLPPSRDELTKNQVSLSCAVKGFYPSDIAVEWE |
| | | | SNGQPENNYKTTPPVLDSDGSFFLVSKLTVDKSRW |
| | | | QQGNVFSCSVMHEALHNHYTQKSLSLSPGGGGGSG |
| | | | GGGSGGGGSGGGGSEIVLTQSPGTLSLSPGERATL |
| | | | SCRASQSVTSSYLAWYQQKPGQAPRLLINVGSRRA |
| | | | TGIPDRFSGSGSGTDFTLTISRLEPEDFAVYYCQQ |
| | | | GIMLPPTFGQGTKVEIK |
| | 106 | (ICOS 1143) VHCH1 | QSLEESGGDLVKPGASLTLTCKASGFDFSSAYDMS |
| | | Fc knob VL (4B9) | WVRQAPGKGLEWIGSVYYGDGITYYATWAKGRFTI |
| | | | SKTSSTTVPLQMTSLTAADTATYFCARGAFLGSSY |
| | | | YLSLWGQGTLVTVSSASTKGPSVFPLAPSSKSTSG |
| | | | GTAALGCLVKDYFPEPVTVSWNSGALTSGVHTFPA |
| | | | VLQSSGLYSLSSVVTVPSSSLGTQTYICNVNHKPS |
| | | | NTKVDKKVEPKSCDKTHTCPPCPAPEAAGGPSVFL |
| | | | FPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALGAPIEKTISKAKGQPREPQ |
| | | | VYTLPPCRDELTKNQVSLWCLVKGFYPSDIAVEWE |
| | | | SNGQPENNYKTTPPVLDSDGSFFLYSKLTVDKSRW |
| | | | QQGNVFSCSVMHEALHNHYTQKSLSLSPGGGGGSG |
| | | | GGGSGGGGSGGGGSEVQLLESGGGLVQPGGSLRLS |
| | | | CAASGFTFSSYAMSWVRQAPGKGLEWVSAIIGSGA |
| | | | STYYADSVKGRFTISRDNSKNTLYLQMNSLRAEDT |
| | | | AVYYCAKGWFGGFNYWGQGTLVTVSS |
| | 107 | (ICOS 1143) VLCL- | AIDMTQTPASVEAAVGGTVTINCQASENIYNWLAW |
| | | light chain | YQQKPGQPPKLLIYDASKLASGVPSRFSASGSGTQ |
| | | | FTLTISAVESADAATYYCQQAYTYGNIDNAFGGGT |
| | | | EVVVSRTVAAPSVFIFPPSDEQLKSGTASVVCLLN |
| | | | NFYPREAKVQWKVDNALQSGNSQESVTEQDSKDST |
| | | | YSLSSTLTLSKADYEKHKVYACEVTHQGLSSPVTK |
| | | | SFNRGEC |
| 21 | 108 | (ICOS 1143v1) | QSLEESGGDLVKPGASLTLTCKASGFDFSSAYDMS |
| | | VHCH1 Fc hole VH | WVRQAPGKGLEWIGVVYYGDGITYYATWAKGRFTI |
| | | (FAP 4B9) | SKTSSTTVPLQMTSLTAADTATYFCARGAFLGSSY |
| | | | YLSLWGQGTLVTVSSASTKGPSVFPLAPSSKSTSG |
| | | | GTAALGCLVKDYFPEPVTVSWNSGALTSGVHTFPA |
| | | | VLQSSGLYSLSSVVTVPSSSLGTQTYICNVNHKPS |
| | | | NTKVDKKVEPKSCDKTHTCPPCPAPEAAGGPSVFL |
| | | | FPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALGAPIEKTISKAKGQPREPQ |
| | | | VCTLPPSRDELTKNQVSLSCAVKGFYPSDIAVEWE |
| | | | SNGQPENNYKTTPPVLDSDGSFFLVSKLTVDKSRW |
| | | | QQGNVFSCSVMHEALHNHYTQKSLSLSPGGGGGSG |
| | | | GGGSGGGGSGGGGSEIVLTQSPGTLSLSPGERATL |
| | | | SCRASQSVTSSYLAWYQQKPGQAPRLLINVGSRRA |
| | | | TGIPDRFSGSGSGTDFTLTISRLEPEDFAVYYCQQ |
| | | | GIMLPPTFGQGTKVEIK |
| | 109 | (ICOS 1143v1) | QSLEESGGDLVKPGASLTLTCKASGFDFSSAYDMS |
| | | VHCH1 Fc knob VL | WVRQAPGKGLEWIGVVYYGDGITYYATWAKGRFTI |
| | | (4B9) | SKTSSTTVPLQMTSLTAADTATYFCARGAFLGSSY |
| | | | YLSLWGQGTLVTVSSASTKGPSVFPLAPSSKSTSG |
| | | | GTAALGCLVKDYFPEPVTVSWNSGALTSGVHTFPA |
| | | | VLQSSGLYSLSSVVTVPSSSLGTQTYICNVNHKPS |
| | | | NTKVDKKVEPKSCDKTHTCPPCPAPEAAGGPSVFL |
| | | | FPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALGAPIEKTISKAKGQPREPQ |
| | | | VYTLPPCRDELTKNQVSLWCLVKGFYPSDIAVEWE |
| | | | SNGQPENNYKTTPPVLDSDGSFFLYSKLTVDKSRW |
| | | | QQGNVFSCSVMHEALHNHYTQKSLSLSPGGGGGSG |
| | | | GGGSGGGGSGGGGSEVQLLESGGGLVQPGGSLRLS |
| | | | CAASGFTFSSYAMSWVRQAPGKGLEWVSAIIGSGA |
| | | | STYYADSVKGRFTISRDNSKNTLYLQMNSLRAEDT |
| | | | AVYYCAKGWFGGFNYWGQGTLVTVSS |
| | 107 | (ICOS 1143v1) VLCL- | See above, corresponds to 1143 |
| | | light chain | |
| 22 | 110 | (ICOS 1143v2) | QSLEESGGDLVKPGASLTLTCKASGFDFSSAYDMS |
| | | VHCH1 Fc hole VH | WVRQAPGKGLEWIGVIYYGDGITYYATSVKGRFTI |
| | | (FAP 4B9) | SKTSSTTVPLQMTSLTAADTATYFCARGAFLGSSY |
| | | | YLSLWGQGTLVTVSSASTKGPSVFPLAPSSKSTSG |
| | | | GTAALGCLVKDYFPEPVTVSWNSGALTSGVHTFPA |
| | | | VLQSSGLYSLSSVVTVPSSSLGTQTYICNVNHKPS |
| | | | NTKVDKKVEPKSCDKTHTCPPCPAPEAAGGPSVFL |
| | | | FPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALGAPIEKTISKAKGQPREPQ |
| | | | VCTLPPSRDELTKNQVSLSCAVKGFYPSDIAVEWE |
| | | | SNGQPENNYKTTPPVLDSDGSFFLVSKLTVDKSRW |
| | | | QQGNVFSCSVMHEALHNHYTQKSLSLSPGGGGGSG |
| | | | GGGSGGGGSGGGGSEIVLTQSPGTLSLSPGERATL |
| | | | SCRASQSVTSSYLAWYQQKPGQAPRLLINVGSRRA |
| | | | TGIPDRFSGSGSGTDFTLTISRLEPEDFAVYYCQQ |
| | | | GIMLPPTFGQGTKVEIK |
| | 111 | (ICOS 1143v2) | QSLEESGGDLVKPGASLTLTCKASGFDFSSAYDMS |
| | | VHCH1 Fc knob VL | WVRQAPGKGLEWIGVIYYGDGITYYATSVKGRFTI |
| | | (FAP 4B9) | SKTSSTTVPLQMTSLTAADTATYFCARGAFLGSSY |
| | | | YLSLWGQGTLVTVSSASTKGPSVFPLAPSSKSTSG |
| | | | GTAALGCLVKDYFPEPVTVSWNSGALTSGVHTFPA |
| | | | VLQSSGLYSLSSVVTVPSSSLGTQTYICNVNHKPS |
| | | | NTKVDKKVEPKSCDKTHTCPPCPAPEAAGGPSVFL |
| | | | FPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALGAPIEKTISKAKGQPREPQ |
| | | | VYTLPPCRDELTKNQVSLWCLVKGFYPSDIAVEWE |
| | | | SNGQPENNYKTTPPVLDSDGSFFLYSKLTVDKSRW |
| | | | QQGNVFSCSVMHEALHNHYTQKSLSLSPGGGGGSG |
| | | | GGGSGGGGSGGGGSEVQLLESGGGLVQPGGSLRLS |
| | | | CAASGFTFSSYAMSWVRQAPGKGLEWVSAIIGSGA |
| | | | STYYADSVKGRFTISRDNSKNTLYLQMNSLRAEDT |
| | | | AVYYCAKGWFGGFNYWGQGTLVTVSS |
| | 107 | (ICOS 1143v2) VLCL- | See above, corresponds to 1143 |
| | | light chain | |

TABLE 17
Biochemical analysis of bispecific constructs with bivalent
binding to ICOS and monovalent binding to FAP
(2 + 1 ICOS/FAP human IgG1 P329GLALA)

| | | CE-SDS | |
|---|---|---|---|
| | Monomer | (non-reduced) | Yield |
| Molecule | [%] | [%] | [mg/l] |
| 9 | 92 | 93 | 1.9 |
| 40 | 98 | 99 | 3.6 |
| 15 | 98 | 100 | 4.1 |
| 19 | 99.3 | 97 | 5.6 |
| 44 | 94.6 | 94 | 3.9 |
| 21 | 98.7 | 99 | 4.4 |
| 22 | 99.5 | 98 | 4.2 |

3.4 Generation of Bispecific Antigen Binding Molecules Targeting ICOS and Fibroblast Activation Protein (FAP) that are Bivalent for ICOS and Monovalent for FAP (2+1 Crossfab-IgG P329G LALA)

Bispecific agonistic ICOS antibodies with bivalent binding for ICOS and monovalent binding for FAP, also termed 2+1 IgG CrossFab (VH/VL exchange in FAP binder), have been prepared as depicted in FIG. 1D.

In this example, the first heavy chain HC1 of the construct was comprised of the following components: VLCH1 of anti-FAP antigen binding domain, followed by VHCH1 of anti-ICOS antigen binding domain and Fc knob. The second heavy chain HC2 was comprised of VHCH1 of anti-ICOS followed by Fc hole. The antibody against ICOS (1167) was generated as described in Example 1. The generation and preparation of the FAP antibody (4B9) is described in WO 2012/020006 A2, which is incorporated herein by reference.

The Pro329Gly, Leu234Ala and Leu235Ala mutations have been introduced in the constant region of the knob and hole heavy chains to abrogate binding to Fcgamma receptors according to the method described in International Patent Appl. Publ. No. WO2012/130831A1.

The bispecific 2+1 anti-ICOS, anti-FAP huIgG1 P329GLALA antibody was produced by co-transfecting HEK293F cells with the mammalian expression vectors using FectoPro (PolyPlus, US). The cells were transfected with the corresponding expression vectors in a 1:2:1:1 ratio (“vector heavy chain (VL-CH1-VH-CH 1-CH2-CH3)”:“vector light chain (VL-CL)”:“vector heavy chain (VH-CH 1-CH2-CH3)”:“vector light chain (VHCL)”. The constructs were produced and purified as described for the bispecific monovalent anti-ICOS and anti-FAP huIgG1 P329GLALA antibody (see Example 3.1).

The amino acid sequences for these 2+1 anti-ICOS, anti-FAP Crossfab-IgG P329G LALA constructs can be found in Table 18.

TABLE 18
Amino acid sequences of mature bispecific 2 + 1 anti-ICOS (1167)/anti-FAP
(4B9) Crossfab-IgG P329G LALA

| | SEQ | | |
|---|---|---|---|
| Molecule | ID NO: | Antigen | Sequence |
| 12 | 112 | (ICOS 1167) VHCH1 | EVRLLESGGGLVQPGGSLRLSCAASGFTFNTYAVH |
| | | Fc hole | WVRQAPGKGLEWVSGIGGSGVRTYYADSVKGRLTI |
| | | | SRDNSKNTLYLQMNSLRAEDTAIYFCAKDIYVADF |
| | | | TGYAFDIWGQGTMVTVSSASTKGPSVFPLAPSSKS |
| | | | ISGGTAALGCLVEDYFPEPVTVSWNSGALTSGVHT |
| | | | FPAVLQSSGLYSLSSVVTVPSSSLGTQTYICNVNH |
| | | | KPSNTKVDEKVEPKSCDKTHTCPPCPAPEAAGGPS |
| | | | VFLFPPKPKDTLMISRTPEVTCVVVDVSHEDPEVK |
| | | | FNWYVDGVEVHNAKTKPREEQYNSTYRVVSVLTVL |
| | | | HQDWLNGKEYKCKVSNKALGAPIEKTISKAKGQPR |
| | | | EPQVCTLPPSRDELTKNQVSLSCAVKGFYPSDIAV |
| | | | EWESNGQPENNYKTTPPVLDSDGSFFLVSKLTVDK |
| | | | SRWQQGNVFSCSVMHEALHNHYTQKSLSLSP |
| | 113 | (ICOS 1167) VLCL- | DIQMTQSPSSVSASVGDRVTITCRASQGINNFLAW |
| | | light chain 1 | YQQKPGKAPKLLIYDASSLQSGVPSRFAGSGSGTD |
| | | | FTLTISSLQPEDFATYYCQQYNFYPLTFGGGTMVE |
| | | | IKRTVAAPSVFIFPPSDRKLKSGTASVVCLLNNFY |
| | | | PREAKVQWKVDNALQSGNSQESVTEQDSKDSTYSL |
| | | | SSTLTLSKADYEKHKVYACEVTHQGLSSPVTKSFN |
| | | | RGEC |
| | 114 | (FAP 4B9) VLCH1- | EIVLTQSPGTLSLSPGERATLSCRASQSVTSSYLA |
| | | (ICOS 1167) VHCH1 | WYQQKPGQAPRLLINVGSRRATGIPDRFSGSGSGT |
| | | Fc knob | DFTLTISRLEPEDFAVYYCQQGIMLPPTFGQGTKV |
| | | | EIKSSASTKGPSVFPLAPSSKSTSGGTAALGCLVK |
| | | | DYFPEPVTVSWNSGALTSGVHTFPAVLQSSGLYSL |
| | | | SSVVTVPSSSLGTQTYICNVNHKPSNTKVDKKVEP |
| | | | KSCDGGGGSGGGGSEVRLLESGGGLVQPGGSLRLS |
| | | | CAASGFTFNTYAVHWVRQAPGKGLEWVSGIGGSGV |
| | | | RTYYADSVKGRLTISRDNSKNTLYLQMNSLRAEDT |
| | | | AIYFCAKDIYVADFTGYAFDIWGQGTMVTVSSAST |
| | | | KGPSVFPLAPSSKSTSGGTAALGCLVEDYFPEPVT |
| | | | VSWNSGALTSGVHTFPAVLQSSGLYSLSSVVTVPS |
| | | | SSLGTQTYICNVNHKPSNTKVDEKVEPKSCDKTHT |
| | | | CPPCPAPEAAGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALGA |
| | | | PIEKTISKAKGQPREPQVYTLPPCRDELTKNQVSL |
| | | | WCLVKGFYPSDIAVEWESNGQPENNYKTTPPVLDS |
| | | | DGSFFLYSKLTVDKSRWQQGNVFSCSVMHEALHNH |
| | | | YTQKSLSLSP |
| | 115 | (FAP 4B9) VHCL-light | EVQLLESGGGLVQPGGSLRLSCAASGFTFSSYAMS |
| | | chain 2 | WVRQAPGKGLEWVSAIIGSGASTYYADSVKGRFTI |
| | | | SRDNSKNTLYLQMNSLRAEDTAVYYCAKGWFGGFN |
| | | | YWGQGTLVTVSSASVAAPSVFIFPPSDEQLKSGTA |
| | | | SVVCLLNNFYPREAKVQWKVDNALQSGNSQESVTE |
| | | | QDSKDSTYSLSSTLTLSKADYEKHKVYACEVTHQG |
| | | | LSSPVTKSFNRGEC |

TABLE 19
Biochemical analysis of bispecific constructs with a bivalent
binding to ICOS and a monovalent binding to FAP
(2 + 1 anti-ICOS, anti-FAP Crossfab-IgG P329G LALA)

| | | CE-SDS | |
|---|---|---|---|
| | Monomer | (non-reduced) | Yield |
| Molecule | [%] | [%] | [mg/l] |
| 12 | 96 | 100 | 0.9 |

3.5 Generation of Bispecific Antigen Binding Molecules Targeting ICOS and Fibroblast Activation Protein (FAP) that are Bivalent for ICOS and Monovalent for FAP (2+1 Crossfab-IgG P329G LALA Inverted)

Bispecific agonistic ICOS antibodies with bivalent binding for ICOS and monovalent binding for FAP, also termed 2+1 IgG CrossFab, inverted (VH/VL exchange in FAP binder), have been prepared as depicted in FIG. 1E.

In this example, the first heavy chain HC1 of the construct was comprised of the following components: VHCH1 of anti-ICOS binder, followed by VLCH1 of anti-FAP binder and Fc knob. The second heavy chain HC2 was comprised of VHCH1 of anti-ICOS followed by Fc hole. Binder against ICOS (1167), was generated as described in Example 1. The generation and preparation of the FAP binder (4B9) is described in WO 2012/020006 A2, which is incorporated herein by reference.

The Pro329Gly, Leu234Ala and Leu235Ala mutations have been introduced in the constant region of the knob and hole heavy chains to abrogate binding to Fcgamma receptors according to the method described in International Patent Appl. Publ. No. WO2012/130831A1.

The bispecific 2+1 anti-ICOS, anti-FAP huIgG1 P329GLALA inverted antibody was produced by co-transfecting HEK293F cells with the mammalian expression vectors using FectoPro (PolyPlus, US). The cells were transfected with the corresponding expression vectors in a 1:2:1:1 ratio (“vector heavy chain (VH-CH 1-VL-CH 1-CH2-CH3)”:“vector light chain (VL-CL)”:“vector heavy chain (VH-CH 1-CH2-CH3)”:“vector light chain (VH-CL)”. The constructs were produced and purified as described for the bispecific monovalent anti-ICOS and anti-FAP huIgG1 P329GLALA antibody (see Example 3.1).

The amino acid sequences for 2+1 anti-ICOS, anti-FAP Crossfab-IgG P329G LALA inverted constructs can be found in Table 20.

TABLE 20
Amino acid sequences of mature bispecific 2 + 1 anti-ICOS (1167)/anti-FAP
(4B9) Crossfab-IgG P329G LALA

| | SEQ | | |
|---|---|---|---|
| Molecule | ID NO: | Name | Sequence |
| 13 | 116 | (ICOS 1167) | EVRLLESGGGLVQPGGSLRLSCAASGFTFNTYAVHWV |
| | | VHCH1 Fc hole | RQAPGKGLEWVSGIGGSGVRTYYADSVKGRLTISRDN |
| | | | SKNTLYLQMNSLRAEDTAIYFCAKDIYVADFTGYAFD |
| | | | IWGQGTMVTVSSASTKGPSVFPLAPSSKSTSGGTAAL |
| | | | GCLVEDYFPEPVTVSWNSGALTSGVHTFPAVLQSSGL |
| | | | YSLSSVVTVPSSSLGTQTYICNVNHKPSNTKVDEKVE |
| | | | PKSCDKTHTCPPCPAPEAAGGPSVFLFPPKPKDTLMI |
| | | | SRTPEVTCVVVDVSHEDPEVKFNWYVDGVEVHNAKTK |
| | | | PREEQYNSTYRVVSVLTVLHQDWLNGKEYKCKVSNKA |
| | | | LGAPIEKTISKAKGQPREPQVCTLPPSRDELTKNQVS |
| | | | LSCAVKGFYPSDIAVEWESNGQPENNYKTTPPVLDSD |
| | | | GSFFLVSKLTVDKSRWQQGNVFSCSVMHEALHNHYTQ |
| | | | KSLSLSP |
| | 117 | (ICOS 1167) VLCL- | DIQMTQSPSSVSASVGDRVTITCRASQGINNFLAWYQ |
| | | light chain 1 | QKPGKAPKLLIYDASSLQSGVPSRFAGSGSGTDFTLT |
| | | | ISSLQPEDFATYYCQQYNFYPLTFGGGTMVEIKRTVA |
| | | | APSVFIFPPSDRKLKSGTASVVCLLNNFYPREAKVQW |
| | | | KVDNALQSGNSQESVTEQDSKDSTYSLSSTLTLSKAD |
| | | | YEKHKVYACEVTHQGLSSPVTKSFNRGEC |
| | 118 | (ICOS 1167) | EVRLLESGGGLVQPGGSLRLSCAASGFTFNTYAVHWV |
| | | VHCH1(FAP 4B9) | RQAPGKGLEWVSGIGGSGVRTYYADSVKGRLTISRDN |
| | | VLCH1 Fc knob | SKNTLYLQMNSLRAEDTAIYFCAKDIYVADFTGYAFD |
| | | | IWGQGTMVTVSSASTKGPSVFPLAPSSKSTSGGTAAL |
| | | | GCLVEDYFPEPVTVSWNSGALTSGVHTFPAVLQSSGL |
| | | | YSLSSVVTVPSSSLGTQTYICNVNHKPSNTKVDEKVE |
| | | | PKSCDGGGGSGGGGSEIVLTQSPGTLSLSPGERATLS |
| | | | CRASQSVTSSYLAWYQQKPGQAPRLLINVGSRRATGI |
| | | | PDRFSGSGSGTDFTLTISRLEPEDFAVYYCQQGIMLP |
| | | | PTFGQGTKVEIKSSASTKGPSVFPLAPSSKSTSGGTA |
| | | | ALGCLVKDYFPEPVTVSWNSGALTSGVHTFPAVLQSS |
| | | | GLYSLSSVVTVPSSSLGTQTYICNVNHKPSNTKVDKK |
| | | | VEPKSCDKTHTCPPCPAPEAAGGPSVFLFPPKPKDTL |
| | | | MISRTPEVTCVVVDVSHEDPEVKFNWYVDGVEVHNAK |
| | | | TKPREEQYNSTYRVVSVLTVLHQDWLNGKEYKCKVSN |
| | | | KALGAPIEKTISKAKGQPREPQVYTLPPCRDELTKNQ |
| | | | VSLWCLVKGFYPSDIAVEWESNGQPENNYKTTPPVLD |
| | | | SDGSFFLYSKLTVDKSRWQQGNVFSCSVMHEALHNHY |
| | | | TQKSLSLSP |
| | 119 | (FAP 4B9) VHCL- | EVQLLESGGGLVQPGGSLRLSCAASGFTFSSYAMSWV |
| | | light chain 2 | RQAPGKGLEWVSAIIGSGASTYYADSVKGRFTISRDN |
| | | | SKNTLYLQMNSLRAEDTAVYYCAKGWFGGFNYWGQGT |
| | | | LVTVSSASVAAPSVFIFPPSDEQLKSGTASVVCLLNN |
| | | | FYPREAKVQWKVDNALQSGNSQESVTEQDSKDSTYSL |
| | | | SSTLTLSKADYEKHKVYACEVTHQGLSSPVTKSFNRG |
| | | | EC |

TABLE 21
Biochemical analysis of bispecific constructs with a bivalent
binding to ICOS and a monovalent binding to FAP
(2 + 1 ICOS/FAP human IgG1 P329GLALA inverted)

| | | CE-SDS | |
|---|---|---|---|
| | Monomer | (non-reduced) | Yield |
| Molecule | [%] | [%] | [mg/l] |
| 13 | 96 | 100 | 1.4 |

### Example 4

### Humanization of Mouse and Rabbit Anti-ICOS Antibodies

### 4.1 Methodology

Suitable human acceptor frameworks were identified by querying a BLASTp database of human V- and J-region sequences for the murine input sequences (cropped to the variable part). Selective criteria for the choice of human acceptor framework were sequence homology, same or similar CDR lengths, and the estimated frequency of the human germline, but also the conservation of certain amino acids at the VH-VL domain interface. Following the germline identification step, the CDRs of the murine input sequences were grafted onto the human acceptor framework regions. Each amino acid difference between these initial CDR grafts and the parental antibodies was rated for possible impact on the structural integrity of the respective variable region, and “back mutations” towards the parental sequence were introduced whenever deemed appropriate. The structural assessment was based on Fv region homology models of both the parental antibody and the humanization variants, created with an in-house antibody structure homology modeling protocol implemented using the Biovia Discovery Studio Environment, version 17R2. In some humanization variants, “forward mutations” were included, i.e., amino acid exchanges that change the original amino acid occurring at a given CDR position of the parental binder to the amino acid found at the equivalent position of the human acceptor germline. The aim is to increase the overall human character of the humanization variants (beyond the framework regions) to further reduce the immunogenicity risk.

An in silico tool developed in-house was used to predict the VH-VL domain orientation of the paired VH and VL humanization variants (WO 2016/062734). The results were compared to the predicted VH-VL domain orientation of the parental binders to select for framework combinations which are close in geometry to the original antibodies. The rational is to detect possible amino acid exchanges in the VH-VL interface region that might lead to disruptive changes in the pairing of the two domains that in turn might have detrimental effects on the binding properties.

### 4.2 Choice of Acceptor Framework and Adaptations Thereof

Humanization of 009

TABLE 1
Acceptor frameworks for ICOS clone 009

| | | | Choice of |
|---|---|---|---|
| | | | human acceptor |
| | Murine V-region | Graft | V-region |
| | germline | variant | germline |
| VH | IGHV6-7*02 | VHG1 | IGHV3-49*04 |
| | | VHG2 | IGHV3-30*13 |
| VL | IGKV19-93*02 | VLG1 | IGKV1-33*01 |
| | | VLG2 | IGKV1-39*01 |

Post-CDR3 framework regions were adapted from human IGHJ germline IGHJ6*01/02 (YYYYYGMDVWGQGTTVTVSS, SEQ ID NO:120) and human IGKJ germline IGKJ2*01 (YTFGQGTKLEIK, SEQ ID NO:121). The part relevant for the acceptor framework is indicated in bold script.

Based on structural considerations, back mutations from the human acceptor framework to the amino acid in the parental binder were introduced at positions H40 (P>S), H42 (G>E), H49 (G>A), H94 (R>W), H105 (K>A) [VH1], H40 (P>S), H42 (G>E), H93 (A>T), H94 (R>W), H105 (K>A) [VH2], L38 (Q>H), L43 (A>G), L49 (Y>W), L100 (Q>S) [VL1], and L38 (Q>H), L43 (A>G), L49 (Y>W), L100 (Q>S) [VL2].

Furthermore, the positions H60 (S>A), H61 (D>A) [VH1], H60 (S>A) [VH2], L24 (K>Q) [VL1], and L24 (K>R) [VL2] were identified as promising candidates for forward mutations (Kabat numbering).

Humanization of 1138

TABLE 23
Acceptor frameworks for ICOS clone 1138

| | | | Choice of |
|---|---|---|---|
| | | | human acceptor |
| | Rabbit V-region | Graft | V-region |
| | germline | variant | germline |
| VH | IGHV1S40*01 | VHG1 | IGHV3-23*03 |
| VL | IGKV1S1*01 | VLG1 | IGKV1-39*01 |

Post-CDR3 framework regions were adapted from human IGHJ germline IGHJ1*01 (AEYFQHWGQGTLVTVSS, SEQ ID NO:122) and human IGKJ germline IGKJ4*01/02 (LTFGGGTKVEIK, SEQ ID NO:1223). The part relevant for the acceptor framework is indicated in bold script.

Based on structural considerations, back mutations from the human acceptor framework to the amino acid in the parental binder were introduced at positions H71 (R>K), H72 (D>T), H73 (N>S), H76 (N>T), H91 (Y>F), H94 (K>R) [VH1], and L1 (D>A), L42 (K>Q), L43 (A>P) [VL1]. In addition, in one variant of VH, the N-terminus was back-mutated (removal of H1 and mutation of H2 from V>Q) and in one variant of VH, the gap at position H75 of the rabbit framework was reintroduced.

Furthermore, the positions H61 (S>D), H62 (W>S), H63 (A>V) [VH1], and L24 (Q>R) [VL1] were identified as promising candidates for forward mutations (Kabat numbering).

Humanization of 1143

TABLE 24
Acceptor frameworks for ICOS clone 1143

| | Rabbit V-region | | Choice of human acceptor |
|---|---|---|---|
| | germline | Graft variant | V-region germline |
| VH | IGHV1S40*01 | VHG1 | IGHV3-23*03 |
| VL | IGKV1S4*01 | VLG1 | IGKV1-39*01 |

Post-CDR3 framework regions were adapted from human IGHJ germline IGHJ1*01 (AEYFQHWGQGTLVTVSS, SEQ ID NO:122) and human IGKJ germline IGKJ4*01/02 (LTFGGGTKVEIK, SEQ ID NO:123). The part relevant for the acceptor framework is indicated in bold script.

Based on structural considerations, back mutations from the human acceptor framework to the amino acid in the parental binder were introduced at positions H48 (V>I), H49 (S>G), H71 (R>K), H72 (D>T), H73 (N>S), H76 (N>T), H91 (Y>F), H94 (K>R) [VH1], and L42 (K>Q), L43 (A>P) [VL1]. In addition, in two variants of VH, the N-terminus was back-mutated (removal of H1 and mutation of H2 from V>Q) and in two variants of VH, the gap at position H75 of the rabbit framework was reintroduced.

Furthermore, the positions H61 (T>D) [VH1], and L24 (Q>R) [VL1] were identified as promising candidates for forward mutations (Kabat numbering).

### 4.3 Humanization Variants

Back mutations are prefixed with b, forward mutations with f, e.g., bM48I refers to a back mutation (human germline amino acid to parental antibody amino acid) from methionine to isoleucine at position 48 (Kabat numbering).

TABLE 25
Variants of Clone 009

| | | Identity to human |
|---|---|---|
| Variant | | V-region germline |
| Name | Back/forward mutations | (BLASTp) |
| VHG1a | bG49A, bR94W | 86.9 % |
| VHG1b | bG49A, bR94W, bK105A | 86.9 % |
| VHG1c | bP40S, bG42E, bG49A, bR94W, bK105A | 84.8 % |
| VHG1d | bG49A, fS60A, fD61A, bR94W | 88.9 % |
| VHG2a | bA93T, bR94W | 86.7 % |
| VHG2b | bA93T, bR94W, bK105A | 86.7 % |
| VHG2c | bP40S, bG42E, bA93T, bR94W, bK105A | 84.7 % |
| VHG2d | fS60A, bA93T, bR94W | 87.8 % |
| VLG1a | fK24Q, bY49W | 87.2 % |
| VLG1b | fK24Q, bQ38H, bA43G, bY49W, bQ100S | 85.1 % |
| VLG2a | fK24R, bY49W | 87.6 % |
| VLG2b | fK24R, bQ38H, bA43G, bY49W, bQ100S | 85.4 % |

TABLE 26
Variants of Clone 1138

| | | Identity to human |
|---|---|---|
| Variant | | V-region germline |
| Name | Back/forward mutations | (BLASTp) |
| VHG1a | bY91F, bK94R | 84.2 % |
| VHG1b | fS61D, fW62S, fA63V, bY91F, bK94R | 87.1 % |
| VHG1c | bR71K, bD72T, bN73S, bN76T, bY91F, | 80.2 % |
| | bK94R | |
| VHG1d | bE1−, bV2Q, bY91F, bK94R | 83.8 % |
| VHG1e | bR71K, bD72T, bN73S, bK75−, bN76T, | 79.2 % |
| | bY91F, bK94R | |
| VLG1a | fQ24R | 94.4 % |
| VLG1b | fQ24R, bK42Q, bA43P | 92.2 % |
| VLG1c | bD1A, fQ24R | 92.1 % |

TABLE 27
Variants of Clone 1143

| | | Identity to human |
|---|---|---|
| Variant | | V-region germline |
| Name | Back/forward mutations | (BLASTp) |
| VHG1a | fT61D, bY91F, bK94R | 90.9% |
| VHG1b | bV48I, bS49G, fT61D, bY91F, bK94R | 88.9 % |
| VHG1e | fT61D, bR71K, bD72T, bN73S, bN76T, | 86.9 % |
| | bY91F, bK94R | |
| VHG1d | bE1_, bV2Q, fT61D, bY91F, bK94R | 90.7 % |
| VHG1c | fT61D, bK75−, bY91F, bK94R | 89.9 % |
| VHG1f | bV48I, bS49G, fT61D, bR71K, bD72T, | 84.8 % |
| | bN73S, bN76T, bY91F, bK94R | |
| VHG1g | bE1−, bV2Q, bV48I, bS49G, fT61D, | 88.7 % |
| | bY91F, bK94R | |
| VHG1h | bV48I, bS49G, fT61D, bK75−, bY91F, | 87.9 % |
| | bK94R | |
| VLG1a | fQ24R | 88.9% |
| VLG1b | fQ24R, bK42Q, bA43P | 87.8 % |

The amino acid sequences of the humanization variants can be found in Table 28 below.

TABLE 28
Amino acid sequences of humanization variants for clones ICOS 009, 1138 and
1143

| ICOS | SEQ ID | Variant | |
|---|---|---|---|
| clone | NO: | Name | Sequence |
| 009 | 124 | VHG1a | EVQLVESGGGLVQPGRSLRLSCTASGFTFSDYWMNWVR |
| | | | QAPGKGLEWVAQIRNKPYNYETYYSDSVKGRFTISRDD |
| | | | SKSIAYLQMNSLKTEDTAVYYCTWPRLRSSDWHEDVWG |
| | | | KGTTVTVSS |
| | 125 | VHG1b | EVQLVESGGGLVQPGRSLRLSCTASGFTFSDYWMNWVR |
| | | | QAPGKGLEWVAQIRNKPYNYETYYSDSVKGRFTISRDD |
| | | | SKSIAYLQMNSLKTEDTAVYYCTWPRLRSSDWHEDVWG |
| | | | AGTTVTVSS |
| | 126 | VHG1c | EVQLVESGGGLVQPGRSLRLSCTASGFTFSDYWMNWVR |
| | | | QSPEKGLEWVAQIRNKPYNYETYYSDSVKGRFTISRDD |
| | | | SKSIAYLQMNSLKTEDTAVYYCTWPRLRSSDWHEDVWG |
| | | | AGTTVTVSS |
| | 127 | VHG1d | EVQLVESGGGLVQPGRSLRLSCTASGFTFSDYWMNWVR |
| | | | QAPGKGLEWVAQIRNKPYNYETYYAASVKGRFTISRDD |
| | | | SKSIAYLQMNSLKTEDTAVYYCTWPRLRSSDWHEDVWG |
| | | | KGTTVTVSS |
| | 128 | VHG2a | QVQLVESGGGVVQPGRSLRLSCAASGFTFSDYWMNWVR |
| | | | QAPGKGLEWVAQIRNKPYNYETYYSDSVKGRFTISRDN |
| | | | SKNRLYLQMNSLRAEDTAVYYCTWPRLRSSDWHEDVWG |
| | | | KGTTVTVSS |
| | 129 | VHG2b | QVQLVESGGGVVQPGRSLRLSCAASGFTFSDYWMNWVR |
| | | | QAPGKGLEWVAQIRNKPYNYETYYSDSVKGRFTISRDN |
| | | | SKNRLYLQMNSLRAEDTAVYYCTWPRLRSSDWHEDVWG |
| | | | AGTTVTVSS |
| | 130 | VHG2c | QVQLVESGGGVVQPGRSLRLSCAASGFTFSDYWMNWVR |
| | | | QSPEKGLEWVAQIRNKPYNYETYYSDSVKGRFTISRDN |
| | | | SKNRLYLQMNSLRAEDTAVYYCTWPRLRSSDWHEDVWG |
| | | | AGTTVTVSS |
| | 131 | VHG2d | QVQLVESGGGVVQPGRSLRLSCAASGFTFSDYWMNWVR |
| | | | QAPGKGLEWVAQIRNKPYNYETYYADSVKGRFTISRDN |
| | | | SKNRLYLQMNSLRAEDTAVYYCTWPRLRSSDWHEDVWG |
| | | | KGTTVTVSS |
| | 132 | VLG1a | DIQMTQSPSSLSASVGDRVTITCQASQDINKNIAWYQQ |
| | | | KPGKAPKLLIWYTSTLQTGVPSRFSGSGSGTDFTFTIS |
| | | | SLQPEDIATYYCLQEDNLYTEGQGTKLEIK |
| | 133 | VLG1b | DIQMTQSPSSLSASVGDRVTITCQASQDINKNIAWYQH |
| | | | KPGKGPKLLIWYTSTLQTGVPSRFSGSGSGTDFTFTIS |
| | | | SLQPEDIATYYCLQEDNLYTEGSGTKLEIK |
| | 134 | VLG2a | DIQMTQSPSSLSASVGDRVTITCRASQDINKNIAWYQQ |
| | | | KPGKAPKLLIWYTSTLQTGVPSRFSGSGSGTDFTLTIS |
| | | | SLQPEDFATYYCLQEDNLYTEGQGTKLEIK |
| | 135 | VLG2b | DIQMTQSPSSLSASVGDRVTITCRASQDINKNIAWYQH |
| | | | KPGKGPKLLIWYTSTLQTGVPSRFSGSGSGTDFTLTIS |
| | | | SLQPEDFATYYCLQEDNLYTEGSGTKLEIK |
| 1138 | 136 | VHG1a | EVQLLESGGGLVQPGGSLRLSCAASGFDLSSYYYMSWV |
| | | | RQAPGKGLEWVSSIYADIYGGTTHYASWAKGRFTISRD |
| | | | NSKNTLYLQMNSLRAEDTAVYFCAREDGSRYGGSGYYN |
| | | | LWGQGTLVTVSS |
| | 137 | VHG1b | EVQLLESGGGLVQPGGSLRLSCAASGFDLSSYYYMSWV |
| | | | RQAPGKGLEWVSSIYADIYGGTTHYADSVKGRFTISRD |
| | | | NSKNTLYLQMNSLRAEDTAVYFCAREDGSRYGGSGYYN |
| | | | LWGQGTLVTVSS |
| | 138 | VHG1c | EVQLLESGGGLVQPGGSLRLSCAASGFDLSSYYYMSWV |
| | | | RQAPGKGLEWVSSIYADIYGGTTHYASWAKGRFTISKT |
| | | | SSKTTLYLQMNSLRAEDTAVYFCAREDGSRYGGSGYYN |
| | | | LWGQGTLVTVSS |
| | 139 | VHG1d | QQLLESGGGLVQPGGSLRLSCAASGFDLSSYYYMSWVR |
| | | | QAPGKGLEWVSSIYADIYGGTTHYASWAKGRFTISRDN |
| | | | SKNTLYLQMNSLRAEDTAVYFCAREDGSRYGGSGYYNL |
| | | | WGQGTLVTVSS |
| | 140 | VHG1e | EVQLLESGGGLVQPGGSLRLSCAASGFDLSSYYYMSWV |
| | | | RQAPGKGLEWVSSIYADIYGGTTHYASWAKGRFTISKT |
| | | | SSTTLYLQMNSLRAEDTAVYFCAREDGSRYGGSGYYNL |
| | | | WGQGTLVTVSS |
| | 141 | VLG1a | DIQMTQSPSSLSASVGDRVTITCRASQNIYSNLAWYQQ |
| | | | KPGKAPKLLIYAASYLTSGVPSRFSGSGSGTDFTLTIS |
| | | | SLQPEDFATYYCQQGHTTDNIDNAFGGGTKVEIK |
| | 142 | VLG1b | DIQMTQSPSSLSASVGDRVTITCRASQNIYSNLAWYQQ |
| | | | KPGQPPKLLIYAASYLTSGVPSRFSGSGSGTDFTLTIS |
| | | | SLQPEDFATYYCQQGHTTDNIDNAFGGGTKVEIK |
| | 143 | VLG1c | AIQMTQSPSSLSASVGDRVTITCRASQNIYSNLAWYQQ |
| | | | KPGKPPKLLIYAASYLTSGVSSRFSGSGSGTDFTLTIS |
| | | | SLQPEDFATYYCQQGHTTDNIDNAFGGGTKVEIK |
| 1143 | 144 | VHG1a | EVQLLESGGGLVQPGGSLRLSCAASGFDFSSAYDMSWV |
| | | | RQAPGKGLEWVSVIYYGDGITYYADSVKGRFTISRDNS |
| | | | KNTLYLQMNSLRAEDTAVYFCARGAFLGSSYYLSLWGQ |
| | | | GTLVTVSS |
| | 145 | VHG1b | EVQLLESGGGLVQPGGSLRLSCAASGFDFSSAYDMSWV |
| | | | RQAPGKGLEWIGVIYYGDGITYYADSVKGRFTISRDNS |
| | | | KNTLYLQMNSLRAEDTAVYFCARGAFLGSSYYLSLWGQ |
| | | | GTLVTVSS |
| | 146 | VHG1c | EVQLLESGGGLVQPGGSLRLSCAASGFDFSSAYDMSWV |
| | | | RQAPGKGLEWVSVIYYGDGITYYADSVKGRFTISKTSS |
| | | | KTTLYLQMNSLRAEDTAVYFCARGAFLGSSYYLSLWGQ |
| | | | GTLVTVSS |
| | 147 | VHG1d | QQLLESGGGLVQPGGSLRLSCAASGFDFSSAYDMSWVR |
| | | | QAPGKGLEWVSVIYYGDGITYYADSVKGRFTISRDNSK |
| | | | NTLYLQMNSLRAEDTAVYFCARGAFLGSSYYLSLWGQG |
| | | | TLVTVSS |
| | 148 | VHG1e | EVQLLESGGGLVQPGGSLRLSCAASGFDFSSAYDMSWV |
| | | | RQAPGKGLEWVSVIYYGDGITYYADSVKGRFTISRDNS |
| | | | NTLYLQMNSLRAEDTAVYFCARGAFLGSSYYLSLWGQG |
| | | | TLVTVSS |
| | 149 | VHG1f | EVQLLESGGGLVQPGGSLRLSCAASGFDFSSAYDMSWV |
| | | | RQAPGKGLEWIGVIYYGDGITYYADSVKGRFTISKTSS |
| | | | KTTLYLQMNSLRAEDTAVYFCARGAFLGSSYYLSLWGQ |
| | | | GTLVTVSS |
| | 150 | VHG1g | QQLLESGGGLVQPGGSLRLSCAASGFDFSSAYDMSWVR |
| | | | QAPGKGLEWIGVIYYGDGITYYADSVKGRFTISRDNSK |
| | | | NTLYLQMNSLRAEDTAVYFCARGAFLGSSYYLSLWGQG |
| | | | TLVTVSS |
| | 151 | VHG1h | EVQLLESGGGLVQPGGSLRLSCAASGFDFSSAYDMSWV |
| | | | RQAPGKGLEWIGVIYYGDGITYYADSVKGRFTISRDNS |
| | | | NTLYLQMNSLRAEDTAVYFCARGAFLGSSYYLSLWGQG |
| | | | TLVTVSS |
| | 152 | VLG1a | DIQMTQSPSSLSASVGDRVTITCRASENIYNWLAWYQQ |
| | | | KPGKAPKLLIYDASKLASGVPSRFSGSGSGTDFTLTIS |
| | | | SLQPEDFATYYCQQAYTYGNIDNAFGGGTKVEIK |
| | 153 | VLG1b | DIQMTQSPSSLSASVGDRVTITCRASENIYNWLAWYQQ |
| | | | KPGQPPKLLIYDASKLASGVPSRFSGSGSGTDFTLTIS |
| | | | SLQPEDFATYYCQQAYTYGNIDNAFGGGTKVEIK |

### 4.4 Cloning and Expression of Humanization Variants

The variable region of heavy and light chain DNA sequences were subcloned in frame with either the constant heavy chain or the constant light chain pre-inserted into the respective recipient mammalian expression vector. Protein expression is driven by an MPSV promoter and a synthetic polyA signal sequence is present at the 3′ end of the CDS. The amino acid sequences of the selected anti-ICOS humanization variants are shown in Table 29.

TABLE 29
Amino acid sequences of parental and selected anti-ICOS humanization
variants in human IgG format

| | SEQ ID | |
|---|---|---|
| Molecule | NO: | Sequence |
| 25 | 155 | DIQMTQSPSSLSASVGDRVTITCQASQDINKNIAWYQQKPGKAPKLLIW |
| (ICOS | (VL) | YTSTLQTGVPSRFSGSGSGTDFTFTISSLQPEDIATYYCLQFDNLYTFG |
| H009v1_1) | | QGTKLEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGEC |
| | 154 | EVQLVESGGGLVQPGRSLRLSCTASGFTFSDYWMNWVRQAPGKGLEWVA |
| | (VH) | QIRNKPYNYETYYSDSVKGRFTISRDDSKSIAYLQMNSLKTEDTAVYYC |
| | | TWPRLRSSDWHFDVWGKGTTVTVSSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPEAAGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALGAPIEKTIS |
| | | KAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNH |
| | | YTQKSLSLSP |
| 26 | 157 | DIQMTQSPSSLSASVGDRVTITCQASQDINKNIAWYQQKPGKAPKLLIW |
| (ICOS | (VL) | YTSTLQTGVPSRFSGSGSGTDFTFTISSLQPEDIATYYCLQFDNLYTFG |
| H009v1_2) | | QGTKLEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGEC |
| | 156 | EVQLVESGGGLVQPGRSLRLSCTASGFTFSDYWMNWVRQAPGKGLEWVA |
| | (VH) | QIRNKPYNYETYYSDSVKGRFTISRDDSKSIAYLQMNSLKTEDTAVYYC |
| | | TWPRLRSSDWHFDVWGAGTTVTVSSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPEAAGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALGAPIEKTIS |
| | | KAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNH |
| | | YTQKSLSLSP |
| 27 | 159 | DIQMTQSPSSLSASVGDRVTITCQASQDINKNIAWYQQKPGKAPKLLIW |
| (ICOS | (VL) | YTSTLQTGVPSRFSGSGSGTDFTFTISSLQPEDIATYYCLQFDNLYTFG |
| H009v1_3) | | QGTKLEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGEC |
| | 158 | EVQLVESGGGLVQPGRSLRLSCTASGFTFSDYWMNWVRQAPGKGLEWVA |
| | (VH) | QIRNKPYNYETYYAASVKGRFTISRDDSKSIAYLQMNSLKTEDTAVYYC |
| | | TWPRLRSSDWHFDVWGKGTTVTVSSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPEAAGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALGAPIEKTIS |
| | | KAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNH |
| | | YTQKSLSLSP |
| 32 | 161 | ALVMTQTPSSVSAAVGGTVTINCQASQNIYSNLAWYQQKPGQPPKLLIY |
| (ICOS 1138) | (VL) | AASYLTSGVSSRFKGSGAGTQFTLTISGVESADAATYYCQQGHTTDNID |
| | | NAFGGGTEVVVKRTVAAPSVFIFPPSDEQLKSGTASVVCLLNNFYPREA |
| | | KVQWKVDNALQSGNSQESVTEQDSKDSTYSLSSTLTLSKADYEKHKVYA |
| | | CEVTHQGLSSPVTKSFNRGEC |
| | 160 | QSLEESGGDLVKPGASLTLTCTASGFDLSSYYYMSWVRQAPGKGLEWIA |
| | (VH) | SIYADIYGGTTHYASWAKGRFTISKTSSTTVTLQMTSLTAADTATYFCA |
| | | REDGSRYGGSGYYNLWGPGTLVTVSSASTKGPSVFPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEAAGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALGAPIEKTI |
| | | SKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHN |
| | | HYTQKSLSLSP |
| 33 | 163 | DIQMTQSPSSLSASVGDRVTITCRASQNIYSNLAWYQQKPGQPPKLLIY |
| (ICOS | (VL) | AASYLTSGVPSRFSGSGSGTDFTLTISSLQPEDFATYYCQQGHTTDNID |
| H1138_1) | | NAFGGGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVYA |
| | | CEVTHQGLSSPVTKSFNRGEC |
| | 162 | EVQLLESGGGLVQPGGSLRLSCAASGFDLSSYYYMSWVRQAPGKGLEWV |
| | (VH) | SSIYADIYGGTTHYASWAKGRFTISKTSSKTTLYLQMNSLRAEDTAVYF |
| | | CAREDGSRYGGSGYYNLWGQGTLVTVSSASTKGPSVFPLAPSSKSTSGG |
| | | TAALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEAAG |
| | | GPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALGAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEAL |
| | | HNHYTQKSLSLSP |
| 34 | 165 | AIQMTQSPSSLSASVGDRVTITCRASQNIYSNLAWYQQKPGKPPKLLIY |
| (ICOS | (VL) | AASYLTSGVSSRFSGSGSGTDFTLTISSLQPEDFATYYCQQGHTTDNID |
| H1138_2) | | NAFGGGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVYA |
| | | CEVTHQGLSSPVTKSFNRGEC |
| | 164 | EVQLLESGGGLVQPGGSLRLSCAASGFDLSSYYYMSWVRQAPGKGLEWV |
| | (VH) | SSIYADIYGGTTHYASWAKGRFTISRDNSKNTLYLQMNSLRAEDTAVYF |
| | | CAREDGSRYGGSGYYNLWGQGTLVTVSSASTKGPSVFPLAPSSKSTSGG |
| | | TAALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEAAG |
| | | GPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALGAPIEK |
| | | TISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEAL |
| | | HNHYTQKSLSLSP |
| 35 | 167 | AIQMTQSPSSLSASVGDRVTITCRASQNIYSNLAWYQQKPGKPPKLLIY |
| (ICOS | (VL) | AASYLTSGVSSRFSGSGSGTDFTLTISSLQPEDFATYYCQQGHTTDNID |
| H1138_3) | | NAFGGGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVYA |
| | | CEVTHQGLSSPVTKSFNRGEC |
| | 166 | EVQLLESGGGLVQPGGSLRLSCAASGFDLSSYYYMSWVRQAPGKGLEWV |
| | (VH) | SSIYADIYGGTTHYASWAKGRFTISKTSSTTLYLQMNSLRAEDTAVYFC |
| | | AREDGSRYGGSGYYNLWGQGTLVTVSSASTKGPSVFPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPEAAGG |
| | | PSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALGAPIEKT |
| | | ISKAKGQPREPQVYTLPPSRDELTKNQVSLTCLVKGFYPSDIAVEWESN |
| | | GQPENNYKTTPPVLDSDGSFELYSKLTVDKSRWQQGNVESCSVMHEALH |
| | | NHYTQKSLSLSP |
| 28 | 169 | AIDMTQTPASVEAAVGGTVTINCQASENTYNWLAWYQQKPGQPPKLLIY |
| (ICOS | (VL) | DASKLASGVPSRFSASGSGTQFTLTISAVESADAATYYCQQAYTYGNID |
| 1143v2) | | NAFGGGTEVVVSRTVAAPSVFIFPPSDEQLKSGTASVVCLLNNFYPREA |
| | | KVQWKVDNALQSGNSQESVTEQDSKDSTYSLSSTLTLSKADYEKHKVYA |
| | | CEVTHQGLSSPVTKSFNRGEC |
| | 168 | QSLEESGGDLVKPGASLTLTCKASGEDESSAYDMSWVRQAPGKGLEWIG |
| | (VH) | VIYYGDGITYYATSVKGRFTISKTSSTTVPLQMTSLTAADTATYFCARG |
| | | AFLGSSYYLSLWGQGTLVTVSSASTKGPSVFPLAPSSKSTSGGTAALGC |
| | | LVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPEAAGGPSVFL |
| | | FPPKPKDTLMISRTPEVTCVVVDVSHEDPEVKFNWYVDGVEVHNAKTKP |
| | | REEQYNSTYRVVSVLTVLHQDWLNGKEYKCKVSNKALGAPIEKTISKAK |
| | | GQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQ |
| | | KSLSLSP |
| 29 | 171 | DIQMTQSPSSLSASVGDRVTITCRASENIYNWLAWYQQKPGKAPKLLIY |
| (ICOS | (VL) | DASKLASGVPSRFSGSGSGTDFTLTISSLQPEDFATYYCQQAYTYGNID |
| H1143v2_1) | | NAFGGGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVYA |
| | | CEVTHQGLSSPVTKSFNRGEC |
| | 170 | EVQLLESGGGLVQPGGSLRLSCAASGFDFSSAYDMSWVRQAPGKGLEWV |
| | (VH) | SVIYYGDGITYYADSVKGRFTISRDNSKNTLYLQMNSLRAEDTAVYFCA |
| | | RGAFLGSSYYLSLWGQGTLVTVSSASTKGPSVFPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPEAAGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALGAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHY |
| | | TQKSLSLSP |
| 30 | 173 | DIQMTQSPSSLSASVGDRVTITCRASENIYNWLAWYQQKPGKAPKLLIY |
| (ICOS | (VL) | DASKLASGVPSRFSGSGSGTDFTLTISSLQPEDFATYYCQQAYTYGNID |
| H1143v2_2) | | NAFGGGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVYA |
| | | CEVTHQGLSSPVTKSFNRGEC |
| | 172 | EVQLLESGGGLVQPGGSLRLSCAASGFDFSSAYDMSWVRQAPGKGLEWV |
| | (VH) | SVIYYGDGITYYADSVKGRFTISKTSSKTTLYLQMNSLRAEDTAVYFCA |
| | | RGAFLGSSYYLSLWGQGTLVTVSSASTKGPSVFPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPEAAGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALGAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHY |
| | | TQKSLSLSP |
| 31 | 175 | DIQMTQSPSSLSASVGDRVTITCRASENIYNWLAWYQQKPGKAPKLLIY |
| (ICOS | (VL) | DASKLASGVPSRFSGSGSGTDFTLTISSLQPEDFATYYCQQAYTYGNID |
| H1143v2_3) | | NAFGGGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVYA |
| | | CEVTHQGLSSPVTKSFNRGEC |
| | 174 | EVQLLESGGGLVQPGGSLRLSCAASGFDFSSAYDMSWVRQAPGKGLEWI |
| | (VH) | GVIYYGDGITYYADSVKGRFTISRDNSNTLYLQMNSLRAEDTAVYFCAR |
| | | GAFLGSSYYLSLWGQGTLVTVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPEAAGGPSVF |
| | | LFPPKPKDTLMISRTPEVTCVVVDVSHEDPEVKFNWYVDGVEVHNAKTK |
| | | PREEQYNSTYRVVSVLTVLHQDWLNGKEYKCKVSNKALGAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYT |
| | | QKSLSLSP |

The humanization variants in human IgG format were produced by co-transfecting Expi293F (Thermo Fisher) cells with the mammalian expression vectors using ExpiFectamine 293 (Thermo Fisher). The cells were transfected with the corresponding expression vectors in a 1:1 ratio (“vector heavy chain”:“vector light chain”).

For production in 48-deep well plates, 2.5e6 cells/mL Expi293F cells were seeded at the day of transfection. A transient transfection was performed with the plasmids encoding the target protein of interest. A MasterMix of DNA/ExpiFectamine 293 was prepared in Opti-MEM medium (Thermo Fisher), incubated for 5 minutes and added to the cell suspension. 24 hours after transfection each well was fed with 10 μL Enhancer 1 (Thermo Fisher) and 100 μL Enhancer 2 (Thermo Fisher).

After culturing for 5 days, the cell supernatant was collected by centrifugation for 50 minutes at 1200×g. The solution was sterile filtered (0.2 μm filter) and kept at 4° C.

The secreted protein is purified from cell culture supernatants by affinity chromatography on a liquid handling platform in 96 well format using Protein A affinity chromatography. For affinity chromatography supernatant is loaded on a ProPlus PhyTip Column (MabSelect SuRe™) (CV=40 μl; Tip volume 500 μl Phynexus) equilibrated with 2 times 290 μl 20 mM sodium phosphate, 20 mM sodium citrate, pH 7.5. Unbound protein is removed by washing with 4 times 300 μl 20 mM sodium phosphate, 20 mM sodium citrate, pH 7.5 and target protein is eluted in 2 times 150 μl 20 mM sodium citrate, 100 mM sodium chloride, 100 mM glycine, pH 3.0. Protein solution is neutralized by adding 30 μl of 0.5 M sodium phosphate, pH 8.0.

Purified proteins were quantified using a Nanodrop spectrophotometer (ThermoFisher) and analyzed by CE-SDS under denaturing and reducing conditions (LabChip GX, Perkin Elmer) and analytical SEC (UP-SW3000, Tosho Bioscience). Under reducing conditions, polypeptide chains related to the IgG were identified with the Lab Chip device by comparison of the apparent molecular sizes to a molecular weight standard.

### Example 5

### Generation of Humanized Variants of Anti-CEA Antibody A5B7

### 5.1 Methodology

Anti-CEA antibody A5B7 is for example disclosed by M. J. Banfield et al, Proteins 1997, 29(2), 161-171 and its structure can be found as PDB ID:1CLO in the Protein structural database PDB (www.rcsb.org, H. M. Berman et al, The Protein Data Bank, Nucleic Acids Research, 2000, 28, 235-242). This entry includes the heavy and the light chain variable domain sequence. For the identification of a suitable human acceptor framework during the humanization of the anti-CEA binder A5B7, a classical approach was taken by searching for an acceptor framework with high sequence homology, grafting of the CDRs on this framework, and evaluating which back-mutations can be envisaged. More explicitly, each amino acid difference of the identified frameworks to the parental antibody was judged for impact on the structural integrity of the binder, and back mutations towards the parental sequence were introduced whenever appropriate. The structural assessment was based on Fv region homology models of both the parental antibody and its humanized versions created with an in-house antibody structure homology modeling tool implemented using the Biovia Discovery Studio Environment, version 4.5.

### 5.2 Choice of Acceptor Framework and Adaptations Thereof

The acceptor framework was chosen as described in Table 30 below:

TABLE 30
Acceptor framework

| | | Choice of human |
|---|---|---|
| | Closest murine | acceptor V-region |
| | V-region germline | germline |
| A5B7 VH | mu-IGHV7-3-02 | IGHV3-23-01 or |
| | | IGHV3-15-01 |
| A5B7 VL | mu-IGKV4-72-01 | IGKV3-11-01 |

Post-CDR3 framework regions were adapted from human J-element germline IGJH6 for the heavy chain, and a sequence similar to the kappa J-element IGKJ2, for the light chain.

Based on structural considerations, back mutations from the human acceptor framework to the amino acid in the parental binder were introduced at positions 93 and 94 of the heavy chain.

### 5.3 VH and VL Regions of the Resulting Humanized CEA Antibodies

The resulting VH domains of humanized CEA antibodies can be found in Table 31 below and the resulting VL domains of humanized CEA antibodies are listed in Table 32 below.

TABLE 31
Amino acid sequences of the VH domains of humanized CEA antibodies, based
on human acceptor framework IGHV3-23 or IGHV3-15

| | | Seq ID |
|---|---|---|
| Description | Sequence | No |
| A5B7 VH | EVKLVESGGGLVQPGGSLRLSCATSGFTFTDYYMNWVRQPPGKALEW | 176 |
| murine donor | LGFIGNKANGYTTEYSASVKGRFTISRDKSQSILYLQMNTLRAEDSA | |
| sequence | TYYCTRDRGLRFYFDYWGQGTTLTVSS | |
| IGHV3-23-02 | EVQLLESGGGLVQPGGSLRLSCAASGFTFSSYAMSWVRQAPGKGLEW | 177 |
| human | VSAISGSGGSTYYGDSVKGRFTISRDNSKNTLYLQMNSLRAEDTAVY | |
| acceptor | YCAK | |
| sequence | | |
| Humanized | | |
| variants | | |
| 3-23A5-1 | EVQLLESGGGLVQPGGSLRLSCAASGFTFTDYYMNWVRQAPGKGLEW | 179 |
| | VGFIGNKANGYTTEYSASVKGRFTISRDNSKNTLYLQMNSLRAEDTA | |
| | VYYCARDRGLRFYFDYWGQGTTVTVSS | |
| 3-23A5-2 | EVQLLESGGGLVQPGGSLRLSCAASGFTFTDYYMNWVRQAPGKGLEW | 180 |
| | VGFIGNKANGYTTYYGDSVKGRFTISRDNSKNTLYLQMNSLRAEDTA | |
| | VYYCARDRGLRFYFDYWGQGTTVTVSS | |
| 3-23A5-3 | EVQLLESGGGLVQPGGSLRLSCAASGFTFTDYYMNWVRQAPGKGLEW | 181 |
| | VGFIGNKGYTTEYSASVKGRFTISRDNSKNTLYLQMNSLRAEDTAVY | |
| | YCARDRGLRFYFDYWGQGTTVTVSS | |
| 3-23A5-4 | EVQLLESGGGLVQPGGSLRLSCAASGFTFTDYYMSWVRQAPGKGLEW | 182 |
| | VGFIGNKANGYTTEYSASVKGRFTISRDNSKNTLYLQMNSLRAEDTA | |
| | VYYCARDRGLRFYFDYWGQGTTVTVSS | |
| 3-23A5-1A | EVQLLESGGGLVQPGGSLRLSCAASGFTFTDYYMNWVRQAPGKGLEW | 183 |
| (all_backmut | LGFIGNKANGYTTEYSASVKGRFTISRDKSKNTLYLQMNSLRAEDTA | |
| ations) | TYYCTRDRGLRFYFDYWGQGTTVTVSS | |
| 3-23A5-1C | EVQLLESGGGLVQPGGSLRLSCAASGFTFTDYYMNWVRQAPGKGLEW | 184 |
| (A93T) | VGFIGNKANGYTTEYSASVKGRFTISRDNSKNTLYLQMNSLRAEDTA | |
| | VYYCTRDRGLRFYFDYWGQGTTVTVSS | |
| 3-23A5-1D | EVQLLESGGGLVQPGGSLRLSCAASGFTFTDYYMNWVRQAPGKGLEW | 185 |
| (K73) | VGFIGNKANGYTTEYSASVKGRFTISRDKSKNTLYLQMNSLRAEDTA | |
| | VYYCARDRGLRFYFDYWGQGTTVTVSS | |
| 3-23A5-1E | EVQLLESGGGLVQPGGSLRLSCAASGFTFTDYYMNWVRQAPGKGLEW | 68 |
| (G54A) | LGFIGNKANAYTTEYSASVKGRFTISRDKSKNTLYLQMNSLRAEDTA | |
| | TYYCTRDRGLRFYFDYWGQGTTVTVSS | |
| IGHV3-15*01 | EVQLVESGGGLVKPGGSLRLSCAASGFTFSNAWMSWVRQAPGKGLEW | 178 |
| human | VGRIKSKTDGGTTDYAAPVKGRFTISRDDSKNTLYLQMNSLKTEDTA | |
| acceptor | VYYCTT | |
| sequence | | |
| Humanized | | |
| variants | | |
| 3-15A5-1 | EVQLVESGGGLVKPGGSLRLSCAASGFTFTDYYMNWVRQAPGKGLEW | 186 |
| | VGFIGNKANGYTTEYSASVKGRFTISRDDSKNTLYLQMNSLKTEDTA | |
| | VYYCTRDRGLRFYFDYWGQGTTVTVSS | |
| 3-15A5-2 | EVQLVESGGGLVKPGGSLRLSCAASGFTFTDYYMNWVRQAPGKGLEW | 187 |
| | VGFIGNKANGYTTEYAAPVKGRFTISRDDSKNTLYLQMNSLKTEDTA | |
| | VYYCTRDRGLRFYFDYWGQGTTVTVSS | |
| 3-15A5-3 | EVQLVESGGGLVKPGGSLRLSCAASGFTFTDYYMNWVRQAPGKGLEW | 188 |
| | VGFIGNKANGGTTDYAAPVKGRFTISRDDSKNTLYLQMNSLKTEDTA | |
| | VYYCTRDRGLRFYFDYWGQGTTVTVSS | |

For the heavy chain, the initial variant 3-23A5-1 was found suitable in binding assays (but showed slightly less binding than the parental murine antibody) and was chosen as starting point for further modifications. The variants based on IGHV3-15 showed less binding activity compared to humanized variant 3-23A5-1.

In order to restore the full binding activity of the parental chimeric antibody, variants 3-23A5-TA, 3-23A5-TC and 3-23A5-TD were created. It was also tested for variant 3-23A5-1 whether the length of CDR-2 could be adapted to the human acceptor sequence, but this construct completely lost binding activity. Since a putative deamidation hotspot was present in CDR-H-2 (Asn53-Gly54), we changed that motif to Asn53-Ala54. Another possible hotspot Asn73-Ser74 was backmutated to Lys73-Ser74. Thus, variant 3-23A5-1E was created.

TABLE 32
Amino acid sequences of the VL domains of humanized CEA antibodies,
based on human acceptor framework IGKV3-11.

| | | Seq ID |
|---|---|---|
| Description | Sequence | No |
| A5B7 VL | QTVLSQSPAILSASPGEKVTMTCRASSSVTYIHWYQQKPGSSPKSWIYA | 189 |
| murine | TSNLASGVPARFSGSGSGTSYSLTISRVEAEDAATYYCQHWSSKPPTFG | |
| donor | GGTKLEIK | |
| sequence | | |
| IGKV3-11 | EIVLTQSPATLSLSPGERATLSCRASQSVSSYLAWYQQKPGQAPRLLIY | 190 |
| human | DASNRATGIPARFSGSGSGTDFTLTISSLEPEDFAVYYCQQRSNWP | |
| acceptor | | |
| sequence | | |
| humanized | | |
| variants | | |
| A5-L1 | EIVLTQSPATLSLSPGERATLSCRASSSVTYIHWYQQKPGQAPRLLIYA | 191 |
| | TSNLASGIPARFSGSGSGTDFTLTISSLEPEDFAVYYCQHWSSKPPTFG | |
| | QGTKLEIK | |
| A5-L2 | EIVLTQSPATLSLSPGERATLSCRASQSVSSYIHWYQQKPGQAPRLLIY | 192 |
| | ATSNLASGIPARFSGSGSGTDFTLTISSLEPEDFAVYYCQHWSSKPPTF | |
| | GQGTKLEIK | |
| A5-L3 | EIVLTQSPATLSLSPGERATLSCRASSSVTYIHWYQQKPGQAPRLLIYD | 193 |
| | ASNRATGIPARFSGSGSGTDFTLTISSLEPEDFAVYYCQHWSSKPPTFG | |
| | QGTKLEIK | |
| A5-L4 | EIVLTQSPATLSLSPGERATLSCRASSSVTYIHWYQQKPGQAPRLLIYA | 194 |
| | TSNLASGIPARFSGSGSGTDFTLTISSLEPEDFAVYYCQQWSSKPPTFG | |
| | QGTKLEIK | |
| A5-L1A | QTVLTQSPATLSLSPGERATLSCRASSSVTYIHWYQQKPGSSPKSWIYA | 195 |
| (all_backm | TSNLASGIPARFSGSGSGTDYTLTISSLEPEDFAVYYCQHWSSKPPTFG | |
| utations) | QGTKLEIK | |
| A5-L1B | QTVLTQSPATLSLSPGERATLSCRASSSVTYIHWYQQKPGQAPRLLIYA | 196 |
| (Q1T2) | TSNLASGIPARFSGSGSGTDFTLTISSLEPEDFAVYYCQHWSSKPPTFG | |
| | QGTKLEIK | |
| A5-L1C | EIVLTQSPATLSLSPGERATLSCRASSSVTYIHWYQQKPGSSPKSWIYA | 197 |
| (FR2) | TSNLASGIPARFSGSGSGTDFTLTISSLEPEDFAVYYCQHWSSKPPTFG | |
| | QGTKLEIK | |
| A5-L1D | EIVLTQSPATLSLSPGERATLSCRASSSVTYIHWYQQKPGQAPRSWIYA | 69 |
| (46,47) | TSNLASGIPARFSGSGSGTDFTLTISSLEPEDFAVYYCQHWSSKPPTFG | |
| | QGTKLEIK | |

The light chain was humanized based on the human IGKV3-11 acceptor framework. In the series A5-L1 to A5-L4, it was learned that variant A5-L1 shows good binding activity (but slightly less than the parental antibody). Partial humanization of CDR-L1 (variant A5-L2; Kabat positions 30 and 31) fully abrogates the binding. Likewise, humanization of CDR-H2 (variant A5-L3; Kabat positions 50 to 56) also fully abrogates the binding. The position 90 (variant A5-L4) shows significant contribution to the binding properties. The Histidine at this position is important for binding. Thus, variant A5-L1 was chosen for further modification.

The series A5-L1A to A5-L1D addressed the question which backmutations are required to restore the full binding potential of the parental chimeric antibody. Variant A5-L1A showed that backmutations at Kabat positions 1, 2, the entire framework 2, and Kabat position 71 do not add any further binding activity. Variants A5-L1B, and A5-L1C addressed subsets of those positions and confirm that they do not alter the binding properties. Variant A5-L1D with back mutations at Kabat positions 46 and 47 showed the best binding activity.

### 5.4 Selection of Humanized A5B7 Antibodies

Based on the new humanization variants of VH and VL new CEA antibodies were expressed as huIgG1 antibodies with an effector silent Fc (P329G; L234, L235A) to abrogate binding to Fcγ receptors according to the method described in WO 2012/130831 A1 and their binding to CEA expressed on MKN45 cells was tested and compared to the respective parental murine A5B7 antibody.

TABLE 33
VH/VL combinations expressed as huIgG1_LALA_PG antibodies

| | A5-L1A | A5-L1B | A5-L1C | A5-L1D |
|---|---|---|---|---|
| 3-23A5-1A | P1AE2164 | P1AE2165 | P1AE2166 | P1AE2167 |
| 3-23A5-1C | — | — | P1AE2176 | P1AE2177 |
| 3-23A5-1D | P1AE2179 | — | P1AE2181 | P1AE2182 |

MKN45 (DSMZ ACC 409) is a human gastric adenocarcinoma cell line expressing CEA. The cells were cultured in advanced RPMI+2% FCS+1% Glutamax. Viability of MKN-45 cells was checked and cells were re-suspended and adjusted to a density of 1 Mio cells/ml. 100 μl of this cell suspension (containing 0.1 Mio cells) were seeded into a 96 well round bottom plate. The plate was centrifuged for 4 min at 400×g and the supernatant was removed. Then 40 μl of the diluted antibodies or FACS buffer were added to the cells and incubated for 30 min at 4° C. After the incubation the cells were washed twice with 150 μl FACS buffer per well. Then 20 μl of the diluted secondary PE anti-human Fc specific secondary antibody (109-116-170, Jackson ImmunoResearch) was added to the cells. The cells were incubated for an additional 30 min at 4° C. To remove unbound antibody, the cells were washed again twice with 150 μl per well FACS buffer. To fix the cells 100 μl of FACS buffer containing 1% PFA were added to the wells. Before measuring the cells were re-suspended in 150 μl FACS buffer. The fluorescence was measured using a BD flow cytometer. All tested binders were able to bind to MKN45 cells but binding capacity was slightly reduced compared to the parental A5B7 antibody. The clone P1AE2167 had the best binding of all tested variants and was selected for further development.

### 5.5 Determination of Affinities of Fab Fragments of Humanized Variants of Murine CEA-Antibody A5B7 to Human CEA Using Surface Plasmon Resonance (BIACORE)

The affinities of Fab fragments of the humanized variants of murine CEA antibody A5B7 to human CEA were assessed by surface plasmon resonance using a BIACORE T200 instrument. On a CM5 chip, human CEA (hu N(A2-B2)A-avi-His B) was immobilized at a 40 nM concentration by standard amine coupling on flow cell 2 for 30 s to about 100RU. The Fab fragments of the humanized variants of murine CEA antibody A5B7 were subsequently injected as analytes in 3-fold dilutions ranging from 500-0.656 nM for a contact time of 120 s, a dissociation time of 250 or 1000 s and at a flow rate of 30 μl/min. Regeneration at the level of human CEA (hu N(A2-B2)A-avi-His B) was achieved by 2 pulses of 10 mM glycine/HCl pH2.0 for 60 s. Data were double-referenced against the unimmobilized flow cell 1 and a zero concentration of the analyte. The sensorgrams of the analytes were fitted to a simple 1:1 Langmuir interaction model. Affinity constants [K_{D}] for human CEA (A2 domain) are summarized in Table 34 below.

TABLE 34
Affinity constants of Fab fragments representing different
humanized variants of murine CEA antibody A5B7 to
human CEA (A2 domain).

| | | Affinity to human |
|---|---|---|
| | | N(A2-B2)A-avi-His B |
| Tapir ID | Name | [M] |
| P1AE0289 | CEA (A5B7) Fab | 5.59 E−10 |
| | (parental murine antibody) | |
| P1AE4135 | Fab derived from P1AE2164 | 1.70 E−09 |
| P1AE4136 | Fab derived from P1AE2165 | 1.25 E−09 |
| P1AE4137 | Fab derived from P1AE2166 | 1.13 E−08 |
| P1AE4138 | Fab derived from P1AE2167 | 1.47 E−09 |
| P1AE4139 | Fab derived from P1AE2176 | 7.58 E−09 |
| P1AE4140 | Fab derived from P1AE2177 | 7.62 E−09 |
| P1AE4141 | Fab derived from P1AE2179 | 1.83 E−09 |
| P1AE4142 | Fab derived from P1AE2181 | 2.64 E−09 |
| P1AE4143 | Fab derived from P1AE2182 | 2.92 E−09 |

The humanized variants of the murine CEA antibody A5B7 are of lower affinities than the parental murine antibody. The Fab fragment P1AE4138, derived from P1AE2167 (heavy chain with VH variant 3-23A5-1A and Ckappa light chain with VL variant A5-L1D) was chosen as final humanized variant. Moreover, a glycine to alanine mutation at Kabat position 54 (G54A) was introduced into the VH domain in order to remove a deamidation site, leading to VL variant 3-23A5-1E. The final humanized antibody (heavy chain with VH variant 3-23A5-1E and Ckappa light chain with VL variant A5-L1D) has been named A5H1EL1D or huA5B7.

### Example 6

### Generation of Bispecific Antigen Binding Molecules Targeting ICOS and Carcinoembryonic Antigen-Related Cell Adhesion Molecule (CEA)

### 6.1 Generation of Bispecific Monovalent Antigen Binding Molecules Targeting ICOS and Carcinoembryonic Antigen-Related Cell Adhesion Molecule (CEA) (1+1 Format)

Bispecific agonistic ICOS antibodies with monovalent binding for ICOS and for CEA were prepared by applying the knob-into-hole technology to allow the assembling of two different heavy chains. The crossmab technology was applied to reduce the formation of wrongly paired light chains as described in International patent application No. WO 2010/145792 A1. A schematic scheme of the bispecific antigen binding molecules that bind monovalently to ICOS and monovalently to CEA is shown in FIGS. 1F to 1H.

For the CEA antigen binding domain, the VH and VL sequences of clone MEDI-565 were obtained from International patent application no. WO 2014/079886 A1. The generation and preparation of the CEA antibody (A5H1EL1D) is described in Example 5. For the ICOS antibody JMab136, the VH and VL sequences of clone JMAb136 were obtained from patent US 2008/0199466 A1.

Molecule 37 contains a crossed Fab unit (VLCH1) of the CEA antibody fused to the knob heavy chain of a huIgG1 (containing the S354C/T366W mutations). The Fc hole heavy chain (containing the Y349C/T366S/L368A/Y407V mutations) is fused to a Fab unit binding to ICOS (FIG. 1F). Molecule 41 contains a crossed Fab unit (VLCH1) of the CEA antibody fused to the hole heavy chain of a huIgG1 (containing the Y349C/T366S/L368A/Y407V mutations). The Fc knob heavy chain (containing the S354C/T366W mutations) is fused to a Fab fragment binding to ICOS (FIG. 1G). Molecule 42 and Molecule 43 contain a crossed Fab unit (VLCH1) of the ICOS antibody fused to the hole heavy chain of a huIgG1 (containing the Y349C/T366S/L368A/Y407V mutations). The Fc knob heavy chain (containing the S354C/T366W mutations) is fused to a Fab fragment binding to CEA (FIG. 1H).

Combination of Fc hole with the Fc knob chain allows generation of a heterodimer, which includes a Fab fragment that specifically binds to CEA and a Fab fragment that specifically binds to ICOS.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831 A1.

The bispecific monovalent anti-ICOS and anti-CEACAM huIgG1 P329GLALA was produced by co-transfecting HEK293F cells with the mammalian expression vectors using FectoPro (PolyPlus, US). The cells were transfected with the corresponding expression vectors in a 1:1:1:1 ratio (“vector knob heavy chain”:“vector light chain1”:“vector hole heavy chain”:“vector light chain2”). The constructs were produced and purified as described for the bispecific monovalent anti-ICOS and anti-FAP huIgG1 P329GLALA antibody (see Example 3.1).

The amino acid sequences of sequences of mature bispecific monovalent anti-ICOS/anti-CEACAM huIgG1 P329GLALA kih antibodies are shown in Table 35.

TABLE 35
Amino acid sequences of mature bispecific 1 + 1 anti-ICOS/anti-CEACAM
human IgG1 P329GLALA antigen binding molecules

| | SEQ ID | | |
|---|---|---|---|
| Molecule | NO: | Name | Sequence |
| 37 | 198 | ICOS (JMAb136) | QVQLVQSGAEVKKPGASVKVSCKASGYTFTGYYM |
| | | VHCH1-Fc hole | HWVRQAPGQGLEWMGWINPHSGGTNYAQKFQGRV |
| | | | TMTRDTSISTAYMELSRLRSDDTAVYYCARTYYY |
| | | | DSSGYYHDAFDIWGQGTMVTVSSASTKGPSVFPL |
| | | | APSSKSTSGGTAALGCLVEDYFPEPVTVSWNSGA |
| | | | LTSGVHTFPAVLQSSGLYSLSSVVTVPSSSLGTQ |
| | | | TYICNVNHKPSNTKVDEKVEPKSCDKTHTCPPCP |
| | | | APEAAGGPSVFLFPPKPKDTLMISRTPEVTCVVV |
| | | | DVSHEDPEVKFNWYVDGVEVHNAKTKPREEQYNS |
| | | | TYRVVSVLTVLHQDWLNGKEYKCKVSNKALGAPI |
| | | | EKTISKAKGQPREPQVCTLPPSRDELTKNQVSLS |
| | | | CAVKGFYPSDIAVEWESNGQPENNYKTTPPVLDS |
| | | | DGSFFLVSKLTVDKSRWQQGNVFSCSVMHEALHN |
| | | | HYTQKSLSLSP |
| | 199 | ICOS (JMAb136) | DIQMTQSPSSVSASVGDRVTITCRASQGISRLLA |
| | | VLCL-Light chain 1 | WYQQKPGKAPKLLIYVASSLQSGVPSRFSGSGSG |
| | | | TDFTLTISSLQPEDFATYYCQQANSFPWTFGQGT |
| | | | KVEIKRTVAAPSVFIFPPSDRKLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| | 200 | CEA (MEDI-565) | QAVLTQPASLSASPGASASLTCTLRRGINVGAYS |
| | | VLCH1-Fc knob | IYWYQQKPGSPPQYLLRYKSDSDKQQGSGVSSRF |
| | | | SASKDASANAGILLISGLQSEDEADYYCMIWHSG |
| | | | ASAVFGGGTKLTVLSSASTKGPSVFPLAPSSKST |
| | | | SGGTAALGCLVKDYFPEPVTVSWNSGALTSGVHT |
| | | | FPAVLQSSGLYSLSSVVTVPSSSLGTQTYICNVN |
| | | | HKPSNTKVDKKVEPKSCDKTHTCPPCPAPEAAGG |
| | | | PSVFLFPPKPKDTLMISRTPEVTCVVVDVSHEDP |
| | | | EVKFNWYVDGVEVHNAKTKPREEQYNSTYRVVSV |
| | | | LTVLHQDWLNGKEYKCKVSNKALGAPIEKTISKA |
| | | | KGQPREPQVYTLPPCRDELTKNQVSLWCLVKGFY |
| | | | PSDIAVEWESNGQPENNYKTTPPVLDSDGSFFLY |
| | | | SKLTVDKSRWQQGNVFSCSVMHEALHNHYTQKSL |
| | | | SLSP |
| | 201 | CEA (MEDI-565) | EVQLVESGGGLVQPGRSLRLSCAASGFTVSSYWM |
| | | VHCL-Light chain 2 | HWVRQAPGKGLEWVGFIRNKANGGTTEYAASVKG |
| | | | RFTISRDDSKNTLYLQMNSLRAEDTAVYYCARDR |
| | | | GLRFYFDYWGQGTTVTVSSASVAAPSVFIFPPSD |
| | | | EQLKSGTASVVCLLNNFYPREAKVQWKVDNALQS |
| | | | GNSQESVTEQDSKDSTYSLSSTLTLSKADYEKHK |
| | | | VYACEVTHQGLSSPVTKSFNRGEC |
| 41 | 202 | CEA (A5H1EL1D) | EIVLTQSPATLSLSPGERATLSCRASSSVTYIHW |
| | | VLCH1-Fc hole | YQQKPGQAPRSWIYATSNLASGIPARFSGSGSGT |
| | | | DFTLTISSLEPEDFAVYYCQHWSSKPPTEGQGTK |
| | | | LEIKSSASTKGPSVFPLAPSSKSTSGGTAALGCL |
| | | | VKDYFPEPVTVSWNSGALTSGVHTFPAVLQSSGL |
| | | | YSLSSVVTVPSSSLGTQTYICNVNHKPSNTKVDK |
| | | | KVEPKSCDKTHTCPPCPAPEAAGGPSVFLFPPKP |
| | | | KDTLMISRTPEVTCVVVDVSHEDPEVKFNWYVDG |
| | | | VEVHNAKTKPREEQYNSTYRVVSVLTVLHQDWLN |
| | | | GKEYKCKVSNKALGAPIEKTISKAKGQPREPQVC |
| | | | TLPPSRDELTKNQVSLSCAVKGFYPSDIAVEWES |
| | | | NGQPENNYKTTPPVLDSDGSFFLVSKLTVDKSRW |
| | | | QQGNVFSCSVMHEALHNHYTQKSLSLSP |
| | 203 | CEA (A5H1EL1D) | EVQLLESGGGLVQPGGSLRLSCAASGFTFTDYYM |
| | | VHCL-Light chain 1 | NWVRQAPGKGLEWLGFIGNKANAYTTEYSASVKG |
| | | | RFTISRDKSKNTLYLQMNSLRAEDTATYYCTRDR |
| | | | GLRFYFDYWGQGTTVTVSSASVAAPSVFIFPPSD |
| | | | EQLKSGTASVVCLLNNFYPREAKVQWKVDNALQS |
| | | | GNSQESVTEQDSKDSTYSLSSTLTLSKADYEKHK |
| | | | VYACEVTHQGLSSPVTKSFNRGEC |
| | 204 | ICOS (1167) VHCH1- | EVRLLESGGGLVQPGGSLRLSCAASGFTFNTYAV |
| | | Fc knob | HWVRQAPGKGLEWVSGIGGSGVRTYYADSVKGRL |
| | | | TISRDNSKNTLYLQMNSLRAEDTAIYFCAKDIYV |
| | | | ADFTGYAFDIWGQGTMVTVSSASTKGPSVFPLAP |
| | | | SSKSTSGGTAALGCLVEDYFPEPVTVSWNSGALT |
| | | | SGVHTFPAVLQSSGLYSLSSVVTVPSSSLGTQTY |
| | | | ICNVNHKPSNTKVDEKVEPKSCDKTHTCPPCPAP |
| | | | EAAGGPSVFLFPPKPKDTLMISRTPEVTCVVVDV |
| | | | SHEDPEVKFNWYVDGVEVHNAKTKPREEQYNSTY |
| | | | RVVSVLTVLHQDWLNGKEYKCKVSNKALGAPIEK |
| | | | TISKAKGQPREPQVYTLPPCRDELTKNQVSLWCL |
| | | | VKGFYPSDIAVEWESNGQPENNYKTTPPVLDSDG |
| | | | SFFLYSKLTVDKSRWQQGNVFSCSVMHEALHNHY |
| | | | TQKSLSLSP |
| | 205 | ICOS (1167) VLCL- | DIQMTQSPSSVSASVGDRVTITCRASQGINNFLA |
| | | Light chain 2 | WYQQKPGKAPKLLIYDASSLQSGVPSRFAGSGSG |
| | | | TDFTLTISSLQPEDFATYYCQQYNEYPLTEGGGT |
| | | | MVEIKRTVAAPSVFIFPPSDRKLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| 42 | 206 | CEA (A5H1EL1D) | EVQLVESGGGLVQPGRSLRLSCTASGFTFSDYWM |
| | | VHCH1-Fc knob | NWVRQAPGKGLEWVAQIRNKPYNYETYYSDSVKG |
| | | | RFTISRDDSKSIAYLQMNSLKTEDTAVYYCTWPR |
| | | | LRSSDWHEDVWGAGTTVTVSSASTKGPSVFPLAP |
| | | | SSKSTSGGTAALGCLVEDYFPEPVTVSWNSGALT |
| | | | SGVHTFPAVLQSSGLYSLSSVVTVPSSSLGTQTY |
| | | | ICNVNHKPSNTKVDEKVEPKSCDKTHTCPPCPAP |
| | | | EAAGGPSVFLFPPKPKDTLMISRTPEVTCVVVDV |
| | | | SHEDPEVKFNWYVDGVEVHNAKTKPREEQYNSTY |
| | | | RVVSVLTVLHQDWLNGKEYKCKVSNKALGAPIEK |
| | | | TISKAKGQPREPQVYTLPPCRDELTKNQVSLWCL |
| | | | VKGFYPSDIAVEWESNGQPENNYKTTPPVLDSDG |
| | | | SFFLYSKLTVDKSRWQQGNVFSCSVMHEALHNHY |
| | | | TQKSLSLSP |
| | 207 | CEA (A5H1EL1D) | DIQMTQSPSSLSASVGDRVTITCQASQDINKNIA |
| | | VLCL-Light chain 1 | WYQQKPGKAPKLLIWYTSTLQTGVPSRFSGSGSG |
| | | | TDFTFTISSLQPEDIATYYCLQFDNLYTFGQGTK |
| | | | LEIKRTVAAPSVFIFPPSDRKLKSGTASVVCLLN |
| | | | NFYPREAKVQWKVDNALQSGNSQESVTEQDSKDS |
| | | | TYSLSSTLTLSKADYEKHKVYACEVTHQGLSSPV |
| | | TKSFNRGEC | |
| | 208 | ICOS (H009v1_2) | EIVLTQSPATLSLSPGERATLSCRASSSVTYIHW |
| | | VLCH1-Fc hole | YQQKPGQAPRSWIYATSNLASGIPARFSGSGSGT |
| | | | DFTLTISSLEPEDFAVYYCQHWSSKPPTFGQGTK |
| | | | LEIKSSASTKGPSVFPLAPSSKSTSGGTAALGCL |
| | | | VKDYFPEPVTVSWNSGALTSGVHTFPAVLQSSGL |
| | | | YSLSSVVTVPSSSLGTQTYICNVNHKPSNTKVDK |
| | | | KVEPKSCDKTHTCPPCPAPEAAGGPSVFLFPPKP |
| | | | KDTLMISRTPEVTCVVVDVSHEDPEVKFNWYVDG |
| | | | VEVHNAKTKPREEQYNSTYRVVSVLTVLHQDWLN |
| | | | GKEYKCKVSNKALGAPIEKTISKAKGQPREPQVC |
| | | | TLPPSRDELTKNQVSLSCAVKGFYPSDIAVEWES |
| | | | NGQPENNYKTTPPVLDSDGSFFLVSKLTVDKSRW |
| | | | QQGNVFSCSVMHEALHNHYTQKSLSLSP |
| | 209 | ICOS (H009v1_2) | EVQLLESGGGLVQPGGSLRLSCAASGFTFTDYYM |
| | | VHCL-Light chain 2 | NWVRQAPGKGLEWLGFIGNKANAYTTEYSASVKG |
| | | | RFTISRDKSKNTLYLQMNSLRAEDTATYYCTRDR |
| | | | GLRFYFDYWGQGTTVTVSSASVAAPSVFIFPPSD |
| | | | EQLKSGTASVVCLLNNFYPREAKVQWKVDNALQS |
| | | | GNSQESVTEQDSKDSTYSLSSTLTLSKADYEKHK |
| | | | VYACEVTHQGLSSPVTKSFNRGEC |
| 43 | 206 | CEA (A5H1EL1D) | See above |
| | | VHCH1-Fc knob | |
| | 207 | CEA (A5H1EL1D) | See above |
| | | VLCL-Light chain 1 | |
| | 210 | ICOS (H1143v2_1) | EIVLTQSPATLSLSPGERATLSCRASSSVTYIHW |
| | | VLCH1-Fc hole | YQQKPGQAPRSWIYATSNLASGIPARFSGSGSGT |
| | | | DFTLTISSLEPEDFAVYYCQHWSSKPPTFGQGTK |
| | | | LEIKSSASTKGPSVFPLAPSSKSTSGGTAALGCL |
| | | | VKDYFPEPVTVSWNSGALTSGVHTFPAVLQSSGL |
| | | | YSLSSVVTVPSSSLGTQTYICNVNHKPSNTKVDK |
| | | | KVEPKSCDKTHTCPPCPAPEAAGGPSVFLFPPKP |
| | | | KDTLMISRTPEVTCVVVDVSHEDPEVKFNWYVDG |
| | | | VEVHNAKTKPREEQYNSTYRVVSVLTVLHQDWLN |
| | | | GKEYKCKVSNKALGAPIEKTISKAKGQPREPQVC |
| | | | TLPPSRDELTKNQVSLSCAVKGFYPSDIAVEWES |
| | | | NGQPENNYKTTPPVLDSDGSFFLVSKLTVDKSRW |
| | | | QQGNVFSCSVMHEALHNHYTQKSLSLSP |
| | 211 | ICOS (H1143v2_1) | EVQLLESGGGLVQPGGSLRLSCAASGFTFTDYYM |
| | | VHCL-Light chain 2 | NWVRQAPGKGLEWLGFIGNKANAYTTEYSASVKG |
| | | | RFTISRDKSKNTLYLQMNSLRAEDTATYYCTRDR |
| | | | GLRFYFDYWGQGTTVTVSSASVAAPSVFIFPPSD |
| | | | EQLKSGTASVVCLLNNFYPREAKVQWKVDNALQS |
| | | | GNSQESVTEQDSKDSTYSLSSTLTLSKADYEKHK |
| | | | VYACEVTHQGLSSPVTKSFNRGEC |

TABLE 36
Biochemical analysis of bispecific antigen binding molecules
with monovalent binding to ICOS and monovalent binding
to CEA (1 + 1 ICOS/CEA human IgG1 P329GLALA
antigen binding molecules)

| | | CE-SDS | |
|---|---|---|---|
| | Monomer | (non-reduced) | Yield |
| Molecule | [%] | [%] | [mg/l] |
| 37 | 94 | 91 | 4.6 |
| 41 | 100 | 90.9 | 3.2 |
| 42 | 100 | 98.7 | 45.9 |
| 43 | 100 | 98.7 | 29.8 |

6.2 Generation of Bispecific Antigen Binding Molecules Targeting ICOS and Carcinoembryonic Antigen-Related Cell Adhesion Molecule (CEA) with Bivalent Binding to ICOS and Monovalent Binding to CEA (2+1 Format)

Bispecific agonistic ICOS antibodies with bivalent binding to ICOS and monovalent binding to CEA, also termed 2+1, have been prepared in analogy to the FAP-targeted ones as depicted in FIG. 1C.

In this example, the first heavy chain HC1 of the construct was comprised of the following components: VHCH1 of anti-ICOS antibody, followed by Fc knob, at which C-terminus a VL of the CEA antibody was fused. The second heavy chain HC2 was comprised of VHCH1 of anti-ICOS antibody followed by Fc hole, at which C-terminus a VH of the CEA antibody was fused. For the CEA antigen binding domain, the VH and VL sequences of clone MEDI-565 were obtained from International patent application no. WO 2014/079886 Al. For the ICOS antibody JMab136, the VH and VL sequences of clone JMAb136 were obtained from patent US 2008/0199466 Al.

The Pro329Gly, Leu234Ala and Leu235Ala mutations were introduced in the constant region of the knob and hole heavy chains to abrogate binding to Fc gamma receptors according to the method described in International Patent Appl. Publ. No. WO2012/130831A1.

The bispecific 2+1 anti-ICOS, anti-CEA huIgG1 P329GLALA antibody was produced by co-transfecting HEK293F cells with the mammalian expression vectors using FectoPro (PolyPlus, US). The cells were transfected with the corresponding expression vectors in a 1:2:1 ratio (“vector knob heavy chain”:“vector light chain”:“vector hole heavy chain”). The constructs were produced and purified as described for the bispecific monovalent anti-ICOS and anti-FAP huIgG1 P329GLALA antibody (see Example 3.1).

The amino acid sequences for 2+1 anti-ICOS, anti-CEA constructs can be found in Tbale 37.

TABLE 37
Amino acid sequences of mature bispecific 2 + 1 anti-ICOS, anti-CEA human
IgG1 P329GLALA.

| | SEQ | | |
|---|---|---|---|
| Molecule | ID NO: | Name | Sequence |
| 36 | 212 | ICOS (JMAb136) | QVQLVQSGAEVKKPGASVKVSCKASGYTFTGYYM |
| | | VHCH1 Fc hole VH | HWVRQAPGQGLEWMGWINPHSGGTNYAQKFQGRV |
| | | CEA (MEDI-565) | TMTRDTSISTAYMELSRLRSDDTAVYYCARTYYY |
| | | | DSSGYYHDAFDIWGQGTMVTVSSASTKGPSVFPL |
| | | | APSSKSTSGGTAALGCLVKDYFPEPVTVSWNSGA |
| | | | LTSGVHTFPAVLQSSGLYSLSSVVTVPSSSLGTQ |
| | | | TYICNVNHKPSNTKVDKKVEPKSCDKTHTCPPCP |
| | | | APEAAGGPSVFLFPPKPKDTLMISRTPEVTCVVV |
| | | | DVSHEDPEVKFNWYVDGVEVHNAKTKPREEQYNS |
| | | | TYRVVSVLTVLHQDWLNGKEYKCKVSNKALGAPI |
| | | | EKTISKAKGQPREPQVCTLPPSRDELTKNQVSLS |
| | | | CAVKGFYPSDIAVEWESNGQPENNYKTTPPVLDS |
| | | | DGSFFLVSKLTVDKSRWQQGNVFSCSVMHEALHN |
| | | | HYTQKSLSLSPGGGGGSGGGGSEVQLVESGGGLV |
| | | | QPGRSLRLSCAASGFTVSSYWMHWVRQAPGKGLE |
| | | | WVGFIRNKANGGTTEYAASVKGRFTISRDDSKNT |
| | | | LYLQMNSLRAEDTAVYYCARDRGLRFYFDYWGQG |
| | | | TTVTVSS |
| | 213 | ICOS (JMAb136) | QVQLVQSGAEVKKPGASVKVSCKASGYTFTGYYM |
| | | VHCH1 Fc knob VL | HWVRQAPGQGLEWMGWINPHSGGTNYAQKFQGRV |
| | | CEA (MEDI-565) | TMTRDTSISTAYMELSRLRSDDTAVYYCARTYYY |
| | | | DSSGYYHDAFDIWGQGTMVTVSSASTKGPSVFPL |
| | | | APSSKSTSGGTAALGCLVKDYFPEPVTVSWNSGA |
| | | | LTSGVHTFPAVLQSSGLYSLSSVVTVPSSSLGTQ |
| | | | TYICNVNHKPSNTKVDKKVEPKSCDKTHTCPPCP |
| | | | APEAAGGPSVFLFPPKPKDTLMISRTPEVTCVVV |
| | | | DVSHEDPEVKFNWYVDGVEVHNAKTKPREEQYNS |
| | | | TYRVVSVLTVLHQDWLNGKEYKCKVSNKALGAPI |
| | | | EKTISKAKGQPREPQVYTLPPCRDELTKNQVSLW |
| | | | CLVKGFYPSDIAVEWESNGQPENNYKTTPPVLDS |
| | | | DGSFFLYSKLTVDKSRWQQGNVFSCSVMHEALHN |
| | | | HYTQKSLSLSPGGGGGSGGGGSGGGGSGGGGSQA |
| | | | VLTQPASLSASPGASASLTCTLRRGINVGAYSIY |
| | | | WYQQKPGSPPQYLLRYKSDSDKQQGSGVSSRFSA |
| | | | SKDASANAGILLISGLQSEDEADYYCMIWHSGAS |
| | | | AVFGGGTKLTVL |
| | 214 | ICOS (JMAb136) | DIQMTQSPSSVSASVGDRVTITCRASQGISRLLA |
| | | VLCL-light chain | WYQQKPGKAPKLLIYVASSLQSGVPSRFSGSGSG |
| | | | TDFTLTISSLQPEDFATYYCQQANSFPWTFGQGT |
| | | | KVEIKRTVAAPSVFIFPPSDEQLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |

TABLE 38
Biochemical analysis of bispecific antigen binding molecules
with bivalent binding to ICOS and monovalent binding to CEA
(2 + 1 ICOS/CEA human IgG1 P329GLALA)

| | | CE-SDS | |
|---|---|---|---|
| | Monomer | (non-reduced) | Yield |
| Molecule | [%] | [%] | [mg/l] |
| 36 | 92 | 93 | 1.9 |

### Example 7

### In Vitro Functional Characterization of the Molecules

### 7.1 Binding of Anti-ICOS Antibodies to ICOS Expressing Cells (Flow Cytometry Analysis)

The binding of several ICOS antibodies as prepared in Example 1 was tested using ICOS expressing CHO cells (ATCC, CCL-61, transfected to stably overexpress human ICOS).

Briefly, suspension cells were harvested, counted, checked for viability and re-suspended at 1 million cells per ml in FACS buffer (PBS with 0.1% BSA). 100 μl of the cell suspension (containing 0.1 million cells) were incubated in round-bottom 96-well plates for 30 min at 4° C. with increasing concentrations of the anti-ICOS (7 pM-120 nM for the binding of FAP-ICOS constructs to T-Cells), cells were washed twice with cold PBS 0.1% BSA, re-incubated for further 30 min at 4° C. with a labeled secondary antibody (Molecules 1, 8 with PE-conjugated, donkey anti human H+L PE from Jackson Immuno Research Lab #709-116-149; Molecules 18 and 20 with donkey anti rabbit H+L PE from Jackson Immuno Research Lab #711-116-152 at a dilution of 1:100, Molecule 14 with donkey anti mouse H+L PE from Jackson Immuno Research Lab #715-116-150) and washed twice with cold PBS 0.1% BSA. The staining was fixed for 20 min at 4° C. in the dark, using 75 μl of 1% PFA in FACS buffer per well.

In addition, binding of the above molecules to human SR cells (ATCC® CRL-2262) was performed as described above apart from the following modifications: SR cells were re-suspended at 2 million cells per ml in FACS buffer (BD). 100 μl of the cell suspension (containing 0.2 million cells) were incubated in 96-well PP plate for 1 h at 4° C. with increasing concentrations of the anti-ICOS (7 pM-510 nM), cells were washed twice with cold PBS 0.1% BSA, re-incubated for further 30 min at 4° C. with a labeled secondary antibody as described above.

Fluorescence was analyzed by FACS using a FACS Fortessa (Software FACS Diva). Binding curves and EC50 values were obtained using GraphPadPrism 7.

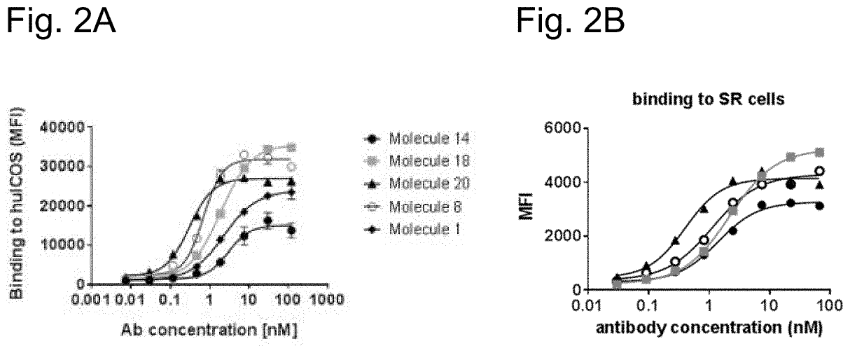

The results show that the ICOS molecules are able to bind to human ICOS in a concentration dependent manner (FIGS. 2A and 2B). EC₅₀ Values are depicted in Table 39. Best binding was observed for Molecules 20 and 8.

TABLE 39
EC₅₀ values of binding of different anti-ICOS IgGs to
ICOS⁺ CHO or SR cells

| | CHO-huICOS cells | SR cells |
|---|---|---|
| Molecule | EC₅₀ [pM] | EC₅₀ [pM] |
| Molecule 14 | 2.97 | 1.40 |
| (IgG of 009) | | |
| Molecule 18 | 1.92 | 2.16 |
| (IgG of 1138) | | |
| Molecule 20 | 0.30 | 0.40 |
| (IgG of 1143) | | |
| Molecule 8 | 0.69 | 1.13 |
| (IgG of 1167) | | |
| Molecule 1 | 2.3 | n.d. |
| (JMab136 IgG) | | |

Additionally, humanized variants of the ICOS antibodies 009, 1143v2 and 1138 as prepared in Example 4 were tested (in the form of molecules 15, 28 and 32) for their binding to human ICOS as described above.

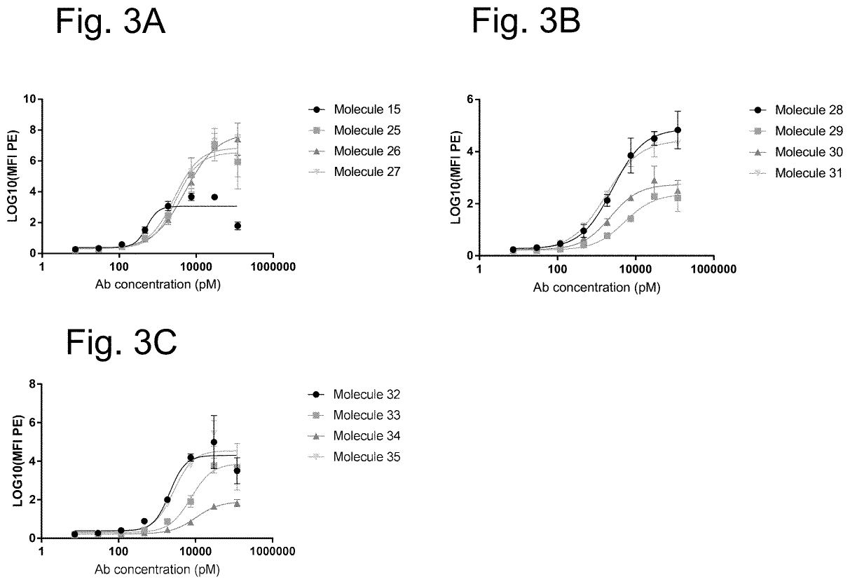

The results show that the molecules are able to bind to human ICOS in a concentration dependent manner (FIGS. 3A to 3C). EC₅₀ values are depicted in Table 40. For antibody 009 (Molecule 14) a different reference molecule had to be used for the assay (Molecule 15, FAP-targeted 2+1 ICOS antigen binding molecule) which exhibited altered binding characteristics.

Comparison to the parental molecule is therefore difficult. The three variants exhibited comparable binding profiles (FIG. 3A) with Molecule 26 exhibiting slightly impaired binding compared to Molecule 25 and 27.

For Molecule 28, it was shown that Molecule 31 displays a comparable binding to the parent antibody (Molecule 28) and a higher absolute binding compared to Molecule 29 and 30 (FIG. 3B).

Molecule 35 shows similar binding behavior compared to the parental one, whereas Molecule 33 and Molecule 34 exhibit higher EC₅₀ values and lower overall binding compared to the parental antibody (Molecule 32). (FIG. 3C).

TABLE 40
EC₅₀ values of binding of different anti-ICOS IgGs to ICOS⁺

| | EC₅₀ | Max |
|---|---|---|
| | (pM) | (Log10(MFI)) |
| Molecule 15 | 516.1 | 3.059 |
| Molecule 25 | 2919 | 6.547 |
| Molecule 26 | 5257 | 7.804 |
| Molecule 27 | 2698 | 6.852 |
| Molecule 28 | 2558 | 4.881 |
| Molecule 29 | 5195 | 2.378 |
| Molecule 30 | 2251 | 2.747 |
| Molecule 31 | 1746 | 4.444 |
| Molecule 32 | 2116 | 4.303 |
| Molecule 33 | 7578 | 3.888 |
| Molecule 34 | 10160 | 1.914 |
| Molecule 35 | 2721 | 4.55 |

### 7.2 Activation of Jurkat-NFAT Reporter Cells (Luminescence Based Analysis)

The Dependency on a simultaneous TCR engagement was assessed by using an engineered Jurkat Cell Line expressing Luciferase in response to NFAT nuclear translocation.

GloResponse Jurkat NFAT-RE-luc2P (Promega #CS176501) reporter cell line was preactivated to induce ICOS expression using either Cell Culture Flasks coated with 1.5 μg/ml aCD3 (BioLegend #317304) and 2 μg/ml aCD28 (BioLegend, #302914) or PHA-L (Sigma #, 1 μg/ml) and IL-2 (Proleukin, Novartis; 200 U/ml) in JurkatNFAT culture Medium (RPMI1640 medium containing 10% FCS, 1% GluMax, 25 mM HEPES, 1×NEAA, 1% So-Pyruvate; selection: 200 ug/ml Hygromycin B).

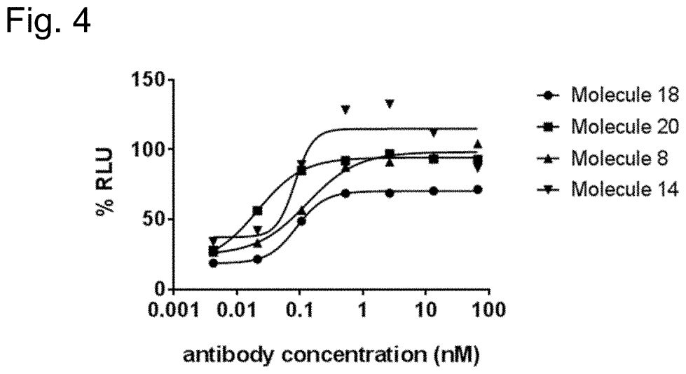

Cells were starved (JurkatNFAT culture Medium without Stimulation) overnight before the assay. Assay plates StreptaWelll High Bind (transparent, 96-wells, Roche #11989685001) were coated (4° C. overnight) simultaneously with a mixture of Bi<huIgG F(ab′)2>(JIR, #109-066-097) 1 μg/ml and Bi<mIgG F(ab′)2>(JIR, #115-066-072) 1 μg/ml at a ratio of 1:1. The next day plates were washed and either 0.25 μg/ml aCD3 (BioLegend #317315) plus anti-ICOS molecules at the indicated concentrations (range of 29 pM-120000 pM) were added and the plates incubated for 2 h at room temperature. The plates were washed once with DPBS (Gibco, #14190136) and 0.15 Mio stimulated and starved GloResponse Jurkat NFAT-RE-luc2P were added. NFAT mediated signaling was assessed after 5 h of incubation at 37° C., 5% CO₂ by Luminescence Reading using Promega OneGlo Assay System (Promega, #E6120) according to manufacturer instructions. Plates were reformatted to Sterile 96-well flat bottom white plates (Costar, #3917) and read on a Tecan Spark10M Plate Reader (Luminescence Reading, 1000 ms Integration Time, Auto Attenuation Setting). Curves and EC50 values were obtained using GraphPadPrism 7The results show that the all tested ICOS antibodies (in the wild-type IgG format) are able to activate Jurkat-NFAT reporter cells in a concentration dependent manner (FIG. 4). EC₅₀ values are depicted in Table 41. Strongest activation was observed for Molecule 20.

TABLE 41
EC₅₀ values of activation of Jurkat-NFAT reporter cell line using
different anti-ICOS IgGs

| Molecule | EC50 [nM] |
|---|---|
| Molecule 14 | 0.08 |
| Molecule 18 | 0.09 |
| Molecule 20 | 0.02 |
| Molecule 8 | 0.13 |

Additionally, humanized variants of the ICOS antibodies 009, 1143v2 and 1138 as prepared in Example 4 were tested (in the form of molecules 15, 28 and 32) for their ability to activate Jurkat-NFAT reporter cells as described above.

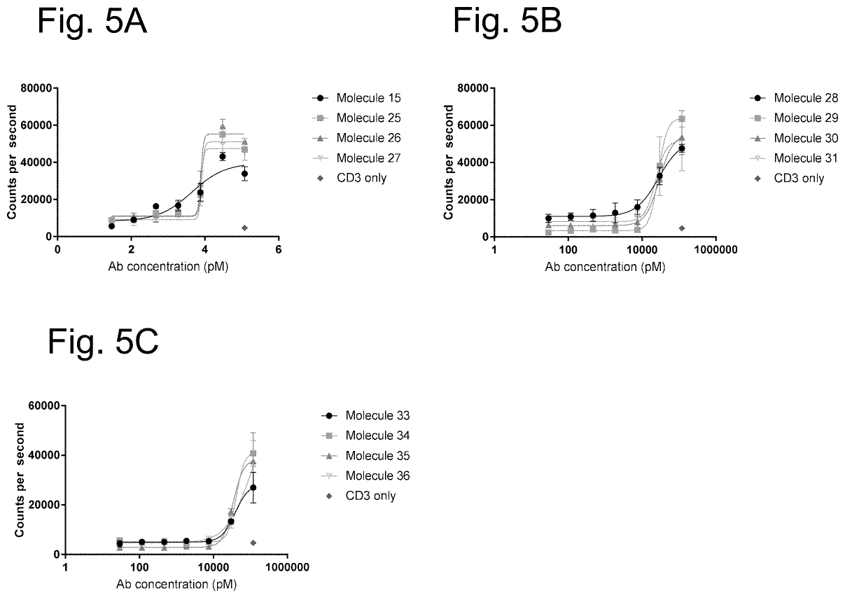

The results show that the molecules are able to activate Jurkat-NFAT reporter cells in a concentration dependent manner (FIGS. 5A to 5C). EC₅₀ values are depicted in Table 42. For Molecule 14 a different reference molecule had to be used for the assay (Molecule 15) which exhibited lower overall agonistic activity compared with the variants. Comparison to the parental molecule is therefore difficult. The three variants exhibited very comparable agonistic activities as was previously already the case for binding to human ICOS.

Molecule 28 and its variants exhibited comparable EC₅₀ values and only slight difference in the maximal agonistic activity with a ranking of Molecule 29>Molecule 30>Molecule 28=Molecule 31, also in line with the results from binding to human ICOS as described previously.

Also only small differences in the agonistic activity were observed for Molecule 32 and its humanized variants: the ranking in terms of maximal agonistic activity is Molecule 35>Molecule 33>Molecule 34>Molecule 32. In terms of EC₅₀ the ranking is Molecule 34>Molecule 32>Molecule 33>Molecule 35.

TABLE 42
EC₅₀ values of activation of Jurkat-NFAT reporter cell
line using different anti-ICOS IgGs

| | EC₅₀ | Max |
|---|---|---|
| | (pM) | (Counts/sec) |
| Molecule 15 | 4441 | 39677 |
| Molecule 25 | ~8109 | 51026 |
| Molecule 26 | ~7769 | 55313 |
| Molecule 27 | ~7988 | 47406 |
| Molecule 28 | 27990 | 52267 |
| Molecule 29 | 27639 | 63793 |
| Molecule 30 | 29363 | 55347 |
| Molecule 31 | 23204 | 52002 |
| Molecule 32 | 38680 | 28475 |
| Molecule 33 | 41350 | 41367 |
| Molecule 34 | 33975 | 38376 |
| Molecule 35 | 99402 | 59456 |

7.3 Competition with ICOS-Ligand (Flow Cytometry Analysis)

The competition of several anti-ICOS antibodies prepared in Example 1 with the human ICOS Ligand (SEQ ID NO:215, UniProt No. 075144) was tested on on ICOS⁺ CHO transfectant cells (see Example 2.2).

Briefly, cells were harvested, counted, checked for viability and re-suspended at 1 million cells per ml in FACS buffer (PBS with 0.1% BSA). 100 μl of the cell suspension (containing 0.1 million cells) were incubated in round-bottom 96-well plates for 30 min at 4° C. with 120 nM ICOSL labeled with Alexa-Fluor 647 (=ICOSL pre-bound) or anti-ICOS molecules labeled with Alexa-Fluor 488 (=ICOS IgG pre-bound). Cells were washed twice with cold PBS 0.1% BSA, and incubated with increasing concentrations (7 pM-120) of the anti-ICOS A488 molecules (for wells where ICOSL was pre-bound) or of the ICOSL-A647 (for wells where anti-ICOS molecules were pre-bound). Cells were washed again twice with cold PBS 0.1% BSA and then re-incubated and fixed for 20 min at 4° C. in the dark, using 75 μl of 1% PFA in FACS buffer per well. Fluorescence was analyzed by FACS using a FACS Fortessa (Software FACS Diva. Data was analyzed using GraphPadPrism 7.

Table 43 shows Median Fluorescence Intensity (MFI) and % relative binding at 120 nM concentration (calculated as MFI(ICOSL+anti-ICOS)/MFI(anti-ICOS only)*100, all MFIs were baseline-corrected using signal from wells with cells and secondary antibody only as baseline) of anti-ICOS molecules for different conditions. It is shown that all anti-ICOS antibodies, except the non-competing control molecule, remain bound to huICOS, even when 120 nM ICOSL are added.

TABLE 43
Absolute and Relative Binding of anti-ICOS IgGs in
presence/absence of hu ICOS-Ligand (ICOSL)

| | 120 nM anti-ICOS IgG pre-bound | | |
|---|---|---|---|
| | IgG + ICOSL | IgG Only | Relative |
| Antibody | (MFI, A488) | (MFI, A488) | Binding (%) |
| Molecule 14 (A488) | 6997 | 7194 | 97.26 |
| Molecule 18 (A488) | 6213 | 6324 | 98.24 |
| Molecule 20 (A488) | 6340 | 6324 | 100.25 |
| Molecule 8 (A488) | 7662 | 7800 | 98.23 |
| Non-competing | 291 | 779 | 37.41 |
| control (A488) | | | |

### 7.4 Binding of Bispecific Tumor Targeted ICOS Molecules to ICOS-, FAP- or CEA-Overexpressing Cells (Flow Cytometry Analysis)

The binding of several bispecific tumor-targeted ICOS antigen binding molecules as prepared in Example 3 or 6 was tested using ICOS expressing CHO cells (ATCC, CCL-61, transfected to stably overexpress human ICOS).

Briefly, suspension cells were harvested, counted, checked for viability and re-suspended at 1 million cells per ml in FACS buffer (PBS with 0.1% BSA). 100 μl of the cell suspension (containing 0.1 million cells) were incubated in round-bottom 96-well plates for 30 min at 4° C. with increasing concentrations of the anti-ICOS (7 pM-120 nM), cells were washed twice with cold PBS 0.1% BSA, re-incubated for further 30 min at 4° C. with a labeled secondary antibody (Fab Fcy specific AF647 (1:100), 190-606-008 Jackson Immuno Research) and washed twice with cold PBS 0.1% BSA. The staining was fixed for 20 min at 4° C. in the dark, using 75 μl of 1% PFA in FACS buffer per well.

Fluorescence was analyzed by FACS using a FACS Fortessa (Software FACS Diva). Binding curves and EC₅₀ values were obtained using GraphPadPrism 7.

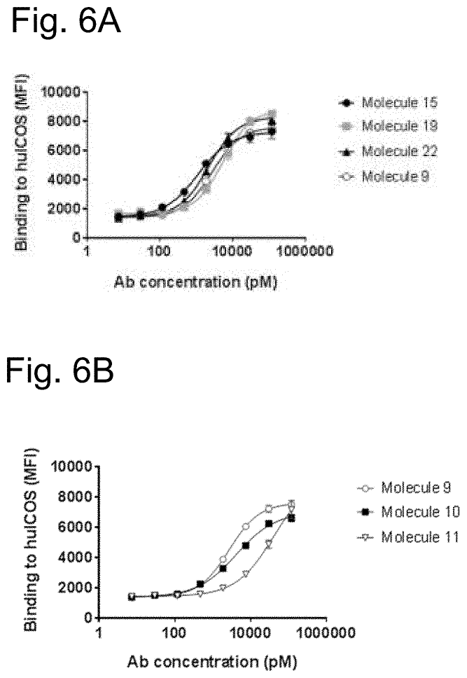

The results show that the bispecific ICOS antigen binding molecules are able to bind to human ICOS in a concentration dependent manner (FIG. 6A). EC₅₀ values are depicted in Table 44. Best binding was observed for Molecule 15. Furthermore, the results indicate a ranking of the binding of the three different formats indicated in FIGS. 1A to 1C, showing superior binding of the 2+1 format (FIG. 1C) compared to 1+1 formats (FIG. 6B), as expected due to the bivalent over monovalent binding to ICOS.

TABLE 44
EC₅₀ values of binding of different
tumor targeted anti-ICOS molecules
to ICOS⁺ CHO cells

| Molecule | EC50 [pM] |
|---|---|
| Molecule 15 | 1123 |
| Molecule 19 | 5444 |
| Molecule 22 | 2186 |
| Molecule 9 | 2718 |
| Molecule 10 | 41710 |
| Molecule 11 | 4169 |

In addition, binding of the same molecules to human NIH/3t3-huFAP clone 19 cells (parental cell line ATCC #CCL-92, modified to stably overexpress human FAP) was performed the same way as described above.

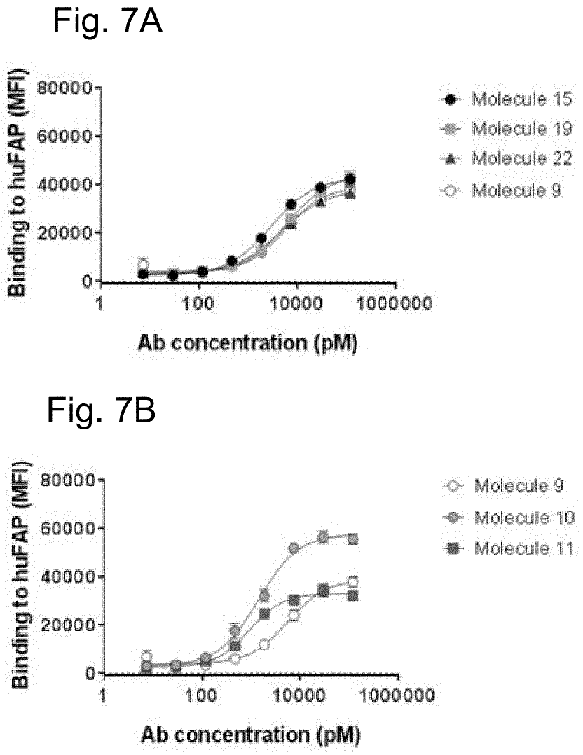

The results show that the bispecific tumor-targeted ICOS antigen binding molecules are able to bind to human FAP in a concentration dependent manner (FIG. 7A). EC₅₀ values are depicted in Table 45. Molecule 9, 15, 19 and 22 exhibit very similar binding. On the other hand, results indicate superior binding of the 1+1 molecule format (FIG. 1A), compared to the 2+1 (FIG. 1C) and 1+1 HT format (FIG. 1B) (see FIG. 7B). This might be driven by different binding affinities of the FAP-targeting part as VH-VL versus Fab fusion.

TABLE 45
EC₅₀ values of binding of different
tumor targeted anti-ICOS molecules to
FAP⁺ NIH/3t3-huFAP clone 19 cells

| Molecule | EC50 [pM] |
|---|---|
| Molecule 15 | 2967 |
| Molecule 19 | 6155 |
| Molecule 22 | 4680 |
| Molecule 9 | 5730 |
| Molecule 10 | 1403 |
| Molecule 11 | 1154 |

Furthermore, binding of the same molecules to cynomolgus ICOS was assessed on preactivated cynomolgus PBMCs.

Briefly, cynomolgus PBMCs were activated for 48 hours using Dynabeads™ Human T-Activator CD3/CD28 (Thermo Fischer #11131D) according to manufacturer instruction and stored in RPMI1640 medium containing 10% FCS and 1% Glutamax (Gibco 35050061) at 37° C., in a humidified incubator until subsequent binding experiment was performed, as described above.

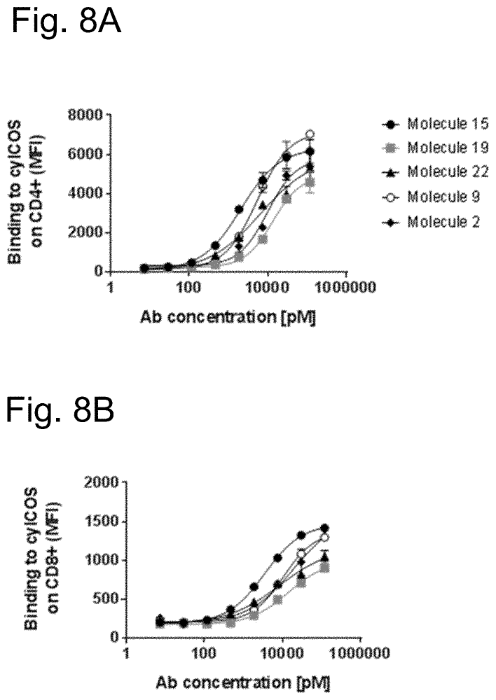

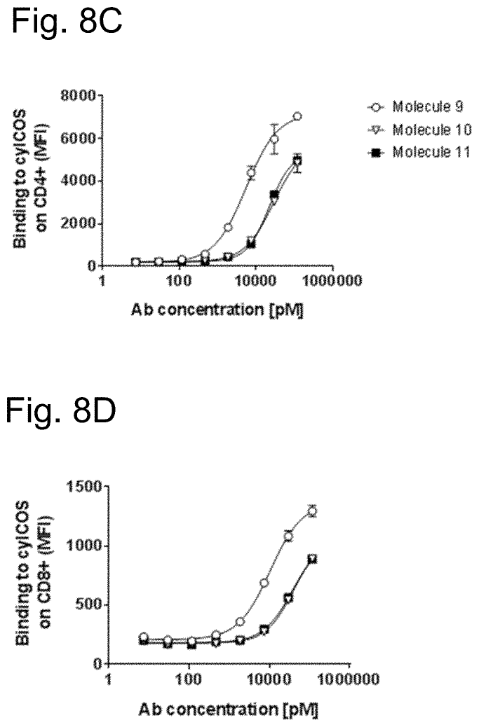

The results show that the tumor-targeted ICOS antigen binding molecules are able to bind to cynomolgus ICOS in a concentration dependent manner (FIGS. 8A and 8B). EC₅₀ Values are depicted in Table 46. Best binding was observed for Molecule 15 on both CD4⁺ and CD8⁺ T-Cell subsets.

TABLE 46
EC₅₀ values of binding of different
bispecific tumor targeted anti-ICOS
molecules to cynomolgus PBMCs

| | EC50 [pM] | EC50 [pM] |
|---|---|---|
| Molecule | CD4+ | CD8+ |
| Molecule 15 | 2092 | 3501 |
| Molecule 19 | 13490 | 13958 |
| Molecule 22 | 6533 | 8337 |
| Molecule 9 | 5500 | 10515 |
| Molecule 2 | 9982 | 18704 |

Comparing the formats described in FIGS. 1A, 1B and 1C in their ability to bind to cynomolgus ICOS, the bivalent binding to ICOS of the format described in FIG. 1C proved to be superior to the monovalent binding of the formats described in FIG. 1A and FIG. 1B (FIGS. 8C and 8D and Table 47).

TABLE 47
EC₅₀ values of binding of different formats of bispecific tumor
targeted anti-ICOS molecules to cynomolgus PBMCs

| | EC50 [pM] | EC50 [pM] |
|---|---|---|
| Molecule | CD4+ | CD8+ |
| Molecule 9 | 5500 | 10515 |
| Molecule 10 | n.c. | n.c. |
| Molecule 11 | n.c. | n.c. |

Additionally, the binding of the mouse cross-reactive molecules 9, 10 and 11 to murine ICOS was assessed using murine splenocytes with the following alterations to the protocol described above: BrSpleens of C57Bl/6 mice or hCEA(HO)Tg mice were transferred into gentleMACS C-tubes (Miltenyi) and MACS buffer (PBS+0.5% BSA+2 mM EDTA) was added to each tube. Spleens were dissociated using the GentleMACS Dissociator, tubes were spun down shortly and cells were passed through a 100 μm nylon cell strainer. Thereafter, tubes were rinsed with 3 ml RPMI1640 medium (SIGMA, Cat.-No. R7388) and centrifuged for 8 min at 350×g. The supernatant was discarded, the cell suspension passed through a 70 μm nylon cell strainer and washed with medium. After another centrifugation (350×g, 8 min), supernatants were discarded and 5 ml ACK Lysis Buffer was added. After 5 min incubation at RT cells were washed with RPMI medium. Afterwards the cells were re-suspended and the pellets pooled in assay medium (RPMI1640, 2% FBS, 1% Glutamax), for cell counting (Vi-Cell-Settings leukocytes, 1:10 dilution). Then, splenocytes were pre-activated for 48 h with PHA-L (Sigma #, 2 μg/ml) and IL-2 (Proleukin, Novartis; 200 U/ml) to upregulate the expression of murine ICOS and then used for a subsequent binding experiment, as described above.

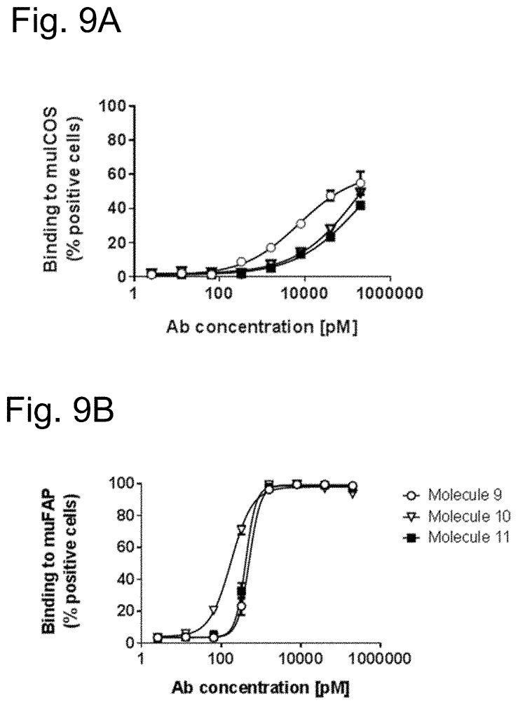

The results show that the molecules are able to bind to murine ICOS in a concentration dependent manner (FIG. 9A). EC₅₀ values are depicted in Table 48. Again the 2+1 format shows superior binding to murine ICOS, while the 1+1 format shows superior binding to murine FAP compared to 2+1 and 1+1 HT formats (FIG. 9B).

TABLE 48
EC₅₀ values of binding of different tumor targeted anti-ICOS
molecules to murine splenocytes

| | EC50 [pM] | EC50 [pM] |
|---|---|---|
| Molecule | Murine ICOS | Murine FAP |
| Molecule 9 | 7211 | 505.4 |
| Molecule 10 | n.c. | 176.9 |
| Molecule 11 | n.c. | 412.8 |

Another set of formats for bispecific tumor-targeted anti ICOS molecules prepared in Examples 3.4 and 3.5 and depicted in FIGS. 1D and 1E were tested for their binding properties to human ICOS and human FAP as described above apart from the modification that pre-activated human PBMCs were used as target cells for the binding to human ICOS.

Briefly, Peripheral blood mononuclear cells (PBMCs) were prepared by Histopaque (Sigma-Aldrich, Cat No. 10771-500ML Histopaque-1077) density centrifugation of enriched lymphocyte preparations of heparinized blood obtained from a Buffy Coat (“Blutspende Zürich”). The blood was diluted 1:2 with sterile DPBS and layered over Histopaque gradient (Sigma, #H8889). After centrifugation (450×g, 30 minutes, room temperature), the plasma above the PBMC-containing interphase was discarded and PBMCs transferred in a new falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (Cedex HiRes) and stored in RPMI1640 medium containing 10% FCS and 1% Glutamax (Gibco 35050061). PBMCs were pre-activated for 48 h with PHA-L (Sigma #, 2 μg/ml) and IL-2 (Proleukin, Novartis; 200 U/ml) to upregulate the expression of human ICOS at 37° C., in a humidified incubator. After incubation the PBMCs were used for a subsequent binding experiment, as described above.

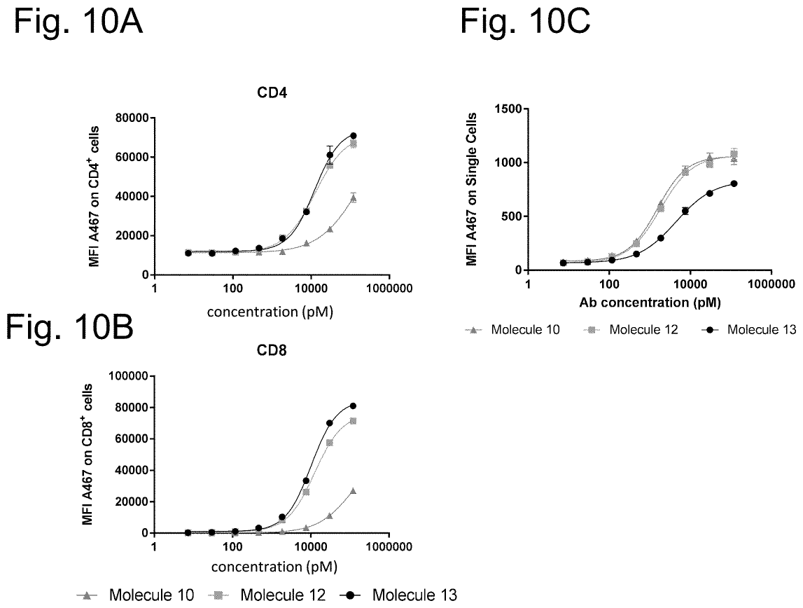

The results show that the bispecific FAP-targeted ICOS molecules are able to bind to human ICOS and human FAP in a concentration dependent manner (FIGS. 10A to 10C). EC₅₀ values are depicted in Table 49. Molecule 12 and 13 exhibit superior binding to human ICOS on both CD4⁺ and CD8⁺ T-Cell Subsets format (FIGS. 10A and 10B). On the other hand, Molecule 13 exhibits inferior binding to human FAP (FIG. 10C).

TABLE 49
EC₅₀ values of binding of different FAP-targeted anti-
ICOS molecules to FAP+ NIH/3t3-huFAP cl. 19 cells or
pre-activated CD4+ and CD8+ subsets of human PBMCs

| | Human ICOS | | |
|---|---|---|---|
| | CD4+ | CD8+ | Human FAP |
| Molecule | EC₅₀ [pM] | | EC₅₀ [pM] |
| Molecule 10 | ~58018 | ~124038 | 1540 |
| Molecule 12 | ~12585 | ~12612 | 1821 |
| Molecule 13 | n.c. | n.c. | 4450 |

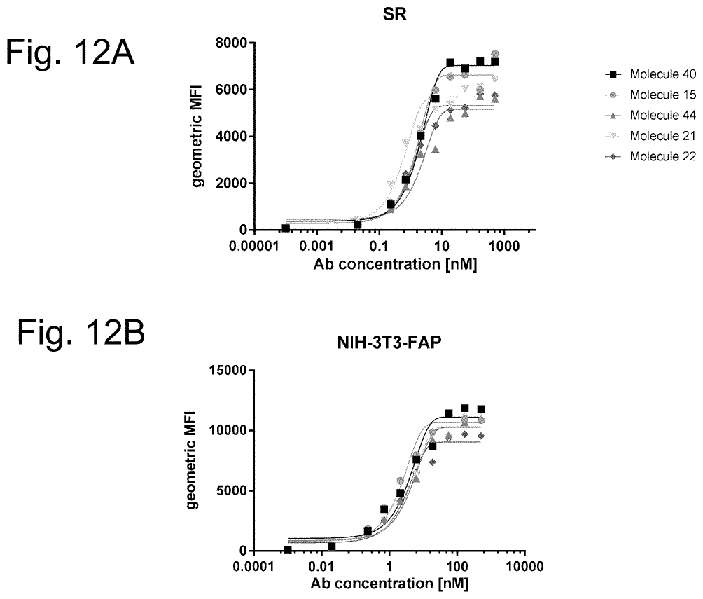

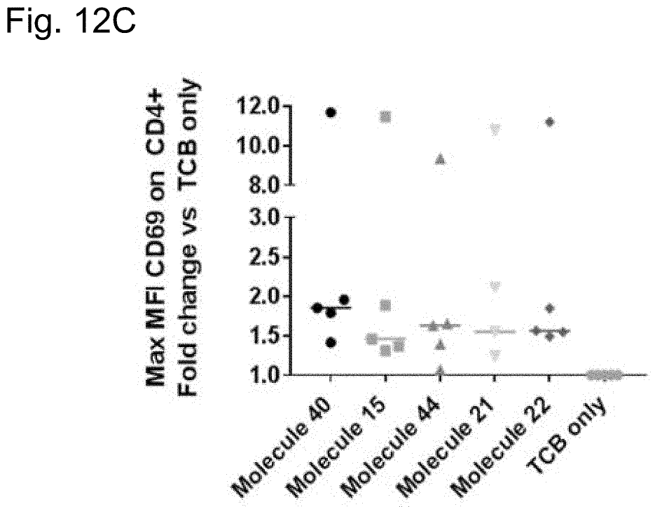

Additionally, the binding of FAP-targeted ICOS molecules 40, 15, 44, 21 and 22 to ICOS on SR cells and to FAP⁺ NIH/3t3-huFAP cl. 19 cells has been tested in a further experiment (see FIGS. 12A and 12B). The data are shown in Table 49A below.

TABLE 49A
EC₅₀ values of binding of different FAP-
targeted anti-ICOS molecules to ICOS on SR
cells and to FAP+ NIH/3t3-huFAP cl. 19 cells

| | Human ICOS | |
|---|---|---|
| | on SR cells | Human FAP |
| Molecule | EC₅₀ [pM] | EC₅₀ [pM] |
| Molecule 40 | 1.74 | 3.85 |
| Molecule 15 | 1.39 | 1.98 |
| Molecule 44 | 2.17 | 4.34 |
| Molecule 21 | 0.57 | 5.01 |
| Molecule 22 | 1.17 | 3.24 |

Another set of tumor targeted anti ICOS molecules prepared in Example 6, targeted to CEA instead of FAP, were tested for their binding properties to human ICOS and human CEA as described above. Binding to human ICOS was tested on human PBMCs pre-activated as described before. Binding to CEA was assessed using MKN-45 cells (human gastric adenocarcinoma cell line, DSMZ ACC 409).

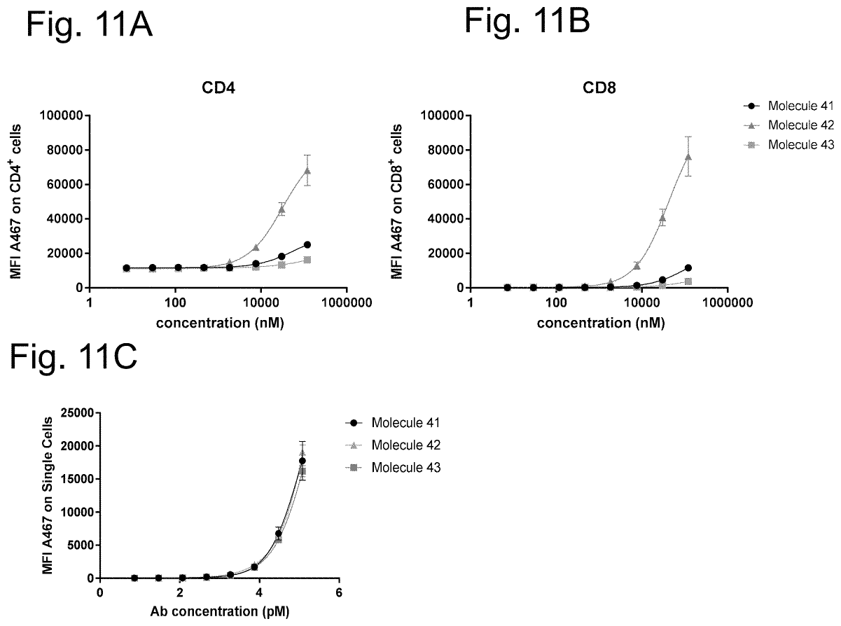

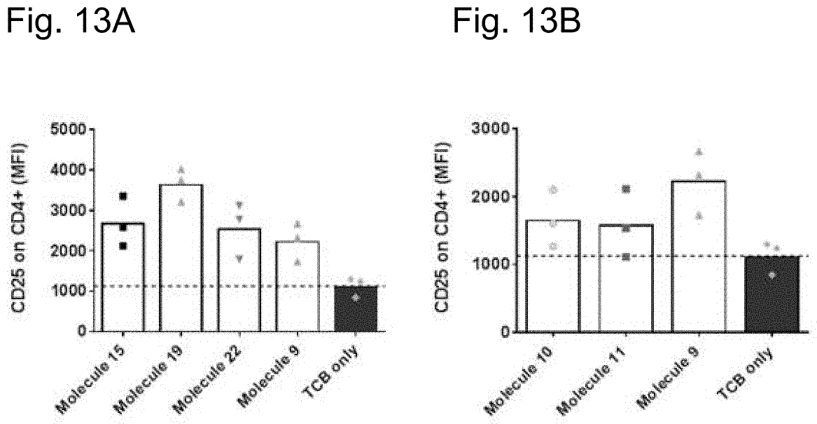

The results show that the CEA-targeted bispecific ICOS molecules are able to bind to human ICOS and human CEA in a concentration dependent manner (FIGS. 11A to 11C). Molecule 42 exhibits superior binding to human ICOS (FIGS. 11A and 11B), while all three molecules show comparable binding to human CEA (FIG. 13C).

### 7.5 Increased TCB-Mediated T-Cell Activation in the Presence of Tumor-Targeted ICOS Antigen Binding Molecules (Flow Cytometry Analysis)

The capacity of either FAP- or CEA-targeted bispecific agonistic ICOS molecules to further boost CEACAM5-TCB-mediated activation of T-cells was assessed in a co-culture assay of CEA positive MKN-45 and FAP expressing NIH/3T3-huFAP clone 19 cells (ATCC, CCL-92, transfected to stably overexpress human FAP), as well as human PBMCs.

Briefly, adherent target cells were harvested with Cell Dissociation Buffer and plated at a density of 10 000 cells/well in flat-bottom 96-well plates one day before the experiment (Gibco, 13151014). Hereby, NIH/3T3-huFAP clone 19 cells were additionally irradiated before plating, using X-Ray Irradiator RS 2000 (Rad source) with 5000 rad (irradiation without filter, level 5). Target cells were left to adhere overnight. Peripheral blood mononuclear cells (PBMCs) were prepared by Histopaque (Sigma-Aldrich, Cat No. 10771-500ML Histopaque-1077) density centrifugation of enriched lymphocyte preparations from a Buffy Coat (“Blutspende Zürich”), The blood was diluted 1:2 with sterile DPBS and layered over Histopaque gradient (Sigma, #H8889). After centrifugation (450×g, 30 minutes, room temperature), the plasma above the PBMC-containing interphase was discarded and PBMCs transferred in a new falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (Cedex HiRes) and stored in RPMI1640 medium containing 10% FCS and 1% Glutamax (Gibco 35050061) at 37° C. in a humidified incubator until the assay was started.

PBMCs were added to target cells and Fibroblasts to obtain a final E:T ratio of 5:1:1 in presence of a fixed concentration of 80 pM CEACAM5-TCB and increasing concentrations of the FAP- or CEA-targeted ICOS molecules (0.11 pM-5000 pM in triplicates). T-Cell Activation was assessed after 48 h of incubation at 37° C., 5% CO₂ by flow cytometric analysis, using antibodies recognizing the T cell activation markers CD69 (early activation marker) and CD25 (late activation marker).

Briefly, PBMCs were centrifuged at 400×g for 4 min and washed twice with PBS containing 0.1% BSA (FACS buffer). Surface staining for CD8 (PerCP/Cy5.5 anti-human CD8a, BioLegend #301032), CD4 (APC/Cy7 anti-human CD4, BioLegend #300518), CD69 (BV421 anti-human CD69, BioLegend #310930), CD25 (PE anti-human CD25, BioLegend #356104) was performed according to the suppliers' indications. Cells were thenn washed twice with 150 μl/well PBS containing 0.1% BSA and fixed for 15-30 min at 4° C. using 75 μl/well FACS buffer, containing 1% PFA. After centrifugation, the samples were re-suspended in 150 μl/well FACS buffer. Fluorescence was analyzed by FACS using a FACS Fortessa (Software FACS Diva). Graphs were obtained using GraphPadPrism 7.

The agonistic activity of several FAP-ICOS molecules prepared in Example 3 were compared on up to five PBMC donors as described above (FIG. 12C). The results indicate comparable activity for molecule 44 and its variants molecule 21 and 22 and a slightly decreased activity of molecule 15, the variant of molecule 40.

In another example, the agonistic activity of selected FAP-ICOS molecules were compared on three PBMC donors (FIGS. 13A and 13B) as described above apart from the following modifications: instead of 80 pM CEACAM5 TCB 5 pM of MCSP TCB were used in conjunction with the replacement of the cell lines with MCSP⁺ and FAP⁺ MV-3 cells (Accession No. CVCL_W280) in an Effector to Target Ratio of 5:1 (50'000 effectors and 10'000 target cells per well).

All tested FAP-ICOS molecules were able to boost TCB mediated T-Cell activation (FIG. 13A). Strongest activation was observed with Molecule 19. When comparing three different formats of FAP-ICOS, the format described in FIG. 1C induced the strongest activation (FIG. 13B).

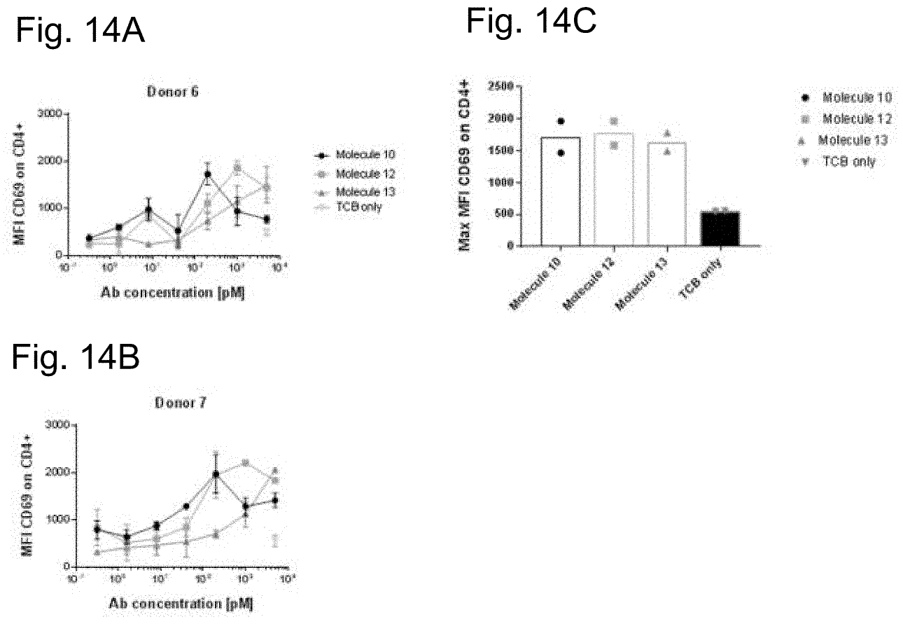

In a separate assay, the formats described in FIGS. 1A, 1D and 1E were compared as described above on two healthy PBMC donors (FIGS. 14A to 14C).

The results show that all three formats can induce additional T-Cell activation when compared to TCB treatment alone. No difference in the maximal agonistic activity can be found between the three formats tested (FIG. 14C). However, the three formats reach their maximal agonistic activity at different concentrations with a ranking (from lower to higher concentration) of FIG. 1A>FIG. 1E>FIG. 1D.

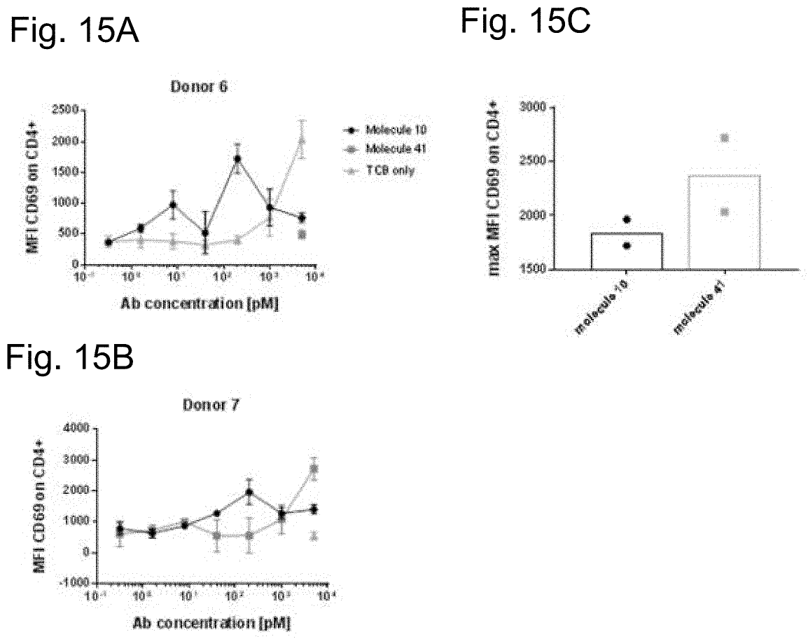

To assess the difference of targeting TCB and tumor targeted ICOS molecules to the same target cells (“cis-setting”) or to two different cells (“trans-settings”) two ICOS molecules either targeted to FAP (trans-setting) or CEA (cis-setting) were tested in the assay described above on two healthy PBMC donors (FIGS. 15A to 15C).

The results show a higher overall agonistic activity of the CEA-targeted molecule 41 (FIG. 15C). However, the FAP targeted molecule 10 seems to reach its maximal agonistic activity at a lower concentration (FIGS. 15A to 15B).

Additionally, a set of CEA-ICOS molecules was tested on three PBMC donors as described before using NIH/3t3-huFAP clone 19, MKN-45 cells as targets and 80 pM CEACAM5 TCB as first stimulus.

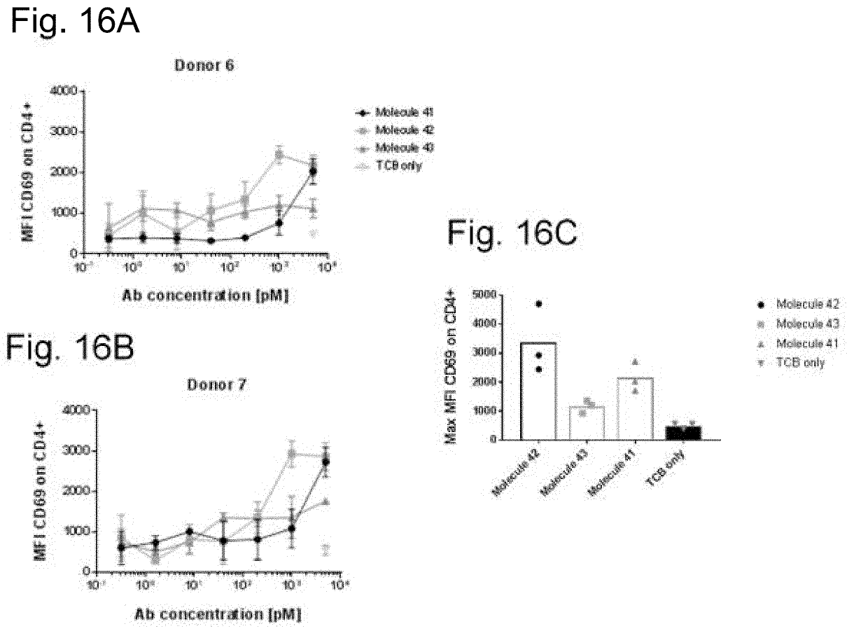

The results show that all three molecules tested are able to further boost T-Cell activation compared to TCB stimulation alone (FIGS. 16A to 16C). Molecule 42 shows the highest additional stimulation.

### Example 8

### Preparation, Purification and Characterization of T-Cell Bispecific (TCB) Antibodies

4.1 Preparation of TCB Antibodies with Human or Humanized Binders

TCB molecules have been prepared according to the methods described in WO 2014/131712 A1 or WO 2016/079076 A1.

The preparation of the anti-CEA/anti-CD3 bispecific antibody (CEA CD3 TCB or CEA TCB) used in the experiments is described in Example 3 of WO 2014/131712 A1. CEA CD3 TCB is a “2+1 IgG CrossFab” antibody and is comprised of two different heavy chains and two different light chains. Point mutations in the CH3 domain (“knobs into holes”) were introduced to promote the assembly of the two different heavy chains. Exchange of the VH and VL domains in the CD3 binding Fab were made in order to promote the correct assembly of the two different light chains. 2+1 means that the molecule has two antigen binding domains specific for CEA and one antigen binding domain specific for CD3. CEACAM5 CD3 TCB has the same format, but comprises another CEA binder and comprises point mutations in the CH and CL domains of the CD3 binder in order to support correct pairing of the light chains.

CEA CD3 TCB comprises the amino acid sequences of SEQ ID NO:242, SEQ ID NO:243, SEQ ID NO:244 and SEQ ID NO:245. CEACAM5 CD TCB comprises the amino acid sequences of SEQ ID NO:246, SEQ ID NO:247, SEQ ID NO:249 and SEQ ID NO:249.

### 4.2 Preparation of Anti-CEA/Anti-CD3 T Cell Bispecific Antibody in 2+1 Format (Bivalent for Murine CEA and Monovalent for Murine CD3)

The anti-CEA(CH1A1A 98/99 2F1)/anti-CD3(2C11) T cell bispecific 2+1 surrogate molecule was prepared consisting of one CD3-Fab, and two CEA-Fabs and a Fc domain, wherein the two CEA-Fabs are linked via their C-termini to the hinge region of said Fc part and wherein the CD3-Fab is linked with its C-terminus to the N-terminus of one CEA-Fab. The CD3 binding moiety is a crossover Fab molecule wherein either the variable or the constant regions of the Fab light chain and the Fab heavy chain are exchanged.

The Fc domain of the murine surrogate molecule is a mu IgG1 Fc domain, wherein DDKK mutations have been introduced to enhance antibody Fc heterodimer formation as inter alia described by Gunasekaran et al., J. Biol. Chem. 2010, 19637-19646. The Fc part of the first heavy chain comprises the mutations Lys392Asp and Lys409Asp (termed Fc-DD) and the Fc part of the second heavy chain comprises the mutations Glu356Lys and Asp399Lys (termed Fc-KK). The numbering is according to Kabat EU index. Furthermore, DAPG mutations were introduced in the constant regions of the heavy chains to abrogate binding to mouse Fc gamma receptors according to the method described e.g. in Baudino et al. J. Immunol. (2008), 181, 6664-6669, or in WO 2016/030350 A1. Briefly, the Asp265Ala and Pro329Gly mutations have been introduced in the constant region of the Fc-DD and Fc-KK heavy chains to abrogate binding to Fc gamma receptors (numbering according to Kabat EU index; i.e. D265A, P329G).

Anti-CEA(CH1A1A 98/99 2F1)/anti-CD3(2C11) T cell bispecific 2+1 surrogate molecule thus comprises the amino acid sequences of SEQ ID NO:250, SEQ ID NO:251, SEQ ID NO:252 and SEQ ID NO:253.

### Example 9

### In Vivo Functional Characterization of Tumor-Targeted ICOS Antigen Binding Molecules in Combination with CEACAM5-TCB

9.1 Pharmacokinetic Profile of Bispecific FAP-ICOS (1167) Bispecific Antibodies after Single Injection in NSG Mice

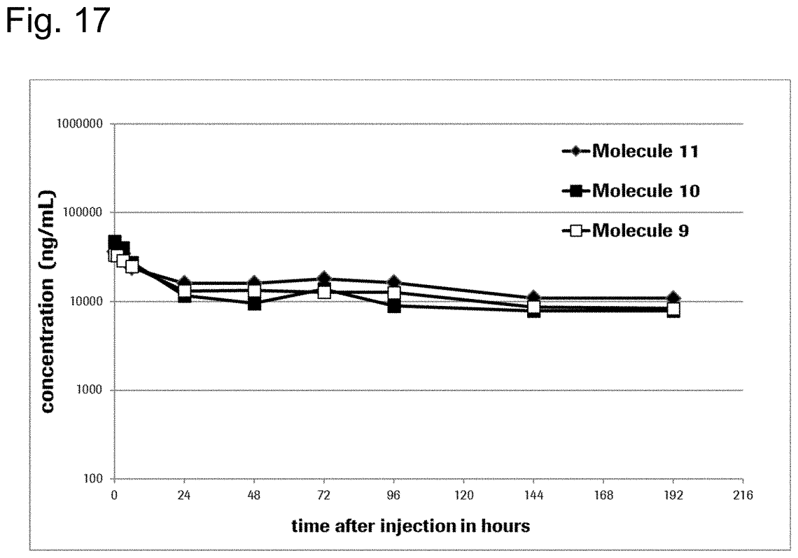

A single dose of 2.5 mg/kg of FAP-ICOS molecules were injected into NSG mice. All mice were injected i.v. with 200 μl of the appropriate solution. To obtain the proper amount of compounds per 200 μl, the stock solutions (Table 50) were diluted with histidine buffer. Three mice per time point and group were bled at 10 min, 1 hr, 3 hr, 6 hr, 24 hr, 48 hr, 72 hr, 96 hr, 6 days, 8 days, 10 days and 12 days. The injected compounds were analyzed in serum samples by ELISA. Detection of the molecules were carried out by huICOS ELISA (detection via human ICOS binding). The plates were washed three times after each step to remove unbound substances. Finally, the peroxidase-bound complex is visualized by adding ABTS substrate solution to form a colored reaction product. The reaction product intensity, which is photometrically determined at 405 nm (with reference wavelength at 490 nm), is proportional to the analyte concentration in the serum sample. The results (FIG. 17) showed a stable PK-behavior for all molecules which suggested a once weekly schedule for subsequent efficacy studies.

TABLE 50
Description of tested compositions

| | Formulation | Concentration |
|---|---|---|
| Compound | buffer | (mg/mL) |
| FAP-ICOS | 20 mM Histidine, | 1.05 |
| (HT) | 140 mM NaCl, | (=stock solution) |
| | pH 6.0 | |
| FAP-ICOS | 20 mM Histidine, | 2.9 |
| (1 + 1) | 140 mM NaCl, | (=stock solution) |
| | pH 6.0 | |
| FAP-ICOS | 20 mM Histidine, | 2.0 |
| (2 + 1) | 140 mM NaCl, | (=stock solution) |
| | pH 6.0 | |

9.2 In Vivo Efficacy Study FAP-ICOS Antibodies in Combination with CEACAM5-TCB in MKN45 Xenograft in Humanized Mice

The efficacy study described in here was aimed to understand the format dependent potency of the FAP-ICOS molecules in combination with CEACAM5-TCB in terms of tumor regression and Immuno-PD in fully humanized NSG mice.

Human MKN45 cells (human gastric carcinoma) were originally obtained from ATCC and after expansion deposited in the Glycart internal cell bank. Cells were cultured in DMEM containing 10% FCS at 37° C. in a water-saturated atmosphere at 5% CO₂. In vitro passage 12 was used for subcutaneous injection at a viability of 97%. Human fibroblasts NIH-3T3 were originally obtained from ATCC, engineered at Roche Nutley to express human FAP and cultured in DMEM containing 10% Calf serum, 1× Sodium Pyruvate and 1.5 ug/ml Puromycin. Clone 39 was used at an in vitro passage number 18 and at a viability of 98.2%.

50 microliters cell suspension (1×10⁶ MKN45 cells+1×10⁶ 3T3-huFAP) mixed with 50 microliters Matrigel were injected subcutaneously in the flank of anaesthetized mice with a 22 G to 30 G needle.

Female NSG mice, age 4-5 weeks at start of the experiment (Jackson Laboratory) were maintained under specific-pathogen-free condition with daily cycles of 12 h light/12 h darkness according to committed guidelines (GV-Solas; Felasa; TierschG). The experimental study protocol was reviewed and approved by local government (P 2011/128). After arrival, animals were maintained for one week to get accustomed to the new environment and for observation. Continuous health monitoring was carried out on a regular basis.

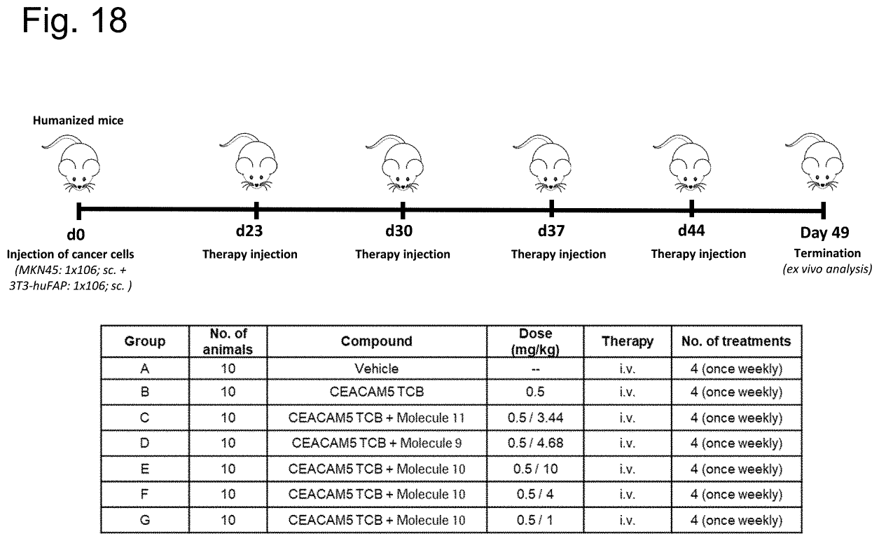

Female NSG mice were injected i.p. with 15 mg/kg of Busulfan followed one day later by an i.v. injection of 1×10⁵ human hematopoietic stem cells isolated from cord blood. At week 14-16 after stem cell injection mice were bled sublingual and blood was analyzed by flow cytometry for successful humanization. Efficiently engrafted mice were randomized according to their human T cell frequencies into the different treatment groups. At that time, mice were injected with tumor cells and fibroblasts s.c. as described (FIG. 18) and treated once weekly with the compounds or Histidine buffer (Vehicle) when tumor size reached appr. 250 mm³ (day 23). All mice were injected i.v. with 200 μl of the appropriate solution. To obtain the proper amount of compounds per 200 μl, the stock solutions (Table 51) were diluted with Histidine buffer when necessary. Doses of the different FAP-ICOS molecules were adapted according to their molecular weight (matched molarity, Groups C, D, F). For the 1+1 Format, three doses have been used (Groups E-G). For combination therapies (Groups C-G, FIG. 1) with FAP-ICOS and CEACAM5 TCB constructs were injected concomitant. Tumor growth was measured twice weekly using a caliper (FIG. 18) and tumor volume was calculated as followed:

$${T}_{v} \text{:} ( {W}^{2} \text{/} 2 ) \times L ( W \text{:} Width , L \text{:} Length )$$

At termination (day 50), mice were sacrificed, tumors and spleen were removed, weighted and single cell suspensions were prepared through an enzymatic digestion with Collagenase V and DNAse for subsequent FACS-analysis. Single cells where stained for human CD45, CD3, CD8, CD4, CD25, CD19 and FoxP3 (intracellular) and analyzed at FACS Fortessa.

Small pieces (30 mg) of tumor tissues were snap frozen and whole protein was isolated. Protein suspensions were analysed for cytokine content by Multiplex analysis.

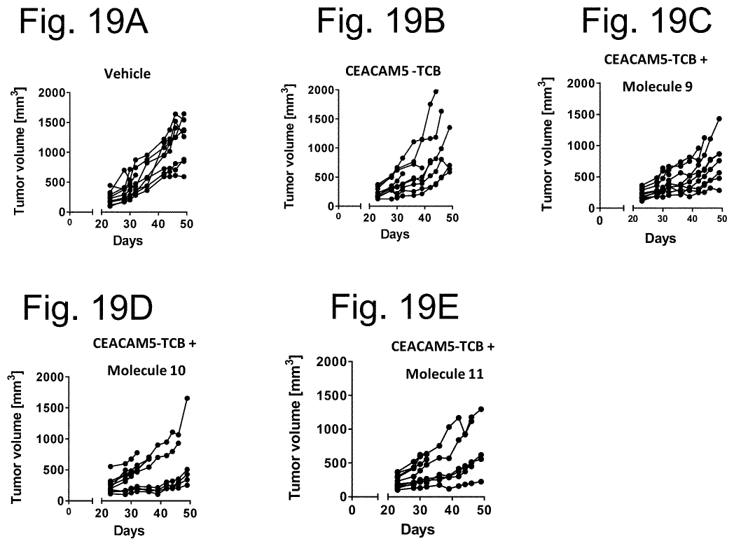

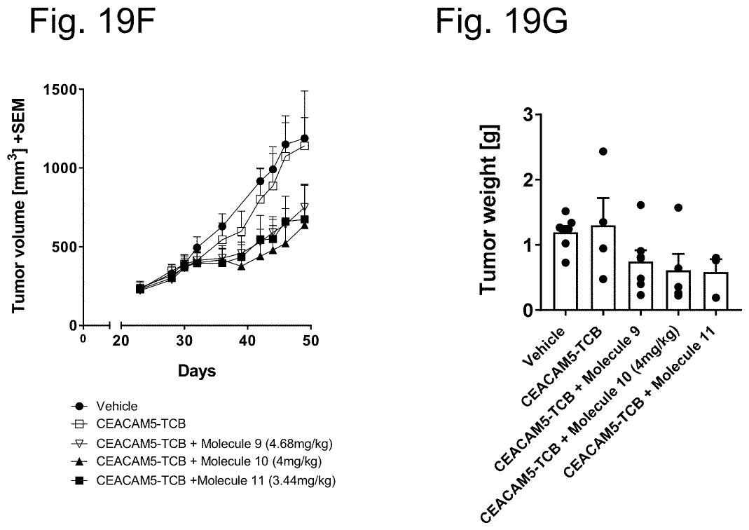

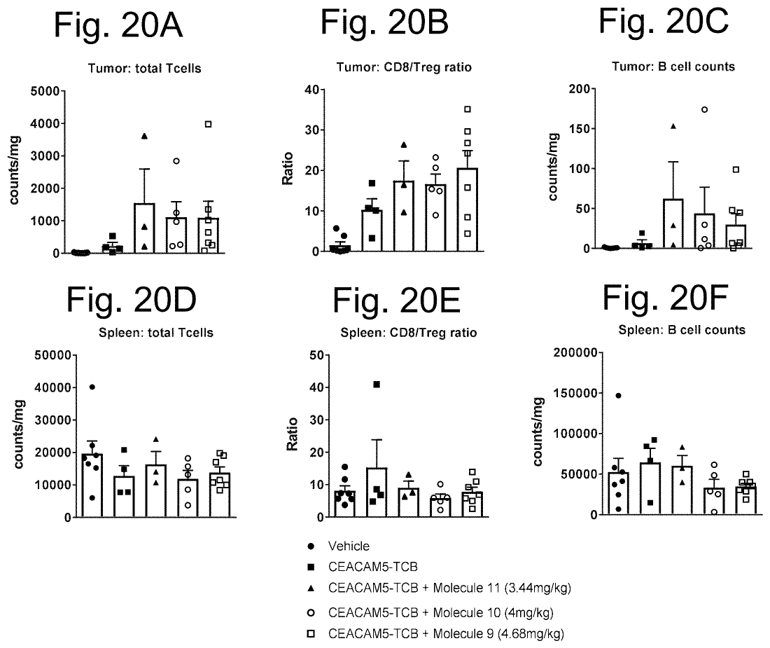

FIGS. 19A to 19G show the tumor growth kinetics (Mean, +SEM) in the molarity matched combination treatment groups as well as the individual tumor growth per mouse and the tumor weights at study termination. As described here, CEACAM5 TCB, as a single agent induced little initial tumor growth inhibition. However, the combinations with all FAP-ICOS molecules showed significant improved tumor growth inhibition that was also reflected by tumor weight at study termination (FIG. 19G). Interestingly, the Immuno-PD data (FIGS. 20A to 20F) of tumors from animals sacrificed at study termination, revealed an increase of intratumoral T and B cell frequencies in all combination groups. The increased T cell infiltration in the tumor shifted the CD8/Treg ratio towards CD8 cells in the combination treatments. No effects have been detected in spleen at termination. However, no statistical differences were observed in terms of Tumor growth and ImmunoPD between the different types of bispecific FAP-ICOS antibodies used.

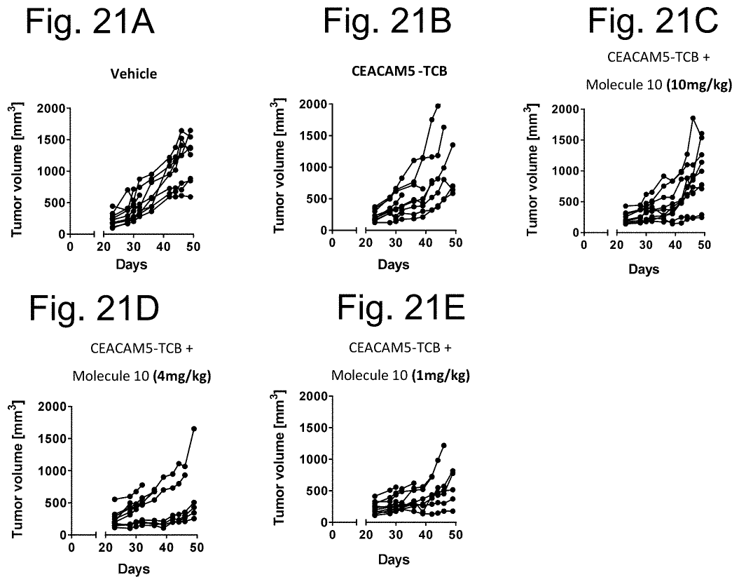

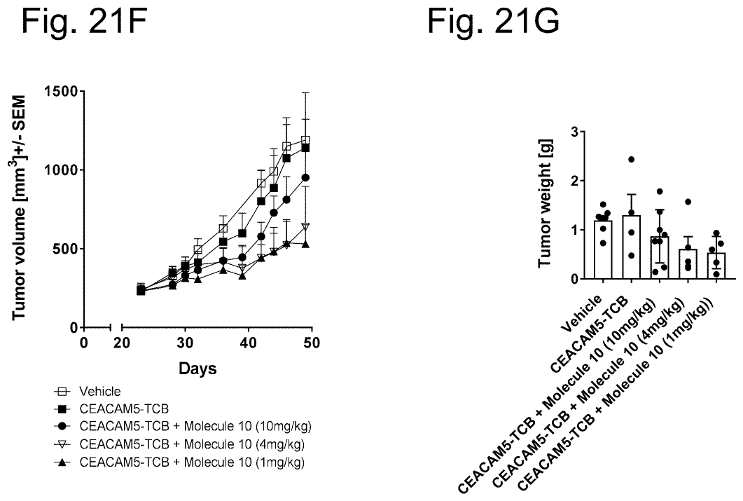

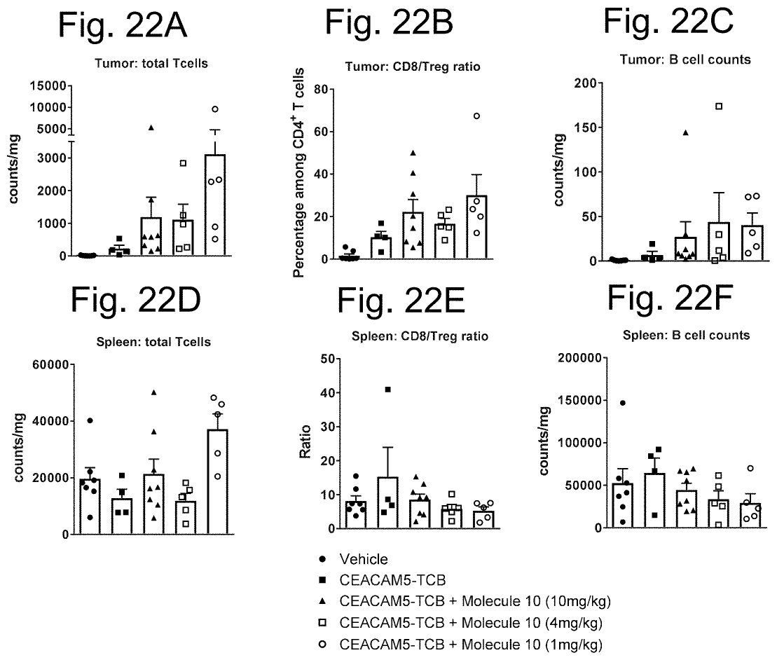

FIGS. 21A to 21G show the tumor growth kinetics (Mean, +SEM) for the dose response groups of the 1+1 FAP-ICOS format as well as the individual tumor growth per mouse and the tumor weights at study termination. The tumor growth data for the different doses revealed that the strongest effects have been seen with 4 and 1 mg/kg doses whereas the highest dose tested, 10 mg/kg, showed a weaker response. Interestingly, the Immuno-PD data (FIGS. 22A to 22F) of tumors from animals sacrificed at study termination, revealed that all doses of FAP-ICOS 1+1 format increased intratumoral T and B cell frequencies. The increased T cell infiltration in the tumor shifted the CD8/Treg ratio towards CD8 cells in the combination treatments. The strongest Immuno-PD effects have been detected with the lowest dose tested (1 mg/kg).

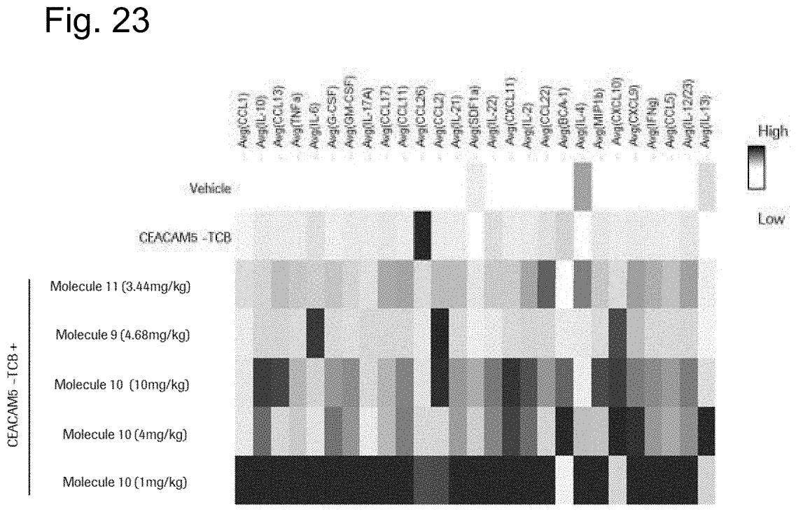

Furthermore, the cytokine/chemokine analyses, shown in FIG. 23, on whole tumor protein lysates revealed the strongest upregulation of cytokines/chemokine with the lowest dose of the bispecific FAP-ICOS antibody in 1+1 format over all other treatment group tested.

TABLE 51
Description of tested compositions

| | Formulation | Concentration |
|---|---|---|
| Compound | buffer | (mg/mL) |
| CEACAM5- | 20 mM Histidine, | 4.7 |
| TCB | 140 mM NaCl, | (=stock solution) |
| | pH 6.0 | |
| FAP-ICOS | 20 mM Histidine, | 1.05 |
| (HT) | 140 mM NaCl, | (=stock solution) |
| | pH 6.0 | |
| FAP-ICOS | 20 mM Histidine, | 2.9 |
| (1 + 1) | 140 mM NaCl, | (=stock solution) |
| | pH 6.0 | |
| FAP-ICOS | 20 mM Histidine, | 2.0 |
| (2 + 1) | 140 mM NaCl, | (=stock solution) |
| | pH 6.0 | |

### LITERATURE REFERENCES

Allen F., Bobanga J., et al., CCL3 in the tumor microenvironment augments the antitumor immune response. J Immunol May 1, 2016, 196 (75.11)
Allison J, Sharma P, Quezada, S. A., Fu T. Combination immunotherapy for the treatment of cancer, WO2011/041613A2 2009
Bacac M, Fauti T, Sam J, et al. A Novel Carcinoembryonic Antigen T-Cell Bispecific Antibody (CEA TCB) for the Treatment of Solid Tumours. Clin Cancer Res. 2016 Jul. 1; 22(13):3286-97.
Bacac M, Klein C, Umana P. CEA TCB: A novel head-to-tail 2:1 T cell bispecific antibody for treatment of CEA-positive solid tumours. Oncoimmunology. 2016 Jun. 24; 5(8).
Carthon B C et al., “Preoperative CTLA-4 blockade: Tolerability and immune monitoring in the setting of a presurgical clinical trial Clin Cancer Res. 2010 16(10); 2861-71.
Dammeijer F., Lau S. P., van Eijck C. H. J., van der Burg S. H., Aerts J. G. J. V. Rationally combining immunotherapies to improve efficacy of immune checkpoint blockade in solid tumours. Cytokine & Growth Factor Reviews 2017 August; 36: 5-15.
Davidson E. H., Hood L., Dimitrov K., Direct multiplexed measurement of gene expression with color-coded probe pairs. Nature biotechnology 2008; 26:317-325.
Fu T et al., The ICOS/ICOSL pathway is required for optimal antitumour responses mediated by anti-CTLA-4 therapy. Cancer Res. 2011, 71(16); 5445-54.
Geiss G. K. et al., Direct multiplexed measurement of gene expression with color-coded probe pairs. Nat Biotechnol. 2008 March; 26(3):317-25.
Giacomo A M D et al., “Long-term survival and immunological parameters in metastatic melanoma patients who respond to ipilimumab 10 mg/kg within an expanded access program”, Cancer Immunol Immunother. 2013, 62(6); 1021-8.
Gu-Trantien C., Migliori E., et al., CXCL13-producing TFH cells link immune suppression and adaptive memory in human breast cancer. JCI Insight. 2017 Jun. 2; 2(11).
Hutloff A., Dittrich A. M., Beier K. C., Eljaschewitsch B., Kraft R., Anagnostopoulos I., Kroczek R. A. ICOS is an inducible T-cell co-stimulator structurally and functionally related to CD28. Nature. 1999 Jan. 21; 397(6716):263-6.
Im S. J., Hashimoto M., et al. Defining CD8+ T cells that provide the proliferative burst after PD-1 therapy. Nature. 2016 Sep. 15; 537(7620): 417-421.
Liakou C I et al., CTLA-4 blockade increases IFN-gamma producing CD4+ICOShi cells to shift the ratio of effector to regulatory T cells in cancer patients. Proc Natl Acad Sci USA 2008,105(39); 14987-92.
Manzoor A. M., Developing Costimulatory Molecules for Immunotherapy of Diseases, Academic Press, 2015; eBook ISBN 9780128026755
Paulos C. M., Carpenito C., Plesa G., Suhoski M. M., Varela-Rohena A., Golovina T. N., Carroll R. G., Riley J. L., June C. H. The inducible costimulator (ICOS) is critical for the development of human T(H)17 cells. Sci Transl Med. 2010 Oct. 27; 2(55):55ra78.
Sharma P, Allison J 2015. The future of immune checkpoint therapy. Science 2015; 348: 56-61
Simpson T. R., Quezada S. A., Allison J. P. Regulation of CD4 T cell activation and effector function by inducible costimulator (ICOS). Current Opinion in Immunology 2010, 22.
Vonderheide R H et al., Tremelimumab in combination with exemestane in patients with advanced breast cancer and treatment-associated modulation of inducible costimulator expression on patient T cells, Clin Cancer Res. 2010, 16(13); 3485-94.
Wakamatsu E., Mathis D., Benoist C. Convergent and divergent effects of costimulatory molecules in conventional and regulatory CD4+ T cells. Proc Natl Acad Sci USA. 2013 Jan. 15; 110(3):1023-8.
Warnatz K., et al., Human ICOS deficiency abrogates the germinal center reaction and provides a monogenic model for common variable immunodeficiency. Blood 2006 107:3045-3052
Yao S., Zhu Y., Zhu G., Augustine M., Zheng L., Goode D. J., Broadwater M., Ruff W., Flies S., Xu H., Flies D., Luo L., Wang S., Chen L. B7-h2 is a costimulatory ligand for CD28 in human. Immunity. 2011 May 27; 34(5):729-40.
Young, M. R. I., Th17 Cells in Protection from Tumor or Promotion of Tumor Progression. J Clin Cell Immunol. 2016 June; 7(3): 431.
Yuraszeck et al., Translation and Clinical Development of Bispecific T-cell Engaging Antibodies for Cancer Treatment. Clinical Pharmacology & Therapeutics 2017, 101

1. An agonistic ICOS antigen binding molecule comprising at least one antigen binding domain capable of specific binding to a tumor-associated antigen and at least one antigen binding domain capable of specific binding to ICOS comprising
(a) a heavy chain variable region (V_{H}ICOS) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}ICOS) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}ICOS) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}ICOS) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or
(c) a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25, or
(d) a heavy chain variable region (V_{H}ICOS) comprising (i) CDR-H1 comprising the amino acid sequence of SEQ ID NO:28, (ii) CDR-H2 comprising the amino acid sequence of SEQ ID NO:29, and (iii) CDR-H3 comprising the amino acid sequence of SEQ ID NO:30, and a light chain variable region (V_{L}ICOS) comprising (iv) CDR-L1 comprising the amino acid sequence of SEQ ID NO:31, (v) CDR-L2 comprising the amino acid sequence of SEQ ID NO:32, and (vi) CDR-L3 comprising the amino acid sequence of SEQ ID NO:33.

2. The agonistic ICOS antigen binding molecule of claim 1, further comprising a Fc domain composed of a first and a second subunit capable of stable association which comprises one or more amino acid substitution that reduces the binding affinity of the antigen binding molecule to an Fc receptor and/or effector function.

3. The agonistic ICOS antigen binding molecule of claim 1 or 2, comprising a Fc domain of human IgG1 subclass which comprises the amino acid mutations L234A, L235A and P329G (numbering according to Kabat EU index).

4. The agonistic ICOS antigen binding molecule of any one of claims 1 to 3, wherein the antigen binding domain capable of specific binding to a tumor-associated antigen is an antigen binding domain capable of specific binding to Carcinoembryonic Antigen (CEA).

5. The agonistic ICOS antigen binding molecule of any one of claims 1 to 4, wherein wherein the antigen binding domain capable of specific binding to CEA comprises
(a) a heavy chain variable region (V_{H}CEA) comprising (i) CDR-H1 comprising the amino acid sequence of SEQ ID NO:52, (ii) CDR-H2 comprising the amino acid sequence of SEQ ID NO:53, and (iii) CDR-H3 comprising the amino acid sequence of SEQ ID NO:54, and a light chain variable region (V_{L}CEA) comprising (iv) CDR-L1 comprising the amino acid sequence of SEQ ID NO:55, (v) CDR-L2 comprising the amino acid sequence of SEQ ID NO:56, and (vi) CDR-L3 comprising the amino acid sequence of SEQ ID NO:57, or
(b) a heavy chain variable region (V_{H}CEA) comprising (i) CDR-H1 comprising the amino acid sequence of SEQ ID NO:60, (ii) CDR-H2 comprising the amino acid sequence of SEQ ID NO:61, and (iii) CDR-H3 comprising the amino acid sequence of SEQ ID NO:62, and a light chain variable region (V_{L}CEA) comprising (iv) CDR-L1 comprising the amino acid sequence of SEQ ID NO:63, (v) CDR-L2 comprising the amino acid sequence of SEQ ID NO:64, and (vi) CDR-L3 comprising the amino acid sequence of SEQ ID NO:65.

6. The agonistic ICOS antigen binding molecule of any one of claims 1 to 5, wherein the antigen binding domain capable of specific binding to CEA comprises a heavy chain variable region (V_{H}CEA) comprising an amino acid sequence that is at least about 95%, 96%, 97%, 98%, 99% or 100% identical to the amino acid sequence of SEQ ID NO:58, and a light chain variable region (V_{L}CEA) comprising an amino acid sequence that is at least about 95%, 96%, 97%, 98%, 99% or 100% identical to the amino acid sequence of SEQ ID NO:59, or a heavy chain variable region (V_{H}CEA) comprising an amino acid sequence that is at least about 95%, 96%, 97%, 98%, 99% or 100% identical to the amino acid sequence of SEQ ID NO:68, and a light chain variable region (V_{L}CEA) comprising an amino acid sequence that is at least about 95%, 96%, 97%, 98%, 99% or 100% identical to the amino acid sequence of SEQ ID NO:69.

7. The agonistic ICOS antigen binding molecule of any one of claims 1 to 6, wherein the antigen binding domain capable of specific binding to CEA comprises a heavy chain variable region (V_{H}CEA) comprising the amino acid sequence of SEQ ID NO:68, and a light chain variable region (V_{L}CEA) comprising the amino acid sequence of SEQ ID NO:69.

8. The agonistic ICOS antigen binding molecule of any one of claims 1 to 3, wherein the antigen binding domain capable of specific binding to a tumor-associated antigen is an antigen binding domain capable of specific binding to Fibroblast Activation Protein (FAP).

9. The agonistic ICOS antigen binding molecule of any one of claims 1 to 3 or 8, wherein the antigen binding domain capable of specific binding to FAP comprises
(a) a heavy chain variable region (V_{H}FAP) comprising (i) CDR-H1 comprising the amino acid sequence of SEQ ID NO:36, (ii) CDR-H2 comprising the amino acid sequence of SEQ ID NO:37, and (iii) CDR-H3 comprising the amino acid sequence of SEQ ID NO:38, and a light chain variable region (V_{L}FAP) comprising (iv) CDR-L1 comprising the amino acid sequence of SEQ ID NO:39, (v) CDR-L2 comprising the amino acid sequence of SEQ ID NO:40, and (vi) CDR-L3 comprising the amino acid sequence of SEQ ID NO:41, or
(b) a heavy chain variable region (V_{H}FAP) comprising (i) CDR-H1 comprising the amino acid sequence of SEQ ID NO:44, (ii) CDR-H2 comprising the amino acid sequence of SEQ ID NO:45, and (iii) CDR-H3 comprising the amino acid sequence of SEQ ID NO:46, and a light chain variable region (V_{L}FAP) comprising (iv) CDR-L1 comprising the amino acid sequence of SEQ ID NO:47, (v) CDR-L2 comprising the amino acid sequence of SEQ ID NO:48, and (vi) CDR-L3 comprising the amino acid sequence of SEQ ID NO:49.

10. The agonistic ICOS antigen binding molecule of any one of claims 1 to 3 or 8 or 9, wherein the antigen binding domain capable of specific binding to FAP comprises
(a) a heavy chain variable region (V_{H}FAP) comprising an amino acid sequence that is at least about 95%, 96%, 97%, 98%, 99% or 100% identical to the amino acid sequence of SEQ ID NO:42, and a light chain variable region (V_{L}FAP) comprising an amino acid sequence that is at least about 95%, 96%, 97%, 98%, 99% or 100% identical to the amino acid sequence of SEQ ID NO:43, or
(b) a heavy chain variable region (V_{H}FAP) comprising an amino acid sequence that is at least about 95%, 96%, 97%, 98%, 99% or 100% identical to the amino acid sequence of SEQ ID NO:50, and a light chain variable region (V_{L}FAP) comprising an amino acid sequence that is at least about 95%, 96%, 97%, 98%, 99% or 100% identical to the amino acid sequence of SEQ ID NO:51.

11. The agonistic ICOS antigen binding molecule of any one of claims 1 to 3 or 8 to 10, wherein the antigen binding domain capable of specific binding to FAP comprises a heavy chain variable region (V_{H}FAP) comprising the amino acid sequence of SEQ ID NO:42, and a light chain variable region (V_{L}FAP) comprising the amino acid sequence of SEQ ID NO:43.

12. The agonistic ICOS antigen binding molecule of any one of claims 1 to 11, wherein the antigen binding domain capable of specific binding to ICOS comprises
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

13. The agonistic ICOS antigen binding molecule of any one of claims 1 to 12, comprising
(a) one antigen binding domain capable of specific binding to a tumor-associated antigen,
(b) one Fab fragment capable of specific binding to ICOS, and
(c) a Fc domain composed of a first and a second subunit capable of stable association comprising one or more amino acid substitution that reduces the binding affinity of the antigen binding molecule to an Fc receptor and/or effector function.

14. The agonistic ICOS antigen binding molecule of any one of claims 1 to 12, comprising
(a) one antigen binding domain capable of specific binding to a tumor-associated antigen,
(b) two Fab fragments capable of specific binding to ICOS, and
(c) a Fc domain composed of a first and a second subunit capable of stable association comprising one or more amino acid substitution that reduces the binding affinity of the antigen binding molecule to an Fc receptor and/or effector function.

15. The agonistic ICOS antigen binding molecule of claim 13 or 14, wherein the antigen binding domain capable of specific binding to a tumor-associated antigen is a crossFab fragment.

16. An agonistic ICOS antigen binding molecule, wherein the antigen binding molecule comprises
(a) a heavy chain variable region (V_{H}ICOS) comprising (i) CDR-H1 comprising the amino acid sequence of SEQ ID NO:4, (ii) CDR-H2 comprising the amino acid sequence of SEQ ID NO:5, and (iii) CDR-H3 comprising the amino acid sequence of SEQ ID NO:6, and a light chain variable region (V_{L}ICOS) comprising (iv) CDR-L1 comprising the amino acid sequence of SEQ ID NO:7, (v) CDR-L2 comprising the amino acid sequence of SEQ ID NO:8, and (vi) CDR-L3 comprising the amino acid sequence of SEQ ID NO:9, or
(b) a heavy chain variable region (V_{H}ICOS) comprising (i) CDR-H1 comprising the amino acid sequence of SEQ ID NO:12, (ii) CDR-H2 comprising the amino acid sequence of SEQ ID NO:13, and (iii) CDR-H3 comprising the amino acid sequence of SEQ ID NO:14, and a light chain variable region (V_{L}ICOS) comprising (iv) CDR-L1 comprising the amino acid sequence of SEQ ID NO:15, (v) CDR-L2 comprising the amino acid sequence of SEQ ID NO:16, and (vi) CDR-L3 comprising the amino acid sequence of SEQ ID NO:17, or
(c) a heavy chain variable region (V_{H}ICOS) comprising (i) CDR-H1 comprising the amino acid sequence of SEQ ID NO:20, (ii) CDR-H2 comprising the amino acid sequence of SEQ ID NO:21, and (iii) CDR-H3 comprising the amino acid sequence of SEQ ID NO:22, and a light chain variable region (V_{L}ICOS) comprising (iv) CDR-L1 comprising the amino acid sequence of SEQ ID NO:23, (v) CDR-L2 comprising the amino acid sequence of SEQ ID NO:24, and (vi) CDR-L3 comprising the amino acid sequence of SEQ ID NO:25, or
(d) a heavy chain variable region (V_{H}ICOS) comprising (i) CDR-H1 comprising the amino acid sequence of SEQ ID NO:28, (ii) CDR-H2 comprising the amino acid sequence of SEQ ID NO:29, and (iii) CDR-H3 comprising the amino acid sequence of SEQ ID NO:30, and a light chain variable region (V_{L}ICOS) comprising (iv) CDR-L1 comprising the amino acid sequence of SEQ ID NO:31, (v) CDR-L2 comprising the amino acid sequence of SEQ ID NO:32, and (vi) CDR-L3 comprising the amino acid sequence of SEQ ID NO:33.

17. The agonistic ICOS antigen binding molecule, wherein the antigen binding molecule comprises
(a) a heavy chain variable region (V_{H}ICOS) comprising an amino acid sequence that is at least about 95%, 96%, 97%, 98%, 99% or 100% identical to the amino acid sequence of SEQ ID NO:10, and a light chain variable region (V_{L}ICOS) comprising an amino acid sequence that is at least about 95%, 96%, 97%, 98%, 99% or 100% identical to the amino acid sequence of SEQ ID NO:11, or
(b) a heavy chain variable region (V_{H}ICOS) comprising an amino acid sequence that is at least about 95%, 96%, 97%, 98%, 99% or 100% identical to the amino acid sequence of SEQ ID NO:18, and a light chain variable region (V_{L}ICOS) comprising an amino acid sequence that is at least about 95%, 96%, 97%, 98%, 99% or 100% identical to the amino acid sequence of SEQ ID NO:19, or
(c) a heavy chain variable region (V_{H}ICOS) comprising an amino acid sequence that is at least about 95%, 96%, 97%, 98%, 99% or 100% identical to the amino acid sequence of SEQ ID NO:26, and a light chain variable region (V_{L}ICOS) comprising an amino acid sequence that is at least about 95%, 96%, 97%, 98%, 99% or 100% identical to the amino acid sequence of SEQ ID NO:27, or
(d) a heavy chain variable region (V_{H}ICOS) comprising an amino acid sequence that is at least about 95%, 96%, 97%, 98%, 99% or 100% identical to the amino acid sequence of SEQ ID NO:34, and a light chain variable region (V_{L}ICOS) comprising an amino acid sequence that is at least about 95%, 96%, 97%, 98%, 99% or 100% identical to the amino acid sequence of SEQ ID NO:35.

18. An isolated nucleic acid encoding the agonistic ICOS antigen binding molecule of any one of claims 1 to 17.

19. A host cell comprising the nucleic acid of claim 18.

20. A method of producing an agonistic ICOS antigen binding molecule comprising culturing the host cell of claim 19 under conditions suitable for the expression of the agonistic ICOS antigen binding molecule.

21. The method of claim 20, further comprising recovering the antigen binding molecule from the host cell.

22. An agonistic ICOS antigen binding molecule produced by the method of claim 21.

23. A pharmaceutical composition comprising the agonistic ICOS antigen binding molecule of any one of claims 1 to 17 and at least one pharmaceutically acceptable excipient.

24. The pharmaceutical composition of claim 23 for use in the treatment of cancer.

25. The agonistic ICOS antigen binding molecule of any one of claims 1 to 17, or the pharmaceutical composition of claim 23, for use as a medicament.

26. The agonistic ICOS antigen binding molecule of any one of claims 1 to 17, or the pharmaceutical composition of claim 23, for use in the treatment of cancer.

27. The agonistic ICOS antigen binding molecule of any one of claims 1 to 17 for use in the treatment of cancer, wherein the agonistic ICOS antigen binding molecule is for administration in combination with a chemotherapeutic agent, radiation therapy and/or other agents for use in cancer immunotherapy.

28. The agonistic ICOS antigen binding molecule of any one of claims 1 to 17 for use in the treatment of cancer, wherein the agonistic ICOS antigen binding molecule is for administration in combination with a T-cell activating anti-CD3 bispecific antibody.

29. The agonistic ICOS antigen binding molecule of any one of claims 1 to 17 for use of claim 28, wherein the T-cell activating anti-CD3 bispecific antibody is an anti-CEA/anti-CD3 bispecific antibody.

30. The agonistic ICOS antigen binding molecule of any one of claims 1 to 17 for use in the treatment of cancer, wherein the agonistic ICOS antigen binding molecule is for use in combination with an agent blocking PD-L1/PD-1 interaction.

31. The agonistic ICOS antigen binding molecule of any one of claims 1 to 17 for use of claim 30, wherein the agent blocking PD-L1/PD-1 interaction is atezolizumab.

32. Use of the agonistic ICOS antigen binding molecule of any one of claims 1 to 17, or the pharmaceutical composition of claim 23, in the manufacture of a medicament for the treatment of cancer.

33. A method of inhibiting the growth of tumor cells in an individual comprising administering to the individual an effective amount of the agonistic ICOS antigen binding molecule of any one of claims 1 to 17, or the pharmaceutical composition of claim 23, to inhibit the growth of the tumor cells.

34. A method of treating cancer comprising administering to the individual a therapeutically effective amount of the agonistic ICOS antigen binding molecule of any one of claims 1 to 17, or the pharmaceutical composition of claim 23.
